(19) United States
(12) Patent Application Publication
(10) Pub. No.: US 2023/0374017 A1
(43) Pub. Date: Nov. 23, 2023

(54) COMPOSITIONS AND METHODS FOR ACTIVATING PYRUVATE KINASE

(71) Applicant: The Regents of the University of Michigan, Ann Arbor, MI (US)

(72) Inventors: CAGRI BESIRLI, Ann Arbor, MI (US); JASON RECH, Ann Arbor, MI (US); THOMAS WUBBEN, Ann Arbor, MI (US); BRENNAN WATCH, Ann Arbor, MI (US)

(21) Appl. No.: 18/017,108

(22) PCT Filed: Jul. 21, 2021
(86) PCT No.: PCT/US2021/042489

§ 371 Date: Jan. 20, 2023

Related U.S. Application Data
(60) Provisional application No. 63/054,478, filed on Jul. 21, 2020.

(51) Int. Cl. C07D 487/04; C07D 519/00; C07D 495/14; C07D 471/14; C07D 513/14; C07D 498/14

(52) U.S. Cl. CPC C07D 487/04; C07D 519/00; C07D 495/14; C07D 471/14; C07D 513/14; C07D 498/14

(57) ABSTRACT

Provided herein are compositions and methods for activating pyruvate kinase (e.g., in a subject). In particular, provided herein are compositions and methods for treating a disease or condition (e.g., eye disease, blood disorders, or cancer) using pyruvate kinase activators.

### FIELD

Provided herein are compositions and methods for activating pyruvate kinase (e.g., in a subject). In particular, provided herein are compositions and methods for treating a disease or condition (e.g., eye disease, blood disorders, or cancer) using pyruvate kinase activators.

### BACKGROUND

Photoreceptor death is the ultimate cause of vision loss in many retinal disorders, including retinal detachment, retinal dystrophies and age-related macular degeneration (AMD). AMD affects 17 million people in the U.S. with a potential annual market size of $40 billion (Wong, W. L. et al. Lancet Glob. Health 2, e106-116 (2014)). Retinal dystrophies including Retinitis Pigmentosa (RP) affect 100,000 people in the U.S with a potential annual market size of $480 million. Retinal detachment affects 750,000 people worldwide with a market size of $150 million (Haimann, M. H., et al. Arch. Ophthalmol. Chic. Ill 1960 100, 289-292 (1982)). The poor visual function and blindness patients experience results in life-long vision services, loss of productivity for patients and caregivers, and a reduced quality of life. The value of medical complementary costs attributable to low vision is $5.5 billion per year and the value of lost quality of life is $10.5 billion per year in the U.S (Frick, K. D., et al. Arch. Ophthalmol. Chic. Ill 1960 125, 544-550 (2007)). No successful treatment options exist to prevent photoreceptor death in retinal diseases.

There is an urgent unmet need for neuroprotective modalities to improve photoreceptor survival and related disorders.

### SUMMARY

Provided herein are compositions and methods for activating pyruvate kinase (e.g., in a subject). In particular, provided herein are compositions and methods for treating a disease or condition (e.g., eye disease, blood disorders, or cancer) using pyruvate kinase activators.

Metabolic reprograming of photoreceptors is a therapeutic solution for vision loss associated with age-related macular degeneration, retinal dystrophies, retinal degenerations, diabetic retinopathy, and retinal detachment (Aït-Ali, N. et al. Cell 161, 817-832 (2015); Zhang, L. et al. J. Clin. Invest. 126, 4659-4673 (2016)). Activation of PKM2, the key regulator of aerobic glycolysis and energy metabolism in photoreceptors, by small molecule activators including ML-265 reprograms metabolism and enhances energy production by favoring catabolic activity in the cells (Anastasiou, D. et al. Nat. Chem. Biol. 8, 839-847 (2012; Wubben et al. Sci Rep. 2017 and Wubben et al. Sci Rep. 2020)). Limitation of PKM2 expression and aerobic glycolysis to photoreceptors in the eye reduces any potential off-target effects after ocular delivery, increasing the specificity of the treatment and enhancing the therapeutic window (Rajala, R. V. S., et al. Sci. Rep. 6, 37727 (2016); Lindsay, K. J. et al. Proc. Natl. Acad. Sci. U.S.A 111, 15579-15584 (2014)).

Accordingly, in some embodiments, provided herein are pyruvate kinase activators for use in, for example, treating diseases or conditions (e.g., eye disorders). For example, in some embodiments, provided is a composition, comprising: a compound selected from

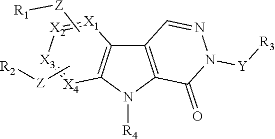

Formula I or

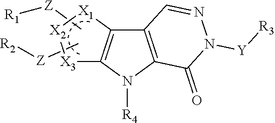

Formula II:
Wherein X₁, X₂, X₃, and X₄ are independently selected from CH, CO, N, NH, S, or O;
Z is null, a bond, optionally substituted C1-6 alkyl, —O—, —S—, —CH₂—, —CHR₅—, —CR₅R₆—, —(CH₂)ₙ—, —(CHR₅)ₙ—, —(CR₅R₆)ₙ—, —S(═O)CH₂, —S(═O)₂CH₂—, —NR₅—, —NR₅C(═O)—, —C(O)NR₅—, —C(═O)—, —OC(═O)—, —C(═O)O—, —NR₅C(═O)O—, —OC(═O)NR₅—, —NR₅C(═O)NR₅—, —OC(R₅)₂—, —C(R₅)₂O—, —NR₄C(R₅)₂—, C(R₆)₂NR₅—, —S(═O)—, S(═O)₂—, —S(═O)₂O—, —OS(═O)₂—, —S(═O)₂NR₅—, —NR₅S(═O)₂—, —S(═O)NR₅—, —NR₅S(═O)—, —OS(═O)NR₅—, —NR₅S(═O)O—, or —S(═O)(═NR₅)—, where in the point of attachment to R₁ or R₂ is on the left and n=1-6;
R₅ and R₆ are each independently hydrogen, a halogen, —CN, OR₇, NR₇R₈, —N(R₇)C(═O)R₈, —C(═O)N(R₇), —C(═O)R₇, —C(═O)OR₇, —SR₇, —S(═O)R₇, —S(═O)₂R₇, or any optimally substituted —C₁-C₆ alkyl;
R₇ and R₈ are each independently hydrogen, any optimally substituted —C₁-C₆ alkyl; or alternatively R₇ and R₈ are taken together as an optionally substituted C₁-C₆ monocyclic cycloalkyl ring or an optionally substituted monocyclic heterocyclic ring;
Y is null, —CH₂—, —CHR₉—, —CR₉R₁₀—, —(CH₂)ₙ—, —(CHR₉)ₙ—, —(CR₉R₁₀)ₙ—, —C(═O)—, —S(═O)—, —S(═O)₂—, wherein n=1-6;
R₉ and R₁₀ are each independently hydrogen, a halogen, —CN, or any optimally substituted —C₁-C₆ alkyl; or alternatively R₉ and R₁₀ can be taken together as an optionally substituted C₁-C₆ monocyclic cycloalkyl ring or an optionally substituted monocyclic heterocyclic ring;
R₁ is —H, —F, —Cl, —Br, —NO₂, —CN, —NH₂, —NHR₁₁, —NR₁₁R₁₂, —OH, —OR₁₁, optionally substituted C₁-C₆ alkyl, optionally substituted monocyclic cycloalkyl, optionally substituted bicyclic cycloalkyl, optionally substituted tricyclic cycloalkyl, optionally substituted monocyclic heterocycle, optionally substituted bicyclic heterocycle, or optionally substituted tricyclic heterocycle;
R₂ is —H, —F, —Cl, —Br, —NO₂, —CN, —NO₂, —NH₂, —NHR₁₁, —NR₁₁R₁₂, —OH, —OR₁₁, R₂ is —H, —F, —Cl, —Br, —NO₂, —CN, —NO₂, —NH₂, —NHR₁₁, —NR₁₁R₁₂, —OH, —OR₁₁, optionally substituted C₁-C₆ alkyl, optionally substituted monocyclic cycloalkyl, optionally substituted bicyclic cycloalkyl, optionally substituted tricyclic cycloalkyl, optionally substituted monocyclic heterocycle, optionally substituted bicyclic heterocycle, optionally substituted tricyclic heterocycle;
R₃ is —H, —F, —Cl, —Br, —NO₂, —CN, —NO₂, —NH₂, —NHR₁₁, —NR₁₁R₁₂, —OH, —OR₁₁, optionally substituted C₁-C₆ alkyl, optionally substituted monocyclic cycloalkyl, optionally substituted bicyclic cycloalkyl, optionally substituted tricyclic cycloalkyl, optionally substituted monocyclic heterocycle, optionally substituted bicyclic heterocycle, optionally substituted tricyclic heterocycle;
R₁₁ and R₁₂ are each independently optionally substituted C₁-C₆ alkyl, optionally substituted monocyclic cycloalkyl, optionally substituted bicyclic cycloalkyl, optionally substituted tricyclic cycloalkyl, optionally substituted monocyclic heterocycle, optionally substituted bicyclic heterocycle, or optionally substituted tricyclic heterocycle;
R₄ is hydrogen, —CH₃, —CHR₁₃, —CR₁₃R₁₄, —S(═O)R₁₃, —S(═O)₂R₁₃, optionally substituted alkyl, an optimally substituted haloalkyl, an optimally substituted alkenyl, an optimally substituted alkynyl, an optimally substituted cycloalkyl, an optimally substituted heterocycle, an optimally substituted aryl, —C(═O)R₁₅, or a nitrogen protecting group; wherein;
R₁₃ and R₁₄ are each independently optionally substituted C₁-C₆ alkyl, optionally substituted monocyclic cycloalkyl, optionally substituted bicyclic cycloalkyl, optionally substituted tricyclic cycloalkyl, optionally substituted monocyclic heterocycle, optionally substituted bicyclic heterocycle, optionally substituted tricyclic heterocycle;
R₁₅ is hydrogen, —CH₃, optionally substituted alkyl, an optimally substituted haloalkyl, an optimally substituted alkenyl, an optimally substituted alkynyl, an optimally substituted cycloalkyl, an optimally substituted heterocycle, an optimally substituted aryl or a pharmaceutically acceptable salt thereof.

In some embodiments R₂—Z— is H and R₁—Z— is selected from;
Z is null, a bond, optionally substituted C₁₋₆ alkyl, —O—, —S—, —CH₂—, —CHR₅—, —CR₅R₆—, —(CH₂)ₙ—, —(CHR₅)ₙ—, —(CR₅R₆)ₙ—, —S(═O)CH₂, —NR₅—, —NR₅C(═O)—, —C(O)NR₅—, —C(═O)—, —S(═O)—, where in the point of attachment to R₁ is on the left and n=1-6;
R₅ and R₆ are each independently hydrogen, —CN, OR₇, NR₇R₈, or any optimally substituted —C₁-C₆ alkyl; or alternatively R₇ and R₈ can be taken together as an optionally substituted C₁-C₆ monocyclic cycloalkyl ring or an optionally substituted monocyclic heterocyclic ring; wherein;
R₇ and R₈ are each independently hydrogen, any optimally substituted —C₁-C₆ alkyl; or alternatively R₇ and R₈ can be taken together as an optionally substituted C₁-C₆ monocyclic cycloalkyl ring or an optionally substituted monocyclic heterocyclic ring;
Y is null, —CH₂—, —CHR₉—, —CR₉R₁₀—, —(CH₂)ₙ—, —(CHR₉)ₙ—, —(CR₉R₁₀)ₙ—, wherein n=1-3;
R₉ and R₁₀ are each independently hydrogen, or any optimally substituted —C₁-C₆ alkyl; or alternatively R₉ and R₁₀ can be taken together as an optionally substituted C₁-C₆ monocyclic cycloalkyl ring or an optionally substituted monocyclic heterocyclic ring;
R₁ is —H, —F, —Cl, —Br, —NO₂, —CN, —NO₂, —NH₂, —NHR₁₁, —NR₁₁R₁₂, —OH, —OR₁₁, optionally substituted C₁-C₆ alkyl, optionally substituted monocyclic cycloalkyl, optionally substituted bicyclic cycloalkyl, optionally substituted tricyclic cycloalkyl, optionally substituted monocyclic heterocycle, optionally substituted bicyclic heterocycle, or optionally substituted tricyclic heterocycle;
R₂ is —H, —F, —Cl, —Br, —NO₂, —CN, —NO₂, —NH₂, —NHR₁₁, —NR₁₁R₁₂, —OH, —OR₁₁, optionally substituted C₁-C₆ alkyl, optionally substituted monocyclic cycloalkyl, optionally substituted bicyclic cycloalkyl, optionally substituted tricyclic cycloalkyl, optionally substituted monocyclic heterocycle, optionally substituted bicyclic heterocycle, or optionally substituted tricyclic heterocycle;
R₃ is —H, —F, —Cl, —Br, —NO₂, —CN, —NO₂, —NH₂, —NHR₁₁, —NR₁₁R₁₂, —OH, —OR₁₁, optionally substituted C₁-C₆ alkyl, optionally substituted monocyclic cycloalkyl, optionally substituted bicyclic cycloalkyl, optionally substituted tricyclic cycloalkyl, optionally substituted monocyclic heterocycle, optionally substituted bicyclic heterocycle, optionally substituted tricyclic heterocycle;
R₁₁ and R₁₂ are each independently optionally substituted C₁-C₆ alkyl, optionally substituted monocyclic cycloalkyl, optionally substituted bicyclic cycloalkyl, optionally substituted tricyclic cycloalkyl, optionally substituted monocyclic heterocycle, optionally substituted bicyclic heterocycle, or optionally substituted tricyclic heterocycle;
R₄ is —CH₃.

In some exemplary embodiments Z—R₁, Z—R₂, Z—R₃, and Y—R₃ individually or in combination are selected from, for example,

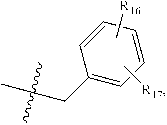

wherein R₁₆ and R₁₇ can be located in the ortho, meta or para positions on the aryl ring and are selected from H, —OCH₃, C₁-C₄ alkyl, —NH₂, -halogen, —CN, —OH, —S(═O)Me, —S(═O)₂Me, —CH₂OMe, —CH₂NR₁₈R₁₉ wherein R₁₈ and R₁₉ are selected from —H, C₁-C₄ alkyl, an optimally substituted aryl or heterocycle or taken together to form a carbocycle or heterocycle

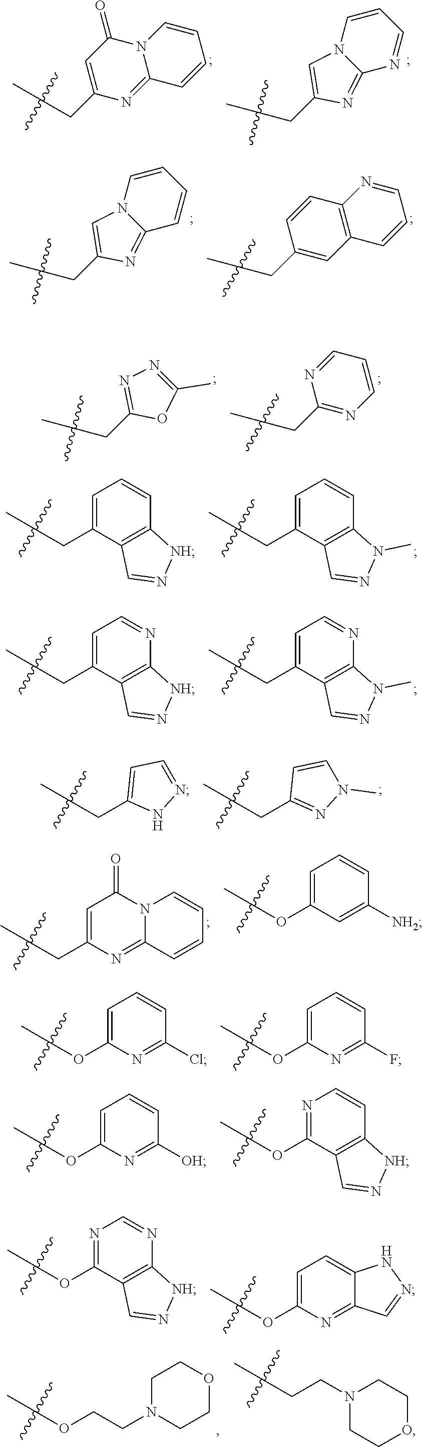

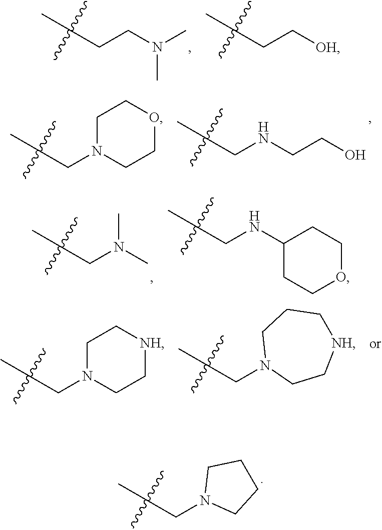

In some embodiments, the compound is a compound of Formula I where Z—R₂ is H, Z—R₁ is not 8-OCH₃, R₄ is not

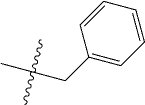 [SMILES: *Cc1ccccc1]

### and Y—R₃ is not

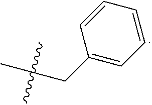 [SMILES: *Cc1ccccc1]

In some embodiments, the compound is a compound of Formula I where Z—R₂ is H, Z—R₁ is not 8-Cl, R₄ is not CH₃ or

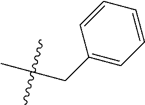 [SMILES: *Cc1ccccc1]

and Y—R₃ is not CH₃ or

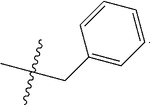 [SMILES: *Cc1ccccc1]

In some embodiments, the compound is a compound of Formula I and R₂ is not

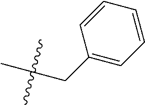 [SMILES: *Cc1ccccc1]

or CH₃, and Y—R₃ is not

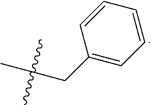 [SMILES: *Cc1ccccc1]

In some embodiments, the compound is a compound of Formula I and Z—R₁ is not

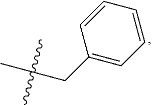 [SMILES: *Cc1ccccc1]

X₁ is not CO, R₂ is not CH₃, and Y—R₃ is not

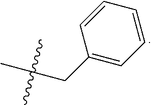 [SMILES: *Cc1ccccc1]

In some embodiments, the compound is not

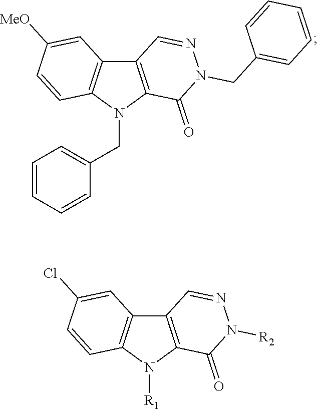

wherein R₁ and R₂ are independently CH₃ or

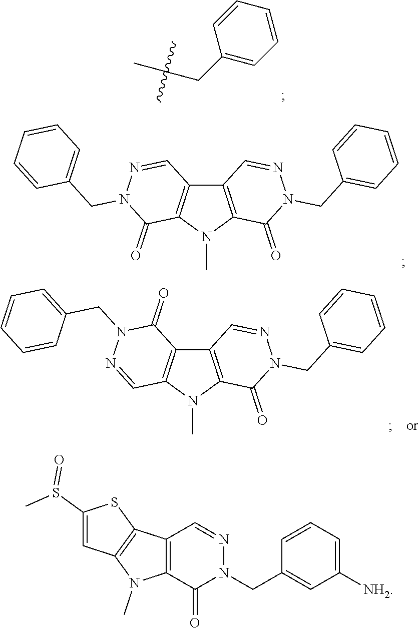

In some embodiments, the compound is selected from the compounds described in Table 2. In some embodiments, the compound is:

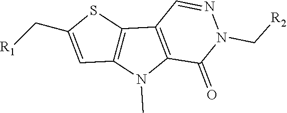

wherein R₁ is a substituted mono or bi-cyclic carbocyclic or heterocyclic moiety and R₂ is a substituted mono or bi-cyclic heterocyclic moiety.

In some exemplary embodiments, the compound is

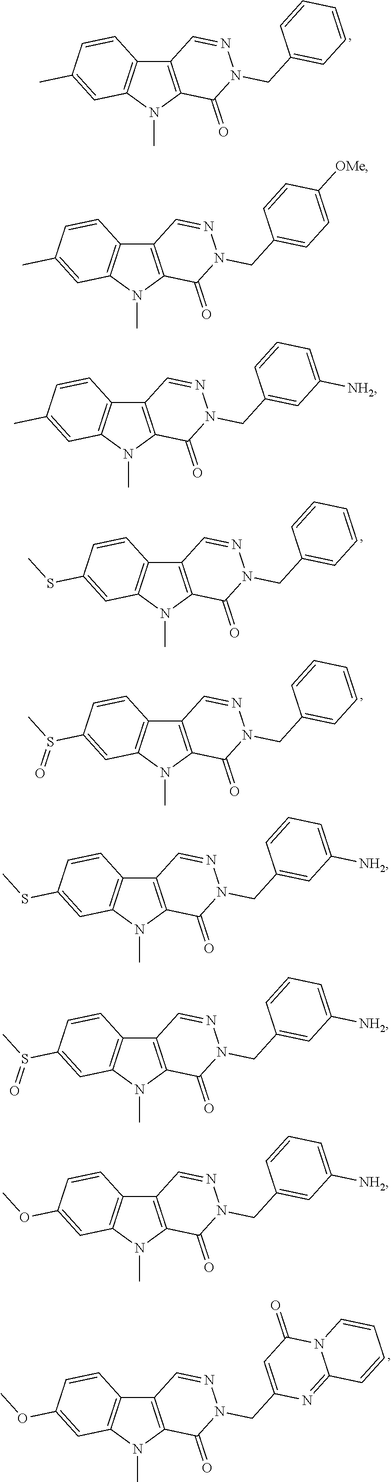

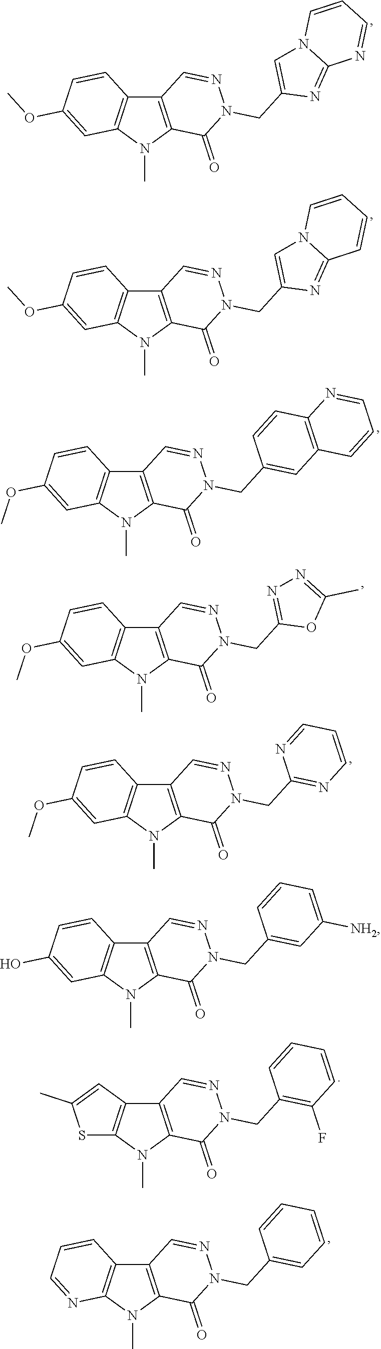

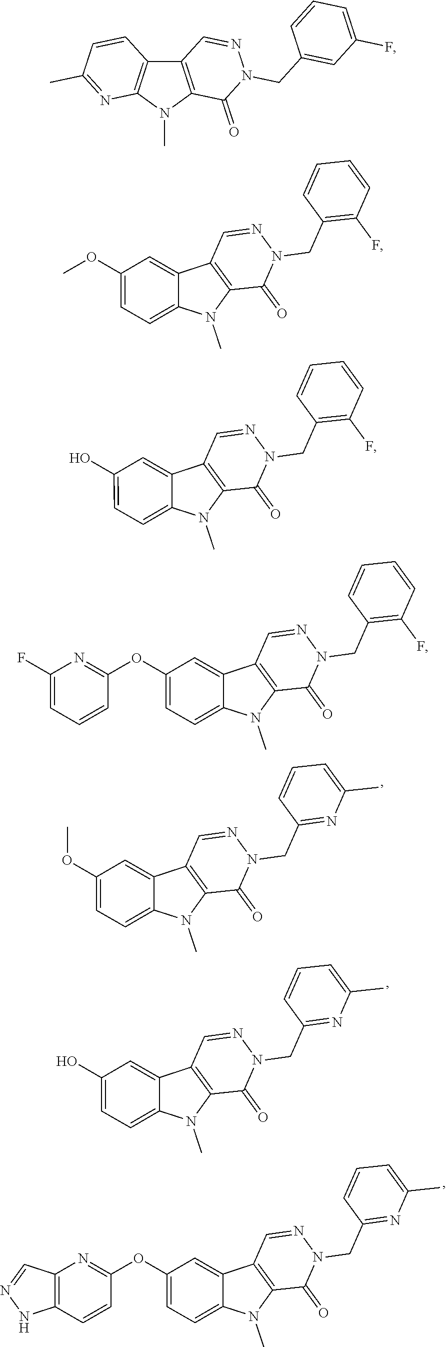

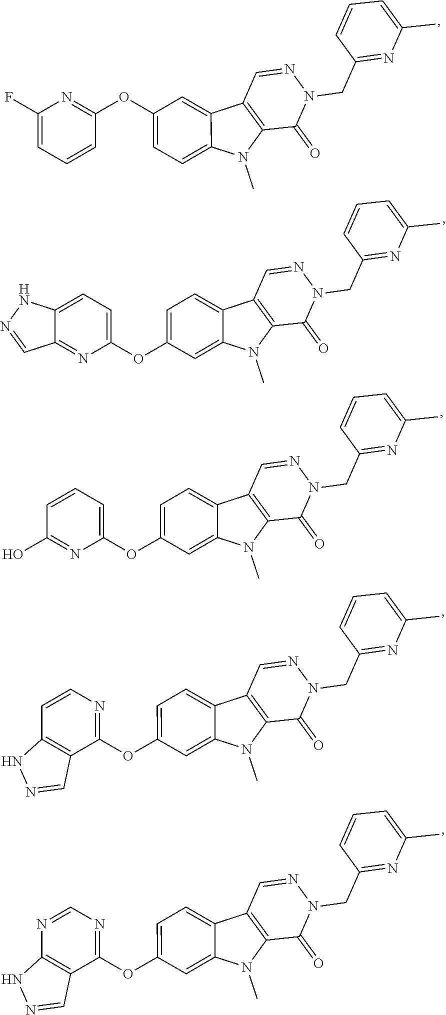 [SMILES: Cc1cccc(Cn2ncc3c4cc(Oc5cccc(F)n5)ccc4n(C)c3c2=O)n1 ; Cc1cccc(Cn2ncc3c4ccc(Oc5ccc6[nH]ncc6n5)cc4n(C)c3c2=O)n1 ; Cc1cccc(Cn2ncc3c4ccc(Oc5cccc(O)n5)cc4n(C)c3c2=O)n1 ; Cc1cccc(Cn2ncc3c4ccc(Oc5nccc6[nH]ncc56)cc4n(C)c3c2=O)n1 ; Cc1cccc(Cn2ncc3c4ccc(Oc5ncnc6[nH]ncc56)cc4n(C)c3c2=O)n1]

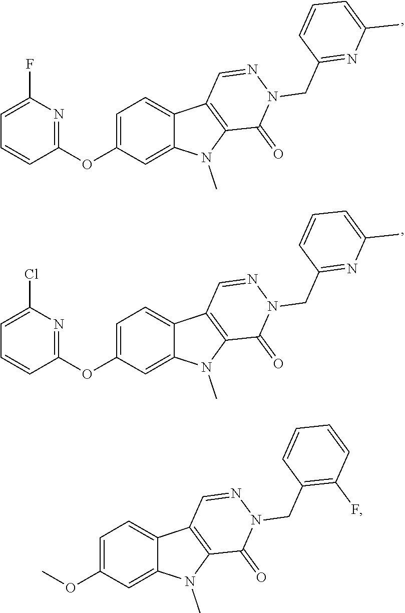

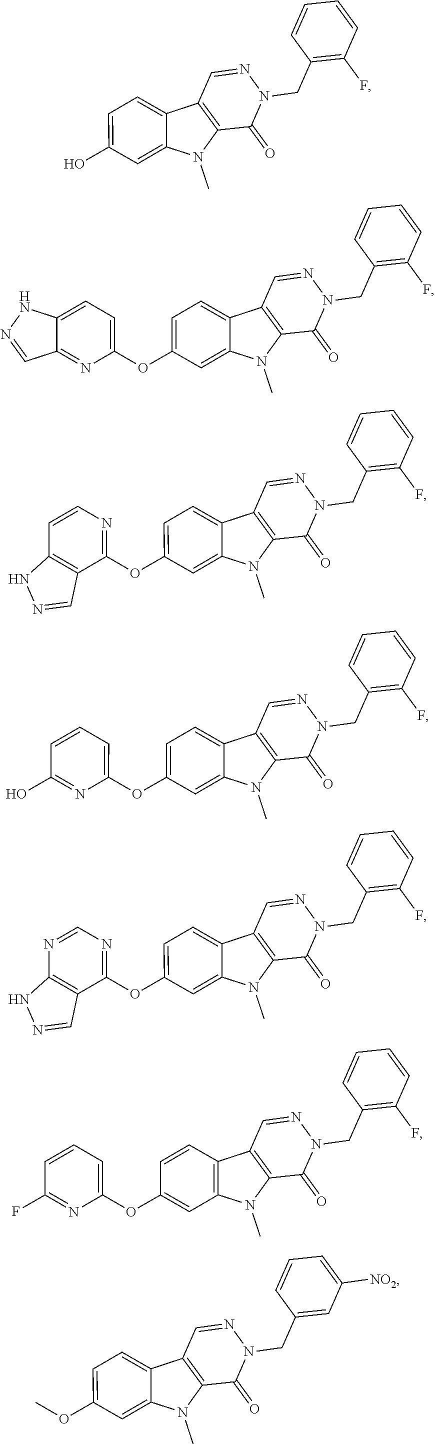

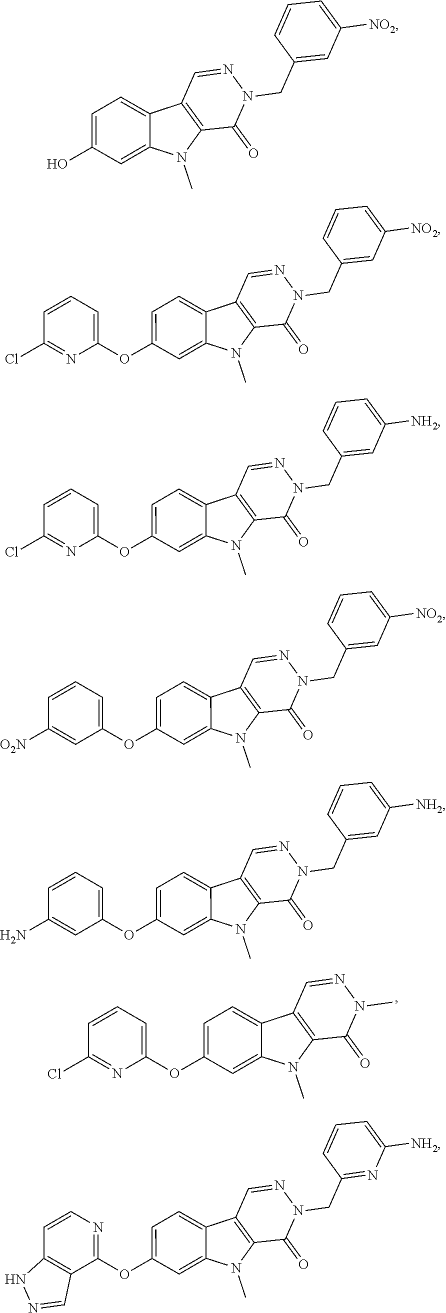

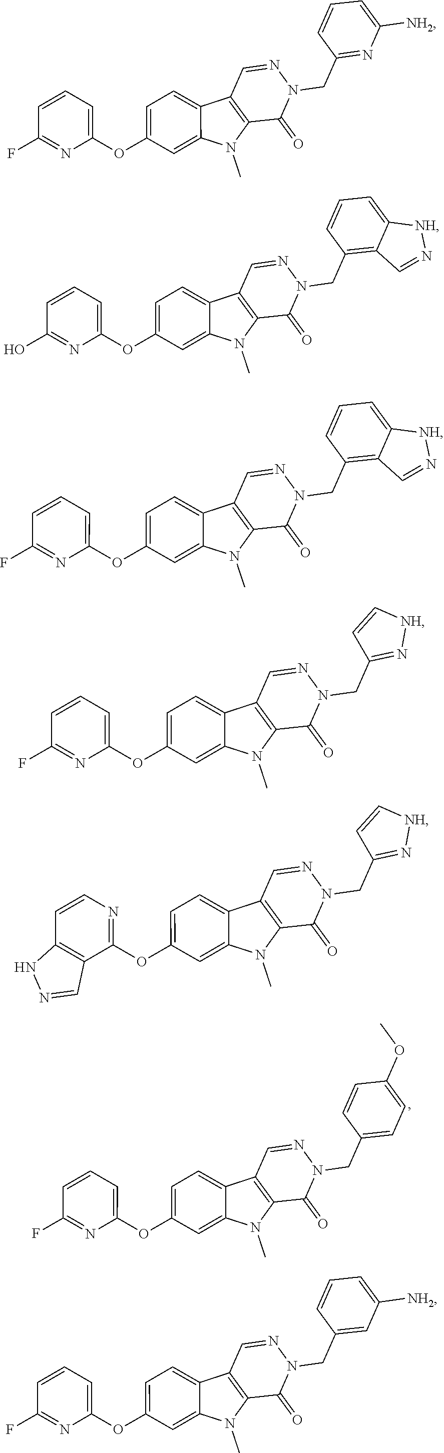

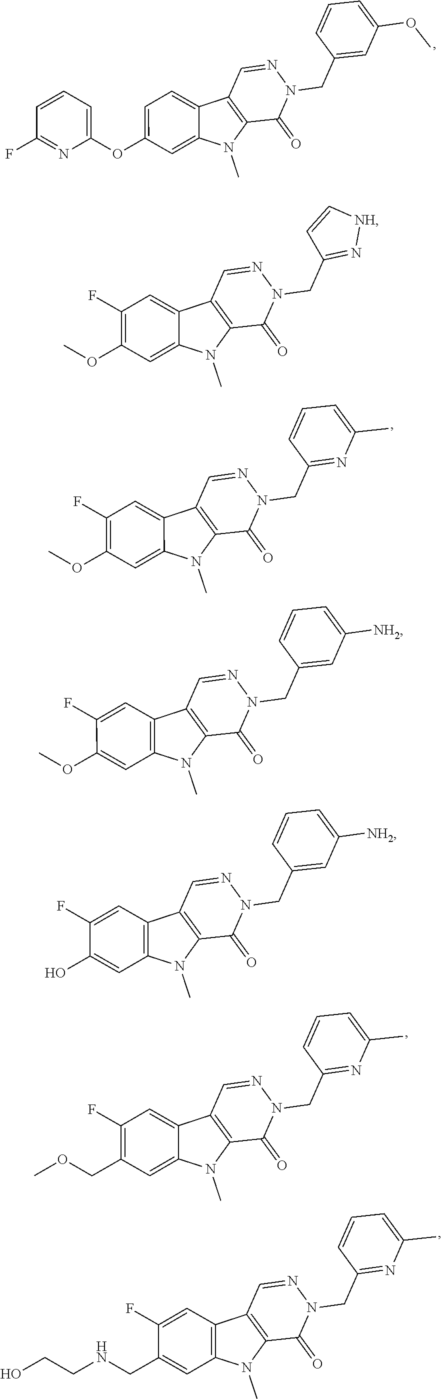

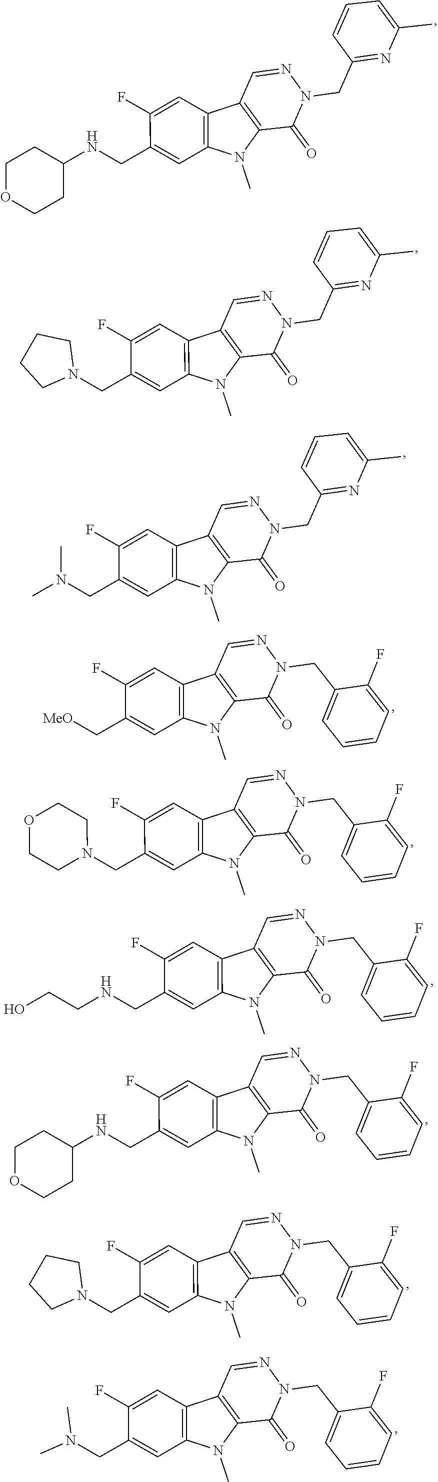

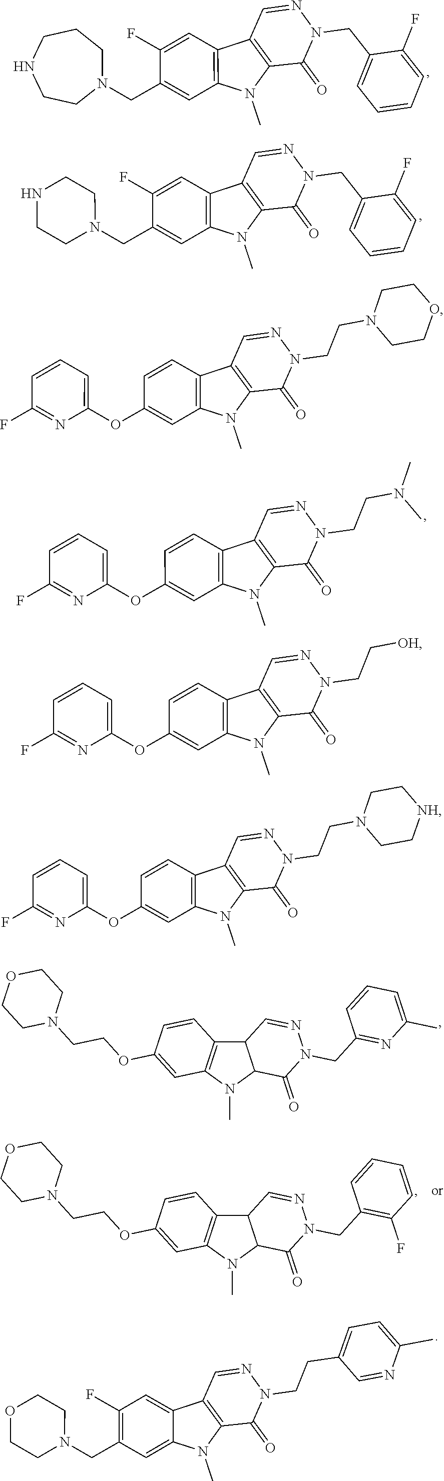

In some embodiments, the compound is

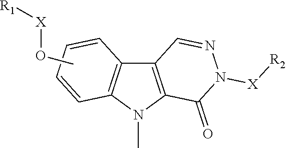

wherein
X is null or is selected from —H, —CH₂—, —CHR₃—, —CR₃R₄—, —(CH₂)ₙ—, —(CHR₃)ₙ—, —(CR₃R₄)ₙ—, wherein n-1-6;
R₁ is selected from, for example, —H, —CN, —NO₂, —NH₂, —NHR₃, NR₃R₄, —OH, OR₃, —SOR₃, —SO₂R₃, optionally substituted C₁-C₆ alkyl, optionally substituted monocyclic cycloalkyl, optionally substituted bicyclic cycloalkyl, optionally substituted tricyclic cycloalkyl, optionally substituted monocyclic heterocycle, optionally substituted bicyclic heterocycle, or optionally substituted tricyclic heterocycle;
R₂ is selected from, for example, —H, —CN, —NO₂, —NH₂, —NHR₃, NR₃R₄, —OH, OR₃, —SOR₃, —SO₂R₃, optionally substituted C₁-C₆ alkyl, optionally substituted monocyclic cycloalkyl, optionally substituted bicyclic cycloalkyl, optionally substituted monocyclic heterocycle, optionally substituted bicyclic heterocycle, or optionally substituted tricyclic heterocycle;
R₃ and R₄ are each independently selected from the group, for example, optionally substituted C₁-C₆ alkyl, optionally substituted monocyclic cycloalkyl, optionally substituted bicyclic cycloalkyl, optionally substituted tricyclic cycloalkyl, optionally substituted monocyclic heterocycle, optionally substituted bicyclic heterocycle, or optionally substituted tricyclic heterocycle; and.

In some embodiments, R₁ is selected from, for example,

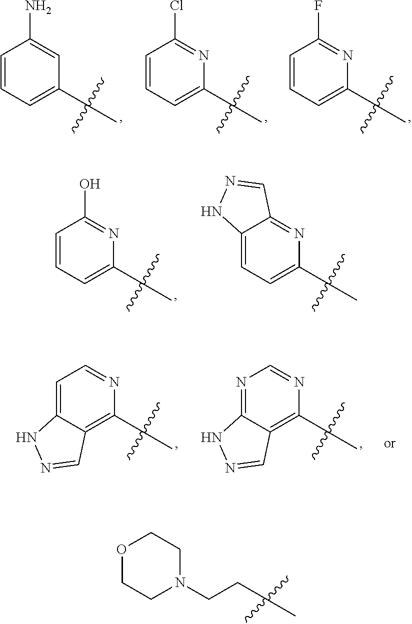

and R₂ is selected from, for example,

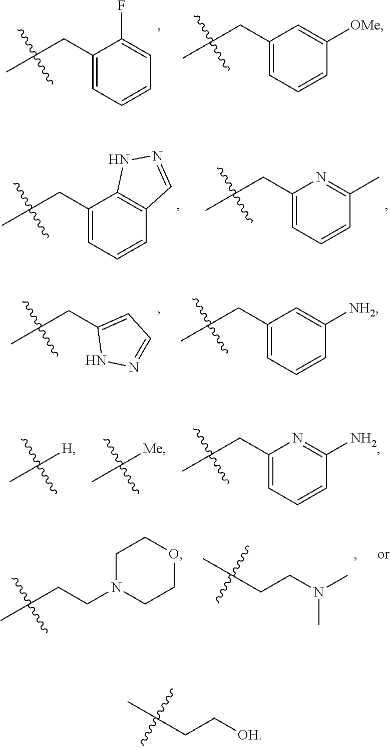

In further embodiments, the compound is

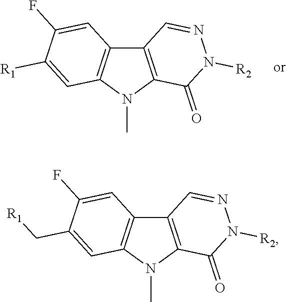

wherein R₁ is selected from, for example —H, —F, —Cl, —Br, —CN, —NO₂, —NH₂, —NHR₃, —NR₃R₄, —OH, —OR₃, optionally substituted C₁-C₆ alkyl, optionally substituted monocyclic cycloalkyl, optionally substituted bicyclic cycloalkyl, optionally substituted tricyclic cycloalkyl, optionally substituted monocyclic heterocycle, optionally substituted bicyclic heterocycle, or optionally substituted tricyclic heterocycle;
R₂ is selected from, for example, —H, —CH₃, —(CH₂)ₙ—R₅—, —(CHR₃)ₙ—R₅—, —(CR₃R₄)ₙ—R₅— optionally substituted C₁-C₆ alkyl, optionally substituted monocyclic cycloalkyl, optionally substituted bicyclic cycloalkyl, optionally substituted monocyclic heterocycle, optionally substituted bicyclic heterocycle, or optionally substituted tricyclic heterocycle;
R₃ and R₄ are each independently selected from, for example, optionally substituted C₁-C₆ alkyl, optionally substituted monocyclic cycloalkyl, optionally substituted bicyclic cycloalkyl, optionally substituted tricyclic cycloalkyl, optionally substituted monocyclic heterocycle, optionally substituted bicyclic heterocycle, or optionally substituted tricyclic heterocycle; and
R₅ is —H, —F, —Cl, —Br, —NO₂, —CN, —NO₂, —NH₂, —NHR₃, —NR₃R₄, —OH, —OR₃, optionally substituted C₁-C₆ alkyl, optionally substituted monocyclic cycloalkyl, optionally substituted bicyclic cycloalkyl, optionally substituted tricyclic cycloalkyl, optionally substituted monocyclic heterocycle, optionally substituted bicyclic heterocycle, optionally substituted tricyclic heterocycle.

In some embodiments, R₁ is selected from, for example, OCH₃, OH, NH₂, NCH₃, N(CH₃)₂,

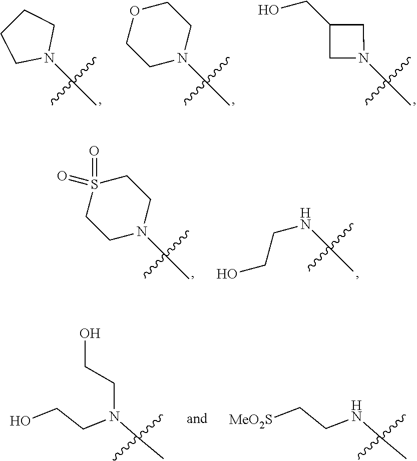

and R₂ is selected from, for example,

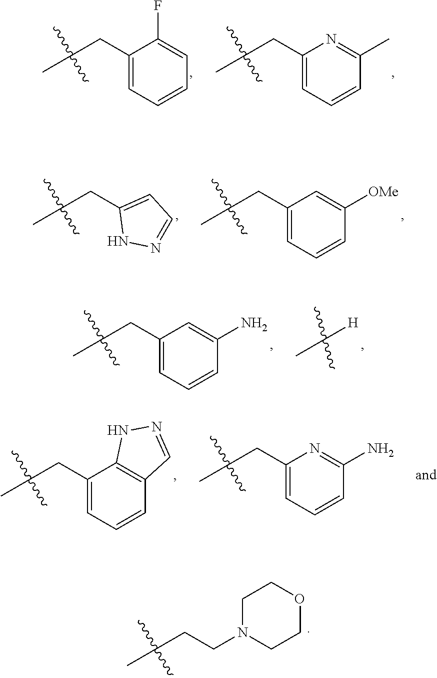

In some embodiments, one or more hydrogens of any of the aforementioned compounds are replaced with deuterium.

In some embodiments, the composition is a pharmaceutical composition. In some embodiments, the composition is formulated for injection, for oral delivery, or as an eye drop. In some embodiments, the composition comprises a pharmaceutically acceptable carrier. In some embodiments, the composition is a pyruvate kinase (e.g., PKM1 or PKM2) activator.

In further embodiments, provided herein is a method of activating a pyruvate kinase in a subject (e.g., in the eye of a subject), comprising: administering a compound described herein to the subject (e.g., to the eye of the subject), wherein the administering activates the pyruvate kinase. In some embodiments, the activating treats or reduces symptoms of a disease or condition in said subject (e.g., an eye disorder, cancer, or a blood disorder). Exemplary eye disorders include but are not limited to, vision loss, retinal dystrophy, macular degeneration, retinal degeneration, diabetic retinopathy, retinal detachment, or proliferative vitreoretinopathy. Exemplary blood disorders include but are not limited to, anemia, hemolytic anemia, sickle cell disease, thalassemia, hereditary spherocytosis, hereditary elliptocytosis, abetalipoproteinemia or Bassen-Kornzweig syndrome.

Additional embodiments provide a method of treating a disease or condition, comprising: administering a pyruvate kinase activator described herein to a subject in need thereof, wherein the administering treats or reduces symptoms of the disease or condition in the subject.

Further embodiments provide a method of treating an eye disorder, comprising: administering a compound described herein to the eye of a subject in need thereof, wherein the administering treats or reduces symptoms of an eye disorder in the subject. In some embodiments, the administering prevents or reduces photoreceptor cell death in the eye of the subject. In some embodiments, the administering prevents or reduces proliferative vitreoretinopathy in the eye of the subject. In some embodiments, the activator is formulated for injection (e.g., intravitreal injection), for oral delivery, or as an eye drop.

Further embodiments provide a method of treating an eye disorder, comprising: administering a compound selected from

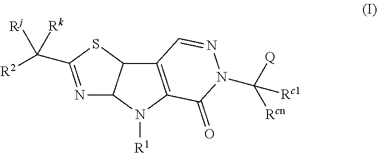

wherein R¹ is hydrogen, an optionally substituted alkyl, an optionally substituted haloalkyl, an optionally substituted alkenyl, an optionally substituted alkynyl, an optionally substituted cycloalkyl, an optionally substituted heterocyclyl, an optionally substituted aryl, —OR^{ol}, —C(═O)R^{cl}, or a nitrogen protecting group; wherein: R⁰¹ is hydrogen, optionally substituted alkyl, or an oxygen protecting group; R^{cl} is optionally substituted alkyl or —N(R^{cn})2, wherein each instance of R^{cn} is independently hydrogen, —C₁₋₆ alkyl, or a nitrogen protecting group; R² and Q are each independently an optionally substituted 5- or 6-membered monocyclic heteroaryl; R^{a} and R^{b} are each independently hydrogen, a halogen, —CN, —NO₂, —N₃, an optionally substituted alkyl, —OR^{o3}—N(Rⁿ¹)₂, —C(═O)N(Rⁿ¹)₂, or —C(═O)R^{c2}; or alternatively R^{a} and R^{b} can be taken together with the carbon atom to which they are attached to form an optionally substituted cycloalkyl or an optionally substituted heterocyclyl; wherein: each instance of Rⁿ¹ is independently hydrogen, an optionally substituted —C₁-C₆ alkyl, or a nitrogen protecting group; R^{o3} is hydrogen, an optionally substituted —C₁-C₆ alkyl, or an oxygen protecting group; and R^{c2} is an optionally substituted —C₁-C₆ alkyl; and RJ and R^{k} are each independently hydrogen, a halogen, —CN, —OR^{o7}, —N(Rⁿ⁵)₂, —N(Rⁿ⁵)C(═O) R^{c5}, —C(═O)N(Rⁿ⁵)₂, —C(═O)R^{c5}, —C(═O)OR^{o7}, —SR^{js}, —S(═O)₂R^{js}, —S(═O)R^{js} or an optionally substituted —C₁-C₆ alkyl; or alternatively R^{j} and R^{k} can be taken together with the carbon atom to which they are attached to form C═O, an optionally substituted C₁-C₆ monocyclic cycloalkyl ring, or an optionally substituted C₃-C₆ monocyclic heterocyclyl ring; wherein: each instance of Rⁿ⁵ is independently hydrogen, an optionally substituted —C₁-C₆ alkyl, —OR^{o8}, or a nitrogen protecting group, wherein R^{o8} is hydrogen, an optionally substituted —C₁-C₆ alkyl, or an oxygen protecting group; each instance of R^{o7} is independently hydrogen, an optionally substituted —C₁-C₆ alkyl, or an oxygen protecting group; each instance of R^{c5} is independently an optionally substituted —C₁-C₆ alkyl; and each instance of R^{js} is independently an optionally substituted —C₁-C₆ alkyl, an optionally substituted C₆₋₁₂ aryl, an optionally substituted heteroaryl, or a sulfur protecting group or a compound comprising the formula

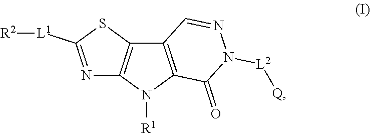

wherein Q is hydrogen, optionally substituted alkyl, optionally substituted alkenyl, optionally substituted alkynyl, optionally substituted cycloalkyl, optionally substituted heterocyclyl, optionally substituted aryl, or optionally substituted heteroaryl; R¹ is hydrogen, optionally substituted alkyl, optionally substituted haloalkyl, optionally substituted alkenyl, optionally substituted alkynyl, optionally substituted cycloalkyl, optionally substituted heterocyclyl, optionally substituted aryl, —OR⁰¹, —C(═O)R^{cl}, or a nitrogen protecting group; L¹ is a bond, optionally substituted alkylene —O—, —S—, —S—CH₂— —S(═O)CH₂— —S(═O)₂CH₂— —NR³—, —NR³C(═O)—, —C(═O)NR³—, —C(═O)—, —OC(═O)—, —C(═O)O— —NR³C(═O)O—, —OC(═O)NR³—, —NR³C(═O)NR³—, —OC(R)₂—, —C(R)₂O—, —NR³C(R⁴)₂—, —C(R⁴)₂NR³—, —S(═O)₂— —S(═O)— —S(═O)₂O— —OS(═O)₂— —S(═O)O— —OS(═O)—, —S(═O)₂NR³—, —NR³S(═O)₂— —S(═O)NR³—, —NR³S(═O)—, —NR³S(═O)₂O—, —OS(═O)₂NR³—, —NR³S(═O)O—, —OS(═O)NR³—, or —S(═O)(═NR³)—, wherein the point of the attachment to R² is on the left-hand side; L² is a bond, optionally substituted alkylene, —C(═O)—, —S(═O)₂— or —S(═O)—, wherein the point of the attachment to Q is on the right-hand side; R² is hydrogen, halogen, optionally substituted alkyl, optionally substituted alkoxy, optionally substituted alkenyl, optionally substituted alkynyl, optionally substituted cycloalkyl, optionally substituted heterocyclyl, optionally substituted aryl, or optionally substituted heteroaryl, or a nitrogen protecting group when L¹-NR³—, —NR³C(═O)—, —NR³C(═O)O— —NR³C(R)₂— —NR³S(═O)₂—, —NR³S(═O)—, —NR³C(═O)NR³—, —NR³S(═O)₂O— or —NR³S(═O)O— an oxygen protecting group when L¹ is —O—, —OC(═O)—, —OC(═O)NR³—, —OC(R⁴)₂— —OS(═O)₂—, —OS(═O)₂NR³—, —OS(═O)NR³—, or —OS(═O)—, or a sulfur protecting group when L¹ is —S—; each instance of R³ is independently hydrogen, —OR^{o2}, optionally substituted alkyl, optionally substituted alkenyl, optionally substituted alkynyl, optionally substituted cycloalkyl, optionally substituted heterocyclyl, optionally substituted aryl, optionally substituted heteroaryl, or a nitrogen protecting group; each instance of R^{o1}, and R^{o2} is independently hydrogen, optionally substituted alkyl, or an oxygen protecting group; each instance of R^{cl} is independently optionally substituted alkyl or —N(R^{cn})₂, wherein each instance of R^{cn} is independently hydrogen, —Ci-₆ alkyl, or a nitrogen protecting group; and each instance of R⁴ is independently hydrogen, optionally substituted alkyl, optionally substituted alkenyl, optionally substituted alkynyl, optionally substituted cycloalkyl, optionally substituted heterocyclyl, optionally substituted aryl, or optionally substituted heteroaryl; provided that Q and R² are not both optionally substituted 5- or 6-membered monocyclic heteroaryl when Li and L₂ are optionally substituted methylene. to the eye of a subject in need thereof, wherein the administering treats or reduces symptoms of an eye disorder in the subject.

Additional embodiments are described herein.

### Definitions

To facilitate an understanding of the present disclosure, a number of terms and phrases are defined below:

As used herein, the term “aliphatic” represents the groups including, but not limited to, alkyl, alkenyl, alkynyl, alicyclic.

The term “halo” or “halogen” refers to any radical of fluorine, chlorine, bromine or iodine.

As used herein, the term “alkyl” refers to an unsaturated carbon chain substituent group. In general, alkyls have the general formula CₙH₂ₙ₊₁. Exemplary alkyls include, but are not limited to, methyl (CH₃), ethyl (C₂H₅), propyl (C₃H₇), butyl (C₄H₉), pentyl (C₅H₁₁), etc.

The term “alkenyl” refers to a monovalent straight or branched hydrocarbon chain containing 2-12 carbon atoms and having one or more double bonds. Examples of alkenyl groups include, but are not limited to, allyl, propenyl, 2-butenyl, 3-hexenyl and 3-octenyl groups. One of the double bond carbons may optionally be the point of attachment of the alkenyl substituent. In certain aspects, the term “alkenyl” refers to a monovalent straight or branched hydrocarbon chain containing 2-6 carbon atoms and having one or more double bonds. In other aspects, the term “alkenyl” refers to a monovalent straight or branched hydrocarbon chain containing 2-4 carbon atoms and having one or more double bonds.

The term “alkynyl” refers to a monovalent straight or branched hydrocarbon chain containing 2-12 carbon atoms and characterized in having one or more triple bonds. Examples of alkynyl groups include, but are not limited to, ethynyl, propargyl, and 3-hexynyl. One of the triple bond carbons may optionally be the point of attachment of the alkynyl substituent.

As used herein, the term “aryl” represents a single aromatic ring such as a phenyl ring, or two or more aromatic rings (e.g., bisphenyl, naphthalene, anthracene), or an aromatic ring and one or more non-aromatic rings. The aryl group can be optionally substituted with a lower aliphatic group (e.g., alkyl, alkenyl, alkynyl, or alicyclic). Additionally, the aliphatic and aryl groups can be further substituted by one or more functional groups including, but not limited to, chemical moieties comprising N, S, O, —NH₂, —NHCOCH₃, —OH, lower alkoxy (C₁-C₄), and halo (—F, —Cl, —Br, or —I).

As used herein, the term “substituted aliphatic” refers to an alkane, alkene, alkyne, or alicyclic moiety where at least one of the aliphatic hydrogen atoms has been replaced by, for example, a halogen, an amino, a hydroxy, a nitro, a thio, a ketone, an aldehyde, an ester, an amide, a lower aliphatic, a substituted lower aliphatic, or a ring (aryl, substituted aryl, cycloaliphatic, or substituted cycloaliphatic, etc.). Examples of such include, but are not limited to, 1-chloroethyl and the like.

As used herein, the term “substituted aryl” refers to an aromatic ring or fused aromatic ring system consisting of at least one aromatic ring, and where at least one of the hydrogen atoms on a ring carbon has been replaced by, for example, a halogen, an amino, a hydroxy, a nitro, a thio, a ketone, an aldehyde, an ester, an amide, a lower aliphatic, a substituted lower aliphatic, or a ring (aryl, substituted aryl, cycloaliphatic, or substituted cycloaliphatic). Examples of such include, but are not limited to, hydroxyphenyl and the like. As used herein, the term “cycloaliphatic” refers to an aliphatic structure containing a fused ring system. Examples of such include, but are not limited to, decalin and the like.

As used herein, the term “substituted cycloaliphatic” refers to a cycloaliphatic structure where at least one of the aliphatic hydrogen atoms has been replaced by a halogen, a nitro, a thio, an amino, a hydroxy, a ketone, an aldehyde, an ester, an amide, a lower aliphatic, a substituted lower aliphatic, or a ring (aryl, substituted aryl, cycloaliphatic, or substituted cycloaliphatic). Examples of such include, but are not limited to, l-chlorodecalyl, bicyclo-heptanes, octanes, and nonanes (e.g., nonrbornyl) and the like.

As used herein, the term “heterocyclic” represents, for example, an aromatic or nonaromatic ring containing one or more heteroatoms. The heteroatoms can be the same or different from each other. Examples of heteroatoms include, but are not limited to nitrogen, oxygen and sulfur. Aromatic and nonaromatic heterocyclic rings are well-known in the art. Some nonlimiting examples of aromatic heterocyclic rings include pyridine, pyrimidine, indole, purine, quinoline and isoquinoline. Nonlimiting examples of nonaromatic heterocyclic compounds include piperidine, piperazine, morpholine, pyrrolidine and pyrazolidine. Examples of oxygen containing heterocyclic rings include, but not limited to furan, oxirane, 2H-pyran, 4H-pyran, 2H-chromene, and benzofuran. Examples of sulfur-containing heterocyclic rings include, but are not limited to, thiophene, benzothiophene, and parathiazine. Examples of nitrogen containing rings include, but not limited to, pyrrole, pyrrolidine, pyrazole, pyrazolidine, imidazole, imidazoline, imidazolidine, pyridine, piperidine, pyrazine, piperazine, pyrimidine, indole, purine, benzimidazole, quinoline, isoquinoline, triazole, and triazine. Examples of heterocyclic rings containing two different heteroatoms include, but are not limited to, phenothiazine, morpholine, parathiazine, oxazine, oxazole, thiazine, and thiazole. The heterocyclic ring is optionally further substituted with one or more groups selected from aliphatic, nitro, acetyl (i.e., —C(═O)—CH₃), or aryl groups.

As used herein, the term “substituted heterocyclic” refers to a heterocylic structure where at least one of the ring carbon atoms is replaced by oxygen, nitrogen or sulfur, and where at least one of the aliphatic hydrogen atoms has been replaced by a halogen, hydroxy, a thio, nitro, an amino, a ketone, an aldehyde, an ester, an amide, a lower aliphatic, a substituted lower aliphatic, or a ring (aryl, substituted aryl, cycloaliphatic, or substituted cycloaliphatic). Examples of such include, but are not limited to 2-chloropyranyl.

As used herein, the term “linker” refers to an organic or inorganic molecule that links multiple functional units of a molecule. In some embodiments, the linker is a single moiety or chain containing up to and including eight contiguous atoms connecting two different structural moieties where such atoms are, for example, carbon, nitrogen, oxygen, or sulfur.

As used herein, the term “lower-alkyl-substituted-amino” refers to any alkyl unit containing up to and including eight carbon atoms where one of the aliphatic hydrogen atoms is replaced by an amino group. Examples of such include, but are not limited to, ethylamino and the like.

The term “derivative” of a compound, as used herein, refers to a chemically modified compound wherein the chemical modification takes place either at a functional group of the compound or backbone.

As used herein, the term “subject” refers to organisms to be treated by the methods of the present disclosure. Such organisms preferably include, but are not limited to, mammals (e.g., murines, simians, equines, bovines, porcines, canines, felines, and the like), and most preferably includes humans. In the context of the disclosure, the term “subject” generally refers to an individual who will receive or who has received treatment (e.g., administration of a compound of the present disclosure and optionally one or more other agents) for a condition characterized by an eye disorder.

The term “diagnosed,” as used herein, refers to the recognition of a disease by its signs and symptoms (e.g., resistance to conventional therapies), or genetic analysis, pathological analysis, histological analysis, and the like.

As used herein, the term “effective amount” refers to the amount of a compound (e.g., a compound of the present disclosure) sufficient to effect beneficial or desired results. An effective amount can be administered in one or more administrations, applications or dosages and is not limited intended to be limited to a particular formulation or administration route. As used herein, the term “co-administration” refers to the administration of at least two agent(s) (e.g., a compound of the present disclosure) or therapies to a subject. In some embodiments, the co-administration of two or more agents/therapies is concurrent. In some embodiments, a first agent/therapy is administered prior to a second agent/therapy. Those of skill in the art understand that the formulations and/or routes of administration of the various agents/therapies used may vary. The appropriate dosage for co-administration can be readily determined by one skilled in the art. In some embodiments, when agents/therapies are co-administered, the respective agents/therapies are administered at lower dosages than appropriate for their administration alone. Thus, co-administration is especially desirable in embodiments where the co-administration of the agents/therapies lowers the requisite dosage of a known potentially harmful (e.g., toxic) agent(s).

As used herein, the term “toxic” refers to any detrimental or harmful effects on a cell or tissue as compared to the same cell or tissue prior to the administration of the toxicant.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants. (See e.g., Martin, Remington's Pharmaceutical Sciences, 15th Ed., Mack Publ. Co., Easton, PA [1975]).

As used herein, the term “pharmaceutically acceptable salt” refers to any pharmaceutically acceptable salt (e.g., acid or base) of a compound of the present disclosure which, upon administration to a subject, is capable of providing a compound of this disclosure or an active metabolite or residue thereof. As is known to those of skill in the art, “salts” of the compounds of the present disclosure may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the disclosure and their pharmaceutically acceptable acid addition salts.

Examples of bases include, but are not limited to, alkali metals (e.g., sodium) hydroxides, alkaline earth metals (e.g., magnesium), hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present disclosure compounded with a suitable cation such as Na⁺, NH₄⁺, and NW₄⁺ (wherein W is a C₁₋₄ alkyl group), and the like.

For therapeutic use, salts of the compounds of the present disclosure are contemplated as being pharmaceutically acceptable. However, salts of acids and bases that are non-pharmaceutically acceptable may also find use, for example, in the preparation or purification of a pharmaceutically acceptable compound.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, and industrial samples. Such examples are not however to be construed as limiting the sample types applicable to the present disclosure.

As used herein, the terms “purified” or “to purify” refer, to the removal of undesired components from a sample. As used herein, the term “substantially purified” refers to molecules that are at least 60% free, preferably 75% free, and most preferably 90%, or more, free from other components with which they usually associated.

The term “test compound” refers to any chemical entity, pharmaceutical, drug, and the like, that can be used to treat or prevent a disease, illness, sickness, or disorder of bodily function, or otherwise alter the physiological or cellular status of a sample (e.g., pyruvate kinase levels). Test compounds comprise both known and potential therapeutic compounds. A test compound can be determined to be therapeutic by using the screening methods of the present disclosure. A “known therapeutic compound” refers to a therapeutic compound that has been shown (e.g., through animal trials or prior experience with administration to humans) to be effective in such treatment or prevention.

### DETAILED DESCRIPTION OF THE DISCLOSURE

Provided herein are compositions and methods for activating pyruvate kinase (e.g., in a subject). In particular, provided herein are compositions and methods for treating a disease or condition (e.g., eye disease, blood disorders, or cancer) using pyruvate kinase activators.

Metabolic reprograming of photoreceptors is a therapeutic solution for vision loss associated with AMD, retinal dystrophies, and retinal detachment (Aït-Ali, N. et al. Cell 161, 817-832 (2015); Zhang, L. et al. J. Clin. Invest. 126, 4659-4673 (2016); Include Wubben et al. Sci Rep. 2017; Wubben et al. Sci Rep. 2020). Activation of PKM2, the key regulator of aerobic glycolysis and energy metabolism in photoreceptors, by small molecule activators (e.g., those described herein) reprograms metabolism and enhances energy production by favoring catabolic activity in the cells. Limitation of PKM2 expression and aerobic glycolysis to photoreceptors in the eye reduces any potential off-target effects after ocular delivery, increasing the specificity of the treatment and enhancing the therapeutic window.

Similar to cells with high metabolic demands including tumor cells, photoreceptors maintain PKM2 expression (Rajala et al., supra; Lindsay et al., supra; Ng, S. K. et al. Clin. Experiment. Ophthalmol. 43, 367-376 (2015)). This is in stark contrast to other terminally differentiated neurons, which express constitutively active PKM1 isoform exclusively (Jurica, M. S. et al. Struct. Lond. Engl. 1993 6, 195-210 (1998)). Unlike PKM1, PKM2 activity is tightly regulated in cells. As a tetramer, PKM2 displays high catalytic activity and is associated with ATP synthesis and catabolic metabolism. The nontetrameric form has low catalytic activity and is associated with anabolic metabolism and the shuttling of metabolic intermediates to biosynthetic pathways (Gui, D. Y., et al. Sci. Signal. 6, pe7 (2013); Wong, N., et al. Cancer Lett. 356, 184-191 (2015); Yang, W. & Lu, Z. J. Cell Sci. 128, 1655-1660 (2015)). A mouse model with selective deletion of PKM2 isoform in photoreceptors demonstrated compensatory PKM1 isoform expression and a net increase in overall PKM activity in the retina (See e.g., WO 2019/079541; herein incorporated by reference in its entirety). Under acute photoreceptor degeneration induced by retinal detachment, this mouse model showed decreased cell death in the retina. The model also showed decreased phosphorylation of PKM2, which promotes increased tetramerization and enzyme activity, in rodent retina during photoreceptor stress. Thus, the metabolic reprogramming observed in the retina of the photoreceptor-specific PKM2 knockout mice mimics the activation of PKM2 after nutrient deprivation by substituting constitutively active PKM1 to circumvent acute apoptotic stress (Wubben et al. Sci Rep. 2017). Additionally, it has been demonstrated that small molecule activation of PKM2 with ML-265 also circumvents photoreceptor apoptosis in models of outer retinal stress without any toxic effects on the retina long-term (Wubben et al. Sci Rep. 2020)

Accordingly, provided herein are compositions and methods for activating a pyruvate kinase (e.g., PKM1 or PKM2) to reprogram photoreceptor metabolism and block apoptosis, thus preventing vision loss in many retinal diseases, and treat other diseases or conditions (e.g., blood disorders or cancers).

### I. Activators

Provided herein are pyruvate kinase (e.g., PKM1 or PKM2) activators. Exemplary, non-limiting examples are provided below.

In some embodiments, the compound is selected from

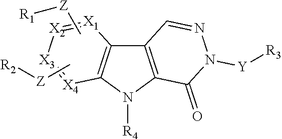

Formula I or

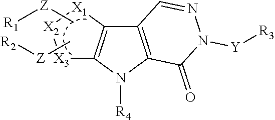

Formula II:
Wherein X₁, X₂, X₃, and X₄ are independently selected from CH, CO, N, NH, S, or O;
Z is null, a bond, optionally substituted C₁₋₆ alkyl, —O—, —S—, —CH₂—, —CHR₅—, —CR₅R₆—, —(CH₂)ₙ—, —(CHR₅)ₙ—, —(CR₅R₆)ₙ—, —S(═O)CH₂, —S(═O)₂CH₂—, —NR₅—, —NR₅C(═O)—, —C(O)NR₅—, —C(═O)—, —OC(═O)—, —C(═O)O—, —NR₅C(═O)O—, —OC(═O)NR₅—, —NR₅C(═O)NR₅—, —OC(R₅)₂—, —C(R₅)₂O—, —NR₄C(R₅)₂—, C(R₆)₂NR₅—, —S(═O)—, S(═O)₂—, —S(═O)₂O—, —OS(═O)₂—, —S(═O)₂NR₅—, —NR₅S(═O)₂—, —S(═O)NR₅—, —NR₅S(═O)—, —OS(═O)NR₅—, —NR₅S(═O)O—, or —S(═O)(═NR₅)—, where in the point of attachment to R₁ or R₂ is on the left and n=1-6;
R₅ and R₆ are each independently hydrogen, a halogen, —CN, OR₇, NR₇R₈, —N(R₇)C(═O)R₈, —C(═O)N(R₇), —C(═O)R₇, —C(═O)OR₇, —SR₇, —S(═O)R₇, —S(═O)₂R₇, or any optimally substituted —C₁-C₆ alkyl;
R₇ and R₈ are each independently hydrogen, any optimally substituted —C₁-C₆ alkyl; or alternatively R₇ and R₈ are taken together as an optionally substituted C₁-C₆ monocyclic cycloalkyl ring or an optionally substituted monocyclic heterocyclic ring;
Y is null, —CH₂—, —CHR₉—, —CR₉R₁₀—, —(CH₂)ₙ—, —(CHR₉)ₙ—, —(CR₉R₁₀)ₙ—, —C(═O)—, —S(═O)—, —S(═O)₂—, wherein n=1-6;
R₉ and R₁₀ are each independently hydrogen, a halogen, —CN, or any optimally substituted —C₁-C₆ alkyl; or alternatively R₉ and R₁₀ can be taken together as an optionally substituted C₁-C₆ monocyclic cycloalkyl ring or an optionally substituted monocyclic heterocyclic ring;
R₁ is —H, —F, —Cl, —Br, —NO₂, —CN, —NH₂, —NHR₁₁, —NR₁₁R₁₂, —OH, —OR₁₁, —CH₂—, —CHR₉—, —CR₉R₁₀, —(CH₂)ₙ—, —(CHR₉)ₙ—, —(CR₉R₁₀)ₙ—, optionally substituted C₁-C₆ alkyl, optionally substituted monocyclic cycloalkyl, optionally substituted bicyclic cycloalkyl, optionally substituted tricyclic cycloalkyl, optionally substituted monocyclic heterocycle, optionally substituted bicyclic heterocycle, or optionally substituted tricyclic heterocycle;
R₂ is —H, —F, —Cl, —Br, —NO₂, —CN, —NO₂, —NH₂, —NHR₁₁, —NR₁₁R₁₂, —OH, —OR₁₁, optionally substituted C₁-C₆ alkyl, optionally substituted monocyclic cycloalkyl, optionally substituted bicyclic cycloalkyl, optionally substituted tricyclic cycloalkyl, optionally substituted monocyclic heterocycle, optionally substituted bicyclic heterocycle, optionally substituted tricyclic heterocycle;
R₃ is —H, —F, —Cl, —Br, —NO₂, —CN, —NO₂, —NH₂, —NHR₁₁, —NR₁₁R₁₂, —OH, —OR₁₁, optionally substituted C₁-C₆ alkyl, optionally substituted monocyclic cycloalkyl, optionally substituted bicyclic cycloalkyl, optionally substituted tricyclic cycloalkyl, optionally substituted monocyclic heterocycle, optionally substituted bicyclic heterocycle, optionally substituted tricyclic heterocycle;
R₁₁ and R₁₂ are each independently optionally substituted C₁-C₆ alkyl, optionally substituted monocyclic cycloalkyl, optionally substituted bicyclic cycloalkyl, optionally substituted tricyclic cycloalkyl, optionally substituted monocyclic heterocycle, optionally substituted bicyclic heterocycle, or optionally substituted tricyclic heterocycle;
R₄ is hydrogen, —CH₃, —CHR₁₃, —CR₁₃R₁₄, —S(═O)R₁₃, —S(═O)₂R₁₃, optionally substituted alkyl, an optimally substituted haloalkyl, an optimally substituted alkenyl, an optimally substituted alkynyl, an optimally substituted cycloalkyl, an optimally substituted heterocycle, an optimally substituted aryl, —C(═O)R₁₅, or a nitrogen protecting group; wherein;
R₁₃ and R₁₄ are each independently optionally substituted C₁-C₆ alkyl, optionally substituted monocyclic cycloalkyl, optionally substituted bicyclic cycloalkyl, optionally substituted tricyclic cycloalkyl, optionally substituted monocyclic heterocycle, optionally substituted bicyclic heterocycle, optionally substituted tricyclic heterocycle;
R₁₅ is hydrogen, —CH₃, optionally substituted alkyl, an optimally substituted haloalkyl, an optimally substituted alkenyl, an optimally substituted alkynyl, an optimally substituted cycloalkyl, an optimally substituted heterocycle, an optimally substituted aryl or a pharmaceutically acceptable salt thereof.

In some embodiments R₂—Z— is H and R₁—Z— is selected from;
Z is null, a bond, optionally substituted C₁₋₆ alkyl, —O—, —S—, —CH₂—, —CHR₅—, —CR₅R₆—, —(CH₂)ₙ—, —(CHR₅)ₙ—, —(CR₅R₆)ₙ—, —S(═O)CH₂, —NR₅—, —NR₅C(═O)—, —C(O)NR₅—, —C(═O)—, —S(═O)—, where in the point of attachment to R₁ is on the left and n=1-6;
R₅ and R₆ are each independently hydrogen, —CN, OR₇, NR₇R₈, or any optimally substituted —C₁-C₆ alkyl; or alternatively R₇ and R₈ can be taken together as an optionally substituted C₁-C₆ monocyclic cycloalkyl ring or an optionally substituted monocyclic heterocyclic ring; wherein;
R₇ and R₈ are each independently hydrogen, any optimally substituted —C₁-C₆ alkyl; or alternatively R₇ and R₈ can be taken together as an optionally substituted C₁-C₆ monocyclic cycloalkyl ring or an optionally substituted monocyclic heterocyclic ring;
Y is null, —CH₂—, —CHR₉—, —CR₉R₁₀—, —(CH₂)ₙ—, —(CHR₉)ₙ—, —(CR₉R₁₀)ₙ—, wherein n=1-3;
R₉ and R₁₀ are each independently hydrogen, or any optimally substituted —C₁-C₆ alkyl; or alternatively R₉ and R₁₀ can be taken together as an optionally substituted C₁-C₆ monocyclic cycloalkyl ring or an optionally substituted monocyclic heterocyclic ring;
R₁ is —H, —F, —Cl, —Br, —NO₂, —CN, —NO₂, —NH₂, —NHR₁₁, —NR₁₁R₁₂, —OH, —OR₁₁, —CH₂—, —CHR₉—, —CR₉R₁₀—, —(CH₂)ₙ—, —(CHR₉)ₙ—, —(CR₉R₁₀)ₙ—, optionally substituted C₁-C₆ alkyl, optionally substituted monocyclic cycloalkyl, optionally substituted bicyclic cycloalkyl, optionally substituted tricyclic cycloalkyl, optionally substituted monocyclic heterocycle, optionally substituted bicyclic heterocycle, or optionally substituted tricyclic heterocycle;
R₂ is —H, —F, —Cl, —Br, —NO₂, —CN, —NO₂, —NH₂, —NHR₁₁, —NR₁₁R₁₂, —OH, —OR₁₁, —CH₂—, —CHR₉—, —CR₉R₁₀—, —(CH₂)ₙ—, —(CHR₉)ₙ—, —(CR₉R₁₀)ₙ—, optionally substituted C₁-C₆ alkyl, optionally substituted monocyclic cycloalkyl, optionally substituted bicyclic cycloalkyl, optionally substituted tricyclic cycloalkyl, optionally substituted monocyclic heterocycle, optionally substituted bicyclic heterocycle, or optionally substituted tricyclic heterocycle;
R₃ is —H, —F, —Cl, —Br, —NO₂, —CN, —NO₂, —NH₂, —NHR₁₁, —NR₁₁R₁₂, —OH, —OR₁₁, —CH₂—, —CHR₉—, —CR₉R₁₀—, —(CH₂)ₙ—, —(CHR₉)ₙ—, —(CR₉R₁₀)ₙ—, optionally substituted C₁-C₆ alkyl, optionally substituted monocyclic cycloalkyl, optionally substituted bicyclic cycloalkyl, optionally substituted tricyclic cycloalkyl, optionally substituted monocyclic heterocycle, optionally substituted bicyclic heterocycle, optionally substituted tricyclic heterocycle;
R₁₁ and R₁₂ are each independently optionally substituted C₁-C₆ alkyl, optionally substituted monocyclic cycloalkyl, optionally substituted bicyclic cycloalkyl, optionally substituted tricyclic cycloalkyl, optionally substituted monocyclic heterocycle, optionally substituted bicyclic heterocycle, or optionally substituted tricyclic heterocycle;
R₄ is —CH₃.

In some exemplary embodiments Z—R₁, Z—R₂, Z—R₃, and Y—R₃ individually or in combination are selected from, for example,

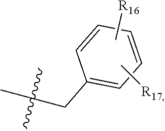

wherein R₁₆ and R₁₇ can be located in the ortho, meta or para positions on the aryl ring and are selected from H, —OCH₃, C₁-C₄ alkyl, —NH₂, -halogen, —CN, —OH, —S(═O)Me, —S(═O)₂Me, —CH₂OMe, —CH₂NR₁₈R₁₉ wherein R₁₈ and R₁₉ are selected from —H, C₁-C₄ alkyl, an optimally substituted aryl or heterocycle or taken together to form a carbocycle or heterocycle;

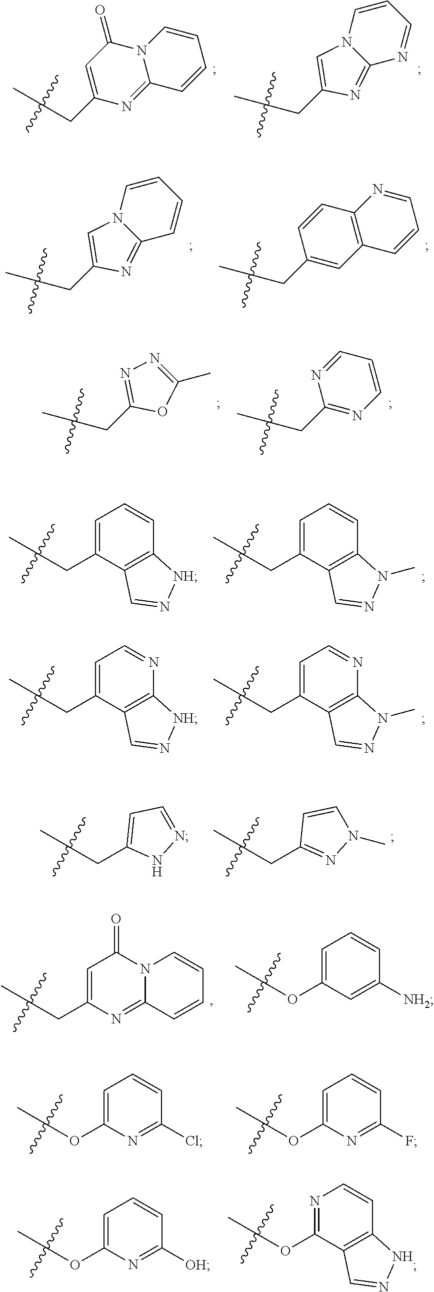

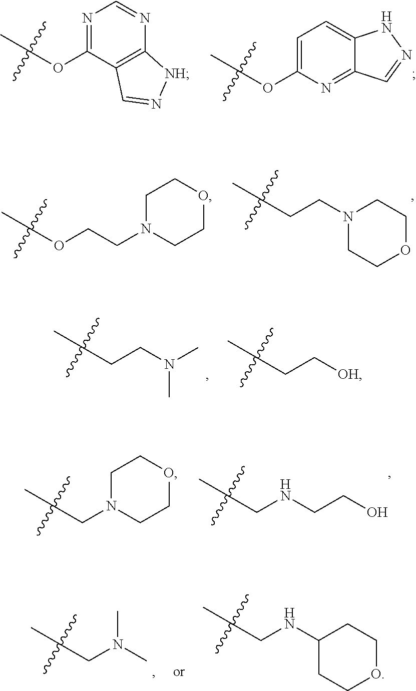

In some embodiments, the compound is a compound of Formula I where Z—R₂ is H, Z—R₁ is not 8-OCH₃, R₄ is not

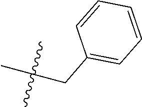 [SMILES: *Cc1ccccc1]

### and Y—R₃ is not

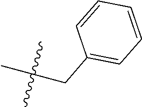 [SMILES: *Cc1ccccc1]

In some embodiments, the compound is a compound of Formula I where Z—R₂ is H, Z—R is not 8-Cl, R₄ is not CH₃ or

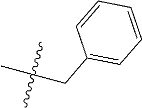 [SMILES: *Cc1ccccc1]

and Y—R₃ is not CH₃ or

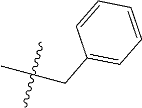 [SMILES: *Cc1ccccc1]

In some embodiments, the compound is a compound of Formula I and R₂ is not

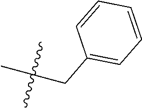 [SMILES: *Cc1ccccc1]

or CH₃, and Y—R₃ is not

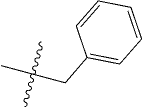 [SMILES: *Cc1ccccc1]

In some embodiments, the compound is a compound of Formula I and Z—R₁ is not

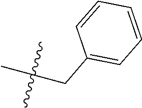 [SMILES: *Cc1ccccc1]

X₁ is not CO, R₂ is not CH₃, and Y—R₃ is not

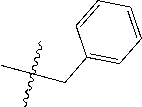 [SMILES: *Cc1ccccc1]

In some embodiments, the compound is not

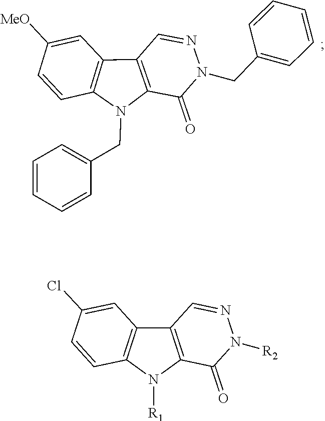

wherein R₁ and R₂ are independently CH₃ or

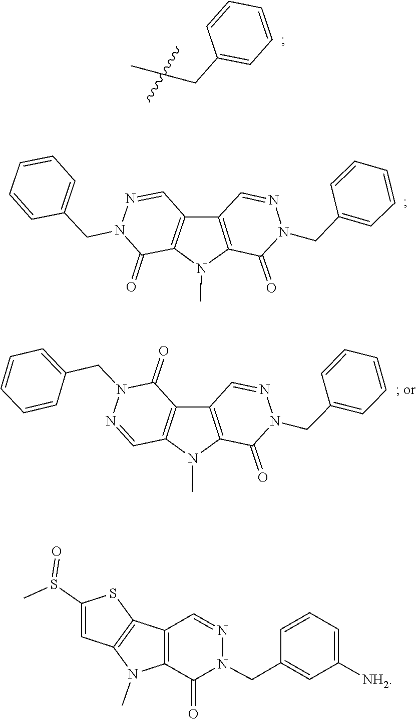 [SMILES: *Cc1ccccc1 ; Cn1c2c(=O)n(Cc3ccccc3)ncc2c2cnn(Cc3ccccc3)c(=O)c21 ; Cn1c2cnn(Cc3ccccc3)c(=O)c2c2cnn(Cc3ccccc3)c(=O)c21 ; Cn1c2cc(S(C)=O)sc2c2cnn(Cc3cccc(N)c3)c(=O)c21]

In some embodiments, the compound is selected from the compounds described in Table 2. In some embodiments, the compound is:

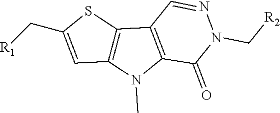

wherein R₁ is a substituted mono or bi-cyclic carbocyclic or heterocyclic moiety and R₂ is a substituted mono or bi-cyclic heterocyclic moiety.

In some exemplary embodiments, the compound is

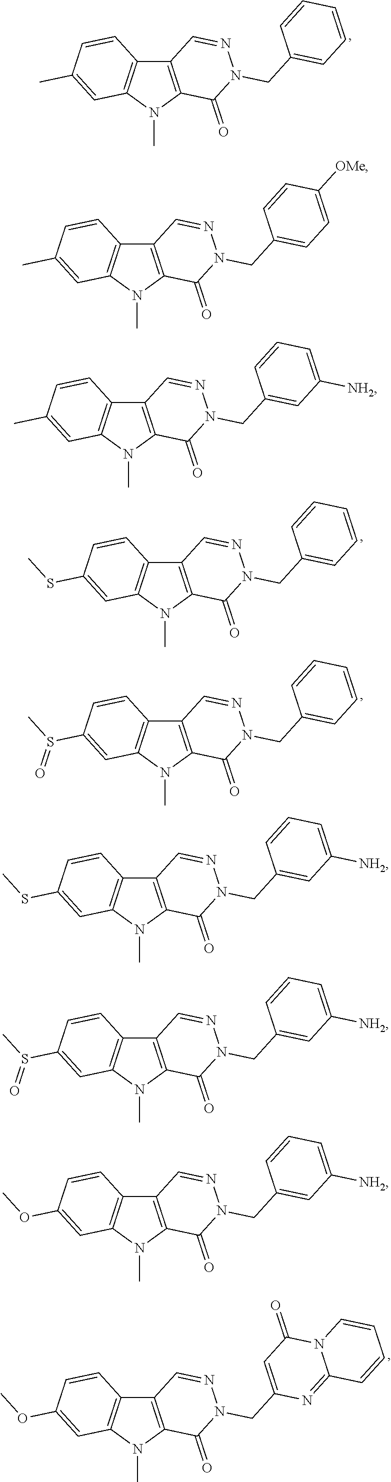

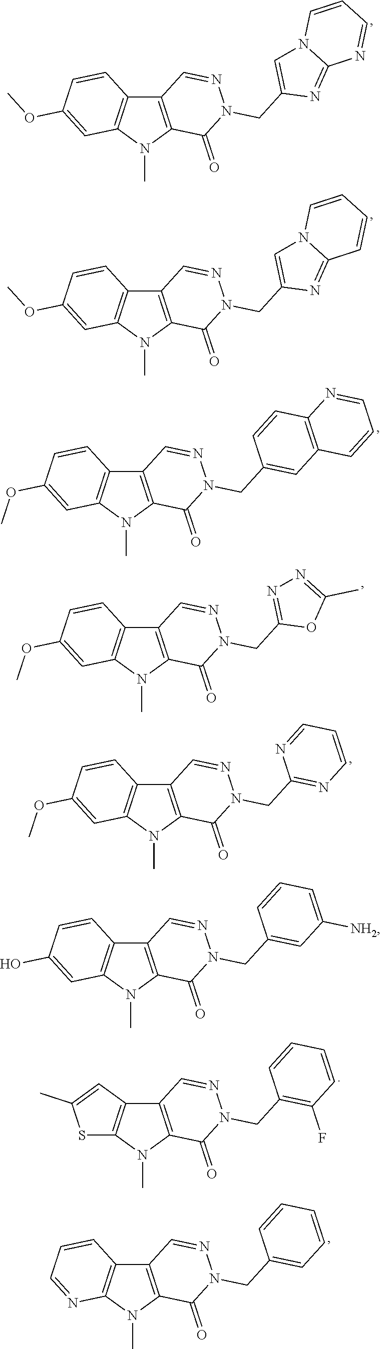

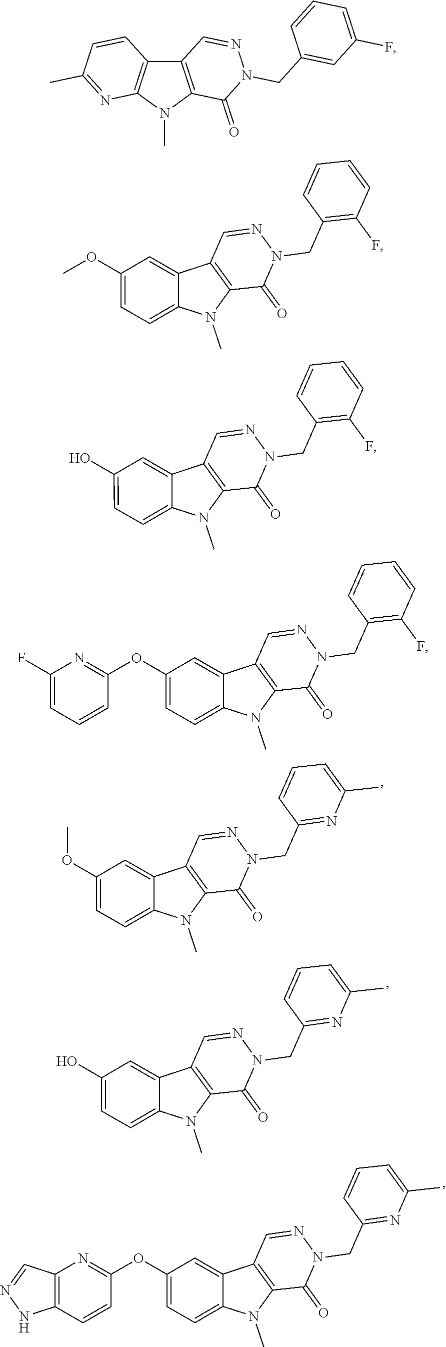

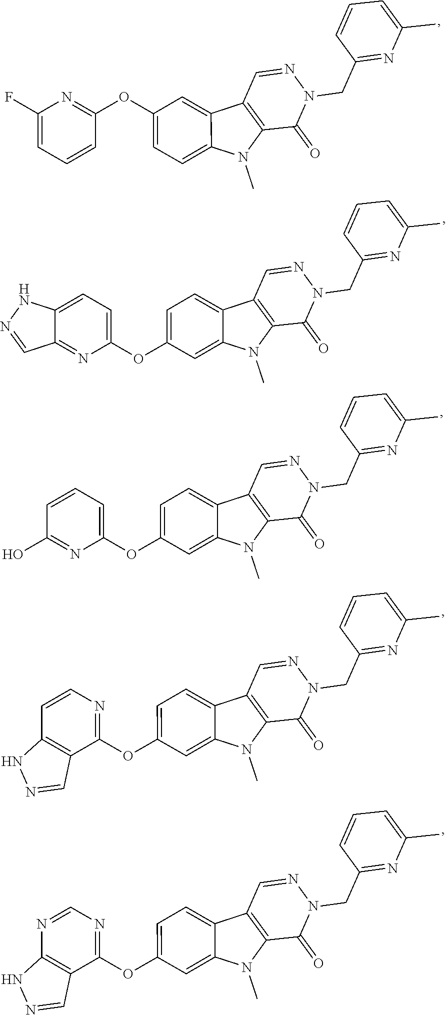 [SMILES: Cc1cccc(Cn2ncc3c4cc(Oc5cccc(F)n5)ccc4n(C)c3c2=O)n1 ; Cc1cccc(Cn2ncc3c4ccc(Oc5ccc6[nH]ncc6n5)cc4n(C)c3c2=O)n1 ; Cc1cccc(Cn2ncc3c4ccc(Oc5cccc(O)n5)cc4n(C)c3c2=O)n1 ; Cc1cccc(Cn2ncc3c4ccc(Oc5nccc6[nH]ncc56)cc4n(C)c3c2=O)n1 ; Cc1cccc(Cn2ncc3c4ccc(Oc5ncnc6[nH]ncc56)cc4n(C)c3c2=O)n1]

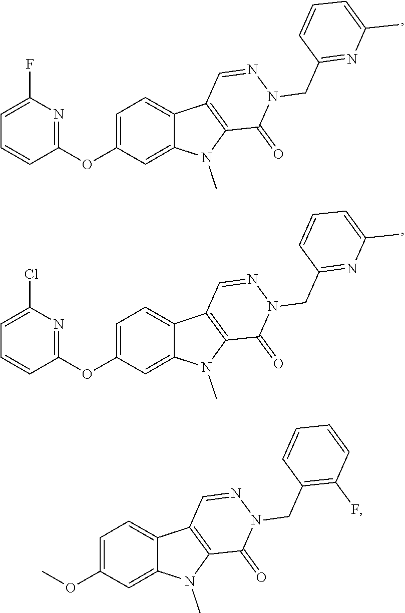

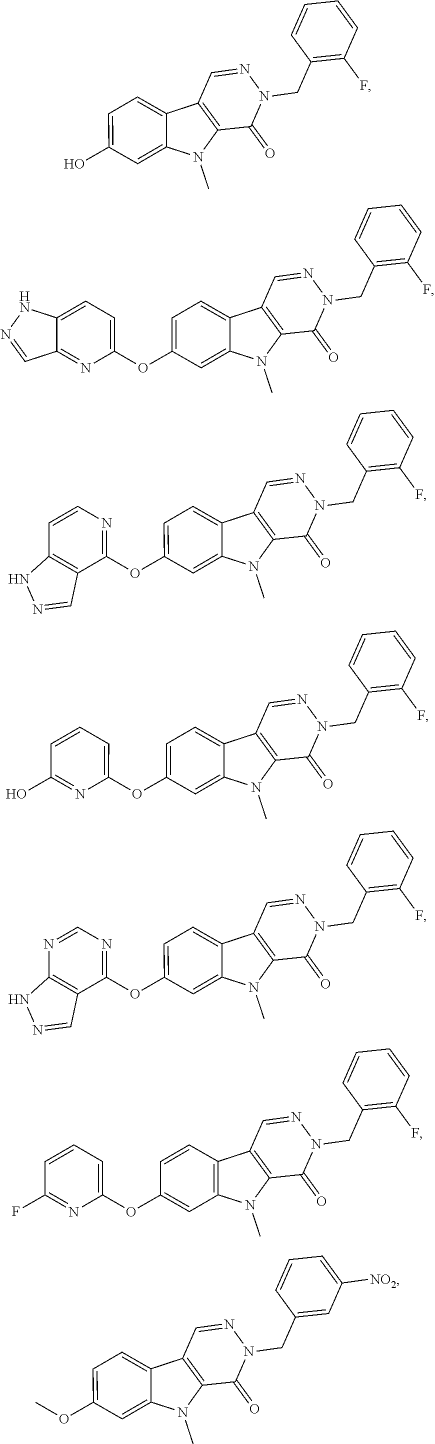

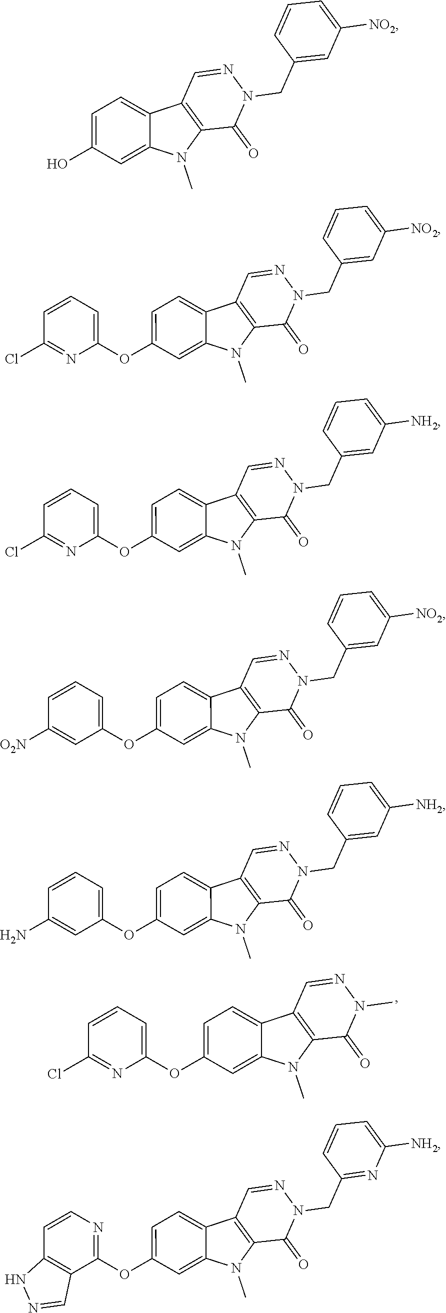

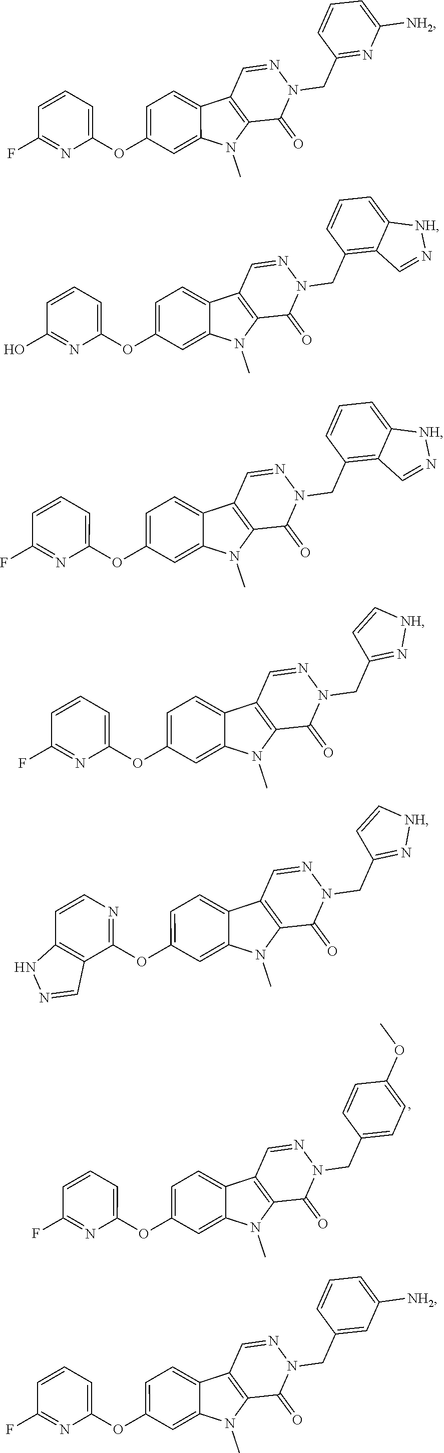

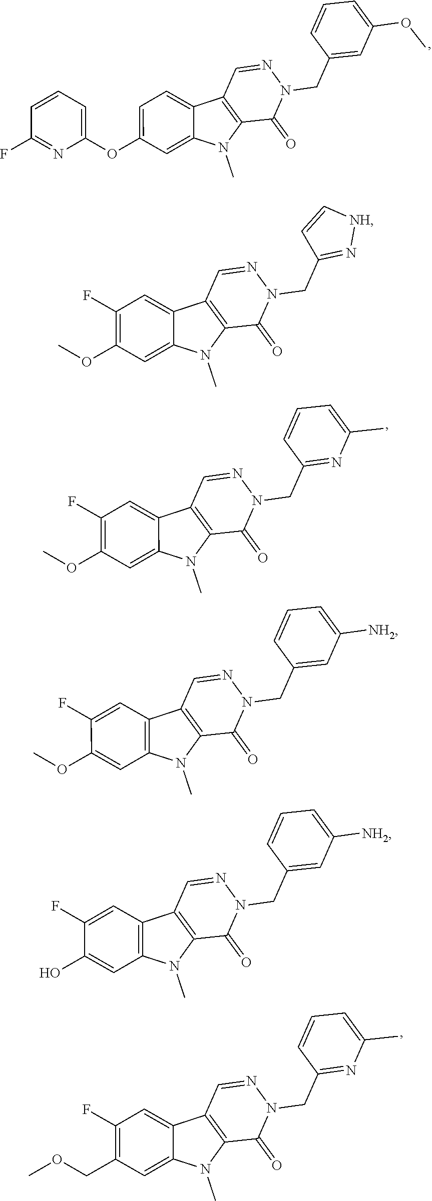

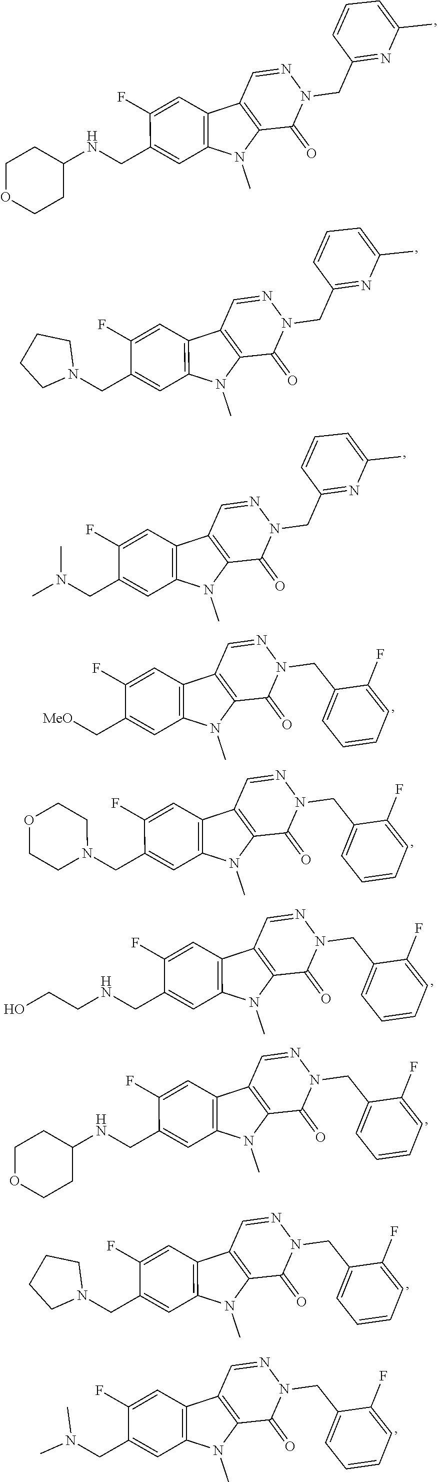

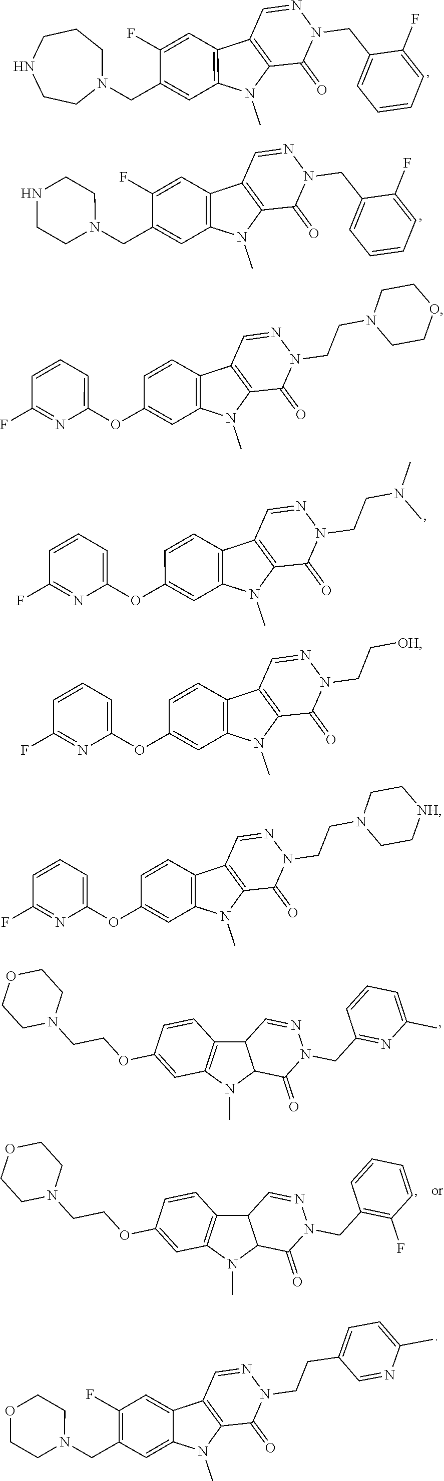

In some embodiments, the compound is

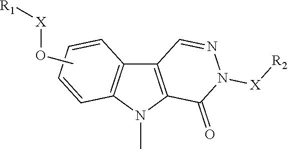

wherein
X is null or is selected from —H, —CH₂—, —CHR₃—, —CR₃R₄—, —(CH₂)ₙ—, —(CHR₃)ₙ—, —(CR₃R₄)ₙ—, wherein n-1-6;
R₁ is selected from, for example, —H, —CN, —NO₂, —NH₂, —NHR₃, NR₃R₄, —OH, OR₃, —SOR₃, —SO₂R₃, optionally substituted C₁-C₆ alkyl, optionally substituted monocyclic cycloalkyl, optionally substituted bicyclic cycloalkyl, optionally substituted tricyclic cycloalkyl, optionally substituted monocyclic heterocycle, optionally substituted bicyclic heterocycle, or optionally substituted tricyclic heterocycle;
R₂ is selected from, for example, —H, —CN, —NO₂, —NH₂, —NHR₃, NR₃R₄, —OH, OR₃, —SOR₃, —SO₂R₃, optionally substituted C₁-C₆ alkyl, optionally substituted monocyclic cycloalkyl, optionally substituted bicyclic cycloalkyl, optionally substituted monocyclic heterocycle, optionally substituted bicyclic heterocycle, or optionally substituted tricyclic heterocycle;
R₃ and R₄ are each independently selected from the group, for example, optionally substituted C₁-C₆ alkyl, optionally substituted monocyclic cycloalkyl, optionally substituted bicyclic cycloalkyl, optionally substituted tricyclic cycloalkyl, optionally substituted monocyclic heterocycle, optionally substituted bicyclic heterocycle, or optionally substituted tricyclic heterocycle; and

In some embodiments, R₁ is selected from, for example,

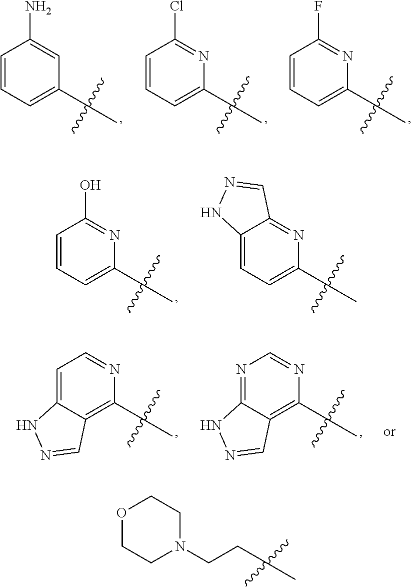

and R₂ is selected from for example,

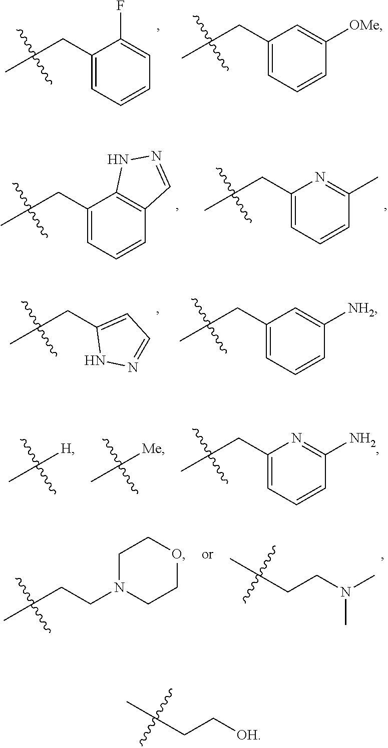

In further embodiments, the compound is

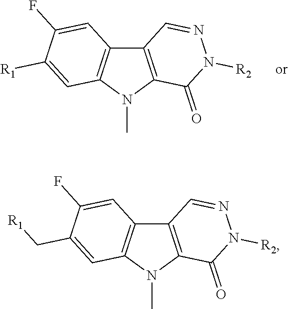

wherein R₁ is selected from, for example —H, —F, —Cl, —Br, —CN, —NO₂, —NH₂, —NHR₃, —NR₃R₄, —OH, —OR₃, optionally substituted C₁-C₆ alkyl, optionally substituted monocyclic cycloalkyl, optionally substituted bicyclic cycloalkyl, optionally substituted tricyclic cycloalkyl, optionally substituted monocyclic heterocycle, optionally substituted bicyclic heterocycle, or optionally substituted tricyclic heterocycle;
R₂ is selected from, for example, —H, —CH₃, —(CH₂)ₙ—R₅, —(CHR₃)ₙ—R₅, —(CR₃R₄)ₙ—R₅, optionally substituted C₁-C₆ alkyl, optionally substituted monocyclic cycloalkyl, optionally substituted bicyclic cycloalkyl, optionally substituted monocyclic heterocycle, optionally substituted bicyclic heterocycle, or optionally substituted tricyclic heterocycle;
R₃ and R₄ are each independently selected from, for example, optionally substituted C₁-C₆ alkyl, optionally substituted monocyclic cycloalkyl, optionally substituted bicyclic cycloalkyl, optionally substituted tricyclic cycloalkyl, optionally substituted monocyclic heterocycle, optionally substituted bicyclic heterocycle, or optionally substituted tricyclic heterocycle; and
R₅ is —H, —F, —Cl, —Br, —NO₂, —CN, —NO₂, —NH₂, —NHR₃, —NR₃R₄, —OH, —OR₃, optionally substituted C₁-C₆ alkyl, optionally substituted monocyclic cycloalkyl, optionally substituted bicyclic cycloalkyl, optionally substituted tricyclic cycloalkyl, optionally substituted monocyclic heterocycle, optionally substituted bicyclic heterocycle, optionally substituted tricyclic heterocycle.

In some embodiments, R₁ is selected from, for example, OCH₃, OH, NH₂, NCH₃, N(CH₃)₂,

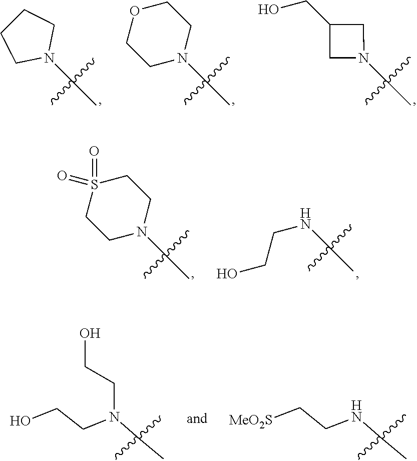

and R₂ is selected from, for example,

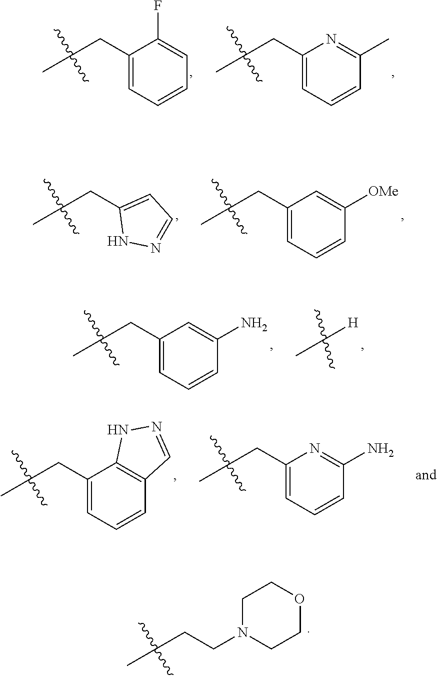

In some embodiments, one or more hydrogens or any of the aforementioned compounds are replaced with deuterium (D) (See e.g., Cargnin et al., Future Medicinal Chemistry; (2019) 11(16), 2039-2042)). Due to the twofold higher mass of D compared with H, the C-D bond is much more resistant toward oxidative processes, for example catalyzed by CYP450 or by other enzymes involved in metabolism, while typically retaining very similar steric properties. Therefore, the H-D isosteric replacement in correspondence of an oxidizable soft-spot usually retains the pharmacodynamics, while improving the pharmacokinetics of a drug with a repercussion on half-life and/or of area under the curve values and, ultimately, on dose and/or dosing regimen.

Example 1 below describes synthesis and activity of the described compounds.

### II. Methods of Treatment

In one embodiment, provided is a method for treating a disease, condition or disorder as described herein (e.g., treating) comprising administering a compound, a pharmaceutically acceptable salt of a compound or pharmaceutical composition comprising a compound described herein.

The compounds and compositions described herein can be administered to cells in culture, e.g. in vitro or ex vivo, or to a subject, e.g., in vivo, to treat, and/or diagnose a variety of disorders, including those described herein below.

In some embodiments, methods and uses of the pyruvate kinase (e.g., PKM2) activators described above in the treatment of eye disorders are provided. In some embodiments, the eye disorder is, for example, retinal dystrophy, vision loss, macular degeneration, retinal detachment, or proliferative vitreoretinopathy. In some embodiments, the administering prevents or reduces photoreceptor cell death in the eye of the subject.

In some embodiments, the present disclosure provides compositions, kits, systems, and/or methods to prevent, inhibit, block, and/or reduce photoreceptor cell death (e.g., in a human subject in need thereof). In some embodiments, the activators inhibit apoptosis of photoreceptors. In some embodiments, photoreceptor death and/or apoptosis is caused by retinal detachment, age-related macular degeneration, trauma, inflammation, uveitis, diabetes, hereditary retinal degeneration, and/or a disease affecting photoreceptor cells. In some embodiments, photoreceptor death and/or apoptosis is caused by retinal detachment. In some embodiments, retinal detachment is caused by one or more underlying diseases, disorders, or conditions (e.g., age-related macular degeneration, trauma, inflammation, uveitis, diabetes, hereditary retinal degeneration, etc.). In some embodiments, the present disclosure finds utility in enhancing photoreceptor viability and/or inhibiting photoreceptor death in a variety of conditions and/or diseases including, but not limited to macular degeneration (e.g. dry, wet, non-exudative, or exudative/neovascular), hereditary retinal degenerations (e.g. retinitis pigmentosa, Stargardt's disease, Usher Syndrome, etc.), ocular inflammatory disease (e.g. uveitis), ocular infection (e.g. bacterial, fungal, viral), autoimmune retinitis (e.g. triggered by infection), trauma, diabetic retinopathy, choroidal neovascularization, retinal ischemia, retinal vascular occlusive disease (e.g. branch retinal vein occlusion, central retinal vein occlusion, branch retinal artery occlusion, central retinal artery occlusion, etc.), pathologic myopia, angioid streaks, macular edema (e.g. of any etiology), and/or central serous chorioretinopathy.

In some embodiments, the present disclosure provides a method for treating patients suffering from such retinal detachment and or retinal disorders and in need of treatment. In some embodiments, a pharmaceutical composition comprising at least one PKM2 activator described herein is delivered to such a patient in an amount and at a location sufficient to treat the disorder or disease. In some embodiments, activators (or pharmaceutical composition comprising such) can be delivered to the patient systemically or locally, and it will be within the ordinary skill of the medical professional treating such patient to ascertain the most appropriate delivery route, time course, and dosage for treatment. It will be appreciated that application of the method of treating a patient most preferably substantially alleviates or even eliminates such symptoms; however, as with many medical treatments, application of the method is deemed successful if, during, following, or otherwise as a result of the method, the symptoms of the disease or disorder in the patient subside to a degree ascertainable.

In some embodiments, compounds described herein find use in the treatment of blood disorders (e.g., those described herein). In one embodiment of the disclosure provided is a method for increasing the lifetime of red blood cells (RBCs) in need thereof comprising contacting blood with an effective amount of a compound described herein or a pharmaceutically acceptable salt thereof; a pharmaceutically acceptable composition comprising a compound described herein or a pharmaceutically acceptable salt thereof, and a pharmaceutically acceptable carrier.

In a further embodiment the compound or pharmaceutical composition is added directly to whole blood or packed red blood cells (e.g. extracorporeally). In another embodiment, the compound or pharmaceutical composition is administered to a subject in need thereof.

In one embodiment of the disclosure provided is a method for regulating 2,3-diphosphoglycerate levels in blood in need thereof contacting blood with an effective amount of a compound described herein or a pharmaceutically acceptable salt thereof; a pharmaceutically acceptable composition comprising a compound described herein or a pharmaceutically acceptable salt thereof, and a pharmaceutically acceptable carrier.

In one embodiment of the disclosure provided is a method for treating sickle cell disease comprising administering to a subject in need thereof with an effective amount of a compound described herein or a pharmaceutically acceptable salt thereof; a pharmaceutically acceptable composition comprising a compound described herein or a pharmaceutically acceptable salt thereof, and a pharmaceutically acceptable carrier.

As used here, sickle cell disease (SCD), Hemoglobin SS disease, and sickle cell anemia are used interchangeably. Sickle cell disease (SCD) describes a group of inherited red blood cell disorders. In certain embodiments, subjects with SCD have abnormal hemoglobin, called hemoglobin S or sickle hemoglobin, in their red blood cells. In certain embodiments, a subject having SCD has at least one abnormal genes causing the body to make hemoglobin S. In certain embodiments, a subject having SCD has two hemoglobin S genes, Hemoglobin SS.

In one embodiment of the disclosure provided is a method of treating pyruvate kinase deficiency (PKD) in a subject comprising administering to the subject an effective amount of a compound described herein or a pharmaceutically acceptable salt thereof; a pharmaceutically acceptable composition comprising a compound described herein or a pharmaceutically acceptable salt thereof, and a pharmaceutically acceptable carrier.

In one embodiment of the disclosure provided is a method of treating anemia in a subject comprising administering to the subject an effective amount of a compound described herein or a pharmaceutically acceptable salt thereof; a pharmaceutically acceptable composition comprising a compound described herein or a pharmaceutically acceptable salt thereof, and a pharmaceutically acceptable carrier. In certain embodiments, the anemia is a dyserythropoietic anemia such as congenital dyserythropoietic anemia type I, II, III, or IV. In certain embodiments, the anemia is hemolytic anemia. In certain embodiments, the hemolytic anemia is a congenital and/or hereditary form of hemolytic anemia such as PKD, sickle cell disease, thalassemias (e.g. alpha or beta), hereditary spherocytosis, hereditary elliptocytosis), paroxysmal nocturnal hemoglobinuria, abeta-liproteinemia (Bassen-Kornzweig syndrome). In certain embodiments, the hemolytic anemia is acquired hemolytic anemia such as autoimmune hemolytic anemia, drug-induced hemolytic anemia. In certain embodiments, the hemolytic anemia is anemia as part of a multi-system disease, such as the anemia of Congenital Erythropoietic Purpura, Fanconi, Diamond-Blackfan.

As used herein, the term “anemia” refers to a deficiency of red blood cells (RBCs) and/or hemoglobin. As used herein, anemia includes all types of clinical anemia, for example (but not limited to): microcytic anemia, iron deficiency anemia, hemoglobinopathies, heme synthesis defect, globin synthesis defect, sideroblastic defect, normocytic anemia, anemia of chronic disease, aplastic anemia, hemolytic anemia, macrocytic anemia, megaloblastic 10 anemia, pemicious anemia, dimorphic anemia, anemia of prematurity, Fanconi anemia, hereditary spherocytosis, sickle cell disease, warm autoimmune hemolytic anemia, cold agglutinin hemolytic anemia, osteopetrosis, thalassemia, and myelodysplastic syndrome.

In certain embodiments, provided herein is a method of increasing amount of hemoglobin in a subject in thereof by administering an effective amount of a compound described herein or a pharmaceutically acceptable salt thereof; a pharmaceutically acceptable composition comprising a compound described herein or a pharmaceutically acceptable salt thereof, and a pharmaceutically acceptable carrier. In certain embodiments, the provided method increases hemoglobin concentration in the subject.

In one embodiment of the disclosure provided is a method for treating hemolytic anemia comprising administering to a subject an effective amount of a compound described herein or a pharmaceutically acceptable salt thereof; a pharmaceutically acceptable composition comprising a compound described herein or a pharmaceutically acceptable salt thereof, and a pharmaceutically acceptable carrier. In a further embodiment, the hemolytic anemia is hereditary and/or congenital hemolytic anemia, acquired hemolytic anemia, or anemia as part of a multisystem disease. In certain embodiments, the hemolytic anemia is congenital anemia. In certain embodiments, the hemolytic anemia is hereditary (e.g. non-spherocytic hemolytic anemia or hereditary spherocytosis).

In one embodiment of the disclosure provided is a method of treating thalassemia; hereditary spherocytosis; hereditary elliptocytosis; abetalipoproteinemia or BassenKomzweig syndrome; paroxysmal nocturnal hemoglobinuria; acquired hemolytic anemia (e.g., congenital anemias (e.g., enzymopathies)); sickle cell disease; or anemia of chronic diseases comprising administering to a subject an effective amount of a compound described herein or a pharmaceutically acceptable salt thereof; a pharmaceutically acceptable composition comprising a compound described herein or a pharmaceutically acceptable salt thereof, and a pharmaceutically acceptable carrier. In one embodiment, the acquired hemolytic anemia comprises congenital anemias. In certain embodiments, the provided method is to treat thalassemia. In certain embodiments, the thalassemia is beta-thalassemia.

In some embodiments, compounds described herein find use in the treatment of proliferative disorders (e.g., those described herein). In some embodiments, provided is a method of treating a proliferative disease comprising administering to a subject an effective amount of a compound described herein or a pharmaceutically acceptable salt thereof; a pharmaceutically acceptable composition comprising a compound described herein or a pharmaceutically acceptable salt thereof, and a pharmaceutically acceptable carrier. As used here, “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, angiogenesis, inflammatory diseases, and autoimmune diseases. In certain embodiments, the proliferative disease is cancer. In certain embodiments, the proliferative disease is an autoimmune disease. In certain embodiments, the proliferative disease is proliferative vitreoretinopathy.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma.

In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “cancer” refers to a class of diseases characterized by the development of abnormal cells that proliferate uncontrollably and have the ability to infiltrate and destroy normal body tissues. See, e.g., Stedman's Medical Dictionary, 25th ed.; Hensyl ed.; Williams & Wilkins: Philadelphia, 1990. Exemplary cancers include solid tumors, soft tissue tumors, and metastases thereof. The disclosed methods are also useful in treating non-solid cancers. Exemplary solid tumors include malignancies (e.g., sarcomas, adenocarcinomas, and carcinomas) of the various organ systems, such as those of lung, breast, lymphoid, gastrointestinal (e.g., colon), and genitourinary (e.g., renal, urothelial, or testicular tumors) tracts, pharynx, prostate, and ovary. Exemplary adenocarcinomas include colorectal cancers, renal-cell carcinoma, liver cancer, non small cell carcinoma of the lung, and cancer of the small intestine. Other exemplary cancers include: Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood; Carcinoid Tumor, Childhood; Carcinoid Tumor, Gastrointestinal; Carcinoma, Adrenocortical; Carcinoma, Islet Cell; Carcinoma of Unknown Primacy; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma, Childhood Brain Stem; Glioma, Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's, Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplasia Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood; Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland Cancer, Childhood; Sarcoma, Ewing's Family of Tumors; Sarcoma, Kaposi's; Sarcoma (Osteosarcoma)/Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; TCell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macro globulinemia; and Wilms' Tumor. Metastases of the aforementioned cancers can also be treated or prevented in accordance with the methods described herein.

The composition can be formulated for local (e.g., ocular; intraocular space; etc.), parenteral, oral, or topical administration. For example, a parenteral formulation could comprise or consist of a prompt or sustained release liquid preparation, dry powder, emulsion, suspension, or any other standard formulation. An oral formulation of the pharmaceutical composition could be, for example, a liquid solution, such as an effective amount of the composition dissolved in diluents (e.g., water, saline, juice, etc.), suspensions in an appropriate liquid, or suitable emulsions. An oral formulation could also be delivered in tablet form, and could include excipients, colorants, diluents, buffering agents, moistening agents, preservatives, flavoring agents, and pharmacologically compatible excipients. A topical formulation could include compounds to enhance absorption or penetration of the active ingredient through the skin or tissue or other affected areas, such as dimethylsulfoxide and related analogs. The pharmaceutical composition could also be delivered topically using a transdermal device, such as a patch or pump, which could include the composition in a suitable solvent system with an adhesive system, such as an acrylic emulsion, and a polyester patch. Compositions could be delivered via eye drops or other topical eye delivery method. Compositions may be delivered intraocularly, anywhere in the eye including, for example, the vitreous cavity, the anterior chamber, etc. Compositions may be delivered intravitreally as is commonly done with intravitreal injections of Lucentis (ranibizumab), Avastin (bevacizumab), triamcinolone acetonide, antibiotics, etc. Compositions may be delivered periocularly (e.g. to the tissue around the eyeball (globe) but within the bony orbit). Compositions may be delivered via intraocular implant (e.g. gancyclovir implant, fluocinolone implant, etc.). In intraocular implant delivery, devices containing compositions of the present disclosure are surgically implanted (e.g. within the vitreous cavity), and the drug is released into the eye (e.g. at a predetermined rate). Compositions may be administered using encapsulated cell technology (e.g. by Neurotech) in which genetically modified cells are engineered to produce and secrete compositions of the present disclosure. Compositions may be delivered via transcleral drug delivery using a device sutured or placed next to the globe that would slowly elute the drug, which would then diffuse into the eye.

In some embodiments, PKM2 activators are co-administered with another treatment for retinal detachment or macular degeneration (e.g., laser or other surgery, Ranibizumab, vitamins, or nutritional supplements), blood disorder, or cancer (e.g., chemotherapy or radiation).

In some embodiments, the present disclosure provides co-administration of two or more anti-apoptotic and/or photoreceptor protective compositions described herein. In some embodiments, the present disclosure provides co-administration of one or more anti-apoptotic and/or photoreceptor protective compositions described herein with one or more additional pharmaceutical compositions for treatment of conditions (e.g., retinal detachment, blood disorders, or cancer) described herein.

The pharmaceutical compound may be administered in the form of a composition which is formulated with a pharmaceutically acceptable carrier and optional excipients, adjuvants, etc. in accordance with good pharmaceutical practice. The pharmaceutical composition may be in the form of a solid, semi-solid or liquid dosage form: such as powder, solution, elixir, syrup, suspension, cream, drops, paste and spray. As those skilled in the art would recognize, depending on the chosen route of administration (e.g. eye drops, injection, etc.), the composition form is determined. In general, it is preferred to use a unit dosage form of the compound or agent in order to achieve an easy and accurate administration of the active pharmaceutical compound. In general, the therapeutically effective pharmaceutical compound is present in such a dosage form at a concentration level ranging from about 0.5% to about 99% by weight of the total composition: e.g., in an amount sufficient to provide the desired unit dose. In some embodiments, the pharmaceutical composition may be administered in single or multiple doses. The particular route of administration and the dosage regimen will be determined by one of skill in keeping with the condition of the individual to be treated and said individual's response to the treatment. In some embodiments, a pharmaceutical composition in a unit dosage form for administration to a subject, comprising a pharmaceutical compound and one or more nontoxic pharmaceutically acceptable carriers, adjuvants or vehicles. The amount of the active ingredient that may be combined with such materials to produce a single dosage form will vary depending upon various factors, as indicated above. A variety of materials can be used as carriers, adjuvants and vehicles in the composition of the disclosure, as available in the pharmaceutical art. Injectable preparations, such as oleaginous solutions, suspensions or emulsions, may be formulated as known in the art, using suitable dispersing or wetting agents and suspending agents, as needed. The sterile injectable preparation may employ a nontoxic parenterally acceptable diluent or solvent such as sterile nonpyrogenic water or 1,3-butanediol. Among the other acceptable vehicles and solvents that may be employed are 5% dextrose injection, Ringer's injection and isotonic sodium chloride injection (as described in the USP/NF). In addition, sterile, fixed oils may be conventionally employed as solvents or suspending media. For this purpose, any bland fixed oil may be used, including synthetic mono-, di- or triglycerides. Fatty acids such as oleic acid can also be used in the preparation of injectable compositions.

In some embodiments, compositions of the present disclosure (e.g., small molecule PKM2 activator) are administered optically, for example, using the techniques described herein, and/or other techniques (e.g. injection, topical administration, etc.) (See, e.g., Janoria et al. Expert Opinion on Drug Delivery. July 2007, Vol. 4, No. 4, Pages 371-388; Ghate & Edelhauser. Expert Opin Drug Deliv. 2006 March; 3(2):275-87; Bourges et al. Adv Drug Deliv Rev. 2006 Nov. 15; 58(11):1182-202. Epub 2006 Sep. 22; Gomes Dos Santos et al. Curr Pharm Biotechnol. 2005 February; 6(1):7-15; herein incorporated by reference in their entireties).

In some embodiments, compositions (e.g., small molecule PKM2 activator) are provided as part of a kit. In some embodiments, a kit of the present disclosure comprises one or more compositions and/or pharmaceutical compositions. In some embodiments, a kit comprises a composition is configured for co-administration with one or more additional compositions (e.g. pharmaceutical compositions). In some embodiments, one or more compositions are co-administered with one or more other agents for effective protection of photoreceptors and/or inhibition of apoptosis or treatment of blood disorders or cancer.

### EXPERIMENTAL

The following examples are provided in order to demonstrate and further illustrate certain preferred embodiments and aspects of the present disclosure and are not to be construed as limiting the scope thereof.

### Example 1

Exemplary compounds are described below by reference to the illustrative synthetic schemes for their general preparation below and the specific examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. Reactions may be performed between the melting point and the reflux temperature of the solvent, and preferably between 0° C. and the reflux temperature of the solvent. Reactions may be heated employing conventional heating or microwave heating. Reactions may also be conducted in sealed pressure vessels above the normal reflux temperature of the solvent.

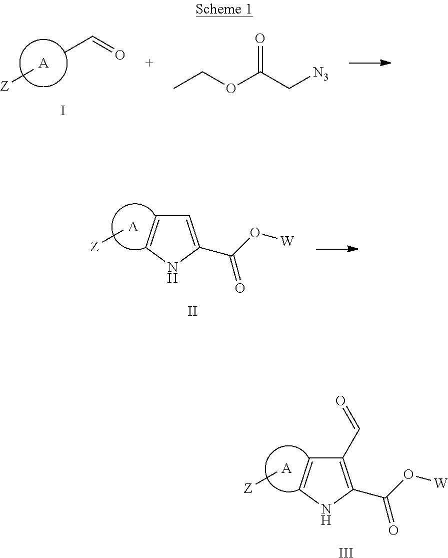

Compound II (Scheme 1) is purchased from commercially available sources or generated from commercially available compound I through reaction with ethyl azidoacetate under standard Hemetsberger conditions. Conversion of compound II to compound III is accomplished through the use of standard conditions employing the Vilsmeier-Haack reagent.

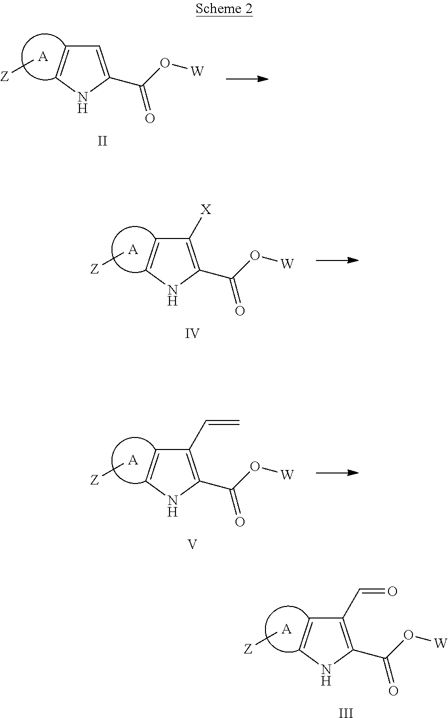

In cases where compound II is not compatible with the Vilsmeier-Haack reaction conditions, compound III is generated through the synthetic sequence shown in Scheme 2. Wherein compound II is treated with N-bromosuccinimide, N-chlorosuccinimide or N-iodosuccinimide generating compound IV where X is Cl, Br or I. Compound IV is converted to compound V by treatment with trimethyl vinyl tin under standard Stille cross coupling. Conversion of compound V to compound III is accomplished by treatment with ozone gas to facilitate olefin cleavage and subsequent treatment with triphenylphosphine provides the desired aldehyde. Alternatively, compound V is converted to compound III through olefin dihydroxylation and metal mediated cleavage. The appropriate reagents compatible with the substrate are selected to accomplish the desired dihydroxylation and cleavage event.

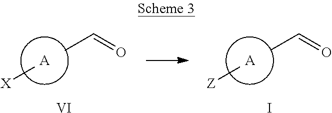

In cases where compound I with the desired “Z” moiety is not commercially available the desired compound can be generated via a SnAR or suitable metal mediated cross coupling reaction from a desired precursor, compound VI, where X is a functional group or protected functional group that will allow for the desired reactivity and functional group incorporation. In some cases the protecting group is removed prior to reaction.

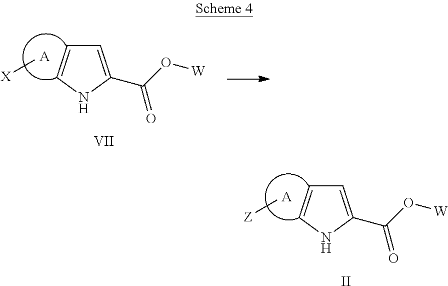

In cases where compound II with the desired “Z” moiety is not commercially available the desired compound can be generated via a SnAR or suitable metal mediated cross coupling reaction from a desired precursor, compound VI, where X is a functional group or protected functional group that will allow for the desired reactivity and functional group incorporation. In some cases the protecting group is removed prior to reaction.

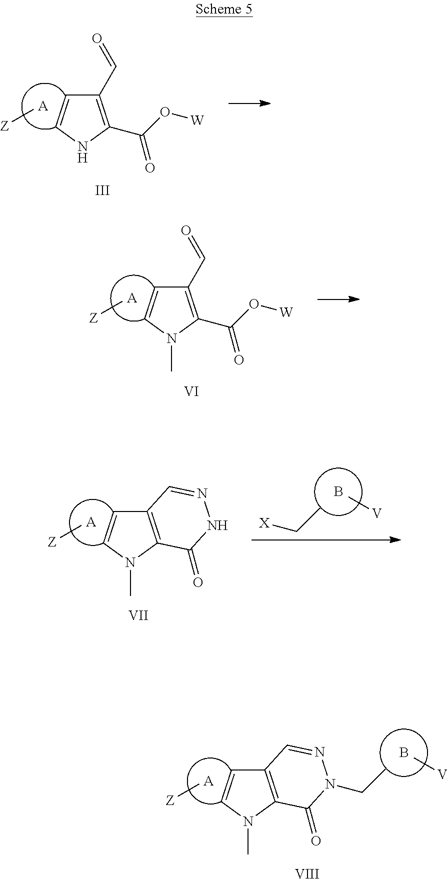

Compound III is converted to compound VIII by treatment with methyliodide and potassium carbonate in dimethylformamide. Other bases such as cesium carbonate, sodium hydride and other provide the same result. Treatment of compound VIII with hydrazine in ethoxyethanol at elevated temperatures results in the formation of compound IX. Generation of compound X is accomplished by treatment of compound IX with potassium tert-butoxide, or equivalent base, and desired alkylating agent. Alkylating agents my contain desired functionality masked by protecting groups to facilitate the desired reactivity.

### Chemistry:

In obtaining the compounds described in the examples below and the corresponding analytical data, the following experimental and analytical protocols were followed unless otherwise indicated.

Unless otherwise stated, reaction mixtures were magnetically stirred at room temperature (rt) under a nitrogen atmosphere. Where solutions were “dried,” they were generally dried over a drying agent such as Na₂SO₄ or MgSO₄. Where mixtures, solutions, and extracts were “concentrated”, they were typically concentrated on a rotary evaporator under reduced pressure.

Normal-phase flash column chromatography (FCC) was performed on silica gel (SiO2) using prepackaged cartridges, eluting with the indicated solvents.

NMR spectra were obtained on a Bruker 400 Ascend™ spectrometer at a ¹H frequency of 400 MHz and a ¹³C frequency of 100 MHz. Chemical shifts (δ) are reported in parts per million (ppm) relative to an internal standard. The final products were purified on a preparative HPLC column (Waters 2545, Quaternary Gradient Module) with a SunFire Prep C18 OBD 5 μm 50×100 mm reverse phase column. The mobile phase was a gradient of solvent A (H2O with 0.1% TFA) and solvent B (MeCN with 0.1% TFA) at a flow rate of 60 mL/min and 1%/min increase of solvent B. All final compounds have purity≥95% as determined by Waters ACQUITY UPLC using reverse phase column (SunFire, C18-5 μm, 4.6×150 mm) and a solvent gradient of A (H2O with 0.1% of TFA) and solvent B (CH3CN with 0.1% of TFA). ESI mass spectral analysis was performed on a Thermo-Scientific LCQ Fleet mass spectrometer.

Chemical names were generated using ChemDraw Ultra 6.0.2 (CambridgeSoft Corp., Cambridge, MA) or ACD/Name Version 9 (Advanced 5 Chemistry Development, Toronto, Ontario, Canada).

### Intermediate A: Methyl 3-formyl-6-methyl-1H-indole-2-carboxylate

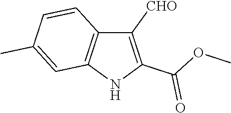

Phosphorous oxychloride (0.30 mL, 3.18 mmol) was added dropwise to a DMF (6 mL) at 0° C. The resulting reaction mixture was allowed to warm to room temperature and stir for 10 minutes. The reaction mixture was cooled to 0° C. and methyl 6-methyl-1h-indole-2-carboxylate (503 mg, 2.66 mmol) in DMF (3 mL) was added dropwise. The reaction mixture was heated to 80° C. and stirred for 1.5 hours, cooled to room temperature and poured into 50 mL of ice water. The resulting precipitate was collected via vacuum filtration to provide the desired product (478 mg, 83%). ¹H NMR (400 MHz, DMSO-d6) δ 12.76 (s, 1H), 10.59 (s, 1H), 8.12 (d, J=8.3 Hz, 1H), 7.37-7.34 (m, 1H), 7.15 (dd, J=8.3, 1.4 Hz, 1H), 3.99 (s, 3H), 2.44 (s, 3H).

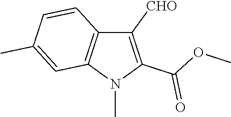

### Intermediate B: Methyl 3-formyl-1,6-dimethyl-1H-indole-2-carboxylate

To a solution of methyl 3-formyl-6-methyl-1H-indole-2-carboxylate (478 mg, 2.20 mmol) in DMF (4 mL) was added K₂CO₃ (911 mg, 6.60 mmol) and iodomethane (0.15 mL, 2.42 mmol). The reaction mixture was stirred at room temperature overnight and poured into water (40 mL). The resulting precipitate was collected via vacuum filtration providing the desired product (451 mg, 87%). ¹H NMR (400 MHz, DMSO-d6) δ 10.41 (s, 1H), 8.18 (d, J=8.2 Hz, 1H), 7.58-7.52 (m, 1H), 7.26-7.17 (m, 1H), 4.02 (s, 3H), 4.00 (s, 3H), 2.49 (s, 3H).

### Intermediate C: 5,7-Dimethyl-3,5-dihydro-4H-pyridazino[4,5-b]indol-4-one

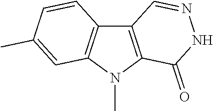 [SMILES: Cc1ccc2c3cn[nH]c(=O)c3n(C)c2c1]

To a solution of methyl 3-formyl-1,6-dimethyl-1H-indole-2-carboxylate (451 mg, 1.95 mmol) in ethoxyethanol (5 mL) was added hydrazine (7.81 mmol) and the reaction mixture was heated to 135° C. overnight. The reaction mixture was cooled to room temperature and water was added. The reaction mixture was extracted with EtOAc (3×10 mL). The organics were combined, dried, concentrated and purified by flash column chromatography (0-20% MeOH in DCM) to provide the desired product (239 mg, 58%). ¹H NMR (400 MHz, DMSO-d6) δ 12.75 (s, 1H), 8.71 (s, 1H), 8.07 (d, J=8.1 Hz, 1H), 7.58-7.52 (m, 1H), 7.23 (dd, J=8.1, 1.4 Hz, 1H), 4.25 (s, 3H), 2.54 (s, 3H).

### Example 1: 3-Benzyl-5,7-dimethyl-3,5-dihydro-4H-pyridazino[4,5-b]indol-4-one

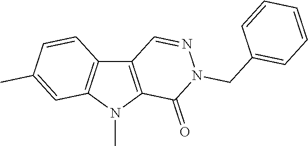 [SMILES: Cc1ccc2c3cnn(Cc4ccccc4)c(=O)c3n(C)c2c1]

To a solution of 5,7-dimethyl-3,5-dihydro-4H-pyridazino[4,5-b]indol-4-one (53 mg, 0.25 mmol) and KOtBu (35 mg, 0.29 mmol) in DMF (5 mL) at 0° C. was added benzyl bromide (51 mg, 0.30 mmol). The reaction mixture was warmed to room temperature and stirred overnight. Water was added and the reaction mixture was extracted with DCM (3×10 mL). The organics were combined, dried, concentrated and purified by flash column chromatography (0-100% EtOAc in hexanes) to provide the desired product (45 mg, 61%). ¹H NMR (400 MHz, DMSO-d6) δ 8.78 (s, 1H), 8.08 (d, J=8.0 Hz, 1H), 7.57 (s, 1H), 7.36-7.31 (m, 4H), 7.31-7.22 (m, 2H), 5.41 (s, 2H), 4.26 (s, 3H), 2.54 (s, 3H).

### Example 2: 3-(4-Methoxybenzyl)-5,7-dimethyl-3,5-dihydro-4H-pyridazino[4,5-b]indol-4-one

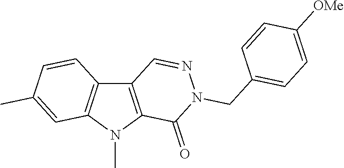 [SMILES: COc1ccc(Cn2ncc3c4ccc(C)cc4n(C)c3c2=O)cc1]

Example 2 was prepared in analogous fashion to Example 1. ¹H NMR (400 MHz, DMSO-d6) δ 8.76 (s, 1H), 8.07 (d, J=8.2 Hz, 1H), 7.56 (s, 1H), 7.35-7.28 (m, 2H), 7.24 (dd, J=8.2, 1.4 Hz, 1H), 6.93-6.86 (m, 2H), 5.33 (s, 2H), 4.26 (s, 3H), 3.72 (s, 3H), 2.54 (s, 3H).

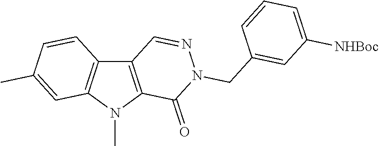 [SMILES: Cc1ccc2c3cnn(Cc4cccc(NC(=O)OC(C)(C)C)c4)c(=O)c3n(C)c2c1]

### Intermediate D: tert-butyl (3-((5,7-Dimethyl-4-oxo-4,5-dihydro-3H-pyridazino[4,5-b]indol-3-yl)methyl)phenyl)carbamate

Intermediate D was prepared in analogous fashion to Example 1. ¹H NMR (400 MHz, DMSO-d6) δ 9.31 (s, 1H), 8.79 (s, 1H), 8.09 (d, J=8.2 Hz, 1H), 7.57 (s, 1H), 7.44-7.38 (m, 1H), 7.39-7.31 (m, 1H), 7.26-7.22 (m, 1H), 7.22-7.16 (m, 1H), 6.95-6.89 (m, 1H), 5.34 (s, 2H), 4.26 (s, 3H), 2.54 (s, 3H), 1.44 (s, 9H).

### Example 3: 3-(3-Aminobenzyl)-5,7-dimethyl-3,5-dihydro-4H-pyridazino[4,5-b]indol-4-one

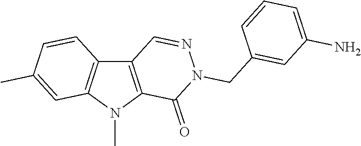 [SMILES: Cc1ccc2c3cnn(Cc4cccc(N)c4)c(=O)c3n(C)c2c1]

To a solution of tert-butyl (3-((5,7-Dimethyl-4-oxo-4,5-dihydro-3H-pyridazino[4,5-b]indol-3-yl)methyl)phenyl)carbamate (53 mg, 0.13 mmol) in DCM (5 mL) was added HCl (1 mL, 4 M HCl in dioxane) and the reaction mixture was stirred at room temperature for 1 hour. The reaction mixture was diluted with DCM (10 mL), washed with saturated aqueous sodium bicarbonate (2×5 mL), dried, concentrated and purified by hplc to provide the desired product (24 mg, 60%).

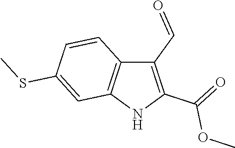

### Intermediate E: Methyl 3-formyl-6-(methylthio)-1H-indole-2-carboxylate

Intermediate E was generated in analogous fashion to Intermediate A. ¹H NMR (400 MHz, DMSO-d6) δ 12.79 (s, 1H), 10.58 (s, 1H), 8.15 (d, J=8.5 Hz, 1H), 7.36 (d, J=1.6 Hz, 1H), 7.23 (dd, J=8.6, 1.7 Hz, 1H), 3.99 (s, 3H), 2.54 (s, 3H).

### Intermediate F: Methyl 3-formyl-1-methyl-6-(methylthio)-1H-indole-2-carboxylate

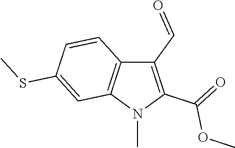

Intermediate F was generated in analogous fashion to Intermediate B. ¹H NMR (400 MHz, DMSO-d6) δ 10.41 (s, 1H), 8.19 (dd, J=8.5, 0.6 Hz, 1H), 7.57 (d, J=1.5 Hz, 1H), 7.27 (dd, J=8.5, 1.6 Hz, 1H), 4.05 (s, 3H), 4.00 (s, 3H), 2.60 (s, 3H).

### Intermediate G: 5-Methyl-7-(methylthio)-3,5-dihydro-4H-pyridazino[4,5-b]indol-4-one

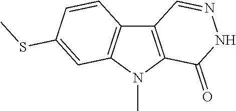 [SMILES: CSc1ccc2c3cn[nH]c(=O)c3n(C)c2c1]

Intermediate G was prepared in analogous fashion to Intermediate C. ¹H NMR (400 MHz, DMSO-d6) δ 12.90-12.71 (m, 1H), 8.72 (s, 1H), 8.11 (dd, J=8.4, 0.6 Hz, 1H), 7.57 (dd, J=1.7, 0.7 Hz, 1H), 7.29 (dd, J=8.4, 1.6 Hz, 1H), 4.27 (s, 3H), 2.63 (s, 3H).

### Example 4: 3-Benzyl-5-methyl-7-(methylthio)-3,5-dihydro-4H-pyridazino[4,5-b]indol-4-one

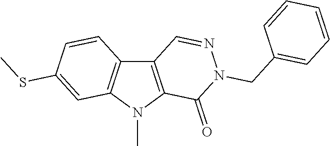 [SMILES: CSc1ccc2c3cnn(Cc4ccccc4)c(=O)c3n(C)c2c1]

Example 4 was prepared in analogous fashion to Example 1. ¹H NMR (400 MHz, DMSO-d6) δ 8.79 (s, 1H), 8.11 (dd, J=8.4, 0.6 Hz, 1H), 7.57 (dd, J=1.6, 0.6 Hz, 1H), 7.36-7.25 (m, 6H), 5.41 (s, 2H), 4.28 (s, 3H), 2.63 (s, 3H).

### Example 5: 3-Benzyl-5-methyl-7-(methylsulfinyl)-3,5-dihydro-4H-pyridazino[4,5-b]indol-4-one

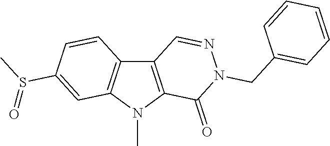 [SMILES: Cn1c2cc(S(C)=O)ccc2c2cnn(Cc3ccccc3)c(=O)c21]

To a solution of 3-Benzyl-5-methyl-7-(methylthio)-3,5-dihydro-4H-pyridazino[4,5-b]indol-4-one (27.0 mg, 0.081 mmol) in dichloromethane (10 mL) was added 3-chloroperoxybenzoic acid (13.9 mg, 0.081 mmol). The reaction mixture was stirred at room temperature overnight. The reaction mixture was diluted with DCM (20 mL), extracted with saturated aqueous sodium bicarbonate (3×10 mL), dried, concentrated and purified by flash column chromatography (0-100% EtOAc in hexanes) to provide the desired product (10 mg, 53%). ¹H NMR (400 MHz, DMSO-d6) δ 8.89 (s, 1H), 8.41 (dd, J=8.4, 0.7 Hz, 1H), 8.11 (dd, J=1.4, 0.7 Hz, 1H), 7.67 (dd, J=8.3, 1.4 Hz, 1H), 7.37-7.31 (m, 4H), 7.31-7.25 (m, 1H), 5.43 (s, 2H), 4.35 (s, 3H), 2.85 (s, 3H).

### Intermediate H: Tert-butyl (3-((5-methyl-7-(methylthio)-4-oxo-4,5-dihydro-3H-pyridazino[4,5-b]indol-3-yl)methyl)phenyl)carbamate

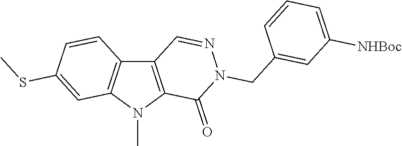 [SMILES: CSc1ccc2c3cnn(Cc4cccc(NC(=O)OC(C)(C)C)c4)c(=O)c3n(C)c2c1]

Intermediate H was prepared in analogous fashion to example 1 from 5-Methyl-7-(methylthio)-3,5-dihydro-4H-pyridazino[4,5-b]indol-4-one. ¹H NMR (400 MHz, DMSO-d6) δ 9.31 (s, 1H), 8.79 (s, 1H), 8.15-8.10 (m, 1H), 7.59-7.54 (m, 1H), 7.44-7.39 (m, 1H), 7.33-7.26 (m, 1H), 7.22-7.16 (m, 1H), 7.17-7.07 (m, 1H), 6.94-6.89 (m, 1H), 5.34 (s, 2H), 4.27 (s, 3H), 2.63 (s, 3H), 1.45 (s, 9H).

### Example 6: 3-Benzyl-5-methyl-7-(methylthio)-3,5-dihydro-4H-pyridazino[4,5-b]indol-4-one

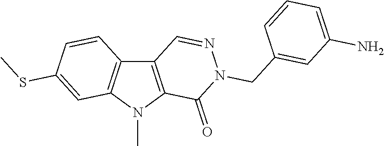 [SMILES: CSc1ccc2c3cnn(Cc4cccc(N)c4)c(=O)c3n(C)c2c1]

Example 6 was prepared in analogous fashion to Example 3 from tert-butyl (3-((5-methyl-7-(methylthio)-4-oxo-4,5-dihydro-3H-pyridazino[4,5-b]indol-3-yl)methyl)phenyl)carbamate. ¹H NMR (400 MHz, DMSO-d6) δ 8.77 (s, 1H), 8.12 (dd, J=8.4, 0.6 Hz, 1H), 7.57 (d, J=1.6 Hz, 1H), 7.30 (dd, J=8.4, 1.6 Hz, 1H), 7.03-6.90 (m, 1H), 6.54-6.39 (m, 3H), 5.24 (s, 2H), 5.05 (s, 2H), 4.28 (s, 3H), 2.63 (s, 3H).

### Intermediate I: Tert-butyl (3-((5-methyl-7-(methylsulfinyl)-4-oxo-4,5-dihydro-3H-pyridazino[4,5-b]indol-3-yl)methyl)phenyl)carbamate

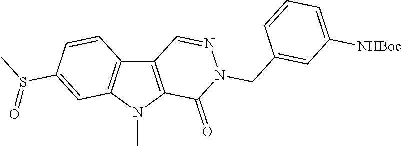 [SMILES: Cn1c2cc(S(C)=O)ccc2c2cnn(Cc3cccc(NC(=O)OC(C)(C)C)c3)c(=O)c21]

Intermediate I was prepared in analogous fashion to Example 5 from tert-butyl (3-((5-methyl-7-(methylthio)-4-oxo-4,5-dihydro-3H-pyridazino[4,5-b]indol-3-yl)methyl)phenyl)carbamate. ¹H NMR (400 MHz, DMSO-d6) δ 9.31 (s, 1H), 8.90 (s, 1H), 8.45-8.39 (m, 1H), 8.14-8.08 (m, 1H), 7.71-7.64 (m, 1H), 7.44-7.39 (m, 1H), 7.39-7.33 (m, 1H), 7.23-7.17 (m, 1H), 6.96-6.89 (m, 1H), 5.36 (s, 2H), 4.34 (s, 3H), 2.86 (s, 3H), 1.45 (s, 9H).

### Example 7: 3-(3-Aminobenzyl)-5-methyl-7-(methylsulfinyl)-3,5-dihydro-4H-pyridazino[4,5-b]indol-4-one

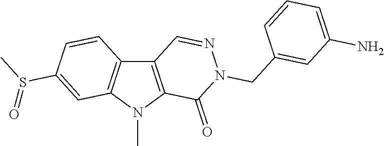 [SMILES: Cn1c2cc(S(C)=O)ccc2c2cnn(Cc3cccc(N)c3)c(=O)c21]

Example 7 was generated in analogous fashion to Example 3 from tert-butyl (3-((5-methyl-7-(methylsulfinyl)-4-oxo-4,5-dihydro-3H-pyridazino[4,5-b]indol-3-yl)methyl)phenyl)carbamate. ¹H NMR (400 MHz, DMSO-d6) δ 8.91 (s, 1H), 8.42 (d, J=8.3 Hz, 1H), 8.12 (d, J=1.3 Hz, 1H), 7.73-7.64 (m, 1H), 7.28-7.20 (m, 1H), 7.03-6.94 (m, 1H), 6.93-6.83 (m, 2H), 5.38 (s, 2H), 4.35 (s, 3H), 2.85 (s, 3H).

### Intermediate J: Methyl 3-formyl-6-methoxy-1H-indole-2-carboxylate

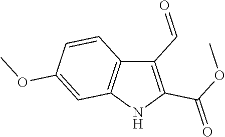 [SMILES: COC(=O)c1[nH]c2cc(OC)ccc2c1C=O]

Intermediate J was generated in analogous fashion to Intermediate A from commercially available methyl 6-methoxy-1h-indole-2-carboxylate. ¹H NMR (400 MHz, DMSO-d6) δ 12.67 (s, 1H), 10.58 (s, 1H), 8.13-8.08 (m, 1H), 7.00-6.93 (m, 2H), 3.98 (s, 3H), 3.82 (s, 3H).

### Intermediate K: Methyl 3-formyl-6-methoxy-1-methyl-1H-indole-2-carboxylate

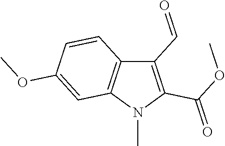 [SMILES: COC(=O)c1c(C=O)c2ccc(OC)cc2n1C]

Intermediate K was generated in analogous fashion to Intermediate B from methyl 3-formyl-6-methoxy-1H-indole-2-carboxylate. ¹H NMR (400 MHz, DMSO-d6) δ 10.42 (s, 1H), 8.16 (d, J=8.8 Hz, 1H), 7.27-7.19 (m, 1H), 7.00 (dd, J=8.9, 2.3 Hz, 1H), 4.03 (d, J=1.4 Hz, 3H), 3.99 (s, 3H), 3.88 (s, 3H).

### Intermediate L: 7-Methoxy-5-methyl-3,5-dihydro-4H-pyridazino[4,5-b]indol-4-one

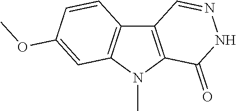 [SMILES: COc1ccc2c3cn[nH]c(=O)c3n(C)c2c1]

Intermediate L was generated in analogous fashion to Intermediate C from methyl 3-formyl-6-methoxy-1-methyl-1H-indole-2-carboxylate. 1H NMR (400 MHz, DMSO-d6) δ 12.69 (s, 1H), 8.67 (s, 1H), 8.06 (d, J=8.8 Hz, 1H), 7.23 (d, J=2.2 Hz, 1H), 7.01 (dd, J=8.7, 2.2 Hz, 1H), 4.24 (s, 3H), 3.91 (s, 3H).

### Intermediate M: Tert-butyl (3-((7-methoxy-5-methyl-4-oxo-4,5-dihydro-3H-pyridazino[4,5-b]indol-3-yl)methyl)phenyl)carbamate

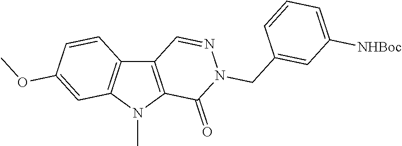 [SMILES: COc1ccc2c3cnn(Cc4cccc(NC(=O)OC(C)(C)C)c4)c(=O)c3n(C)c2c1]

Intermediate M was generated in analogous fashion to Example 1 from 7-Methoxy-5-methyl-3,5-dihydro-4H-pyridazino[4,5-b]indol-4-one. ¹H NMR (400 MHz, DMSO-d6) δ 9.29 (s, 1H), 8.75 (s, 1H), 8.08 (d, J=8.8 Hz, 1H), 7.44-7.31 (m, 2H), 7.25 (d, J=2.2 Hz, 1H), 7.22-7.15 (m, 1H), 7.02 (dd, J=8.8, 2.2 Hz, 1H), 6.96-6.85 (m, 1H), 5.34 (s, 2H), 4.26 (s, 3H), 3.92 (s, 3H), 1.44 (s, 9H).

### Example 8: 3-(3-Aminobenzyl)-7-methoxy-5-methyl-3,5-dihydro-4H-pyridazino[4,5-b]indol-4-one

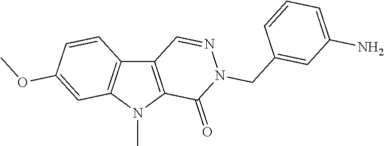 [SMILES: COc1ccc2c3cnn(Cc4cccc(N)c4)c(=O)c3n(C)c2c1]

Example 8 was generated in analogous fashion to Example 3 from tert-butyl (3-((7-methoxy-5-methyl-4-oxo-4,5-dihydro-3H-pyridazino[4,5-b]indol-3-yl)methyl)phenyl)carbamate. ¹H NMR (400 MHz, DMSO-d6) δ 8.73 (s, 1H), 8.08 (d, J=8.8 Hz, 1H), 7.24 (d, J=2.2 Hz, 1H), 7.02 (dd, J=8.8, 2.2 Hz, 1H), 6.99-6.89 (m, 1H), 6.51-6.41 (m, 3H), 5.24 (s, 2H), 5.08 (s, 2H), 4.26 (s, 3H), 3.92 (s, 3H).

### Example 9: 7-Methoxy-5-methyl-3-((4-oxo-4H-pyrido[1,2-a]pyrimidin-2-yl)methyl)-3,5-dihydro-4H-pyridazino[4,5-b]indol-4-one

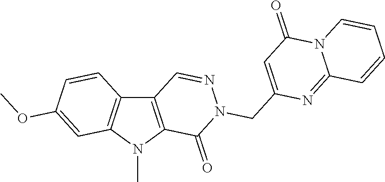 [SMILES: COc1ccc2c3cnn(Cc4cc(=O)n5ccccc5n4)c(=O)c3n(C)c2c1]

Example 9 was generated in analogous fashion to Example 3 from 7-methoxy-5-methyl-3,5-dihydro-4H-pyridazino[4,5-b]indol-4-one. ¹H NMR (400 MHz, DMSO-d6) δ 8.96-8.92 (m, 1H), 8.82 (s, 1H), 8.11 (d, J=8.8 Hz, 1H), 8.02-7.94 (m, 1H), 7.70-7.64 (m, 1H), 7.40-7.33 (m, 1H), 7.29-7.25 (m, 1H), 7.08-7.02 (m, 1H), 5.94 (s, 1H), 5.44-5.35 (m, 2H), 4.26 (s, 3H), 3.93 (s, 3H).

### Example 10: 3-(Imidazo[1,2-a]pyrimidin-2-ylmethyl)-7-methoxy-5-methyl-3,5-dihydro-4H-pyridazino[4,5-b]indol-4-one

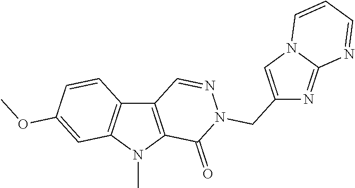 [SMILES: COc1ccc2c3cnn(Cc4cn5cccnc5n4)c(=O)c3n(C)c2c1]

Example 10 was generated in analogous fashion to Example 3 from 7-methoxy-5-methyl-3,5-dihydro-4H-pyridazino[4,5-b]indol-4-one. ¹H NMR (400 MHz, DMSO-d6) δ 8.87 (dd, J=6.8, 2.0 Hz, 1H), 8.77 (s, 1H), 8.49 (dd, J=4.1, 2.0 Hz, 1H), 8.10 (d, J=8.8 Hz, 1H), 7.69 (d, J=0.8 Hz, 1H), 7.26 (d, J=2.2 Hz, 1H), 7.07-6.99 (m, 2H), 5.60-5.50 (m, 2H), 4.27 (s, 3H), 3.92 (s, 3H).

### Example 11: 3-(Imidazo[1,2-a]pyridin-2-ylmethyl)-7-methoxy-5-methyl-3,5-dihydro-4H-pyridazino[4,5-b]indol-4-one

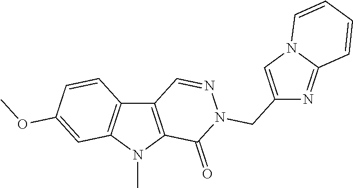 [SMILES: COc1ccc2c3cnn(Cc4cn5ccccc5n4)c(=O)c3n(C)c2c1]

Example 11 was generated in analogous fashion to Example 3 from 7-methoxy-5-methyl-3,5-dihydro-4H-pyridazino[4,5-b]indol-4-one. ¹H NMR (400 MHz, DMSO-d6) δ 8.74 (s, 1H), 8.49-8.43 (m, 1H), 8.08 (dd, J=8.8, 0.5 Hz, 1H), 7.73 (d, J=0.8 Hz, 1H), 7.51-7.44 (m, 1H), 7.25 (d, J=2.1 Hz, 1H), 7.20 (ddd, J=9.1, 6.7, 1.3 Hz, 1H), 7.02 (dd, J=8.7, 2.2 Hz, 1H), 6.90-6.81 (m, 1H), 5.50 (s, 2H), 4.27 (s, 3H), 3.92 (s, 3H).

### Example 12: 7-Methoxy-5-methyl-3-(quinolin-6-ylmethyl)-3,5-dihydro-4H-pyridazino[4,5-b]indol-4-one

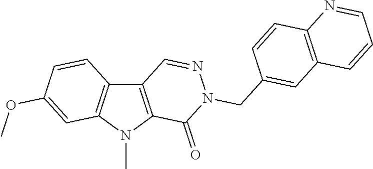 [SMILES: COc1ccc2c3cnn(Cc4ccc5ncccc5c4)c(=O)c3n(C)c2c1]

Example 12 was generated in analogous fashion to Example 3 from 7-methoxy-5-methyl-3,5-dihydro-4H-pyridazino[4,5-b]indol-4-one. ¹H NMR (400 MHz, DMSO-d6) δ 8.87 (dd, J=4.2, 1.7 Hz, 1H), 8.80 (s, 1H), 8.37-8.32 (m, 1H), 8.09 (d, J=8.8 Hz, 1H), 8.00 (d, J=8.7 Hz, 1H), 7.89-7.83 (m, 1H), 7.76 (dd, J=8.7, 2.0 Hz, 1H), 7.51 (dd, J=8.3, 4.2 Hz, 1H), 7.25 (d, J=2.2 Hz, 1H), 7.03 (dd, J=8.7, 2.2 Hz, 1H), 5.61 (s, 2H), 4.27 (s, 3H), 3.92 (s, 3H).

### Example 13: 7-Methoxy-5-methyl-3-((5-methyl-1,3,4-oxadiazol-2-yl)methyl)-3,5-dihydro-4H-pyridazino[4,5-b]indol-4-one

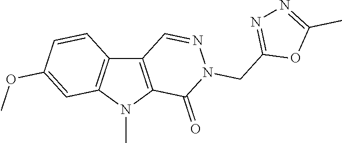

Example 13 was generated in analogous fashion to Example 3 from 7-methoxy-5-methyl-3,5-dihydro-4H-pyridazino[4,5-b]indol-4-one. ¹H NMR (400 MHz, DMSO-d6) δ 8.78 (s, 1H), 8.13-8.04 (m, 1H), 7.26 (d, J=2.1 Hz, 1H), 7.04 (dd, J=8.8, 2.2 Hz, 1H), 5.62 (s, 2H), 4.24 (s, 3H), 3.92 (s, 3H), 2.47 (s, 3H).

### Example 14: 7-Methoxy-5-methyl-3-(pyrimidin-2-ylmethyl)-3,5-dihydro-4H-pyridazino[4,5-b]indol-4-one

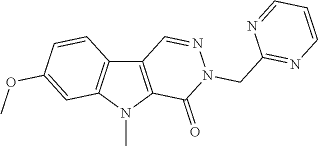 [SMILES: COc1ccc2c3cnn(Cc4ncccn4)c(=O)c3n(C)c2c1]

Example 14 was generated in analogous fashion to Example 3 from 7-methoxy-5-methyl-3,5-dihydro-4H-pyridazino[4,5-b]indol-4-one. ¹H NMR (400 MHz, DMSO-d6) δ 8.80-8.71 (m, 3H), 8.10 (d, J=8.8 Hz, 1H), 7.41 (t, J=4.9 Hz, 1H), 7.26 (d, J=2.2 Hz, 1H), 7.04 (dd, J=8.8, 2.2 Hz, 1H), 5.60 (s, 2H), 4.24 (s, 3H), 3.92 (s, 3H).

### Example 15: 3-(3-Aminobenzyl)-7-hydroxy-5-methyl-3,5-dihydro-4H-pyridazino[4,5-b]indol-4-one

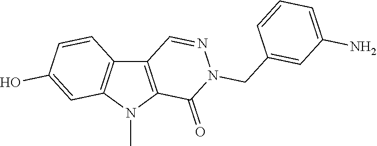 [SMILES: Cn1c2cc(O)ccc2c2cnn(Cc3cccc(N)c3)c(=O)c21]

To a solution of tert-butyl (3-((7-methoxy-5-methyl-4-oxo-4,5-dihydro-3H-pyridazino[4,5-b]indol-3-yl)methyl)phenyl)carbamate (76.4 mg, 0.176 mmol) in DCM (10 mL) at 0° C. was added 1 M tribromoborane in DCM (1.00 mL) and the reaction mixture was warmed to room temperature and 3 hours. Saturated aqueous sodium bicarbonate (10 mL) was added and the resulting reaction mixture was extracted with DCM. The organic layers were combined, dried, concentrated and purified by flash column chromatography (0-20% MeOH in DCM) to provide the desired product (32 mg, 57%). ¹H NMR (400 MHz, DMSO-d6) δ 9.95 (s, 1H), 8.68 (s, 1H), 7.98 (d, J=8.6 Hz, 1H), 7.00-6.85 (m, 3H), 6.50-6.38 (m, 2H), 5.23 (s, 2H), 5.02 (s, 2H), 4.18 (s, 3H).

### Intermediate N: Ethyl 2-methyl-6H-thieno[2,3-b]pyrrole-5-carboxylate

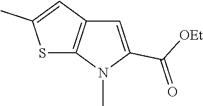

To a solution of 5-methylthiophene-3-carbaldehyde (1.00 g, 7.93 mmol) in ethanol (50 mL) at 0° C. was added ethyl 2-azidoacetate (1.33 g, 10.3 mmol) followed by the dropwise addition of sodium ethoxide in ethanol (10.3 mmol). The reaction mixture was warmed to room temperature and stirred overnight. Saturated aqueous ammonium chloride (50 mL) was added and the resulting reaction mixture was extracted with DCM (3×20 mL). The organics were dried, concentrated, dissolved in toluene and refluxed for 1.5 hours. The reaction mixture was concentrated and purified by flash column chromatography (0-30% EtOAc in hexanes) to provide the desired product. ¹H NMR (400 MHz, DMSO-d6) δ 12.10 (s, 1H), 6.86 (d, J=2.0 Hz, 1H), 6.71 (dt, J=1.6, 1.2 Hz, 1H), 4.26 (q, J=7.1 Hz, 2H), 2.45 (d, J=1.3 Hz, 3H), 1.30 (t, J=7.1 Hz, 3H).

### Intermediate O: Ethyl 4-formyl-2-methyl-6H-thieno[2,3-b]pyrrole-5-carboxylate

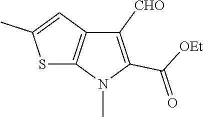 [SMILES: CCOC(=O)c1c(C=O)c2cc(C)sc2n1C]

Intermediate O was generated in analogous fashion to Intermediate A from ethyl 2-methyl-6H-thieno[2,3-b]pyrrole-5-carboxylate. ¹H NMR (400 MHz, DMSO-d6) δ 12.97 (s, 1H), 10.42 (s, 1H), 7.11-6.97 (m, 1H), 4.39 (q, J=7.1 Hz, 2H), 3.30 (s, 3H), 1.37 (t, J=7.1 Hz, 3H).

### Intermediate P: Ethyl 4-formyl-2,6-dimethyl-6H-thieno[2,3-b]pyrrole-5-carboxylate

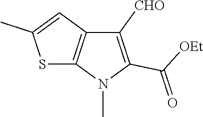 [SMILES: CCOC(=O)c1c(C=O)c2cc(C)sc2n1C]

Intermediate P was generated in analogous fashion to Intermediate B from ethyl 4-formyl-2-methyl-6H-thieno[2,3-b]pyrrole-5-carboxylate. ¹H NMR (400 MHz, DMSO-d6) δ 10.35 (s, 1H), 7.11-7.07 (m, 1H), 4.40 (q, J=7.1 Hz, 2H), 3.99 (s, 3H), 2.56-2.53 (m, 3H), 1.37 (t, J=7.1 Hz, 3H).

Intermediate Q: 2,8-Dimethyl-6,8-dihydro-7H-thieno[3′,2′:4,5]pyrrolo[2,3-d]pyridazin-7-one

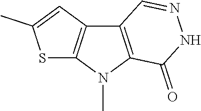

Intermediate Q was generated in analogous fashion to Intermediate C from ethyl 4-formyl-2,6-dimethyl-6H-thieno[2,3-b]pyrrole-5-carboxylate. ¹H NMR (400 MHz, DMSO-d6) δ 12.52 (s, 1H), 8.40 (s, 1H), 7.15 (s, 1H), 4.18 (s, 3H), 2.57 (s, 3H).

### Example 16: 6-(2-Fluorobenzyl)-2,8-dimethyl-6,8-dihydro-7H-thieno[3′,2′:4,5]pyrrolo[2,3-d]pyridazin-7-one

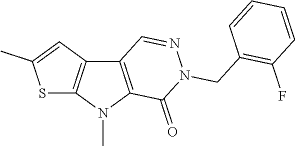 [SMILES: Cc1cc2c3cnn(Cc4ccccc4F)c(=O)c3n(C)c2s1]

Example 16 was generated in analogous fashion to Example 1 from 2,8-dimethyl-6,8-dihydro-7H-thieno[3′,2′:4,5]pyrrolo[2,3-d]pyridazin-7-one. ¹H NMR (400 MHz, DMSO-d6) δ 8.49 (s, 1H), 7.38-7.30 (m, 1H), 7.26-7.11 (m, 4H), 5.42 (s, 2H), 4.20 (s, 3H), 2.58 (s, 3H).

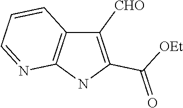 [SMILES: CCOC(=O)c1[nH]c2ncccc2c1C=O]

### Intermediate R: Ethyl 3-formyl-1H-pyrrolo[2,3-b]pyridine-2-carboxylate

To a solution of ethyl 1H-pyrrolo[2,3-B]pyridine-2-carboxylate (1.00 g, 5.25 mmol) in acetic acid (3 mL) and water (6 mL) was added hexamethylenetetramine (812 mg, 5.78) and the resulting reaction mixture was heated to 120° C. for 9 hours. The reaction mixture was cooled to room temperature and the volume was reduced by half. The reaction mixture was cooled to 0° C. and the resulting precipitate was collected via filtration to provide the desired product (52.2 mg, 5%). ¹H NMR (400 MHz, DMSO-d6) δ 13.44 (s, 1H), 10.59 (s, 1H), 8.59 (dd, J=8.0, 1.7 Hz, 1H), 8.55 (dd, J=4.6, 1.7 Hz, 1H), 7.38 (dd, J=8.0, 4.6 Hz, 1H), 4.47 (q, J=7.1 Hz, 2H), 1.41 (t, J=7.1 Hz, 3H).

### Intermediate S: Ethyl 3-formyl-1-methyl-1H-pyrrolo[2,3-b]pyridine-2-carboxylate

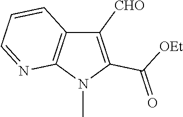 [SMILES: CCOC(=O)c1c(C=O)c2cccnc2n1C]

Intermediate S was generated in analogous fashion to Intermediate B from ethyl 3-formyl-1H-pyrrolo[2,3-b]pyridine-2-carboxylate. ¹H NMR (400 MHz, Chloroform-d) δ 10.64 (s, 1H), 8.78 (dd, J=8.0, 1.7 Hz, 1H), 8.58 (dd, J=4.6, 1.7 Hz, 1H), 7.33 (dd, J=8.0, 4.6 Hz, 1H), 4.57 (q, J=7.2 Hz, 2H), 4.22 (s, 4H), 1.51 (t, J=7.1 Hz, 4H).

Intermediate T: 9-Methyl-7,9-dihydro-8H-pyrido[3′,2′:4,5]pyrrolo[2,3-d]pyridazin-8-one

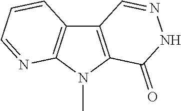

Intermediate T was generated in analogous fashion to Intermediate C from ethyl 3-formyl-1-methyl-1H-pyrrolo[2,3-b]pyridine-2-carboxylate.

### Example 17: 7-(2-Fluorobenzyl)-9-methyl-7,9-dihydro-8H-pyrido[3′,2′:4,5]pyrrolo[2,3-d]pyridazin-8-one

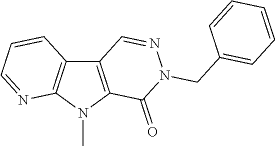 [SMILES: Cn1c2ncccc2c2cnn(Cc3ccccc3)c(=O)c21]

Example 17 was generated in analogous fashion to example 1 from 9-Methyl-7,9-dihydro-8H-pyrido[3′,2′:4,5]pyrrolo[2,3-d]pyridazin-8-one. ¹H NMR (400 MHz, DMSO-d6) δ 8.86 (d, J=1.6 Hz, 1H), 8.73-8.64 (m, 2H), 7.55-7.45 (m, 1H), 7.40-7.24 (m, 5H), 5.44 (s, 2H), 4.29 (d, J=1.5 Hz, 3H).

### Intermediate U: Ethyl 6-methyl-1H-pyrrolo[2,3-b]pyridine-2-carboxylate

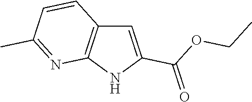

Intermediate U was generated in analogous fashion to Intermediate N from commercially available 3-pyridinecarboxaldehyde. ¹H NMR (400 MHz, Methanol-d4) δ 8.01 (d, J=8.1 Hz, 1H), 7.16 (s, 1H), 7.09 (d, J=8.2 Hz, 1H), 4.42 (q, J=7.1 Hz, 2H), 2.63 (s, 3H), 1.43 (t, J=7.1 Hz, 3H).

### Intermediate V: Ethyl 3-bromo-6-methyl-1H-pyrrolo[2,3-b]pyridine-2-carboxylate

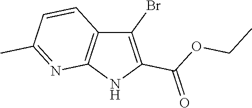 [SMILES: CCOC(=O)c1[nH]c2nc(C)ccc2c1Br]

To a solution of ethyl 6-methyl-1H-pyrrolo[2,3-b]pyridine-2-carboxylate (102 mg, 500 mmol) in DCM (10 mL) was added N-bromosuccinimide (94 mg, 525 mmol). The reaction mixture was stirred at room temperature overnight. Saturated aqueous sodium bicarbonate was added and the resulting reaction mixture was extracted with DCM (3×10 mL). The organic layers were combined, dried, concentrated and purified by flash column chromatography (0-100% EtOAc in hexanes) to provide the desired product (96 mg, 68%). ¹H NMR (400 MHz, DMSO-d6) δ 12.67 (s, 1H), 7.88 (dd, J=8.2, 0.8 Hz, 1H), 7.17 (d, J=8.2 Hz, 1H), 4.37 (q, J=7.1 Hz, 2H), 2.60 (s, 3H), 1.37 (t, J=7.1 Hz, 3H).

### Intermediate W: Ethyl 3-bromo-1,6-dimethyl-TH-pyrrolo[2,3-b]pyridine-2-carboxylate

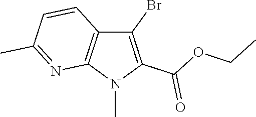 [SMILES: CCOC(=O)c1c(Br)c2ccc(C)nc2n1C]

Intermediate W was generated in analogous fashion to Intermediate B from ethyl 3-bromo-6-methyl-TH-pyrrolo[2,3-b]pyridine-2-carboxylate. ¹H NMR (400 MHz, Methanol-d4) δ 7.91 (d, J=8.2 Hz, 1H), 7.18 (d, J=8.1 Hz, 1H), 4.47 (q, J=7.1 Hz, 2H), 4.10 (s, 3H), 2.68 (s, 3H), 1.47 (t, J=7.1 Hz, 3H).

### Intermediate X: Ethyl 1,6-dimethyl-3-vinyl-TH-pyrrolo[2,3-b]pyridine-2-carboxylate

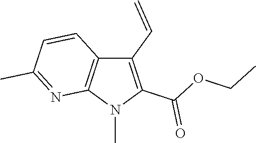 [SMILES: C=Cc1c(C(=O)OCC)n(C)c2nc(C)ccc12]

To a solution of ethyl 3-bromo-1,6-dimethyl-1H-pyrrolo[2,3-b]pyridine-2-carboxylate (71 mg, 0.24 mmol) in DMF was added tributyl(vinyl)stannane (83 mg, 0.26 mmol) and tetrakis(triphenylphosphine)palladium(0) (27 mg, 23 mmol) and nitrogen was bubbled through the reaction mixture for 15 minutes. The reaction mixture was heated to 100° C. overnight. The reaction mixture was cooled to room temperature and water was added. The resulting reaction mixture was extracted with EtOAc (3×15 mL) and the organic layers were combined, dried, concentrated and purified by FCC (0-100% EtOAc in hexanes) to provide the desired product (57 mg, 99%). ¹H NMR (400 MHz, Methanol-d4) δ 8.27 (d, J=8.3 Hz, 1H), 7.43 (dd, J=18.0, 11.5 Hz, 1H), 7.13 (d, J=8.2 Hz, 1H), 5.83 (dd, J=18.0, 1.5 Hz, 1H), 5.43 (dd, J=11.5, 1.5 Hz, 1H), 4.46 (q, J=7.1 Hz, 2H), 4.07 (s, 3H), 1.46 (t, J=7.1 Hz, 3H).

### Intermediate Y: 2,9-Dimethyl-7,9-dihydro-8H-pyrido[3′,2′:4,5]pyrrolo[2,3-d]pyridazin-8-one

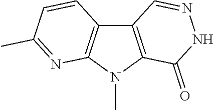 [SMILES: Cc1ccc2c3cn[nH]c(=O)c3n(C)c2n1]

A stream of ozone was gently bubble through a solution of ethyl 1,6-dimethyl-3-vinyl-1H-pyrrolo[2,3-b]pyridine-2-carboxylate (71 mg, 0.29 mmol) in DCM (10 mL) cooled to −78° C. Upon consumption of starting material as indicated by TLC triphenylphosphine (303 mg, 1.15 mmol) was added and the reaction mixture was warmed to room temperature and stir for 4 hours. The reaction mixture was concentrated, dissolved in ethoxyethanol (5 mL) and hydrazine (116 mg, 1.15 mmol) was added and the reaction mixture was heated to 135° C. and stirred overnight. The reaction mixture was cooled to room temperature, water was added and the resulting reaction mixture was extracted with EtOAc (3×10 mL). The organic layers were combined, dried, concentrated and purified by flash column chromatography (0-100% EtOAc in hexanes) to provide the desired product (45 mg, 73%). ¹H NMR (400 MHz, DMSO-d6) δ 12.87 (s, 1H), 8.73 (s, 1H), 8.52 (d, J=8.1 Hz, 1H), 7.34 (d, J=8.1 Hz, 1H), 4.23 (s, 3H), 2.68 (s, 3H).

### Example 18: 7-(3-fluorobenzyl)-2,9-dimethyl-7,9-dihydro-8H-pyrido[3′,2′:4,5]pyrrolo[2,3-d]pyridazin-8-one

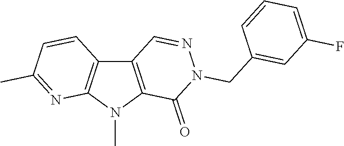 [SMILES: Cc1ccc2c3cnn(Cc4cccc(F)c4)c(=O)c3n(C)c2n1]

Example 18 was generated in analogous fashion to Example 1 from 2,9-dimethyl-7,9-dihydro-8H-pyrido[3′,2′:4,5]pyrrolo[2,3-d]pyridazin-8-one. ¹H NMR (400 MHz, DMSO-d6) δ 8.81 (s, 1H), 8.53 (d, J=8.1 Hz, 1H), 7.44-7.31 (m, 2H), 7.28-7.19 (m, 2H), 7.15 (td, J=7.4, 1.2 Hz, 1H), 5.48 (s, 2H), 4.24 (s, 3H), 2.69 (s, 3H).

### Intermediate Z: Methyl 5-fluoro-3-formyl-6-methoxy-1H-indole-2-carboxylate

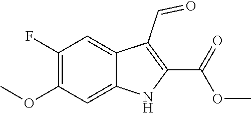

Intermediate E was generated in analogous fashion to Intermediate A from commercially available starting materials. 1H NMR (400 MHz, DMSO-d6) δ 12.85 (s, 1H), 10.55 (s, 1H), 7.89 (d, J=11.6 Hz, 1H), 7.12 (d, J=7.4 Hz, 1H), 3.98 (s, 3H), 3.91 (s, 3H).

### Intermediate AA: Methyl 5-fluoro-3-formyl-6-methoxy-1-methyl-1H-indole-2-carboxylate

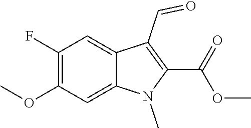

Intermediate AA was generated in analogous fashion to intermediate B. 1H NMR (400 MHz, DMSO-d6) δ 10.38 (s, 1H), 7.93 (d, J=11.5 Hz, 1H), 7.45 (d, J=7.3 Hz, 1H), 4.05 (s, 3H), 3.98 (s, 3H), 3.96 (s, 3H).

### Intermediate AB: 8-fluoro-7-methoxy-5-methyl-3,5-dihydro-4H-pyridazino[4,5-b]indol-4-one

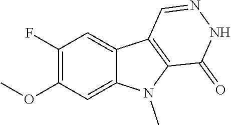 [SMILES: COc1cc2c(cc1F)c1cn[nH]c(=O)c1n2C]

Intermediate E was generated in analogous fashion to Intermediate C. 1H NMR (400 MHz, DMSO-d6) δ 12.72 (s, 1H), 8.65 (s, 1H), 8.06 (d, J=11.3 Hz, 1H), 7.46 (d, J=7.2 Hz, 1H), 4.26 (s, 3H), 4.00 (s, 3H).

### Example 19: Tert-butyl (3-((8-fluoro-7-methoxy-5-methyl-4-oxo-4,5-dihydro-3H-pyridazino[4,5-b]indol-3-yl)methyl)phenyl)carbamate

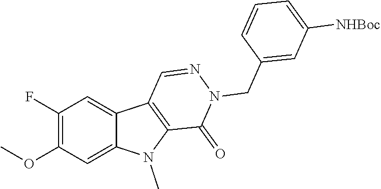 [SMILES: COc1cc2c(cc1F)c1cnn(Cc3cccc(NC(=O)OC(C)(C)C)c3)c(=O)c1n2C]

Example 19 was generated in analogous fashion to intermediate D.

### Example 20: 3-(3-aminobenzyl)-8-fluoro-7-methoxy-5-methyl-3,5-dihydro-4H-pyridazino[4,5-b]indol-4-one

Example 20 was generated in analogous fashion to Example 3. 1H NMR (400 MHz, DMSO-d6) δ 8.71 (s, 1H), 8.08 (d, J=11.3 Hz, 1H), 7.48 (d, J=7.2 Hz, 1H), 6.94 (t, J=7.9 Hz, 1H), 6.50-6.40 (m, 3H), 5.23 (s, 2H), 5.04 (s, 2H), 4.28 (s, 3H), 4.01 (s, 3H).

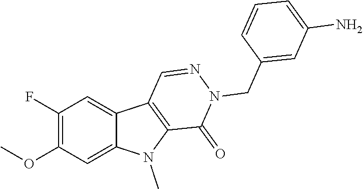 [SMILES: COc1cc2c(cc1F)c1cnn(Cc3cccc(N)c3)c(=O)c1n2C]

### Example 21: 3-(3-aminobenzyl)-8-fluoro-7-hydroxy-5-methyl-3,5-dihydro-4H-pyridazino[4,5-b]indol-4-one

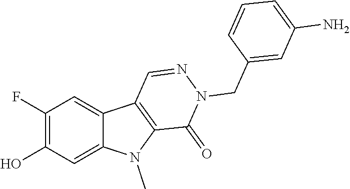 [SMILES: Cn1c2cc(O)c(F)cc2c2cnn(Cc3cccc(N)c3)c(=O)c21]

Example 21 was generated in analogous fashion to Example 15. 1H NMR (400 MHz, Methanol-d4) δ 8.54 (s, 1H), 7.68 (d, J=10.8 Hz, 1H), 7.10-7.02 (m, 1H), 6.95 (d, J=7.4 Hz, 1H), 6.69 (dt, J=8.7, 1.6 Hz, 2H), 6.63 (ddd, J=7.9, 2.2, 1.1 Hz, 1H), 5.51 (s, 3H), 5.40-5.33 (m, 2H), 4.20 (s, 2H), 3.37 (s, 1H).

### Intermediate AC: Ethyl 3-formyl-7-methoxy-1H-indole-2-carboxylate

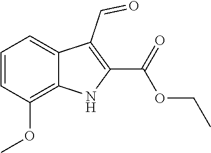 [SMILES: CCOC(=O)c1[nH]c2c(OC)cccc2c1C=O]

Intermediate AC was generated in analogous fashion to Intermediate A. 1H NMR (400 MHz, DMSO-d6) δ 12.92 (s, 1H), 10.59 (s, 1H), 7.85-7.74 (m, 1H), 7.23 (t, J=8.0 Hz, 1H), 6.95 (dd, J=7.8, 0.8 Hz, 1H), 4.43 (q, J=7.1 Hz, 2H), 3.96 (s, 3H), 1.40 (t, J=7.1 Hz, 3H).

### Intermediate AD: Ethyl 3-formyl-7-methoxy-1-methyl-1H-indole-2-carboxylate

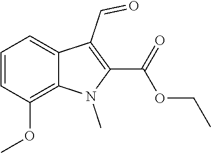 [SMILES: CCOC(=O)c1c(C=O)c2cccc(OC)c2n1C]

Intermediate AD was generated in analogous fashion to Intermediate B. 1H NMR (400 MHz, DMSO-d6) δ 10.36 (s, 1H), 7.89 (dd, J=8.1, 0.9 Hz, 1H), 7.25 (t, J=8.0 Hz, 1H), 6.98 (dd, J=7.9, 0.9 Hz, 1H), 4.47 (q, J=7.1 Hz, 2H), 4.27 (s, 3H), 3.95 (s, 3H), 1.42-1.36 (m, 3H).

### Intermediate AE: 6-methoxy-5-methyl-3,5-dihydro-4H-pyridazino[4,5-b]indol-4-one

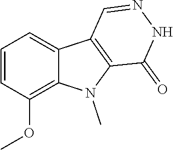 [SMILES: COc1cccc2c3cn[nH]c(=O)c3n(C)c12]

Intermediate AE was generated in analogous fashion to Intermediate C. 1H NMR (400 MHz, DMSO-d6) δ 12.75 (s, 1H), 8.71 (s, 1H), 7.74 (dd, J=8.0, 0.9 Hz, 1H), 7.27 (t, J=7.9 Hz, 1H), 7.09 (dd, J=7.9, 0.9 Hz, 1H), 4.53 (s, 3H), 3.97 (s, 3H).

### Example 22: tert-butyl (3-((6-methoxy-5-methyl-4-oxo-4,5-dihydro-3H-pyridazino[4,5-b]indol-3-yl)methyl)phenyl)carbamate

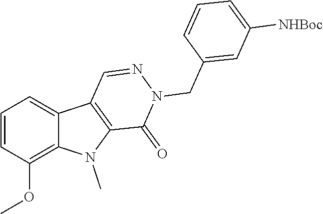 [SMILES: COc1cccc2c3cnn(Cc4cccc(NC(=O)OC(C)(C)C)c4)c(=O)c3n(C)c12]

Example 22 was generated in analogous fashion to Intermediate D. 1H NMR (400 MHz, DMSO-d6) δ 9.31 (s, 1H), 8.79 (s, 1H), 7.76 (dd, J=8.1, 0.9 Hz, 1H), 7.39 (s, 1H), 7.36 (d, J=8.3 Hz, 1H), 7.28 (t, J=7.9 Hz, 1H), 7.19 (t, J=7.8 Hz, 1H), 7.12-7.08 (m, 1H), 6.91 (d, J=7.7 Hz, 1H), 5.33 (s, 2H), 4.54 (s, 3H), 3.97 (s, 3H), 1.44 (s, 9H).

### Example 23: 3-(3-aminobenzyl)-6-methoxy-5-methyl-3,5-dihydro-4H-pyridazino[4,5-b]indol-4-one

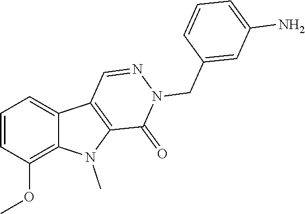 [SMILES: COc1cccc2c3cnn(Cc4cccc(N)c4)c(=O)c3n(C)c12]

Example 23 was generated in analogous fashion to Example 3. 1H NMR (400 MHz, DMSO-d6) δ 8.77 (s, 1H), 7.75 (dd, J=8.1, 0.9 Hz, 1H), 7.28 (t, J=7.9 Hz, 1H), 7.09 (dd, J=7.9, 0.9 Hz, 1H), 6.94 (t, J=7.9 Hz, 1H), 6.50-6.39 (m, 3H), 5.24 (s, 2H), 5.05 (s, 2H), 4.55 (s, 3H), 3.97 (s, 3H).

### Intermediate AF: Methyl 6-bromo-2-methyl-4H-pyrrolo[3,2-d]thiazole-5-carboxylate

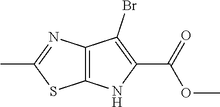

Intermediate AF was generated in analogous fashion to Intermediate V. 1H NMR (400 MHz, DMSO-d6) δ 12.67 (s, 1H), 3.84 (s, 3H), 2.72 (s, 3H).

### Intermediate AG: Methyl 6-bromo-2,4-dimethyl-4H-pyrrolo[3,2-d]thiazole-5-carboxylate

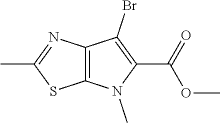

Intermediate AG was generated in analogous fashion to Intermediate W. 1H NMR (400 MHz, DMSO-d6) δ 3.98 (s, 3H), 3.84 (s, 3H), 2.74 (s, 3H).

### Intermediate AH: Methyl 2,4-dimethyl-6-vinyl-4H-pyrrolo[3,2-d]thiazole-5-carboxylate

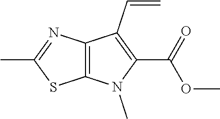

Intermediate AH was generated in analogous fashion to Intermediate X. 1H NMR (400 MHz, DMSO-d6) δ 7.30 (dd, J=17.6, 11.2 Hz, 1H), 6.43 (dd, J=17.6, 2.4 Hz, 1H), 5.43 (dd, J=11.1, 2.4 Hz, 1H), 3.94 (s, 3H), 3.85 (s, 3H), 2.76 (s, 3H).

### Intermediate AI: 2,4-dimethyl-4,6-dihydro-5H-thiazolo[4′,5′:4,5]pyrrolo[2,3-d]pyridazin-5-one

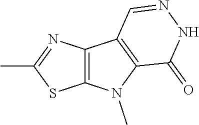

Intermediate AI was generated in analogous fashion to Intermediate C. 1H NMR (400 MHz, DMSO-d6) δ 12.65 (s, 1H), 8.45 (s, 1H), 4.26 (s, 3H), 2.82 (s, 3H).

### Example 24: Tert-butyl (3-((2,4-dimethyl-5-oxo-4,5-dihydro-6H-thiazolo[4′,5′:4,5]pyrrolo[2,3-d]pyridazin-6-yl)methyl)phenyl)carbamate

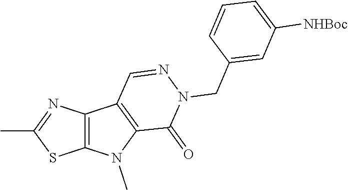 [SMILES: Cc1nc2c3cnn(Cc4cccc(NC(=O)OC(C)(C)C)c4)c(=O)c3n(C)c2s1]

Example 24 was generated in analogous fashion to Intermediate Intermediate D.

### Example 25: 6-(3-aminobenzyl)-2,4-dimethyl-4,6-dihydro-5H-thiazolo[4′,5′:4,5]pyrrolo[2,3-d]pyridazin-5-one

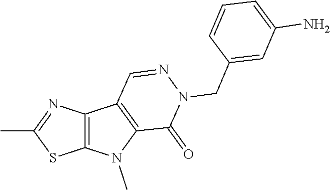 [SMILES: Cc1nc2c3cnn(Cc4cccc(N)c4)c(=O)c3n(C)c2s1]

Example 25 was generated in analogous fashion to Example 3. 1H NMR (400 MHz, DMSO-d6) δ 8.51 (s, 1H), 6.94 (t, J=7.6 Hz, 1H), 6.51-6.39 (m, 3H), 5.20 (s, 2H), 5.05 (s, 2H), 4.28 (s, 3H), 2.82 (s, 3H).

### Intermediate AJ: Ethyl 3-formyl-5-methoxy-1H-indole-2-carboxylate

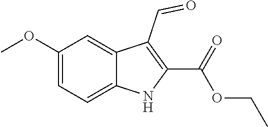 [SMILES: CCOC(=O)c1[nH]c2ccc(OC)cc2c1C=O]

Intermediate AJ was generated in analogous fashion to Intermediate A. 1H NMR (400 MHz, DMSO-d6) δ 12.77 (s, 1H), 10.60 (s, 1H), 7.70 (d, J=2.5 Hz, 1H), 7.48 (dd, J=9.0, 0.5 Hz, 1H), 7.05 (dd, J=9.0, 2.5 Hz, 1H), 4.45 (q, J=7.1 Hz, 2H), 3.81 (s, 3H), 1.40 (t, J=7.1 Hz, 3H).

### Intermediate AK: Ethyl 3-formyl-5-methoxy-1-methyl-1H-indole-2-carboxylate

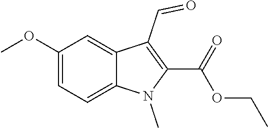 [SMILES: CCOC(=O)c1c(C=O)c2cc(OC)ccc2n1C]

Intermediate AH was generated in analogous fashion to Intermediate B. 1H NMR (400 MHz, DMSO-d6) δ 10.44 (s, 1H), 7.77 (d, J=2.5 Hz, 1H), 7.67 (d, J=9.2 Hz, 1H), 7.11 (dd, J=9.1, 2.6 Hz, 1H), 4.46 (q, J=7.1 Hz, 2H), 4.04 (s, 3H), 3.83 (s, 3H), 1.40 (t, J=7.1 Hz, 3H).

### Intermediate AL: 8-methoxy-5-methyl-3,5-dihydro-4H-pyridazino[4,5-b]indol-4-one

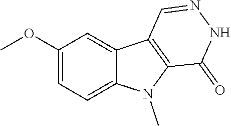 [SMILES: COc1ccc2c(c1)c1cn[nH]c(=O)c1n2C]

Intermediate AL was generated in analogous fashion to Intermediate C. 1H NMR (400 MHz, DMSO-d6) δ 12.71 (s, 1H), 8.73 (s, 1H), 7.75 (d, J=2.4 Hz, 1H), 7.71-7.62 (m, 1H), 7.23 (dd, J=9.0, 2.5 Hz, 1H), 4.25 (s, 3H), 3.87 (s, 3H).

### Example 26: tert-butyl (3-((8-methoxy-5-methyl-4-oxo-4,5-dihydro-3H-pyridazino[4,5-b]indol-3-yl)methyl)phenyl)carbamate

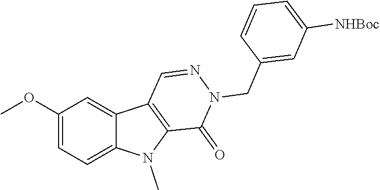 [SMILES: COc1ccc2c(c1)c1cnn(Cc3cccc(NC(=O)OC(C)(C)C)c3)c(=O)c1n2C]

Example 26 was generated in analogous fashion to Intermediate Intermediate D. 1H NMR (400 MHz, DMSO-d6) δ 9.31 (s, 1H), 8.80 (s, 1H), 7.77 (d, J=2.5 Hz, 1H), 7.69 (dd, J=9.2, 0.5 Hz, 1H), 7.39 (s, 1H), 7.35 (d, J=8.3 Hz, 1H), 7.24 (dd, J=9.1, 2.5 Hz, 1H), 7.19 (t, J=7.8 Hz, 1H), 6.91 (dt, J=7.7, 1.2 Hz, 1H), 5.33 (s, 2H), 4.26 (s, 3H), 3.87 (s, 3H), 1.44 (s, 9H).

### Example 27: 3-(3-aminobenzyl)-8-methoxy-5-methyl-3,5-dihydro-4H-pyridazino[4,5-b]indol-4-one

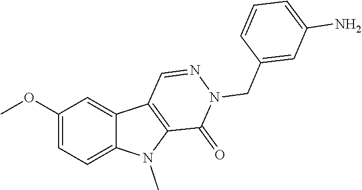 [SMILES: COc1ccc2c(c1)c1cnn(Cc3cccc(N)c3)c(=O)c1n2C]

Example 27 was generated in analogous fashion to Example 3.

### Example 28: 7-methoxy-5-methyl-3-((6-methylpyridin-2-yl)methyl)-3,5-dihydro-4H-pyridazino[4,5-b]indol-4-one

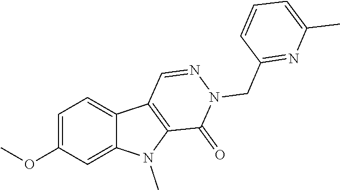

Example 28 was generated in analogous fashion to Example 15. 1H NMR (400 MHz, DMSO-d6) δ 8.77 (s, 1H), 8.09 (d, J=8.8 Hz, 1H), 7.60 (t, J=7.7 Hz, 1H), 7.26 (d, J=2.2 Hz, 1H), 7.14 (d, J=7.6 Hz, 1H), 7.04 (dd, J=8.8, 2.2 Hz, 1H), 6.82 (d, J=7.7 Hz, 1H), 5.45 (s, 2H), 4.25 (s, 3H), 3.92 (s, 3H), 2.45 (s, 3H).

### Example 29: 7-hydroxy-5-methyl-3-((6-methylpyridin-2-yl)methyl)-3,5-dihydro-4H-pyridazino[4,5-b]indol-4-one

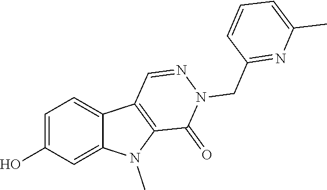 [SMILES: Cc1cccc(Cn2ncc3c4ccc(O)cc4n(C)c3c2=O)n1]

Example 29 was generated in analogous fashion to Example 15. 1H NMR (400 MHz, DMSO-d6) δ 9.97 (s, 1H), 8.72 (s, 1H), 8.00 (d, J=8.6 Hz, 1H), 7.59 (t, J=7.7 Hz, 1H), 7.13 (d, J=7.7 Hz, 1H), 6.97 (d, J=2.0 Hz, 1H), 6.92 (dd, J=8.6, 2.1 Hz, 1H), 6.80 (d, J=7.8 Hz, 1H), 5.44 (s, 2H), 4.17 (s, 3H), 2.45 (s, 3H).

### Intermediate AM: 7-methoxy-5-methyl-3-((1-((2-(trimethylsilyl)ethoxy)methyl)-1H-indazol-4-yl)methyl)-3,5-dihydro-4H-pyridazino[4,5-b]indol-4-one

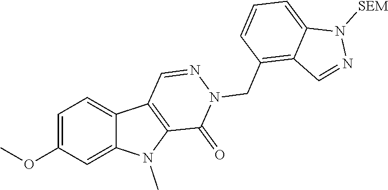 [SMILES: COc1ccc2c3cnn(Cc4cccc5c4cnn5COCC[Si](C)(C)C)c(=O)c3n(C)c2c1]

Intermediate AM was generated in analogous fashion to Intermediate D. 1H NMR (400 MHz, DMSO-d6) δ 8.77 (s, 1H), 8.25 (d, J=1.0 Hz, 1H), 8.07 (d, J=8.7 Hz, 1H), 7.66 (d, J=8.5 Hz, 1H), 7.38 (dd, J=8.5, 7.0 Hz, 1H), 7.24 (d, J=2.0 Hz, 1H), 7.07-6.99 (m, 2H), 5.77-5.70 (m, 4H), 4.26 (s, 3H), 3.91 (s, 3H), 3.56-3.47 (m, 2H), 0.85-0.72 (m, 2H), −0.07-−0.15 (m, 9H).

### Example 30: 3-((1H-indazol-4-yl)methyl)-7-methoxy-5-methyl-3,5-dihydro-4H-pyridazino[4,5-b]indol-4-one

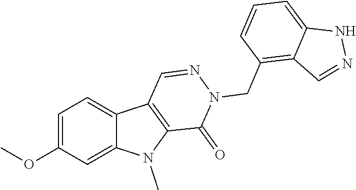 [SMILES: COc1ccc2c3cnn(Cc4cccc5[nH]ncc45)c(=O)c3n(C)c2c1]

To a solution of 7-methoxy-5-methyl-3-((1-((2-(trimethylsilyl)ethoxy)methyl)-1H-indazol-4-yl)methyl)-3,5-dihydro-4H-pyridazino[4,5-b]indol-4-one (38.2 mg, 78.0 μmol) in DCM (5 mL) was added TFA (1 mL) and the reaction mixture was stirred at room temperature for 4 hours. The reaction mixture was diluted with DCM (10 mL), washed with saturated aqueous sodium bicarbonate (2×5 mL), dried, concentrated and purified via flash column chromatography (0-100% EtOAc in Hexanes).

### Intermediate AN: 5-methyl-7-(methylthio)-3-((1-((2-(trimethylsilyl)ethoxy)methyl)-1H-indazol-4-yl)methyl)-3,5-dihydro-4H-pyridazino[4,5-b]indol-4-one

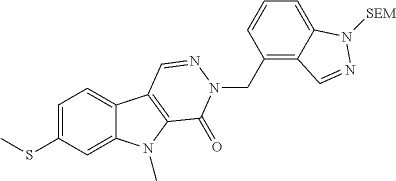 [SMILES: CSc1ccc2c3cnn(Cc4cccc5c4cnn5COCC[Si](C)(C)C)c(=O)c3n(C)c2c1]

Intermediate AN was generated in analogous fashion to to Example 1. 1H NMR (400 MHz, DMSO-d6) δ 8.81 (s, 1H), 8.26 (d, J=0.9 Hz, 1H), 8.11 (d, J=8.5 Hz, 1H), 7.66 (d, J=8.4 Hz, 1H), 7.58 (d, J=1.6 Hz, 1H), 7.38 (dd, J=8.4, 7.1 Hz, 1H), 7.30 (dd, J=8.4, 1.6 Hz, 1H), 7.06 (d, J=7.1 Hz, 1H), 5.74 (d, J=8.2 Hz, 2H), 4.28 (s, 3H), 3.56-3.46 (m, 3H), 2.63 (s, 2H), 0.78 (dd, J=8.6, 7.6 Hz, 2H), −0.12 (s, 9H).

### Example 31: 3-((1H-indazol-4-yl)methyl)-5-methyl-7-(methylthio)-3,5-dihydro-4H-pyridazino[4,5-b]indol-4-one

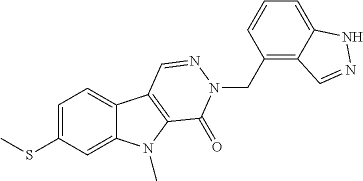 [SMILES: CSc1ccc2c3cnn(Cc4cccc5[nH]ncc45)c(=O)c3n(C)c2c1]

Example 31 was generated in analogous fashion to Example AN.

### Intermediate AO: 5-methyl-7-(methylsulfinyl)-3-((1-((2-(trimethylsilyl)ethoxy)methyl)-1H-indazol-4-yl)methyl)-3,5-dihydro-4H-pyridazino[4,5-b]indol-4-one

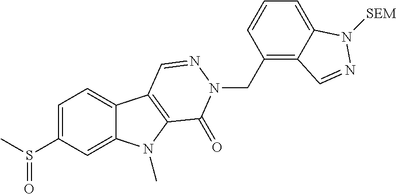 [SMILES: Cn1c2cc(S(C)=O)ccc2c2cnn(Cc3cccc4c3cnn4COCC[Si](C)(C)C)c(=O)c21]

Intermediate AO was generated in analogous fashion to Example 1.

### Example 32: 3-((1H-indazol-4-yl)methyl)-5-methyl-7-(methylsulfinyl)-3,5-dihydro-4H-pyridazino[4,5-b]indol-4-one

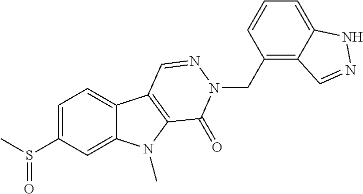 [SMILES: Cn1c2cc(S(C)=O)ccc2c2cnn(Cc3cccc4[nH]ncc34)c(=O)c21]

Example 32 was generated in analogous fashion to Example 31.

### Intermediate AP: methyl 6-chloro-1-methyl-1H-pyrrolo[2,3-b]pyridine-2-carboxylate

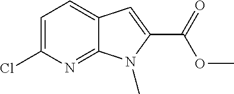

Intermediate AP was generated in a analogous fashion to Intermediate B from commercially available material. 1H NMR (400 MHz, DMSO-d6) δ 8.22 (d, J=8.3 Hz, 1H), 7.33 (s, 1H), 7.29 (d, J=8.3 Hz, 1H), 4.02 (s, 3H), 3.89 (s, 3H).

### Intermediate AQ: methyl 6-(2-fluorobenzyl)-1-methyl-1H-pyrrolo[2,3-b]pyridine-2-carboxylate

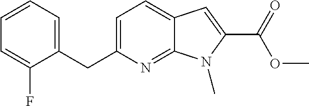 [SMILES: COC(=O)c1cc2ccc(Cc3ccccc3F)nc2n1C]

To a solution of methyl 6-chloro-1-methyl-1H-pyrrolo[2,3-b]pyridine-2-carboxylate (105.9 mg, 471.4 μmol) in a sealed tube under an atmosphere of nitrogen was added tetrakis(triphenylphosphine)palladium(0) (9.602 mg, 8.309 μmol), followed by (2-fluorobenzyl)zinc(II) chloride (111.5 mg, 530.9 μmol) in THF. The reaction mixture was heated to 100° C. for 2 hours. The reaction mixture was cooled and water was added (30 mL). The reaction mixture was extracted with EtOAc (3×30 mL). The organics were combined, dried, concentrated and purified by flash column chromatography (0-50% EtOAc in Hexanes) to afford the desired product (42.1 mg, 30.6%). 1H NMR (400 MHz, DMSO-d6) δ 8.06 (d, J=8.1 Hz, 1H), 7.37 (td, J=7.7, 1.9 Hz, 1H), 7.32-7.26 (m, 1H), 7.25 (s, 1H), 7.22-7.13 (m, 2H), 7.07 (d, J=8.1 Hz, 1H), 4.25 (s, 2H), 4.02 (s, 3H), 3.88 (s, 3H).

Intermediate AR: methyl 3-bromo-6-(2-fluorobenzyl)-1-methyl-1H-pyrrolo[2,3-b]pyridine-2-carboxylate

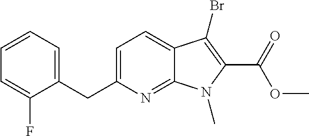 [SMILES: COC(=O)c1c(Br)c2ccc(Cc3ccccc3F)nc2n1C]

Intermediate AR was generated in an analogous fashion to Intermediate V. 1H NMR (400 MHz, DMSO-d6) δ 7.96 (d, J=8.2 Hz, 1H), 7.37 (td, J=7.6, 1.7 Hz, 1H), 7.33-7.27 (m, 1H), 7.22-7.13 (m, 3H), 4.30-4.27 (m, 2H), 4.00 (s, 3H), 3.93 (s, 3H).

### Intermediate AS: methyl 6-(2-fluorobenzyl)-1-methyl-3-vinyl-1H-pyrrolo[2,3-b]pyridine-2-carboxylate

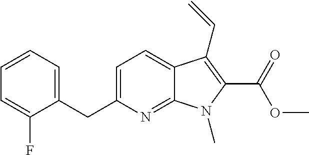 [SMILES: C=Cc1c(C(=O)OC)n(C)c2nc(Cc3ccccc3F)ccc12]

Intermediate AS was generated in analogous fashion to Intermediate AR.

### Intermediate AT: methyl 6-(2-fluorobenzyl)-3-formyl-1-methyl-1H-pyrrolo[2,3-b]pyridine-2-carboxylate

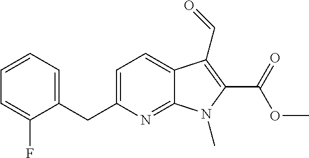 [SMILES: COC(=O)c1c(C=O)c2ccc(Cc3ccccc3F)nc2n1C]

Intermediate AT was generated in analogous fashion to Intermediate Y.

### Intermediate AU: 2-(2-fluorobenzyl)-9-methyl-7,9-dihydro-8H-pyrido[3′,2′:4,5]pyrrolo[2,3-d]pyridazin-8-one

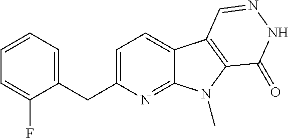 [SMILES: Cn1c2nc(Cc3ccccc3F)ccc2c2cn[nH]c(=O)c21]

Intermediate AU was generated in analogous fashion to Intermediate C.

### Example 33: 2-(2-fluorobenzyl)-9-methyl-7-((6-methylpyridin-2-yl)methyl)-7,9-dihydro-8H-pyrido[3′,2′:4,5]pyrrolo[2,3-d]pyridazin-8-one

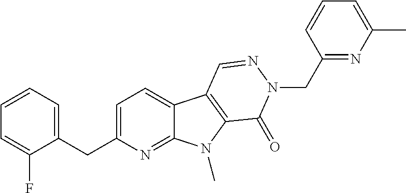 [SMILES: Cc1cccc(Cn2ncc3c4ccc(Cc5ccccc5F)nc4n(C)c3c2=O)n1]

Example 33 was generated in analogous fashion to Example 1.

### Intermediate AV: 2-(2-fluorobenzyl)-9-methyl-7-((1-((2-(trimethylsilyl)ethoxy)methyl)-1H-indazol-4-yl)methyl)-7,9-dihydro-8H-pyrido[3′,2′:4,5]pyrrolo[2,3-d]pyridazin-8-one

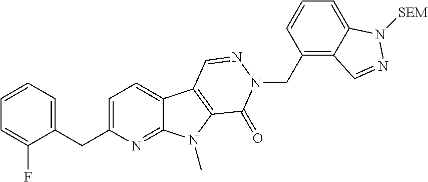 [SMILES: Cn1c2nc(Cc3ccccc3F)ccc2c2cnn(Cc3cccc4c3cnn4COCC[Si](C)(C)C)c(=O)c21]

Intermediate AV was generated in analogous fashion to Example 1.

### Example 34: 7-((1H-indazol-4-yl)methyl)-2-(2-fluorobenzyl)-9-methyl-7,9-dihydro-8H-pyrido[3′,2′:4,5]pyrrolo[2,3-d]pyridazin-8-one

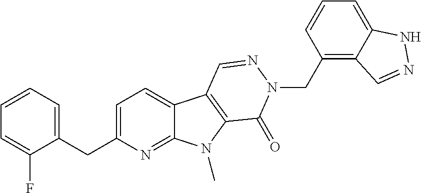 [SMILES: Cn1c2nc(Cc3ccccc3F)ccc2c2cnn(Cc3cccc4[nH]ncc34)c(=O)c21]

Example 34 was generated in analogous fashion to Example 31.

### Intermediate AW: methyl 6-(2-fluorophenoxy)-1-methyl-H-pyrrolo[2,3-b]pyridine-2-carboxylate

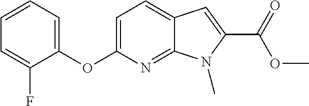 [SMILES: COC(=O)c1cc2ccc(Oc3ccccc3F)nc2n1C]

To a solution of methyl 6-chloro-1-methyl-1H-pyrrolo[2,3-b]pyridine-2-carboxylate (105.9 mg, 471.4 μmol) in a sealed tube under an atmosphere of nitrogen was added 1-fluoro-2-hydroxybenzene (68.70 mg, 54.70 μmol), tris(dibezylideneacetone)dipalladium (34.54 mg, 37.71 μmol), 2-(Dicyclohexylphosphanyl)-2′,4′,6′-tris(isopropyl)biphenyl (35.96 mg, 75.43 μmol) and potassium carbonate (143.3 mg, 1.037 mmol) in toluene. The reaction mixture was heated to 110° C. and stirred overnight. The reaction mixture was cooled and water was added (30 mL). The reaction mixture was extracted with ethyl EtOAc (3×30 mL). The organics were combined, dried, concentrated and purified by flash column chromatography (0-100% EtOAc in Hexanes) to afford the desired product (95.2 mg, 67.2%). 1H NMR (400 MHz, DMSO-d6) δ 8.21 (d, J=8.5 Hz, 1H), 7.47-7.38 (m, 2H), 7.37-7.29 (m, 2H), 7.28 (d, J=5.0 Hz, 1H), 6.96 (d, J=8.5 Hz, 1H), 3.85 (s, 3H), 3.75 (s, 3H).

### Intermediate AX: Methyl 3-bromo-6-(2-fluorophenoxy)-1-methyl-1H-pyrrolo[2,3-b]pyridine-2-carboxylate

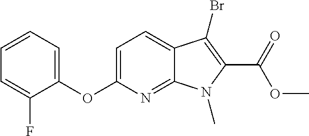 [SMILES: COC(=O)c1c(Br)c2ccc(Oc3ccccc3F)nc2n1C]

Br Intermediate AX was generated in analogous fashion to Intermediate V. 1H NMR (400 MHz, DMSO-d6) δ 8.10 (d, J=8.6 Hz, 1H), 7.46-7.40 (m, 2H), 7.39-7.27 (m, 2H), 7.06 (d, J=8.6 Hz, 1H), 3.91 (s, 3H), 3.74 (s, 3H).

### Intermediate AY: Methyl 6-(2-fluoro henoxy)-1-methyl-3-vinyl-1H-pyrrolo[2,3-b]pyridine-2-carboxylate

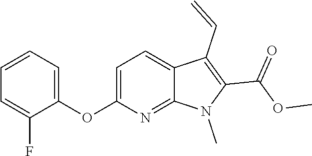 [SMILES: C=Cc1c(C(=O)OC)n(C)c2nc(Oc3ccccc3F)ccc12]

Intermediate AY was generated in analogous fashion to Intermediate X. 1H NMR (400 MHz, DMSO-d6) δ 8.50 (d, J=8.7 Hz, 1H), 7.46-7.42 (m, 1H), 7.42-7.38 (m, 1H), 7.38-7.27 (m, 3H), 6.98 (d, J=8.6 Hz, 1H), 5.91 (dd, J=18.1, 1.4 Hz, 1H), 5.46 (dd, J=11.5, 1.3 Hz, 1H), 3.89 (s, 3H), 3.71 (s, 3H).

### Intermediate AZ: 2-(2-fluorophenoxy)-9-methyl-7,9-dihydro-8H-pyrido[3′,2′:4,5]pyrrolo[2,3-d]pyridazin-8-one

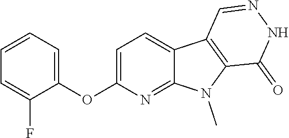 [SMILES: Cn1c2nc(Oc3ccccc3F)ccc2c2cn[nH]c(=O)c21]

Intermediate AZ was generated in analogous fashion to Intermediate C.

### Example 35: 2-(2-fluorophenoxy)-9-methyl-7-((6-methylpyridin-2-yl)methyl)-7,9-dihydro-8H-pyrido[3′,2′:4,5]pyrrolo[2,3-d]pyridazin-8-one

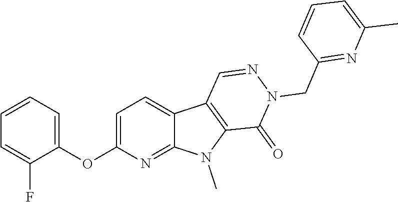 [SMILES: Cc1cccc(Cn2ncc3c4ccc(Oc5ccccc5F)nc4n(C)c3c2=O)n1]

Example 35 was generated in a fashion analogous to Example 1.

Intermediate BA: 2-(2-fluorophenoxy)-9-methyl-7-((1-((2-(trimethylsilyl)ethoxy)methyl)-1H-indazol-4-yl)methyl)-7,9-dihydro-8H-pyrido[3′,2′:4,5]pyrrolo[2,3-d]pyridazin-8-one

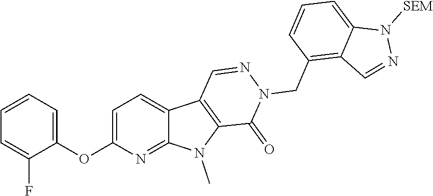 [SMILES: Cn1c2nc(Oc3ccccc3F)ccc2c2cnn(Cc3cccc4c3cnn4COCC[Si](C)(C)C)c(=O)c21]

Intermediate BA was generated in a fashion analogous to Example 1.

### Example 36: 2-(2-fluorophenoxy)-9-methyl-7-((1-((2-(trimethylsilyl)ethoxy)methyl)-1H-indazol-4-yl)methyl)-7,9-dihydro-8H-pyrido[3′,2′:4,5]pyrrolo[2,3-d]pyridazin-8-one

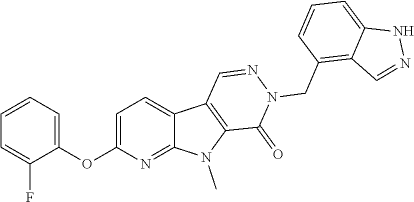 [SMILES: Cn1c2nc(Oc3ccccc3F)ccc2c2cnn(Cc3cccc4[nH]ncc34)c(=O)c21]

Example 36 was generated in a fashion analogous to Intermediate 31. NMR NEEDED. Rxn PLANNED

### Example 37: 7-methoxy-5-methyl-3-(3-nitrobenzyl)-3,5-dihydro-4H-pyridazino[4,5-b]indol-4-one

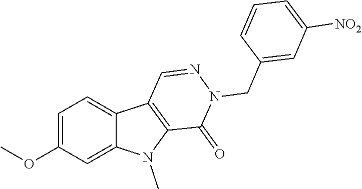 [SMILES: COc1ccc2c3cnn(Cc4cccc([N+](=O)[O-])c4)c(=O)c3n(C)c2c1]

Example 36 was generated in a fashion analogous to Example 1. 1H NMR (400 MHz, DMSO-d6) δ 8.80 (s, 1H), 8.20-8.14 (m, 2H), 8.09 (d, J=8.8 Hz, 1H), 7.78 (dt, J=7.6, 1.4 Hz, 1H), 7.65 (t, J=7.9 Hz, 1H), 7.25 (d, J=2.2 Hz, 1H), 7.03 (dd, J=8.8, 2.2 Hz, 1H), 5.55 (s, 2H), 4.26 (s, 3H), 3.92 (s, 3H).

### Example 38: 7-hydroxy-5-methyl-3-(3-nitrobenzyl)-3,5-dihydro-4H-pyridazino[4,5-b]indol-4-one

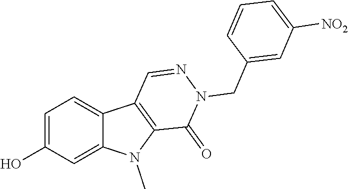 [SMILES: Cn1c2cc(O)ccc2c2cnn(Cc3cccc([N+](=O)[O-])c3)c(=O)c21]

Example 38 was generated in a fashion analogous to Example 15. 1H NMR (400 MHz, DMSO-d6) δ 10.00 (s, 1H), 8.75 (s, 1H), 8.21-8.13 (m, 2H), 7.99 (d, J=8.6 Hz, 1H), 7.77 (dt, J=7.7, 1.3 Hz, 1H), 7.65 (td, J=7.7, 0.8 Hz, 1H), 6.96 (d, J=1.9 Hz, 1H), 6.91 (dd, J=8.6, 2.1 Hz, 1H), 5.54 (s, 2H), 4.17 (s, 3H).

### Example 39: 5-methyl-3-(3-nitrobenzyl)-7-((6-nitropyridin-2-yl)oxy)-3,5-dihydro-4H-pyridazino[4,5-b]indol-4-one

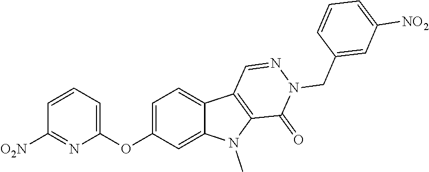 [SMILES: Cn1c2cc(Oc3cccc([N+](=O)[O-])n3)ccc2c2cnn(Cc3cccc([N+](=O)[O-])c3)c(=O)c21]

Example 39 was generated in a fashion analogous to Intermediate XXX2.

### Example 40: 5-methyl-3-(3-nitrobenzyl)-7-((6-nitropyridin-2-yl)oxy)-3,5-dihydro-4H-pyridazino[4,5-b]indol-4-one

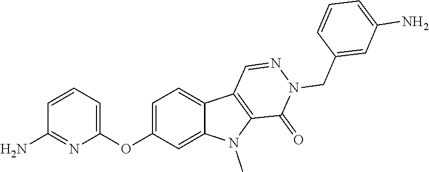 [SMILES: Cn1c2cc(Oc3cccc(N)n3)ccc2c2cnn(Cc3cccc(N)c3)c(=O)c21]

Example 36 was generated in a fashion analogous to Example XXX3.

### Example 41: 5-methyl-3-((6-methylpyridin-2-yl)methyl)-7-(3-nitrophenoxy)-3,5-dihydro-4H-pyridazino[4,5-b]indol-4-one

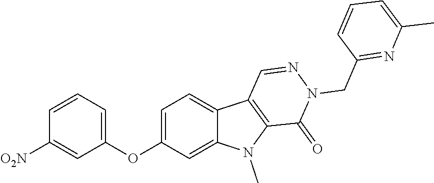 [SMILES: Cc1cccc(Cn2ncc3c4ccc(Oc5cccc([N+](=O)[O-])c5)cc4n(C)c3c2=O)n1]

### Example 42: 7-(3-aminophenoxy)-5-methyl-3-((6-methylpyridin-2-yl)methyl)-3,5-dihydro-4H-pyridazino[4,5-b]indol-4-one

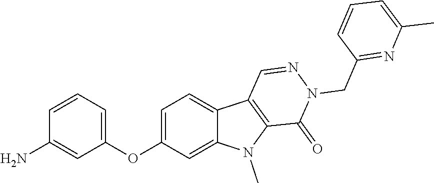 [SMILES: Cc1cccc(Cn2ncc3c4ccc(Oc5cccc(N)c5)cc4n(C)c3c2=O)n1]

### Example 43: 5-methyl-3-((6-methylpyridin-2-yl)methyl)-7-((6-nitropyridin-2-yl)oxy)-3,5-dihydro-4H-pyridazino[4,5-b]indol-4-one

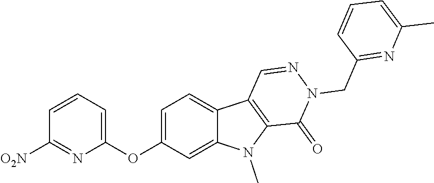 [SMILES: Cc1cccc(Cn2ncc3c4ccc(Oc5cccc([N+](=O)[O-])n5)cc4n(C)c3c2=O)n1]

### Example 44: 7-((6-aminopyridin-2-yl)oxy)-5-methyl-3-((6-methylpyridin-2-yl)methyl)-3,5-dihydro-4H-pyridazino[4,5-b]indol-4-one

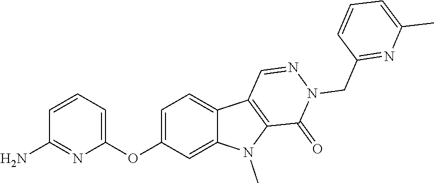 [SMILES: Cc1cccc(Cn2ncc3c4ccc(Oc5cccc(N)n5)cc4n(C)c3c2=O)n1]

### Example 45: 3-(2-fluorobenzyl)-7-methoxy-5-methyl-3,5-dihydro-4H-pyridazino[4,5-b]indol-4-one

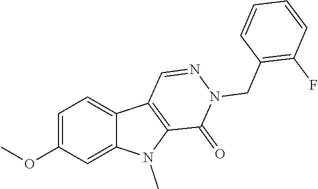 [SMILES: COc1ccc2c3cnn(Cc4ccccc4F)c(=O)c3n(C)c2c1]

### Example 46: 3-(2-fluorobenzyl)-7-hydroxy-5-methyl-3,5-dihydro-4H-pyridazino[4,5-b]indol-4-on

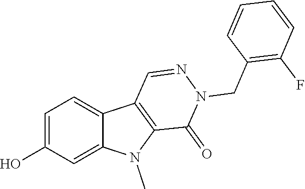 [SMILES: Cn1c2cc(O)ccc2c2cnn(Cc3ccccc3F)c(=O)c21]

### Example 47: 3-(2-fluorobenzyl)-5-methyl-7-((6-nitropyridin-2-yl)oxy)-3,5-dihydro-4H-pyridazino [4,5-b]indol-4-one

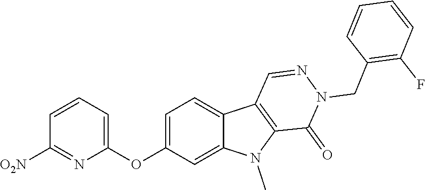 [SMILES: Cn1c2cc(Oc3cccc([N+](=O)[O-])n3)ccc2c2cnn(Cc3ccccc3F)c(=O)c21]

### Example 48: 7-((6-aminopyridin-2-yl)oxy)-3-(2-fluorobenzyl)-5-methyl-3,5-dihydro-4H-pyridazino[4,5-b]indol-4-one

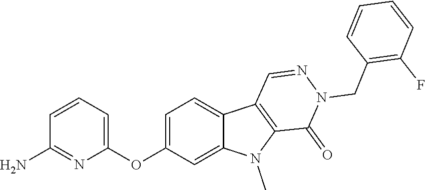 [SMILES: Cn1c2cc(Oc3cccc(N)n3)ccc2c2cnn(Cc3ccccc3F)c(=O)c21]

### Intermediate AC: 7-hydroxy-5-methyl-3-((1-((2-(trimethylsilyl)ethoxy)methyl)-1H-indazol-4-yl)methyl)-3,5-dihydro-4H-pyridazino[4,5-b]indol-4-one

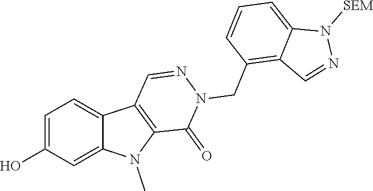 [SMILES: Cn1c2cc(O)ccc2c2cnn(Cc3cccc4c3cnn4COCC[Si](C)(C)C)c(=O)c21]

### Intermediate AD: 5-methyl-7-(3-nitrophenoxy)-3-((1-((2-(trimethylsilyl)ethoxy)methyl)-1H-indazol-4-yl)methyl)-3,5-dihydro-4H-pyridazino[4,5-b]indol-4-one

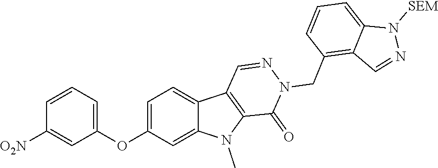 [SMILES: Cn1c2cc(Oc3cccc([N+](=O)[O-])c3)ccc2c2cnn(Cc3cccc4c3cnn4COCC[Si](C)(C)C)c(=O)c21]

### Intermediate AE: 7-(3-aminophenoxy)-5-methyl-3-((1-((2-(trimethylsilyl)ethoxy)methyl)-1H-indazol-4-yl)methyl)-3,5-dihydro-4H-pyridazino[4,5-b]indol-4-one

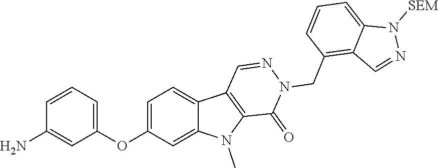 [SMILES: Cn1c2cc(Oc3cccc(N)c3)ccc2c2cnn(Cc3cccc4c3cnn4COCC[Si](C)(C)C)c(=O)c21]

### Example 49: 3-((1H-indazol-4-yl)methyl)-7-(3-aminophenoxy)-5-methyl-3,5-dihydro-4H-pyridazino[4,5-b]indol-4-one

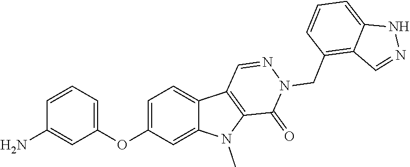 [SMILES: Cn1c2cc(Oc3cccc(N)c3)ccc2c2cnn(Cc3cccc4[nH]ncc34)c(=O)c21]

### Intermediate AF: 5-methyl-7-((6-nitropyridin-2-yl)oxy)-3-((1-((2-(trimethylsilyl)ethoxy)methyl)-1H-indazol-4-yl)methyl)-3,5-dihydro-4H-pyridazino[4,5-b]indol-4-one

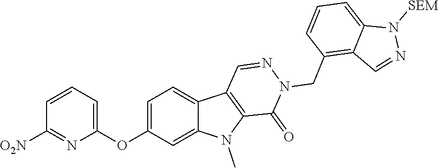 [SMILES: Cn1c2cc(Oc3cccc([N+](=O)[O-])n3)ccc2c2cnn(Cc3cccc4c3cnn4COCC[Si](C)(C)C)c(=O)c21]

### Intermediate AG: 7-((6-aminopyridin-2-yl)oxy)-5-methyl-3-((1-((2-(trimethylsilyl)ethoxy)methyl)-1H-indazol-4-yl)methyl)-3,5-dihydro-4H-pyridazino[4,5-b]indol-4-one

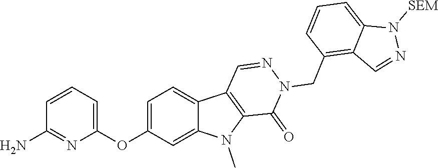 [SMILES: Cn1c2cc(Oc3cccc(N)n3)ccc2c2cnn(Cc3cccc4c3cnn4COCC[Si](C)(C)C)c(=O)c21]

### Example 50: 3-((1H-indazol-4-yl)methyl)-7-((6-aminopyridin-2-yl)oxy)-5-methyl-3,5-dihydro-4H-pyridazino[4,5-b]indol-4-one

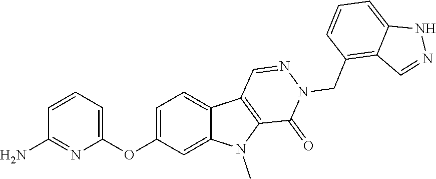 [SMILES: Cn1c2cc(Oc3cccc(N)n3)ccc2c2cnn(Cc3cccc4[nH]ncc34)c(=O)c21]

### Example 51: 3-((1H-pyrazol-3-yl)methyl)-8-fluoro-7-methoxy-5-methyl-3,5-dihydro-4H-pyridazino[4,5-b]indol-4-one

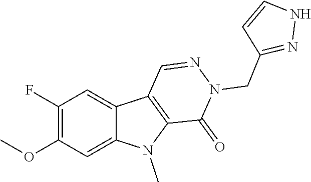 [SMILES: COc1cc2c(cc1F)c1cnn(Cc3cc[nH]n3)c(=O)c1n2C]

Example 51 was generated by treating Intermediate AB (30 mg, 0.121 mmol) with CsCO₃ (119 mg, 0.364 mmol) and (1-(tetrahydro-2H-pyran-2-yl)-1H-pyrazol-3-yl)methyl methanesulfonate (31.6 mg, 0.121 mmol) in DMF (3 mL). The reaction mixture was stirred for 48 hours and water was added. The reaction mixture was extracted with EtOAc (5 mL×3) and the organics were dried, concentrated and purified by FCC (0-100% EtOAc in Hexanes) to provide 8-fluoro-7-methoxy-5-methyl-3-((1-(tetrahydro-2H-pyran-2-yl)-1H-pyrazol-3-yl)methyl)-3,5-dihydro-4H-pyridazino[4,5-b]indol-4-one. 8-fluoro-7-methoxy-5-methyl-3-((1-(tetrahydro-2H-pyran-2-yl)-1H-pyrazol-3-yl)methyl)-3,5-dihydro-4H-pyridazino[4,5-b]indol-4-one was subsequently dissolved in methanol (3 mL) and treated with 0.4 mL of 4M HCl in dioxane. The resulting reaction mixture was diluted with EtOAc and washed with saturated aqueous sodium bicarbonate. The organics were dried, concentrated and purified by FCC (0-100% EtOAc in Hexanes) to provide the desired product. 1H NMR (400 MHz, DMSO-d6) δ 12.63 (s, 1H), 8.68 (s, 1H), 8.07 (d, J=11.2 Hz, 1H), 7.62 (s, 1H), 7.47 (d, J=7.3 Hz, 1H), 6.13 (s, 1H), 5.39 (d, J=19.9 Hz, 2H), 4.28 (s, 3H), 4.00 (s, 3H).

### Example 52: 8-fluoro-7-methoxy-5-methyl-3-((6-methylpyridin-2-yl)methyl)-3,5-dihydro-4H-pyridazino[4,5-b]indol-4-one

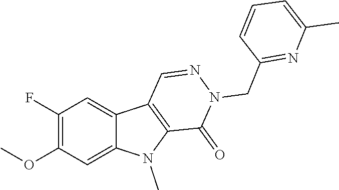

Example 52 was generated in analogous fashion to Example 1 from intermediate AB and commercially available 2-(chloromethyl)-6-methylpyridine. 1H NMR (400 MHz, DMSO-d6) δ 12.63 (s, 1H), 8.68 (s, 1H), 8.07 (d, J=11.2 Hz, 1H), 7.62 (s, 1H), 7.47 (d, J=7.3 Hz, 1H), 6.13 (s, 1H), 5.39 (d, J=19.9 Hz, 2H), 4.28 (s, 3H), 4.00 (s, 3H).

### Example 53: 3-(3-aminobenzyl)-8-fluoro-7-methoxy-5-methyl-3,5-dihydro-4H-pyridazino[4,5-b]indol-4-one

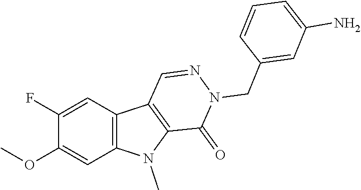 [SMILES: COc1cc2c(cc1F)c1cnn(Cc3cccc(N)c3)c(=O)c1n2C]

Example 53 was generated from intermediate AB by employing analogous reagents and procedures outlined for the generation of example 3. 1H NMR (400 MHz, DMSO-d6) δ 8.71 (s, 1H), 8.08 (d, J=11.3 Hz, 1H), 7.48 (d, J=7.2 Hz, 1H), 6.94 (dd, J=7.9 Hz, 1H), 6.54-6.30 (m, 4H), 5.23 (s, 2H), 5.04 (s, 2H), 4.28 (s, 3H), 4.01 (s, 3H).

### Example 54: 3-(3-aminobenzyl)-8-fluoro-7-hydroxy-5-methyl-3,5-dihydro-4H-pyridazino[4,5-b]indol-4-one

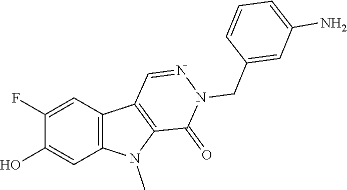 [SMILES: Cn1c2cc(O)c(F)cc2c2cnn(Cc3cccc(N)c3)c(=O)c21]

Example 54 was generated from Example 53 employing an analogous procedure used to generate Example 15. 1H NMR (400 MHz, Methanol-d4) δ 8.54 (s, 1H), 7.68 (d, J=10.8 Hz, 1H), 7.10-7.01 (m, 1H), 6.95 (d, J=7.4 Hz, 1H), 6.75-6.58 (m, 3H), 5.74-5.70 (m, 1H), 5.51 (s, 3H) 5.44-5.32 (m, 2H), 4.20 (s, 3H).

### Example 55: 3-(2-fluorobenzyl)-8-methoxy-5-methyl-3,5-dihydro-4H-pyridazino[4,5-b]indol-4-one

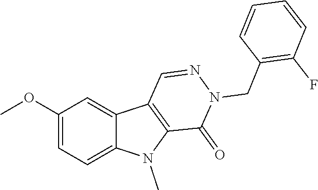 [SMILES: COc1ccc2c(c1)c1cnn(Cc3ccccc3F)c(=O)c1n2C]

Example 55 was generated by treating Intermediate AL (203 mg, 0.89 mmol) with KOtBu (199 mg, 1.77 mmol) and 1-(chloromethyl)-2-fluorobenzene (141 mg, 0.98 mmol) in DMF. The reaction mixture was stirred at rt overnight and water was added. The resulting precipitate was filtered to provide the desired product (205 mg). 1H NMR (400 MHz, DMSO-d6) δ 8.81 (s, 1H), 7.76 (d, J=2.5 Hz, 1H), 7.69 (dd, J=9.1, 0.6 Hz, 1H), 7.34 (dddd, J=8.9, 7.3, 5.4, 2.1 Hz, 1H), 7.30-7.09 (m, 4H), 5.46 (s, 2H), 4.26 (s, 3H), 3.87 (s, 3H).

### Example 56: 3-(2-fluorobenzyl)-8-hydroxy-5-methyl-3,5-dihydro-4H-pyridazino[4,5-b]indol-4-one

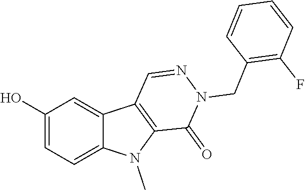 [SMILES: Cn1c2ccc(O)cc2c2cnn(Cc3ccccc3F)c(=O)c21]

Example 56 was generated from Example 53 employing an analogous procedure used to generate Example 15. 1H NMR (400 MHz, DMSO-d6) δ 9.46 (s, 1H), 8.73 (s, 1H), 7.59 (d, J=8.9 Hz, 1H), 7.46 (dd, J=2.4, 0.6 Hz, 1H), 7.39-7.27 (m, 1H), 7.26-7.06 (m, 4H), 5.45 (s, 2H), 4.23 (s, 3H).

### Example 57: 3-(2-fluorobenzyl)-7-((6-fluoropyridin-2-yl)oxy)-5-methyl-3,5-dihydro-4H-pyridazino[4,5-b]indol-4-one

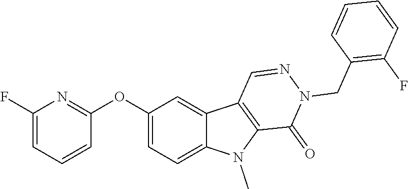 [SMILES: Cn1c2ccc(Oc3cccc(F)n3)cc2c2cnn(Cc3ccccc3F)c(=O)c21]

Example 57 was generated by treating Example 56 with 2,6-difluoropyridine (11.7 mg, 0.102 mmol) and KOtBu (32 mg, 0.232 mmol) in DMF. The reaction mixture was stirred overnight at 100° C., cooled to rt and water was added to the reaction mixture. The reaction mixture was diluted with EtOAc, washed with saturated aqueous sodium bicarbonate, dried, concentrated and purified by DCC (0-100% EtOAc in hexanes) to provide the desired product (36 mg). 1H NMR (400 MHz, DMSO-d6) δ 9.46 (s, 1H), 8.73 (s, 1H), 7.59 (d, J=8.9 Hz, 1H), 7.46 (dd, J=2.4, 0.6 Hz, 1H), 7.39-7.27 (m, 1H), 7.26-7.06 (m, 4H), 5.45 (s, 2H), 4.23 (s, 3H).

### Example 58: 8-methoxy-5-methyl-3-((6-methylpyridin-2-yl)methyl)-3,5-dihydro-4H-pyridazino[4,5-b]indol-4-one

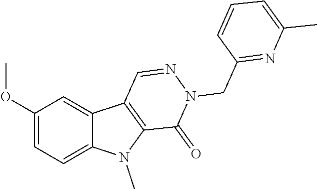

Example 57 was generated by an analogous procedure used to generate Example 55 wherein commercially available 2-(chloromethyl)-6-methylpyridine is employed to provide the desired product. 1H NMR (400 MHz, DMSO-d6) δ 9.44 (s, 1H), 8.74 (s, 1H), 7.80-6.66 (m, 6H), 5.44 (s, 2H), 4.23 (s, 3H), 2.45 (s, 3H).

### Example 59: 8-hydroxy-5-methyl-3-((6-methylpyridin-2-yl)methyl)-3,5-dihydro-4H-pyridazino[4,5-b]indol-4-one

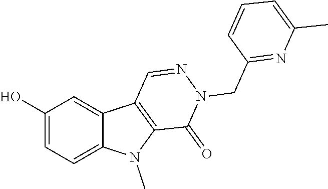 [SMILES: Cc1cccc(Cn2ncc3c4cc(O)ccc4n(C)c3c2=O)n1]

Example 59 was generated from Example 53 employing an analogous procedure used to generate Example 15. 1H NMR (400 MHz, DMSO-d6) δ 9.44 (s, 1H), 8.74 (s, 1H), 7.80-6.66 (m, 6H), 5.44 (s, 2H), 4.23 (s, 3H), 2.45 (s, 3H).

### Intermediate AH: 5-methyl-3-((6-methylpyridin-2-yl)methyl)-8-((1-(tetrahydro-2H-pyran-2-yl)-1H-pyrazolo[4,3-b]pyridin-5-yl)oxy)-3,5-dihydro-4H-pyridazino[4,5-b]indol-4-one

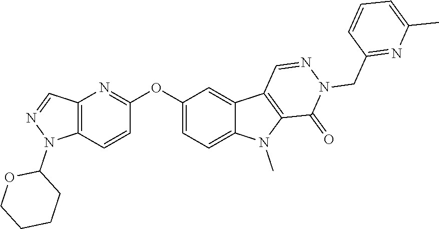 [SMILES: Cc1cccc(Cn2ncc3c4cc(Oc5ccc6c(cnn6C6CCCCO6)n5)ccc4n(C)c3c2=O)n1]

Example AH was generated by treatment of Example 59 (15 mg, 0.47 mmol) with 5-bromo-1-(tetrahydro-2H-pyran-2-yl)-1H-pyrazolo[4,3-b]pyridine (16 mg, 0.56 mmol) and CsCO₃ (31 mg, 94 mmol) in DMF (3 mL). The reaction mixture was heated to 120° C. overnight. The reaction mixture was cooled to rt, diluted with EtOAc, washed with saturated aqueous sodium bicarbonate, dried, concentrated and purified by DCC (0-100% EtOAc in hexanes) to provide the desired product (5 mg). 1H NMR (400 MHz, Chloroform-d) δ 8.52 (s, 1H), 8.14-8.06 (m, 2H), 7.88 (dd, J=2.3, 0.6 Hz, 1H), 7.59 (dd, J=9.0, 0.6 Hz, 1H), 7.55-7.44 (m, 2H), 7.12-7.02 (m, 2H), 6.94-6.87 (m, 1H), 5.66 (s, 2H), 5.63 (dd, J=8.5, 3.7 Hz, 1H), 4.41 (s, 3H), 4.18-4.06 (m, 1H), 3.82-3.71 (m, 1H), 2.58 (s, 3H), 2.29-2.14 (m, 2H), 1.87-1.56 (m, 4H).

### Example 60: 8-((1H-pyrazolo[4,3-b]pyridin-5-yl)oxy)-5-methyl-3-((6-methylpyridin-2-yl)methyl)-3,5-dihydro-4H-pyridazino[4,5-b]indol-4-one

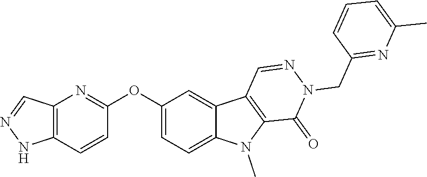 [SMILES: Cc1cccc(Cn2ncc3c4cc(Oc5ccc6[nH]ncc6n5)ccc4n(C)c3c2=O)n1]

Example 57 was generated through the treatment of Intermediate AH (5 mg, 0.009 mmol) in methanol (1 mL) with 4 M HCl in dioxane (0.2 mL). The reaction mixture was stirred overnight, diluted with EtOAc, washed with saturated aqueous sodium bicarbonate, dried, concentrated and purified by FCC (0-100% EtOAc in hexanes). 1H NMR (400 MHz, Chloroform-d) δ 8.52 (s, 1H), 8.05 (s, 1H), 7.92 (dd, J=8.9, 1.0 Hz, 1H), 7.87 (dd, J=2.2, 0.6 Hz, 1H), 7.60 (dd, J=9.0, 0.6 Hz, 1H), 7.57-7.43 (m, 2H), 7.14 (d, J=9.0 Hz, 1H), 7.05 (d, J=7.6 Hz, 1H), 6.91 (d, J=7.7 Hz, 1H), 5.67 (s, 2H), 4.41 (s, 3H), 2.58 (s, 3H).

### Example 61: 7-((6-fluoropyridin-2-yl)oxy)-5-methyl-3-((6-methylpyridin-2-yl)methyl)-3,5-dihydro-4H-pyridazino[4,5-b]indol-4-one

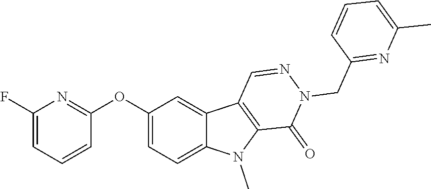 [SMILES: Cc1cccc(Cn2ncc3c4cc(Oc5cccc(F)n5)ccc4n(C)c3c2=O)n1]

Example 61 was generated from Example 59 in an analogous fashion to the generation of Example 57. 1H NMR (400 MHz, DMSO-d6) δ 8.83 (s, 1H), 8.10 (dd, J=2.3, 0.6 Hz, 1H), 8.04 (dt, J=8.6, 7.9 Hz, 1H), 7.86 (dd, J=9.0, 0.6 Hz, 1H), 7.60 (t, J=7.7 Hz, 1H), 7.47 (dd, J=9.0, 2.4 Hz, 1H), 7.14 (d, J=7.7 Hz, 1H), 7.01-6.94 (m, 1H), 6.92-6.87 (m, 1H), 6.84 (d, J=7.8 Hz, 1H), 5.47 (s, 2H), 4.32 (s, 3H), 2.45 (s, 3H).

Intermediate AI: 5-methyl-3-((6-methylpyridin-2-yl)methyl)-7-((1-(tetrahydro-2H-pyran-2-yl)-1H-pyrazolo[4,3-b]pyridin-5-yl)oxy)-3,5-dihydro-4H-pyridazino[4,5-b]indol-4-one

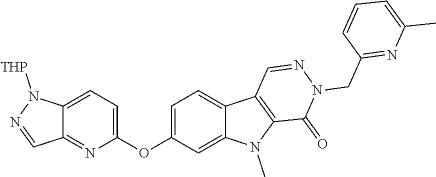

Intermediate AI was generated from Example 29 via an analogous procedure for the generation of Intermediate AH. 1H NMR (400 MHz, DMSO-d6) δ 8.86 (s, 1H), 8.44 (d, J=0.9 Hz, 1H), 8.30-8.21 (m, 2H), 7.67 (d, J=2.0 Hz, 1H), 7.61 (t, J=7.7 Hz, 1H), 7.26 (dd, J=8.6, 2.0 Hz, 1H), 7.16 (t, J=8.7 Hz, 2H), 6.85 (d, J=7.8 Hz, 1H), 5.69 (dd, J=9.6, 2.5 Hz, 1H), 5.48 (s, 2H), 4.25 (s, 3H), 3.96 (d, J=12.0 Hz, 1H), 3.70 (dt, J=11.6, 6.7 Hz, 1H), 2.46 (s, 3H), 2.26-2.11 (m, 1H), 1.99 (d, J=6.1 Hz, 2H), 1.81-1.65 (m, 1H), 1.58 (dq, J=9.8, 5.7, 4.8 Hz, 2H).

### Example 62: 7-((1H-pyrazolo[4,3-b]pyridin-5-yl)oxy)-5-methyl-3-((6-methylpyridin-2-yl)methyl)-3,5-dihydro-4H-pyridazino[4,5-b]indol-4-one

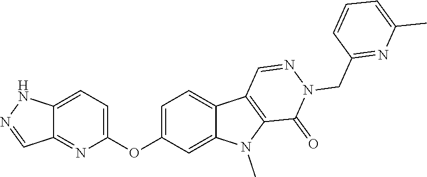 [SMILES: Cc1cccc(Cn2ncc3c4ccc(Oc5ccc6[nH]ncc6n5)cc4n(C)c3c2=O)n1]

Example 62 was generated from Intermediate AI via and analogous procedure for the generation of Example 60. 1H NMR (400 MHz, DMSO-d6) δ 13.32 (s, 1H), 8.85 (s, 1H), 8.26 (dd, J=8.7, 0.6 Hz, 1H), 8.14 (dd, J=8.9, 1.0 Hz, 1H), 8.03-7.99 (m, 1H), 7.66-7.55 (m, 2H), 7.24 (dd, J=8.6, 2.0 Hz, 1H), 7.19 (d, J=8.9 Hz, 1H), 7.15 (d, J=7.6 Hz, 1H), 6.85 (d, J=7.7 Hz, 1H), 5.48 (s, 2H), 4.24 (s, 3H), 2.46 (s, 3H).

Intermediate AJ: 5-methyl-3-((6-methylpyridin-2-yl)methyl)-7-((6-((2-(trimethylsilyl)ethoxy)methoxy)pyridin-2-yl)oxy)-3,5-dihydro-4H-pyridazino[4,5-b]indol-4-one

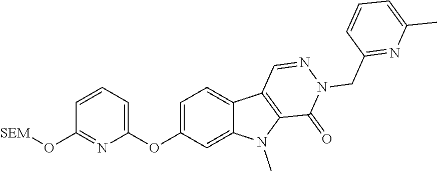 [SMILES: Cc1cccc(Cn2ncc3c4ccc(Oc5cccc(OCOCC[Si](C)(C)C)n5)cc4n(C)c3c2=O)n1]

Intermediate AJ was generated from Example 29 by treatment with 2-chloro-6-((2-(trimethylsilyl)ethoxy)methoxy)pyridine and employing analogous reaction conditions used in the generation of Example 57.

### Example 63: 7-((6-hydroxypyridin-2-yl)oxy)-5-methyl-3-((6-methylpyridin-2-yl)methyl)-3,5-dihydro-4H-pyridazino[4,5-b]indol-4-one

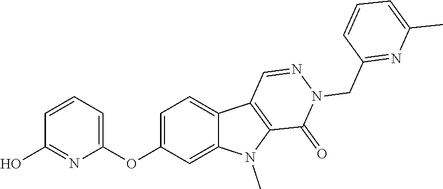 [SMILES: Cc1cccc(Cn2ncc3c4ccc(Oc5cccc(O)n5)cc4n(C)c3c2=O)n1]

Example 57 was generated through the treatment of Intermediate AJ (28.9 mg, 0.53 mmol) in DCM (3 mL) with 4 M HCl in dioxane (1.0 mL). The reaction mixture was stirred for 1 hour at rt. Water and K₂CO₃ (50 mg) were added and the reaction mixture was stirred for 1 hour. The reaction mixture was diluted with DCM and washed with water, dried, concentrated and purified by FCC (0-100% EtOAc in hexanes) to provide the desired product (7.3 mg). 1H NMR (400 MHz, Chloroform-d) δ 8.46 (s, 1H), 7.97 (dd, J=8.6, 0.6 Hz, 1H), 7.63-7.54 (m, 1H), 7.49 (t, J=7.7 Hz, 1H), 7.24 (d, J=2.0 Hz, 1H), 7.17 (dd, J=8.7, 2.1 Hz, 1H), 7.05 (d, J=7.7 Hz, 1H), 6.84 (d, J=7.8 Hz, 1H), 6.48 (dd, J=8.3, 0.7 Hz, 1H), 6.13 (d, J=7.7 Hz, 1H), 5.59 (s, 2H), 4.28 (s, 3H), 2.58 (s, 3H).

### Intermediate AK: 5-methyl-3-((6-methylpyridin-2-yl)methyl)-7-((1-(tetrahydro-2H-pyran-2-yl)-1H-pyrazolo[4,3-c]pyridin-4-yl)oxy)-3,5-dihydro-4H-pyridazino[4,5-b]indol-4-one

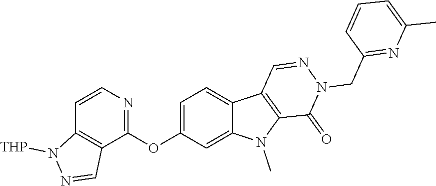

Intermediate AK was generated from Example 29 using a procedure analogous to that which was used to generate Intermediate AI. 1H NMR (400 MHz, DMSO-d6) δ 8.88 (s, 1H), 8.35 (d, J=0.8 Hz, 1H), 8.29 (dd, J=8.7, 0.6 Hz, 1H), 7.88 (d, J=6.0 Hz, 1H), 7.78-7.73 (m, 1H), 7.61 (t, J=7.7 Hz, 1H), 7.51 (dd, J=6.1, 1.0 Hz, 1H), 7.31 (dd, J=8.6, 2.0 Hz, 1H), 7.15 (d, J=7.6 Hz, 1H), 6.86 (d, J=7.8 Hz, 1H), 5.95-5.89 (m, 1H), 5.48 (s, 2H), 3.92 (d, J=11.2 Hz, 1H), 3.86-3.71 (m, 1H), 2.55 (d, J=5.3 Hz, 1H), 2.45 (s, 3H), 2.43-2.30 (m, 1H), 2.01 (d, J=14.8 Hz, 2H), 1.77 (s, 1H), 1.61 (s, OH).

Example 64: 7-((1H-pyrazolo[4,3-c]pyridin-4-yl)oxy)-5-methyl-3-((6-methylpyridin-2-yl)methyl)-3,5-dihydro-4H-pyridazino[4,5-b]indol-4-one

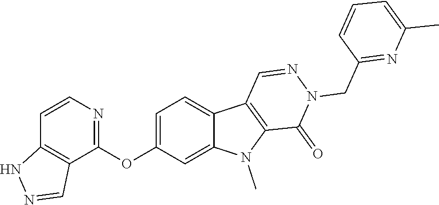 [SMILES: Cc1cccc(Cn2ncc3c4ccc(Oc5nccc6[nH]ncc56)cc4n(C)c3c2=O)n1]

Example 64 was generated from Intermediate AK in an analogous fashion to the procedure used in the generation of Example 60. 1H NMR (400 MHz, Chloroform-d) δ 10.55 (s, 1H), 8.58 (s, 1H), 8.29 (s, 1H), 8.09 (dd, J=8.6, 0.6 Hz, 1H), 7.95 (d, J=6.0 Hz, 1H), 7.52 (t, J=7.7 Hz, 1H), 7.48 (dd, J=2.0, 0.6 Hz, 1H), 7.32 (dd, J=8.6, 2.0 Hz, 1H), 7.15 (d, J=6.1 Hz, 1H), 7.06 (d, J=7.7 Hz, 1H), 6.92 (d, J=7.7 Hz, 1H), 5.68 (s, 2H), 4.35 (s, 3H), 2.58 (s, 3H).

### Intermediate AL: 5-methyl-3-((6-methylpyridin-2-yl)methyl)-7-((1-(tetrahydro-2H-pyran-2-yl)-1H-pyrazolo[3,4-d]pyrimidin-4-yl)oxy)-3,5-dihydro-4H-pyridazino[4,5-b]indol-4-one

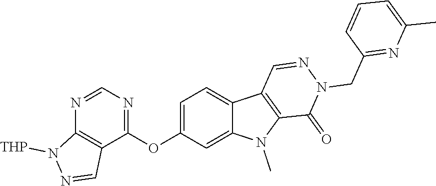

Intermediate AL was generated from Example 29 through the treatment with 4-chloro-1-(tetrahydro-2H-pyran-2-yl)-1H-pyrazolo[3,4-d]pyrimidine in an analogous procedure to that which was used to generate Intermediate AI. 1H NMR (400 MHz, DMSO-d6) δ 8.90 (s, 1H), 8.60 (s, 1H), 8.38-8.30 (m, 1H), 8.26 (d, J=0.5 Hz, 1H), 7.92-7.85 (m, 1H), 7.61 (t, J=7.7 Hz, 1H), 7.40 (dd, J=8.6, 2.0 Hz, 1H), 7.15 (d, J=7.6 Hz, 1H), 6.87 (d, J=7.8 Hz, 1H), 6.01 (d, J=10.3 Hz, 1H), 5.48 (s, 2H), 4.26 (s, 3H), 3.98 (d, J=11.8 Hz, 1H), 3.74 (d, J=12.8 Hz, 1H), 2.71-2.65 (m, 1H), 2.58-2.54 (m, 1H), 2.45 (s, 3H), 2.35-2.31 (m, 1H), 2.13-1.41 (m, 2H).

### Example 65: 7-((1H-pyrazolo[3,4-d]pyrimidin-4-yl)oxy)-5-methyl-3-((6-methylpyridin-2-yl)methyl)-3,5-dihydro-4H-pyridazino[4,5-b]indol-4-one

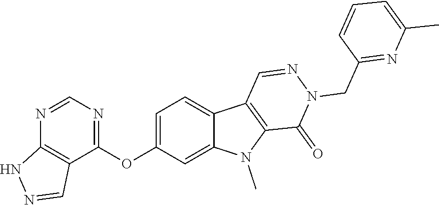 [SMILES: Cc1cccc(Cn2ncc3c4ccc(Oc5ncnc6[nH]ncc56)cc4n(C)c3c2=O)n1]

Example 57 was generated from Intermediate AL in an analogous fashion to the procedure used in the generation of Example 60. 1H NMR (400 MHz, DMSO-d6) δ 14.20 (s, 1H), 8.89 (s, 1H), 8.53 (s, 1H), 8.34 (d, J=8.6 Hz, 1H), 8.17 (d, J=1.3 Hz, 1H), 7.87 (d, J=2.0 Hz, 1H), 7.61 (t, J=7.7 Hz, 1H), 7.39 (dd, J=8.6, 2.0 Hz, 1H), 7.15 (d, J=7.6 Hz, 1H), 6.86 (d, J=7.8 Hz, 1H), 5.48 (s, 2H), 4.26 (s, 3H), 2.45 (s, 3H).

### Example 66: 7-((6-fluoropyridin-2-yl)oxy)-5-methyl-3-((6-methylpyridin-2-yl)methyl)-3,5-dihydro-4H-pyridazino[4,5-b]indol-4-one

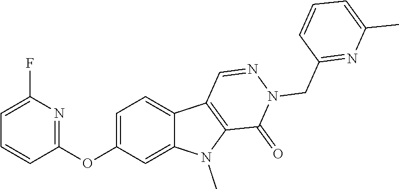 [SMILES: Cc1cccc(Cn2ncc3c4ccc(Oc5cccc(F)n5)cc4n(C)c3c2=O)n1]

Example 66 was generated from Example 29 using a procedure analogous to that employed in the generation of Example 57. 1H NMR (400 MHz, DMSO-d6) δ 8.87 (s, 1H), 8.29 (d, J=8.6 Hz, 1H), 8.06 (q, J=8.2 Hz, 1H), 7.68 (d, J=2.0 Hz, 1H), 7.61 (t, J=7.7 Hz, 1H), 7.24 (dd, J=8.6, 2.1 Hz, 1H), 7.15 (d, J=7.7 Hz, 1H), 7.01 (dd, J=7.9, 1.6 Hz, 1H), 6.92 (dd, J=7.9, 2.4 Hz, 1H), 6.84 (d, J=7.8 Hz, 1H), 5.47 (s, 2H), 4.25 (s, 3H), 2.45 (s, 3H).

### Example 67: 7-((6-chloropyridin-2-yl)oxy)-5-methyl-3-((6-methylpyridin-2-yl)methyl)-3,5-dihydro-4H-pyridazino[4,5-b]indol-4-one

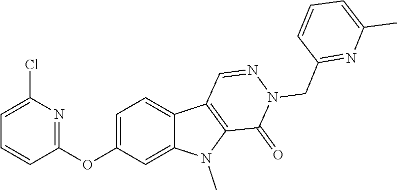 [SMILES: Cc1cccc(Cn2ncc3c4ccc(Oc5cccc(Cl)n5)cc4n(C)c3c2=O)n1]

Example 57 was generated by treating Example 29 with 2-chloro-6-nitropyridine in a procedure analogous to that employed in the generation of Example 57. 1H NMR (400 MHz, DMSO-d6) δ 8.88 (s, 1H), 8.29 (d, J=8.7 Hz, 1H), 8.00-7.92 (m, 1H), 7.73 (s, 1H), 7.68 (d, J=2.0 Hz, 1H), 7.35-7.20 (m, 3H), 7.10 (d, J=8.2 Hz, 1H), 6.95 (s, 1H), 5.52 (s, 2H), 4.25 (s, 3H), 2.45 (s, 3H).

### Example 68: 3-(2-fluorobenzyl)-7-methoxy-5-methyl-3,5-dihydro-4H-pyridazino[4,5-b]indol-4-one

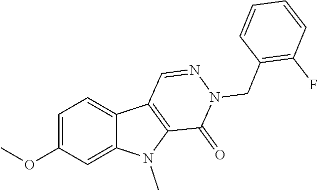 [SMILES: COc1ccc2c3cnn(Cc4ccccc4F)c(=O)c3n(C)c2c1]

Example 57 was generated from the treatment of Intermediate L with 1-(chloromethyl)-2-fluorobenzene and KOtBu in DMF. The reaction mixture was stirred overnight at rt, water was added and the resulting precipitate was collected via filtration to provide the desired product. 1H NMR (400 MHz, DMSO-d6) δ 8.76 (s, 1H), 8.08 (dd, J=8.8, 0.5 Hz, 1H), 7.37-7.30 (m, 1H), 7.27-7.10 (m, 5H), 7.03 (dd, J=8.8, 2.2 Hz, 1H), 5.46 (s, 2H), 4.25 (s, 3H), 3.92 (s, 3H).

### Example 69: 3-(2-fluorobenzyl)-7-hydroxy-5-methyl-3,5-dihydro-4H-pyridazino[4,5-b]indol-4-one

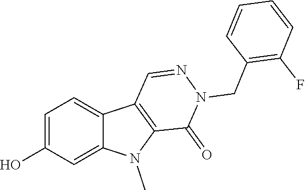 [SMILES: Cn1c2cc(O)ccc2c2cnn(Cc3ccccc3F)c(=O)c21]

Example 69 was generated from Example 68 employing an analogous procedure used to generate Example 15. 1H NMR (400 MHz, DMSO-d6) δ 9.99 (s, 1H), 8.71 (s, 1H), 8.02-7.95 (m, 1H), 7.39-7.26 (m, 1H), 7.27-7.10 (m, 3H), 6.96 (dd, J=2.1, 0.6 Hz, 1H), 6.91 (dd, J=8.6, 2.1 Hz, 1H), 5.45 (s, 2H), 4.17 (s, 3H).

### Intermediate AM: 3-(2-fluorobenzyl)-5-methyl-7-((1-(tetrahydro-2H-pyran-2-yl)-1H-pyrazolo[4,3-b]pyridin-5-yl)oxy)-3,5-dihydro-4H-pyridazino[4,5-b]indol-4-one

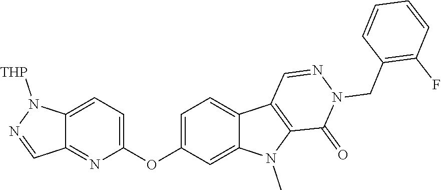

Intermediate AM was generated from Example 69 via a procedure analogous to the generation of Intermediate AI.

### Example 70: 7-((1H-pyrazolo[4,3-b]pyridin-5-yl)oxy)-3-(2-fluorobenzyl)-5-methyl-3,5-dihydro-4H-pyridazino[4,5-b]indol-4-one

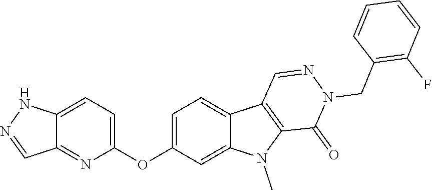 [SMILES: Cn1c2cc(Oc3ccc4[nH]ncc4n3)ccc2c2cnn(Cc3ccccc3F)c(=O)c21]

Example 70 was generated from Intermediate AM in an analogous fashion to the procedure used in the generation of Example 60. 1H NMR (400 MHz, DMSO-d6) δ 13.32 (s, 1H), 8.84 (s, 1H), 8.24 (dd, J=8.6, 0.6 Hz, 1H), 8.14 (dd, J=8.9, 1.0 Hz, 1H), 8.01 (t, J=1.2 Hz, 1H), 7.64-7.58 (m, 1H), 7.40-7.31 (m, 1H), 7.28-7.09 (m, 5H), 5.49 (s, 2H), 4.24 (s, 3H).

### Intermediate AN: 3-(2-fluorobenzyl)-5-methyl-7-((1-(tetrahydro-2H-pyran-2-yl)-1H-pyrazolo[4,3-c]pyridin-4-yl)oxy)-3,5-dihydro-4H-pyridazino[4,5-b]indol-4-one

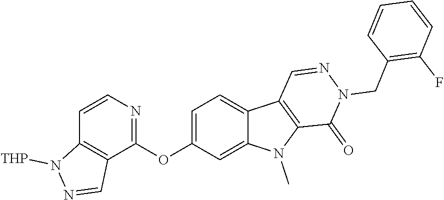

Intermediate AN was generated by treating Example 69 with 5-bromo-1-(tetrahydro-2H-pyran-2-yl)-1H-pyrazolo[4,3-b]pyridine via an analogous procedure that was employed in the generation of Intermediate AI. 1H NMR (400 MHz, DMSO-d6) δ 8.86 (s, 1H), 8.34 (d, J=0.7 Hz, 1H), 8.27 (d, J=8.6 Hz, 1H), 7.88 (d, J=6.1 Hz, 1H), 7.75 (d, J=2.0 Hz, 1H), 7.50 (dd, J=6.1, 0.9 Hz, 1H), 7.41-7.11 (m, 5H), 5.96-5.88 (m, 1H), 5.49 (s, 2H), 4.25 (s, 3H), 3.92 (d, J=11.6 Hz, 1H), 3.82 (d, J=28.1 Hz, 1H), 2.70-2.65 (m, 1H), 2.55 (d, J=5.3 Hz, 2H), 2.46 (s, OH), 2.37-2.28 (m, 1H), 2.03 (s, 2H), 1.61 (s, 1H).

### Example 71: 7-((1H-pyrazolo[4,3-c]pyridin-4-yl)oxy)-3-(2-fluorobenzyl)-5-methyl-3,5-dihydro-4H-pyridazino[4,5-b]indol-4-one

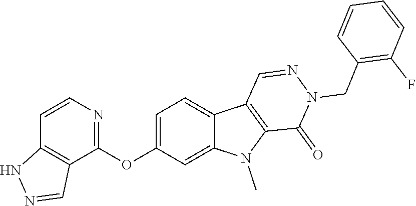 [SMILES: Cn1c2cc(Oc3nccc4[nH]ncc34)ccc2c2cnn(Cc3ccccc3F)c(=O)c21]

Example 57 was generated from Intermediate AN in analogous fashion to Example 60. 1H NMR (400 MHz, Chloroform-d) δ 10.72 (s, 1H), 8.54 (s, 1H), 8.26 (d, J=1.0 Hz, 1H), 8.06 (dd, J=8.6, 0.6 Hz, 1H), 7.94 (d, J=6.0 Hz, 1H), 7.47 (dd, J=2.1, 0.5 Hz, 1H), 7.36-7.23 (m, 3H), 7.17-7.04 (m, 3H), 5.62 (s, 2H), 4.34 (s, 3H).

Intermediate AO: 3-(2-fluorobenzyl)-5-methyl-7-((6-((2-(trimethylsilyl)ethoxy)methoxy)pyridin-2-yl)oxy)-3,5-dihydro-4H-pyridazino[4,5-b]indol-4-one

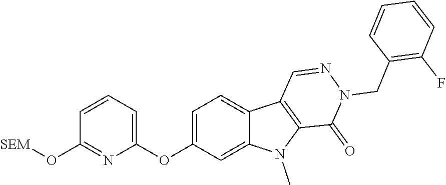 [SMILES: Cn1c2cc(Oc3cccc(OCOCC[Si](C)(C)C)n3)ccc2c2cnn(Cc3ccccc3F)c(=O)c21]

Intermediate AO was generated from Example 69 via procedure employed to generate intermediate AJ. 1H NMR (400 MHz, DMSO-d6) δ 8.87 (s, 1H), 8.34 (d, J=8.7 Hz, 1H), 7.73 (d, J=2.1 Hz, 1H), 7.40-7.11 (m, 6H), 6.16 (dd, J=9.2, 1.1 Hz, 1H), 5.58 (s, 2H), 5.48 (s, 2H), 5.33 (dd, J=7.6, 1.1 Hz, 1H), 4.26 (s, 3H), 3.72 (dd, J=8.5, 7.5 Hz, 2H), 0.91 (dd, J=8.4, 7.6 Hz, 2H), −0.02 (s, 9H).

### Example 72: 3-(2-fluorobenzyl)-7-((6-hydroxypyridin-2-yl)oxy)-5-methyl-3,5-dihydro-4H-pyridazino [4,5-b]indol-4-one

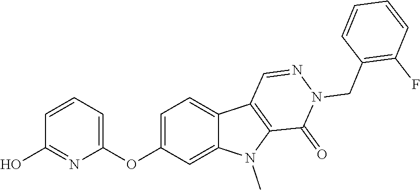 [SMILES: Cn1c2cc(Oc3cccc(O)n3)ccc2c2cnn(Cc3ccccc3F)c(=O)c21]

Example 72 was generated from Intermediate AO via analogous procedure employed in the generation of Example 63. 1H NMR (400 MHz, DMSO-d6) δ 10.79 (s, 1H), 8.83 (s, 1H), 8.23 (d, J=8.7 Hz, 1H), 7.69 (s, 1H), 7.58 (s, 1H), 7.43-7.29 (m, 1H), 7.28-7.10 (m, 4H), 6.55-6.31 (m, 2H), 5.48 (s, 2H), 4.24 (s, 3H).

### Intermediate AP: 3-(2-fluorobenzyl)-5-methyl-7-((1-(tetrahydro-2H-pyran-2-yl)-1H-pyrazolo[3,4-d]pyrimidin-4-yl)oxy)-3,5-dihydro-4H-pyridazino[4,5-b]indol-4-one

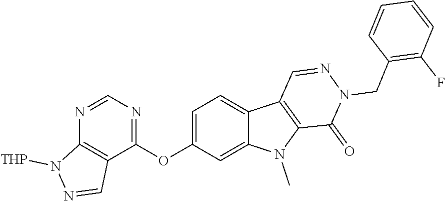

Intermediate AP was generated from Example 69 via an analogous procedure employed for the generation of Intermediate AN. 1H NMR (400 MHz, DMSO-d6) δ 8.89 (s, 1H), 8.60 (s, 1H), 8.33 (d, J=8.7 Hz, 1H), 8.25 (s, 1H), 7.87 (d, J=2.1 Hz, 1H), 7.43-7.32 (m, 2H), 7.29-7.11 (m, 3H), 6.01 (dd, J=10.2, 2.5 Hz, 1H), 5.49 (s, 2H), 4.26 (s, 3H), 3.98 (d, J=12.0 Hz, 1H), 3.72 (t, J=12.6 Hz, 1H), 2.70-2.65 (m, 1H), 2.58-2.54 (m, 1H), 2.36-2.30 (m, 1H), 2.05 (d, J=13.0 Hz, 1H), 1.94 (d, J=17.0 Hz, 1H), 1.60 (s, 1H).

### Example 73: 7-((1H-pyrazolo[3,4-d]pyrimidin-4-yl)oxy)-3-(2-fluorobenzyl)-5-methyl-3,5-dihydro-4H-pyridazino[4,5-b]indol-4-one

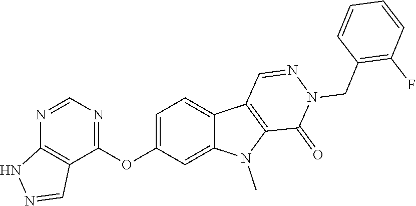 [SMILES: Cn1c2cc(Oc3ncnc4[nH]ncc34)ccc2c2cnn(Cc3ccccc3F)c(=O)c21]

Example 73 was generated from Intermediate AP via analogous procedure employed in the generation of Example 71. 1H NMR (400 MHz, Chloroform-d) δ 11.38 (s, 1H), 8.63 (s, 1H), 8.57 (s, 1H), 8.13 (t, J=4.3 Hz, 2H), 7.48 (d, J=2.0 Hz, 1H), 7.36-7.24 (m, 3H), 7.17-7.06 (m, 2H), 5.63 (s, 2H), 4.38 (s, 3H).

### Example 74: 3-(2-fluorobenzyl)-7-((6-fluoropyridin-2-yl)oxy)-5-methyl-3,5-dihydro-4H-pyridazino[4,5-b]indol-4-one

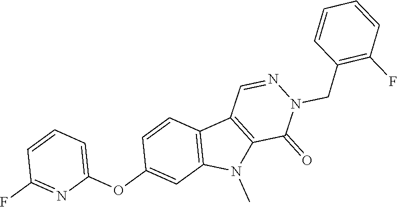 [SMILES: Cn1c2cc(Oc3cccc(F)n3)ccc2c2cnn(Cc3ccccc3F)c(=O)c21]

Example 74 was generated from Example 69 using a procedure analogous to that employed in the generation of Example 57. 1H NMR (400 MHz, DMSO-d6) δ 8.86 (s, 1H), 8.28 (d, J=8.6 Hz, 1H), 8.06 (q, J=8.1 Hz, 1H), 7.67 (d, J=1.9 Hz, 1H), 7.41-7.29 (m, 1H), 7.29-7.10 (m, 4H), 7.00 (dd, J=8.0, 1.6 Hz, 1H), 6.92 (dd, J=7.8, 2.5 Hz, 1H), 5.48 (s, 2H), 4.25 (s, 3H).

### Example 75: 7-methoxy-5-methyl-3-(3-nitrobenzyl)-3,5-dihydro-4H-pyridazino[4,5-b]indol-4-one

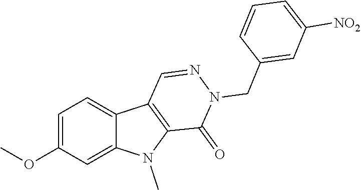 [SMILES: COc1ccc2c3cnn(Cc4cccc([N+](=O)[O-])c4)c(=O)c3n(C)c2c1]

Example 75 was generated by the treatment of Intermediate L with 1-(chloromethyl)-3-nitrobenzene via a protocol analogous that use in the generation of Example 69. 1H NMR (400 MHz, DMSO-d6) δ 8.80 (s, 1H), 8.21-8.13 (m, 2H), 8.09 (d, J=8.8 Hz, 1H), 7.82-7.75 (m, 1H), 7.69-7.59 (m, 1H), 7.25 (d, J=2.2 Hz, 1H), 7.03 (dd, J=8.8, 2.2 Hz, 1H), 5.55 (s, 2H), 4.26 (s, 3H), 3.92 (s, 3H).

### Example 76: 7-hydroxy-5-methyl-3-(3-nitrobenzyl)-3,5-dihydro-4H-pyridazino[4,5-b]indol-4-one

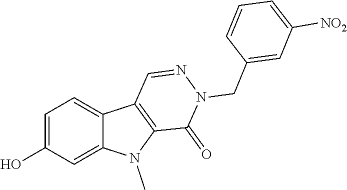 [SMILES: Cn1c2cc(O)ccc2c2cnn(Cc3cccc([N+](=O)[O-])c3)c(=O)c21]

Example 76 was generated from Example 75 employing an analogous procedure used to generate Example 15. 1H NMR (400 MHz, DMSO-d6) δ 10.00 (s, 1H), 8.75 (s, 1H), 8.20-8.12 (m, 2H), 7.99 (d, J=8.6 Hz, 1H), 7.82-7.71 (m, 1H), 7.71-7.59 (m, 1H), 6.96 (d, J=2.0 Hz, 1H), 6.94-6.87 (m, 1H), 5.54 (s, 2H), 4.17 (s, 3H).

### Example 77: 7-((6-chloropyridin-2-yl)oxy)-5-methyl-3-(3-nitrobenzyl)-3,5-dihydro-4H-pyridazino[4,5-b]indol-4-one

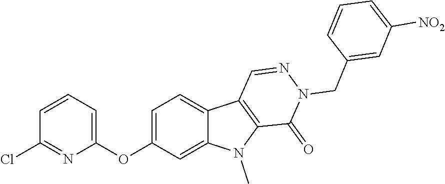 [SMILES: Cn1c2cc(Oc3cccc(Cl)n3)ccc2c2cnn(Cc3cccc([N+](=O)[O-])c3)c(=O)c21]

Example 77 was generated by treating Example 76 with 2-chloro-6-nitropyridine in a procedure analogous to that employed in the generation of Example 57. 1H NMR (400 MHz, DMSO-d6) δ 8.89 (s, 1H), 8.28 (dd, J=8.7, 0.6 Hz, 1H), 8.22-8.12 (m, 2H), 7.94 (dd, J=8.1, 7.7 Hz, 1H), 7.79 (d, J=7.9 Hz, 1H), 7.70-7.61 (m, 2H), 7.29 (dd, J=7.6, 0.6 Hz, 1H), 7.23 (dd, J=8.6, 2.1 Hz, 1H), 7.09 (dd, J=8.2, 0.6 Hz, 1H), 5.57 (s, 2H), 4.25 (s, 3H).

### Example 78: 3-(3-aminobenzyl)-7-((6-chloropyridin-2-yl)oxy)-5-methyl-3,5-dihydro-4H-pyridazino[4,5-b]indol-4-one

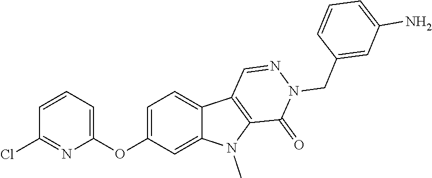 [SMILES: Cn1c2cc(Oc3cccc(Cl)n3)ccc2c2cnn(Cc3cccc(N)c3)c(=O)c21]

Example 75 was generated by treating Example 77 (23.5 mg, 0.051 mmol) with stannous chloride, dihydrate (57.4 mg, 0.254 mmol) 1,4-dioxane (3 mL), ethanol (3 mL) and water (0.1 mL). The reaction mixture was heated to 100° C. for 5 hours and cooled to rt. The reaction mixture was filtered, diluted with EtOAc, washed with saturated aqueous bicarbonate, dried, concentrated and purified by FCC (0-10% MeOH in DCM) to provide the desired product (11 mg). 1H NMR (400 MHz, DMSO-d6) δ 8.83 (s, 1H), 8.27 (dd, J=8.7, 0.6 Hz, 1H), 7.99-7.90 (m, 1H), 7.66 (d, J=2.0 Hz, 1H), 7.28 (dd, J=7.6, 0.6 Hz, 1H), 7.22 (dd, J=8.6, 2.1 Hz, 1H), 7.09 (dd, J=8.2, 0.6 Hz, 1H), 7.00-6.89 (m, 1H), 6.52-6.37 (m, 3H), 5.27 (s, 2H), 5.03 (s, 2H), 4.26 (s, 3H).

### Example 79: 5-methyl-3-(3-nitrobenzyl)-7-(3-nitrophenoxy)-3,5-dihydro-4H-pyridazino[4,5-b]indol-4-one

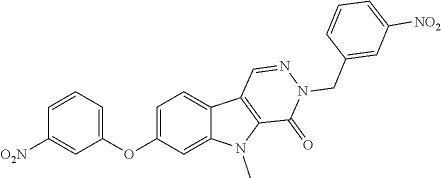 [SMILES: Cn1c2cc(Oc3cccc([N+](=O)[O-])c3)ccc2c2cnn(Cc3cccc([N+](=O)[O-])c3)c(=O)c21]

Example 79 was generated by treating Example 76 with m-Nitrobenzofluoride under condition analogous to the generation of 57. 1H NMR (400 MHz, Chloroform-d) δ 8.55 (s, 1H), 8.32 (t, J=2.0 Hz, 1H), 8.17 (ddd, J=8.2, 2.3, 1.1 Hz, 1H), 8.06 (dd, J=8.7, 0.6 Hz, 1H), 8.02 (ddd, J=8.2, 2.2, 1.0 Hz, 1H), 7.85 (t, J=2.3 Hz, 1H), 7.82 (ddd, J=7.7, 1.8, 1.1 Hz, 1H), 7.55 (td, J=8.1, 7.3 Hz, 2H), 7.41 (ddd, J=8.3, 2.5, 1.0 Hz, 1H), 7.22-7.18 (m, 1H), 7.15 (dd, J=8.6, 2.1 Hz, 1H).

### Example 80: 3-(3-aminobenzyl)-7-(3-aminophenoxy)-5-methyl-3,5-dihydro-4H-pyridazino[4,5-b]indol-4-one

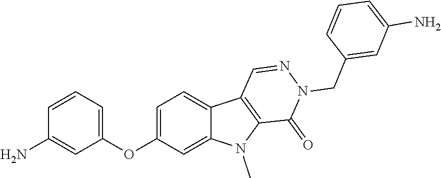 [SMILES: Cn1c2cc(Oc3cccc(N)c3)ccc2c2cnn(Cc3cccc(N)c3)c(=O)c21]

Example 80 was generated from Example 79 via an analogous procedure used to generate Example 78. 1H NMR (400 MHz, Chloroform-d) δ 8.48 (s, 1H), 7.97-7.87 (m, 1H), 7.23-7.03 (m, 4H), 6.96-6.74 (m, 2H), 6.67-6.56 (m, 1H), 6.55-6.30 (m, 3H), 5.43 (s, 2H), 4.27 (s, 3H).

### Intermediate AO: 7-methoxy-3,5-dimethyl-3,5-dihydro-4H-pyridazino[4,5-b]indol-4-one

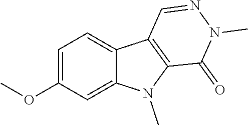

Intermediate AO was generated from treating Intermediate I (110 mg, 0.48 mmol) with iodomethane (75.6 mg, 0.53 mmol) and KOtBu (109 mg, 0.97 mmol) in DMF. The reaction mixture was heated to 60° C. for 7 hours, cooled to rt and water was added. The resulting reaction mixture was extracted with DCM and the organics were combined, dried, concentrated and purified by FCC (0-5% MeOH in DCM) to provide the desired product (64 mg). 1H NMR (400 MHz, DMSO-d6) δ 8.68 (d, J=0.6 Hz, 1H), 8.06 (d, J=8.7 Hz, 1H), 7.23 (d, J=2.2 Hz, 1H), 7.01 (dd, J=8.7, 2.2 Hz, 1H), 4.25 (s, 3H), 3.91 (s, 3H), 3.78 (s, 3H).

Intermediate AQ: 7-hydroxy-3,5-dimethyl-3,5-dihydro-4H-pyridazino[4,5-b]indol-4-one

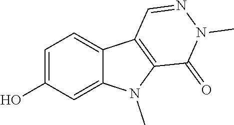 [SMILES: Cn1ncc2c3ccc(O)cc3n(C)c2c1=O]

Intermediate AQ was generated from Intermediate AO employing an analogous procedure used to generate Example 15. 1H NMR (400 MHz, DMSO-d6) δ 9.94 (s, 1H), 8.63 (s, 1H), 8.02-7.91 (m, 1H), 6.97-6.92 (m, 1H), 6.92-6.85 (m, 1H), 4.17 (s, 3H), 3.77 (s, 3H).

### Example 81: 7-((6-chloropyridin-2-yl)oxy)-3,5-dimethyl-3,5-dihydro-4H-pyridazino[4,5-b]indol-4-one

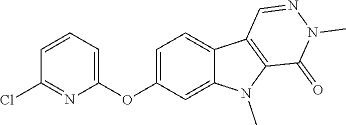 [SMILES: Cn1ncc2c3ccc(Oc4cccc(Cl)n4)cc3n(C)c2c1=O]

Example 81 was generated by treating Intermediate AQ with 2-chloro-6-nitropyridine in a procedure analogous to that employed in the generation of Example 57. 1H NMR (400 MHz, DMSO-d6) δ 8.78 (s, 1H), 8.27 (d, J=8.6 Hz, 1H), 7.98-7.90 (m, 1H), 7.65 (d, J=2.0 Hz, 1H), 7.28 (d, J=7.7 Hz, 1H), 7.24-7.19 (m, 1H), 7.11-7.05 (m, 1H), 4.25 (s, 3H), 3.81 (s, 3H).

### Intermediate AR: methyl 6-hydroxy-1-methyl-1H-indole-2-carboxylate

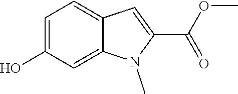

Intermediate AQ was generated from commercially available methyl 6-methoxy-1-methyl-1H-indole-2-carboxylate by employing an analogous procedure used to generate Example 15. 1H NMR (400 MHz, DMSO-d6) δ 9.56 (s, 1H), 7.47 (d, J=8.5 Hz, 1H), 7.16 (d, J=0.9 Hz, 1H), 6.79 (dt, J=2.1, 0.7 Hz, 1H), 6.69 (dd, J=8.6, 2.1 Hz, 1H), 3.90 (s, 3H), 3.82 (s, 3H).

### Intermediate AS: methyl 6-(benzyloxy)-1-methyl-1H-indole-2-carboxylate

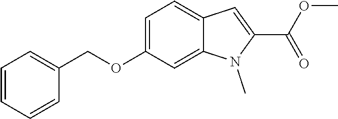 [SMILES: COC(=O)c1cc2ccc(OCc3ccccc3)cc2n1C]

Intermediate AS was generated by treating Intermediate AR (1.12 g, 4.56 mmol) with sodium hydride (327 mg, 8.18 mmol) in DMF followed by treatment with benzyl bromide (1.12 g, 6.55 mmol). The reaction mixture was stirred at rt overnight and water was added. The resulting reaction mixture was extracted with EtOAc and the organics were combined, dried and purified by FCC (0-50% EtOAc in hexanes) to provide the desired product (1.37 g). 1H NMR (400 MHz, DMSO-d6) δ 7.57 (d, J=8.7 Hz, 1H), 7.54-7.46 (m, 2H), 7.46-7.28 (m, 3H), 7.25-7.17 (m, 2H), 6.86 (dd, J=8.7, 2.2 Hz, 1H), 5.20 (s, 2H), 3.99 (s, 3H), 3.83 (s, 3H).

### Intermediate AT: methyl 6-(benzyloxy)-3-formyl-1-methyl-1H-indole-2-carboxylate

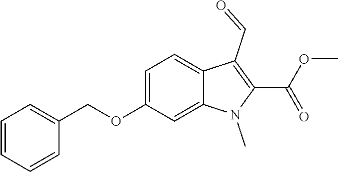 [SMILES: COC(=O)c1c(C=O)c2ccc(OCc3ccccc3)cc2n1C]

Intermediate AT was generated from intermediate AS via a protocol analogous to that which was employed in the generation of intermediate B. 1H NMR (400 MHz, DMSO-d6) δ 10.42 (s, 1H), 8.17 (d, J=8.8 Hz, 1H), 7.68-7.31 (m, 6H), 7.08 (dd, J=8.9, 2.2 Hz, 1H), 5.22 (s, 2H), 4.03 (s, 3H), 3.99 (s, 3H).

### Intermediate AU: 7-(benzyloxy)-5-methyl-3,5-dihydro-4H-pyridazino[4,5-b]indol-4-one

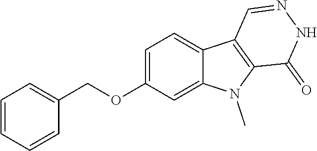 [SMILES: Cn1c2cc(OCc3ccccc3)ccc2c2cn[nH]c(=O)c21]

Intermediate AU was generated from intermediate AT via a protocol analogous to that which was employed in the generation of intermediate C. 1H NMR (400 MHz, DMSO-d6) δ 12.72 (s, 1H), 8.69 (s, 1H), 8.09 (d, J=8.8 Hz, 1H), 7.68-7.28 (m, 6H), 7.09 (dd, J=8.8, 2.2 Hz, 1H), 5.26 (s, 2H), 4.24 (s, 3H).

### Intermediate AV: Bis-tert-butyl (6-((7-(benzyloxy)-5-methyl-4-oxo-4,5-dihydro-3H-pyridazino[4,5-b]indol-3-yl)methyl)pyridin-2-yl)carbamate

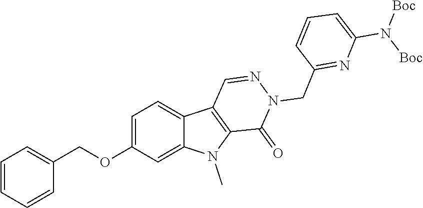 [SMILES: Cn1c2cc(OCc3ccccc3)ccc2c2cnn(Cc3cccc(N(C(=O)OC(C)(C)C)C(=O)OC(C)(C)C)n3)c(=O)c21]

Intermediate AV was generated by treating Intermediate AU with 2-[Bis(tert-butoxycarbonyl)amino]-6-(bromomethyl)pyridine under reaction conditions analogous to those used in the generation of Example 1. 1H NMR (400 MHz, DMSO-d6) δ 8.75 (s, 1H), 8.11 (dd, J=8.7, 2.2 Hz, 1H), 7.82 (t, J=7.8 Hz, 1H), 7.56-7.50 (m, 2H), 7.48-7.33 (m, 4H), 7.25 (dd, J=7.9, 0.8 Hz, 1H), 7.15-7.08 (m, 2H), 5.46 (s, 2H), 5.27 (s, 2H), 4.24 (s, 3H), 1.31 (s, 18H).

### Intermediate AW: bis-tert-butyl (6-((7-hydroxy-5-methyl-4-oxo-4,5-dihydro-3H-pyridazino[4,5-b]indol-3-yl)methyl)pyridin-2-yl)carbamate

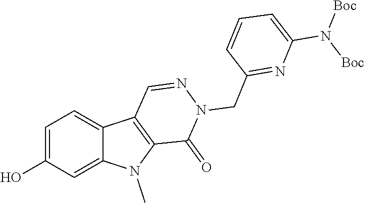 [SMILES: Cn1c2cc(O)ccc2c2cnn(Cc3cccc(N(C(=O)OC(C)(C)C)C(=O)OC(C)(C)C)n3)c(=O)c21]

Intermediate AW was generated by treating Intermediate AV (408 mg, 0.67 mmol) with 10% Pd/C (41 mg) in ethanol under a hydrogen atmosphere for 12 hours. The reaction mixture was filtered through celite and concentrated to provide the desired product which was used without purification. 1H NMR (400 MHz, DMSO-d6) δ 8.85 (s, 1H), 8.33-8.24 (m, 2H), 7.91-7.78 (m, 2H), 7.77-7.72 (m, 1H), 7.51 (dd, J=6.1, 0.9 Hz, 1H), 7.34-7.24 (m, 2H), 7.17-7.12 (m, 1H), 5.97-5.89 (m, 1H), 5.49 (s, 2H), 4.24 (s, 3H), 3.98-3.86 (m, 1H), 3.84-3.70 (m, 1H), 2.14-1.57 (m, 6H), 1.32 (s, 18H).

### Intermediate AX: bis-tert-butyl (6-((5-methyl-4-oxo-7-((1-(tetrahydro-2H-pyran-2-yl)-1H-pyrazolo[4,3-c]pyridin-4-yl)oxy)-4,5-dihydro-3H-pyridazino[4,5-b]indol-3-yl)methyl)pyridin-2-yl)carbamate

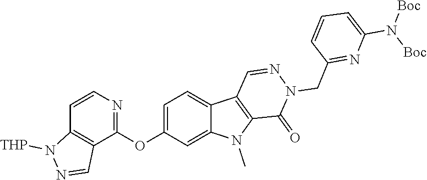

Intermediate AX was generated by treating Intermediate AW with 5-bromo-1-(tetrahydro-2H-pyran-2-yl)-1H-pyrazolo[4,3-b]pyridine via an analogous procedure that was employed in the generation of Intermediate AI. 1H NMR (400 MHz, DMSO-d6) δ 8.85 (s, 1H), 8.33-8.24 (m, 2H), 7.91-7.78 (m, 2H), 7.77-7.72 (m, 1H), 7.51 (dd, J=6.1, 0.9 Hz, 1H), 7.34-7.24 (m, 2H), 7.17-7.12 (m, 1H), 5.97-5.89 (m, 1H), 5.49 (s, 2H), 4.24 (s, 3H), 3.98-3.86 (m, 1H), 3.84-3.70 (m, 1H), 2.14-1.57 (m, 6H), 1.32 (s, 18H).

### Example 82: 7-((1H-pyrazolo[4,3-c]pyridin-4-yl)oxy)-3-((6-aminopyridin-2-yl)methyl)-5-methyl-3,5-dihydro-4H-pyridazino[4,5-b]indol-4-one

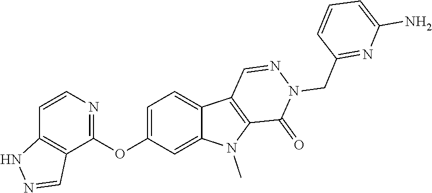 [SMILES: Cn1c2cc(Oc3nccc4[nH]ncc34)ccc2c2cnn(Cc3cccc(N)n3)c(=O)c21]

Example 82 was generated by treating Intermediate AX (16 mg, 0.22 mmol) in methanol (2 mL) with 4 M HCl in dioxane (0.3 mL) and the reaction mixture was stirred at rt for 8 hours. The reaction mixture was purified by hplc to provide the desired product (3.2 mg). 1H NMR (400 MHz, Methanol-d4) δ 8.76 (s, 1H), 8.25 (d, J=8.6 Hz, 1H), 8.13 (s, 1H), 7.84 (d, J=6.1 Hz, 1H), 7.66 (d, J=2.0 Hz, 1H), 7.39 (dd, J=8.3, 7.3 Hz, 1H), 7.34-7.27 (m, 2H), 6.48 (d, J=8.1 Hz, 1H), 6.34 (d, J=7.4 Hz, 1H), 5.42 (s, 2H), 4.33 (s, 3H).

Intermediate AY: bis-tert-butyl (6-((7-((6-fluoropyridin-2-yl)oxy)-5-methyl-4-oxo-4,5-dihydro-3H-pyridazino[4,5-b]indol-3-yl)methyl)pyridin-2-yl)carbamate

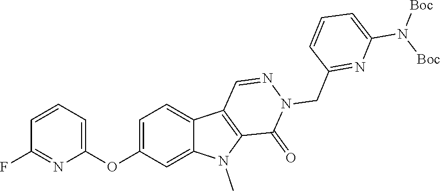 [SMILES: Cn1c2cc(Oc3cccc(F)n3)ccc2c2cnn(Cc3cccc(N(C(=O)OC(C)(C)C)C(=O)OC(C)(C)C)n3)c(=O)c21]

Intermediate AY was generated from Intermediate AW using a procedure analogous to that employed in the generation of Example 57. 1H NMR (400 MHz, Chloroform-d) δ 8.55 (s, 1H), 8.04 (dd, J=8.6, 0.6 Hz, 1H), 7.83 (q, J=8.0 Hz, 1H), 7.38 (dd, J=8.2, 7.4 Hz, 1H), 7.34 (dd, J=2.1, 0.6 Hz, 1H), 7.19 (dd, J=8.6, 2.0 Hz, 1H), 6.85 (ddd, J=7.9, 1.5, 0.6 Hz, 1H), 6.68 (ddd, J=7.8, 2.7, 0.6 Hz, 1H), 6.52 (dq, J=7.4, 0.6 Hz, 1H), 6.40 (dd, J=8.2, 0.8 Hz, 1H), 5.50 (s, 2H), 4.46 (s, 2H), 4.34 (s, 3H).

### Example 83: 3-((6-aminopyridin-2-yl)methyl)-7-((6-fluoropyridin-2-yl)oxy)-5-methyl-3,5-dihydro-4H-pyridazino[4,5-b]indol-4-one

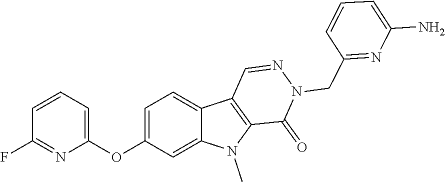 [SMILES: Cn1c2cc(Oc3cccc(F)n3)ccc2c2cnn(Cc3cccc(N)n3)c(=O)c21]

Example 83 was generated from Intermediate AY using a procedure analogous to that employed in the generation of Example 82. 1H NMR (400 MHz, Chloroform-d) δ 8.55 (s, 1H), 8.04 (dd, J=8.6, 0.6 Hz, 1H), 7.83 (q, J=8.0 Hz, 1H), 7.38 (dd, J=8.2, 7.4 Hz, 1H), 7.34 (dd, J=2.1, 0.6 Hz, 1H), 7.19 (dd, J=8.6, 2.0 Hz, 1H), 6.85 (ddd, J=7.9, 1.5, 0.6 Hz, 1H), 6.68 (ddd, J=7.8, 2.7, 0.6 Hz, 1H), 6.52 (dq, J=7.4, 0.6 Hz, 1H), 6.40 (dd, J=8.2, 0.8 Hz, 1H), 5.50 (s, 2H), 4.46 (s, 2H), 4.34 (s, 3H).

Intermediate AZ: 7-(benzyloxy)-5-methyl-3-((1-((2-(trimethylsilyl)ethoxy)methyl)-1H-indazol-4-yl)methyl)-3,5-dihydro-4H-pyridazino[4,5-b]indol-4-one

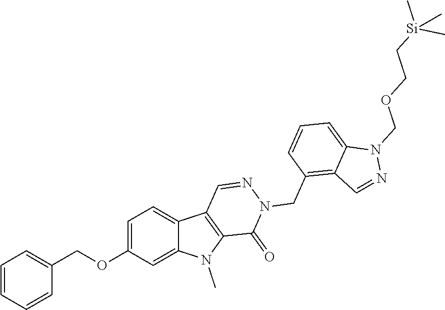 [SMILES: Cn1c2cc(OCc3ccccc3)ccc2c2cnn(Cc3cccc4c3cnn4COCC[Si](C)(C)C)c(=O)c21]

Intermediate AZ was generated by treating intermediate AU (215 mg, 0.70 mmol) with 4-(bromomethyl)-1-((2-(trimethylsilyl)ethoxy)methyl)-1H-indazole (289 mg, 0.85 mmol) and K₂CO₃ (243 mg, 1.79 mmol) in DMF. The reaction mixture was stirred at rt overnight and water was added. The reaction mixture was diluted with EtOAc, washed with saturated aqueous bicarbonate, dried, concentrated and purified by FCC (0-100% EtOAc in hexanes) to provide the desired product (220 mg). 1H NMR (400 MHz, DMSO-d6) δ 8.78 (s, 1H), 8.26 (d, J=0.9 Hz, 1H), 8.09 (d, J=8.8 Hz, 1H), 7.66 (d, J=8.5 Hz, 1H), 7.56-7.33 (m, 7H), 7.14-7.00 (m, 2H), 5.75 (d, J=2.0 Hz, 2H), 5.72 (s, 2H), 5.26 (s, 2H), 4.25 (s, 3H), 3.56-3.46 (m, 2H), 0.85-0.66 (m, 2H), −0.12 (s, 9H).

### Intermediate BA: 7-hydroxy-5-methyl-3-((1-((2-(trimethylsilyl)ethoxy)methyl)-1H-indazol-4-yl)methyl)-3,5-dihydro-4H-pyridazino[4,5-b]indol-4-one

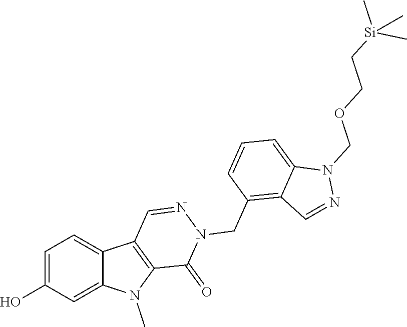 [SMILES: Cn1c2cc(O)ccc2c2cnn(Cc3cccc4c3cnn4COCC[Si](C)(C)C)c(=O)c21]

Intermediate BA was generated via analogous procedure to that which was used to generate Intermediate AW. 1H NMR (400 MHz, DMSO-d6) δ 9.98 (s, 1H), 8.72 (s, 1H), 8.24 (d, J=0.9 Hz, 1H), 7.98 (d, J=8.6 Hz, 1H), 7.66 (d, J=8.4 Hz, 1H), 7.38 (dd, J=8.5, 7.1 Hz, 1H), 7.07-7.00 (m, 1H), 6.95 (d, J=2.0 Hz, 1H), 6.90 (dd, J=8.6, 2.1 Hz, 1H), 5.74 (s, 2H), 5.71 (s, 2H), 4.18 (s, 3H), 3.57-3.46 (m, 2H), 0.84-0.72 (m, 2H), −0.12 (s, 9H).

### Intermediate BB: 5-methyl-7-((6-((2-(trimethylsilyl)ethoxy)methoxy)pyridin-2-yl)oxy)-3-((1-((2-(trimethylsilyl)ethoxy)methyl)-1H-indazol-4-yl)methyl)-3,5-dihydro-4H-pyridazino[4,5-b]indol-4-one

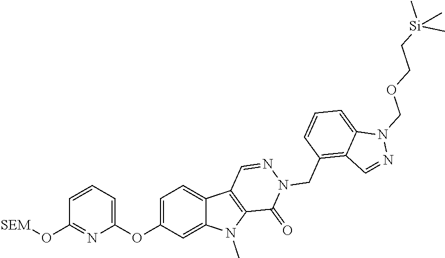 [SMILES: Cn1c2cc(Oc3cccc(OCOCC[Si](C)(C)C)n3)ccc2c2cnn(Cc3cccc4c3cnn4COCC[Si](C)(C)C)c(=O)c21]

Intermediate BB was generated from Intermediate BA via an analogous procedure employed to generate intermediate AJ. 1H NMR (400 MHz, DMSO-d6) δ 8.85 (s, 1H), 8.53 (d, J=1.1 Hz, 1H), 8.32 (dd, J=8.7, 0.5 Hz, 1H), 7.72 (d, J=2.1 Hz, 1H), 7.58 (d, J=8.7 Hz, 1H), 7.35 (dd, J=9.1, 7.6 Hz, 1H), 7.29-7.20 (m, 2H), 6.95 (dd, J=6.8, 0.9 Hz, 1H), 6.15 (dd, J=9.1, 1.1 Hz, 1H), 5.74 (d, J=12.0 Hz, 2H), 5.65 (s, 2H), 5.57 (s, 2H), 5.30 (dd, J=7.6, 1.1 Hz, 1H), 4.27 (s, 3H), 3.76-3.68 (m, 2H), 3.59-3.50 (m, 2H), 0.97-0.87 (m, 2H), 0.84-0.74 (m, 2H), −0.02 (s, 9H), −0.17 (s, 9H).

### Example 84: 3-((1H-indazol-4-yl)methyl)-7-((6-hydroxypyridin-2-yl)oxy)-5-methyl-3,5-dihydro-4H-pyridazino[4,5-b]indol-4-one

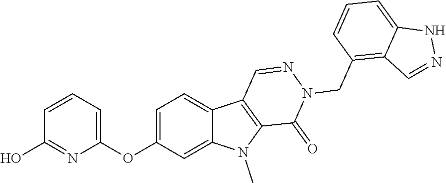 [SMILES: Cn1c2cc(Oc3cccc(O)n3)ccc2c2cnn(Cc3cccc4[nH]ncc34)c(=O)c21]

Intermediate BB (21.2 mg, 0.030 mmol) in methanol (2 mL) with 4 M HCl in dioxane (0.5 mL). The reaction mixture was stirred at rt for 3 hours. The reaction mixture was diluted with EtOAc, washed with saturated aqueous sodium bicarbonate, dried, concentrated and purified by FCC (0-10% MeOH in DCM) to provide the desired product (4.2 mg). 1H NMR (400 MHz, DMSO-d6) δ 13.12 (s, 1H), 10.77 (s, 1H), 8.84 (s, 1H), 8.22 (d, J=8.7 Hz, 1H), 8.19-8.13 (m, 1H), 7.68 (t, J=7.8 Hz, 1H), 7.57 (d, J=2.0 Hz, 1H), 7.47 (d, J=8.4 Hz, 1H), 7.30 (dd, J=8.4, 7.0 Hz, 1H), 7.15 (dd, J=8.7, 2.1 Hz, 1H), 7.06-6.93 (m, 1H), 6.57-6.29 (m, 2H), 5.72 (s, 2H), 4.25 (s, 3H).

### Intermediate BC: 7-((6-fluoropyridin-2-yl)oxy)-5-methyl-3-((1-((2-(trimethylsilyl)ethoxy)methyl)-1H-indazol-4-yl)methyl)-3,5-dihydro-4H-pyridazino[4,5-b]indol-4-one

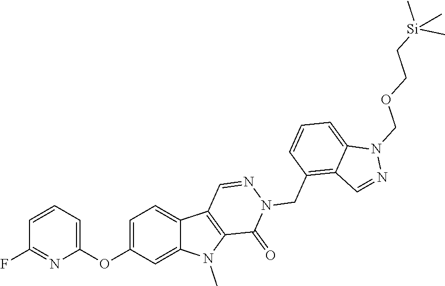 [SMILES: Cn1c2cc(Oc3cccc(F)n3)ccc2c2cnn(Cc3cccc4c3cnn4COCC[Si](C)(C)C)c(=O)c21]

Intermediate BC was generated from Intermediate BA using a procedure analogous to that employed in the generation of Example 82. 1H NMR (400 MHz, Chloroform-d) δ 8.52 (s, 1H), 8.44 (d, J=1.0 Hz, 1H), 8.00 (dd, J=8.6, 0.6 Hz, 1H), 7.87-7.78 (m, 1H), 7.74-7.66 (m, 1H), 7.35-7.30 (m, 2H), 7.25-7.20 (m, 1H), 7.18 (dd, J=8.6, 2.0 Hz, 1H), 6.88-6.82 (m, 1H), 6.69-6.65 (m, 1H), 5.74 (s, 2H), 5.73 (s, 2H), 4.34 (s, 3H), 3.68-3.59 (m, 2H), 0.99-0.90 (m, 2H), 0.03 (s, 9H).

### Example 85: 3-((1H-indazol-4-yl)methyl)-7-((6-fluoropyridin-2-yl)oxy)-5-methyl-3,5-dihydro-4H-pyridazino[4,5-b]indol-4-one

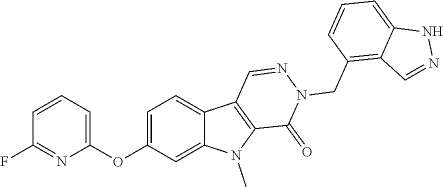 [SMILES: Cn1c2cc(Oc3cccc(F)n3)ccc2c2cnn(Cc3cccc4[nH]ncc34)c(=O)c21]

Example 85 was generated by employing a procedure analogous to the protocol used to generate Example 84. 1H NMR (400 MHz, DMSO-d6) δ 13.12 (s, 1H), 8.86 (s, 1H), 8.26 (dd, J=8.6, 0.5 Hz, 1H), 8.19-8.14 (m, 1H), 8.11-8.01 (m, 1H), 7.66 (dd, J=2.1, 0.6 Hz, 1H), 7.47 (d, J=8.4 Hz, 1H), 7.30 (dd, J=8.4, 7.0 Hz, 1H), 7.22 (dd, J=8.6, 2.1 Hz, 1H), 7.06-6.96 (m, 2H), 6.92 (dd, J=7.8, 2.4 Hz, 1H), 5.73 (s, 2H), 4.26 (s, 3H).

### Intermediate BD: 7-(benzyloxy)-5-methyl-3-((1-(tetrahydro-2H-pyran-2-yl)-1H-pyrazol-3-yl)methyl)-3,5-dihydro-4H-pyridazino[4,5-b]indol-4-one

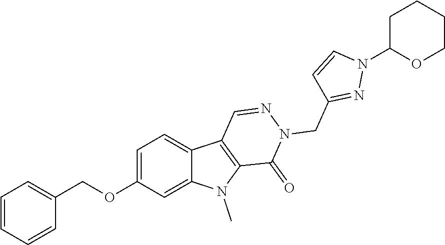 [SMILES: Cn1c2cc(OCc3ccccc3)ccc2c2cnn(Cc3ccn(C4CCCCO4)n3)c(=O)c21]

Intermediate BD was generated by treating Intermediate AU (637 mg, 2.09 mmol) with CsCO₃ (1.70 g, 5.22 mmol) and (1-(tetrahydro-2H-pyran-2-yl)-1H-pyrazol-3-yl)methyl methanesulfonate (652 mg, 2.52 mmol) in DMF (8 mL). The reaction mixture was stirred for 48 hours and water was added. The reaction mixture was extracted with EtOAc (5 mL×3) and the organics were dried, concentrated and purified by FCC (0-100% EtOAc in Hexanes) to provide the desired product. 1H NMR (400 MHz, DMSO-d6) δ 8.72 (s, 1H), 8.08 (d, J=8.8 Hz, 1H), 7.77 (dd, J=3.3, 2.4 Hz, 1H), 7.57-7.51 (m, 2H), 7.47-7.31 (m, 3H), 7.15-7.07 (m, 1H), 6.23 (d, J=2.4 Hz, 1H), 6.16 (d, J=2.4 Hz, 1H), 5.34-5.29 (m, 1H), 5.26 (s, 2H), 4.40 (d, J=5.9 Hz, 2H), 4.25 (s, 3H), 3.98-3.81 (m, 1H), 3.68-3.53 (m, 1H), 2.16-1.40 (m, 6H).

Intermediate BE: 7-hydroxy-5-methyl-3-((1-(tetrahydro-2H-pyran-2-yl)-1H-pyrazol-3-yl)methyl)-3,5-dihydro-4H-pyridazino[4,5-b]indol-4-one

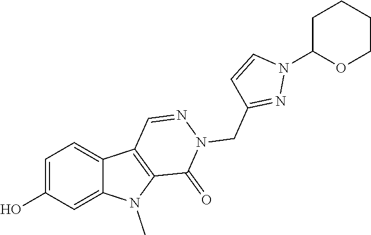 [SMILES: Cn1c2cc(O)ccc2c2cnn(Cc3ccn(C4CCCCO4)n3)c(=O)c21]

Intermediate BE was generated via analogous procedure to that which was used to generate Intermediate AW. 1H NMR (400 MHz, DMSO-d6) δ 9.95 (s, 1H), 8.66 (s, 1H), 8.03-7.94 (m, 1H), 6.95 (dd, J=2.1, 0.6 Hz, 1H), 6.90 (dd, J=8.6, 2.1 Hz, 1H), 6.23 (d, J=2.4 Hz, 1H), 6.15 (d, J=2.4 Hz, 1H), 4.40 (d, J=5.8 Hz, 2H), 4.18 (s, 3H), 3.98-3.85 (m, 1H), 3.67-3.49 (m, 1H), 2.16-1.39 (m, 6H).

### Intermediate BF: 7-((6-fluoropyridin-2-yl)oxy)-5-methyl-3-((1-(tetrahydro-2H-pyran-2-yl)-1H-pyrazol-3-yl)methyl)-3,5-dihydro-4H-pyridazino[4,5-b]indol-4-one

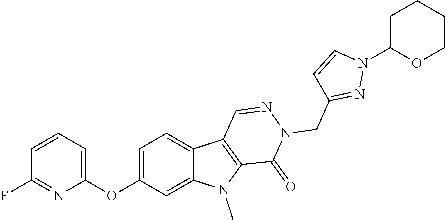 [SMILES: Cn1c2cc(Oc3cccc(F)n3)ccc2c2cnn(Cc3ccn(C4CCCCO4)n3)c(=O)c21]

Intermediate BF was generated from Intermediate BE using a procedure analogous to that employed in the generation of Example 82. 1H NMR (400 MHz, DMSO-d6) δ 8.81 (s, 1H), 8.27 (dd, J=8.7, 0.6 Hz, 1H), 8.06 (dt, J=8.6, 7.9 Hz, 1H), 7.79 (d, J=2.4 Hz, 1H), 7.66 (dd, J=2.1, 0.5 Hz, 1H), 7.22 (dd, J=8.6, 2.1 Hz, 1H), 7.03-6.96 (m, 1H), 6.92 (dd, J=7.9, 2.4 Hz, 1H), 6.17 (d, J=2.4 Hz, 1H), 5.76 (s, 2H), 5.39-5.27 (m, 1H), 4.26 (s, 3H), 3.91 (d, J=11.7 Hz, 1H), 3.64-3.53 (m, 1H), 2.19-1.45 (m, 6H).

### Example 86: 3-((1H-pyrazol-3-yl)methyl)-7-((6-fluoropyridin-2-yl)oxy)-5-methyl-3,5-dihydro-4H-pyridazino[4,5-b]indol-4-one

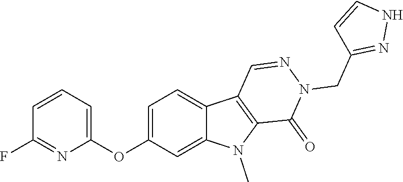 [SMILES: Cn1c2cc(Oc3cccc(F)n3)ccc2c2cnn(Cc3cc[nH]n3)c(=O)c21]

Example 86 was generated from Intermediate BF in an analogous fashion to the procedure used in the generation of Example 60. 1H NMR (400 MHz, DMSO-d6) δ 12.65 (s, 1H), 8.80 (s, 1H), 8.27 (d, J=8.6 Hz, 1H), 8.14-7.99 (m, 1H), 7.70-7.56 (m, 2H), 7.22 (dd, J=8.6, 2.1 Hz, 1H), 7.00 (dd, J=7.9, 1.6 Hz, 1H), 6.92 (dd, J=7.9, 2.4 Hz, 1H), 6.14 (s, 1H), 5.40 (s, 2H), 4.26 (s, 3H).

### Intermediate BG: 5-methyl-3-((1-(tetrahydro-2H-pyran-2-yl)-1H-pyrazol-3-yl)methyl)-7-((1-(tetrahydro-2H-pyran-2-yl)-1H-pyrazolo[4,3-c]pyridin-4-yl)oxy)-3,5-dihydro-4H-pyridazino[4,5-b]indol-4-one

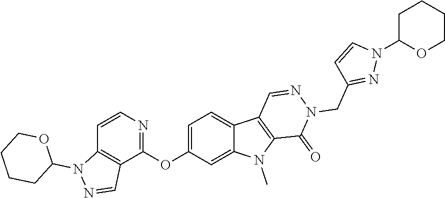 [SMILES: Cn1c2cc(Oc3nccc4c3cnn4C3CCCCO3)ccc2c2cnn(Cc3ccn(C4CCCCO4)n3)c(=O)c21]

Intermediate BG was generated from Intermediate BE in an analogous fashion to the procedure used in the generation of Intermediate AX.

### Example 87: 3-((1H-pyrazol-3-yl)methyl)-7-41H-pyrazolo[4,3-c]pyridin-4-yl)oxy)-5-methyl-3,5-dihydro-4H-pyridazino[4,5-b]indol-4-one

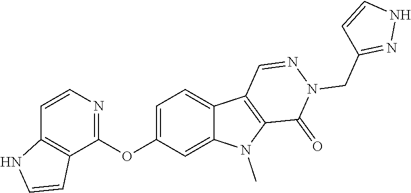 [SMILES: Cn1c2cc(Oc3nccc4[nH]ccc34)ccc2c2cnn(Cc3cc[nH]n3)c(=O)c21]

Example 87 was generated from Intermediate BG in an analogous fashion to the procedure used in the generation of Example 60. 1H NMR (400 MHz, DMSO-d6) δ 13.63 (s, 1H), 12.65 (s, 1H), 8.80 (s, 1H), 8.33-8.23 (m, 2H), 7.84-7.78 (m, 1H), 7.76-7.70 (m, 1H), 7.64 (s, 1H), 7.33-7.24 (m, 3H), 5.42 (d, J=21.1 Hz, 2H), 4.26 (s, 3H).

### Intermediate BH: 7-(benzyloxy)-3-(4-methoxybenzyl)-5-methyl-3,5-dihydro-4H-pyridazino[4,5-b]indol-4-one

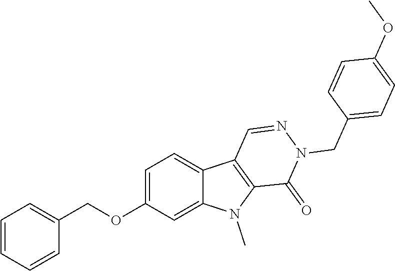 [SMILES: COc1ccc(Cn2ncc3c4ccc(OCc5ccccc5)cc4n(C)c3c2=O)cc1]

Intermediate BH was generated by treating Intermediate AU (260 mg, 0.85 mmol) in DMF (6 mL) with 1-(chloromethyl)-4-methoxybenzene (134 mg, 0.85 mmol) and K₂CO₃ (294 mg, 2.13 mmol). The reaction mixture was stirred at rt overnight, diluted with EtOAc, washed with water, dried, concentrated and purified by FCC (0-100% EtOAc in hexanes) to provide the desired product (270 mg). 1H NMR (400 MHz, DMSO-d6) δ 8.73 (s, 1H), 8.08 (d, J=8.8 Hz, 1H), 7.56-7.49 (m, 2H), 7.47-7.40 (m, 1H), 7.40-7.33 (m, 1H), 7.33-7.25 (m, 2H), 7.27-7.20 (m, 2H), 7.09 (dd, J=8.8, 2.2 Hz, 1H), 6.92-6.84 (m, 2H), 5.32 (s, 2H), 5.26 (s, 2H), 4.25 (s, 3H), 3.72 (s, 3H).

### Intermediate BI: 7-hydroxy-3-(4-methoxybenzyl)-5-methyl-3,5-dihydro-4H-pyridazino[4,5-b]indol-4-one

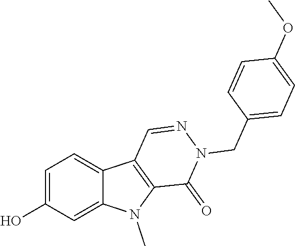 [SMILES: COc1ccc(Cn2ncc3c4ccc(O)cc4n(C)c3c2=O)cc1]

Intermediate BI was generated via analogous procedure to that which was used to generate Intermediate AW. 1H NMR (400 MHz, DMSO-d6) δ 9.97 (s, 1H), 8.68 (s, 1H), 7.97 (d, J=8.6 Hz, 1H), 7.32-7.23 (m, 2H), 6.93-6.82 (m, 4H), 5.31 (s, 2H), 4.17 (s, 3H), 3.72 (s, 3H).

### Example 88: 7-((6-fluoropyridin-2-yl)oxy)-3-(4-methoxybenzyl)-5-methyl-3,5-dihydro-4H-pyridazino[4,5-b]indol-4-one

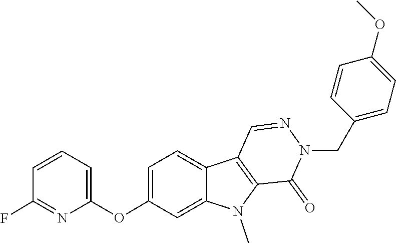 [SMILES: COc1ccc(Cn2ncc3c4ccc(Oc5cccc(F)n5)cc4n(C)c3c2=O)cc1]

Example 88 was generated from Intermediate BI using a procedure analogous to that employed in the generation of Example 82. 1H NMR (400 MHz, DMSO-d6) δ 8.83 (d, J=2.9 Hz, 1H), 8.26 (dd, J=8.5, 2.7 Hz, 1H), 8.06 (q, J=8.5, 7.9 Hz, 1H), 7.66 (s, 1H), 7.31 (d, J=8.1 Hz, 2H), 7.22 (d, J=9.0 Hz, 1H), 7.00 (d, J=8.2 Hz, 1H), 6.96-6.86 (m, 3H), 5.35 (d, J=3.0 Hz, 2H), 4.25 (d, J=2.9 Hz, 3H), 3.73 (d, J=2.8 Hz, 3H).

### Intermediate BJ: 7-((6-fluoropyridin-2-yl)oxy)-5-methyl-3,5-dihydro-4H-pyridazino[4,5-b]indol-4-one

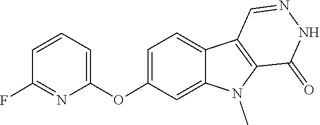 [SMILES: Cn1c2cc(Oc3cccc(F)n3)ccc2c2cn[nH]c(=O)c21]

Intermediate BJ was generated by dissolving Example 88 (75.2 mg, 0.175 mmol) in trifluoroacetic acid (3 mL) and heating the resulting reaction mixture to 60° C. for 5 hours. The reaction mixture was concentrated, dissolved in DCM, washed with saturated aqueous sodium bicarbonate, dried, concentrated and purified by FCC (0-100% EtOAc in hexanes) to provide the desired product (43 mg). 1H NMR (400 MHz, DMSO-d6) δ 12.82 (s, 1H), 8.77 (s, 1H), 8.26 (dd, J=8.6, 0.6 Hz, 1H), 8.06 (dt, J=8.6, 7.9 Hz, 1H), 7.65 (dd, J=2.1, 0.6 Hz, 1H), 7.21 (dd, J=8.6, 2.1 Hz, 1H), 6.99 (ddd, J=8.1, 1.8, 0.6 Hz, 1H), 6.92 (ddd, J=8.0, 2.5, 0.5 Hz, 1H), 4.24 (s, 3H).

### Intermediate BK: Bis-tert-butyl (3-((7-((6-fluoropyridin-2-yl)oxy)-5-methyl-4-oxo-4,5-dihydro-3H-pyridazino[4,5-b]indol-3-yl)methyl)phenyl)carbamate

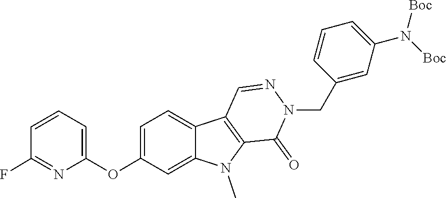 [SMILES: Cn1c2cc(Oc3cccc(F)n3)ccc2c2cnn(Cc3cccc(N(C(=O)OC(C)(C)C)C(=O)OC(C)(C)C)c3)c(=O)c21]

Intermediate BK was generated via an analogous procedure to the one employed to generate Intermediate AV.

### Example 89: 3-(3-aminobenzyl)-7-((6-fluoropyridin-2-yl)oxy)-5-methyl-3,5-dihydro-4H-pyridazino[4,5-b]indol-4-one

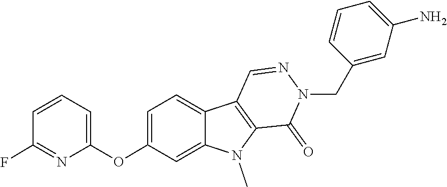 [SMILES: Cn1c2cc(Oc3cccc(F)n3)ccc2c2cnn(Cc3cccc(N)c3)c(=O)c21]

Example 89 was generated from Intermediate BK using a procedure analogous to that employed in the generation of Example 82. 1H NMR (400 MHz, Chloroform-d) δ 8.50 (s, 1H), 8.04-7.99 (m, 1H), 7.86-7.78 (m, 1H), 7.34-7.30 (m, 1H), 7.21-7.16 (m, 1H), 7.16-7.09 (m, 1H), 6.89-6.81 (m, 2H), 6.81-6.77 (m, 1H), 6.71-6.65 (m, 1H), 6.64-6.59 (m, 1H), 5.43 (s, 2H), 4.33 (s, 3H).

### Example 90: 7-((6-fluoropyridin-2-yl)oxy)-3-(3-methoxybenzyl)-5-methyl-3,5-dihydro-4H-pyridazino[4,5-b]indol-4-one

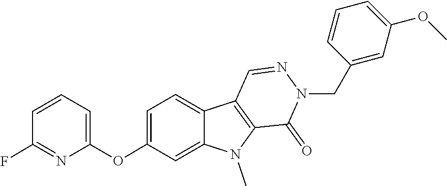 [SMILES: COc1cccc(Cn2ncc3c4ccc(Oc5cccc(F)n5)cc4n(C)c3c2=O)c1]

Example 90 was generated from Intermediate BJ using a procedure analogous to that employed in the generation of Intermediate BH. 1H NMR (400 MHz, DMSO-d6) δ 8.84 (s, 1H), 8.30-8.24 (m, 1H), 8.06 (dt, J=8.6, 7.9 Hz, 1H), 7.66 (dd, J=2.1, 0.5 Hz, 1H), 7.28-7.20 (m, 2H), 7.00 (dd, J=8.0, 1.6 Hz, 1H), 6.94-6.76 (m, 4H), 5.39 (s, 2H), 4.25 (s, 3H), 3.73 (d, J=1.2 Hz, 3H).

### Intermediate BL: 4-fluoro-2-iodo-5-(methoxymethyl)aniline

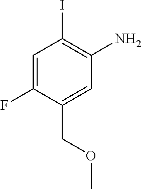

Intermediate BL was generated by treating 4-fluoro-3-(methoxymethyl)aniline (1.84 g, 11.89 mmol) in acetic acid with 1-iodopyrrolidine-2,5-dione (3.08 g, 13.78 mmol) and the reaction mixture was stirred at rt for 3 hours. The reaction mixture was diluted with EtOAc, washed with saturated aqueous sodium thiosulfate and purified by FCC (0-50% EtOAc in hexanes) to provide the desired product. 1H NMR (400 MHz, DMSO-d6) δ 7.39 (d, J=9.3 Hz, 1H), 6.83-6.75 (m, 1H), 5.08 (s, 2H), 4.36-4.26 (m, 2H), 3.29 (s, 3H).

### Intermediate BM: N-(4-fluoro-2-iodo-5-(methoxymethyl)phenyl)-4-methylbenzenesulfonamide

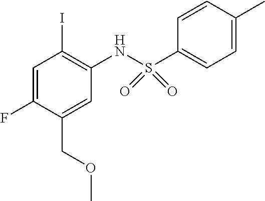 [SMILES: COCc1cc(NS(=O)(=O)c2ccc(C)cc2)c(I)cc1F]

Intermediate BM was generated by treating Intermediate BL (2.30 g, 8.18 mmol) in DCM (50 mL) with pyridine (1.00 mL, 12.27 mmol) and 4-methylbenzenesulfonyl chloride (1.56 g, 8.18 mmol) and the reaction mixture stirred at rt overnight. The reaction mixture was washed with saturated aqueous sodium bicarbonate, dried, concentrated and purified by FCC (0-100% EtOAc in hexanes) to provide the desired product. 1H NMR (400 MHz, DMSO-d6) δ 9.69 (s, 1H), 7.73-7.66 (m, 1H), 7.62-7.53 (m, 2H), 7.43-7.34 (m, 2H), 7.02-6.96 (m, 1H), 4.36-4.27 (m, 2H), 3.19 (s, 3H), 2.38 (s, 3H).

### Intermediate BN: ethyl 5-fluoro-6-(methoxymethyl)-1-tosyl-1H-indole-2-carboxylate

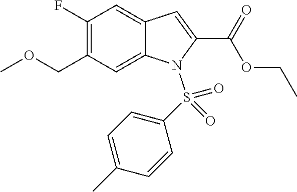 [SMILES: CCOC(=O)c1cc2cc(F)c(COC)cc2n1S(=O)(=O)c1ccc(C)cc1]

Intermediate BN was generated by treating Intermediate BM (3.11 g, 7.15 mmol) in degassed THF (50 mL) with Pd(P(Ph₃)₄) (413 mg, 0.36 mmol), ethyl propiolate (2.10 g, 21.44 mol), DIPEA (4.62 g, 35.73 mmol) and ZnBr₂ (4.83 g, 21.44 mmol). The reaction mixture was heated to 80° C. overnight, cooled to rt and filtered through celite. The filtrate was washed with saturated sodium bicarbonate, dried, concentrated and purified by FCC (0-100% EtOAc in hexanes) to provide the desired product (1.76 g). 1H NMR (400 MHz, DMSO-d6) δ 8.07-8.02 (m, 1H), 7.85-7.81 (m, 2H), 7.51 (d, J=9.8 Hz, 1H), 7.46-7.41 (m, 2H), 7.35 (d, J=0.8 Hz, 1H), 4.60 (d, J=1.2 Hz, 2H), 4.36 (q, J=7.1 Hz, 2H), 3.35 (s, 3H), 2.36 (s, 3H), 1.32 (t, J=7.1 Hz, 3H).zz

### Intermediate BO: ethyl 5-fluoro-6-(methoxymethyl)-1H-indole-2-carboxylate

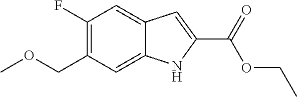 [SMILES: CCOC(=O)c1cc2cc(F)c(COC)cc2[nH]1]

Intermediate BO was generated by treating Intermediate BN (2.30 g, 5.68 mmol) in ethanol (25 mL) and THF (25 mL) with potassium hydroxide (796 mg, 14.19 mmol). The reaction mixture was stirred at rt for 2 hours. The reaction mixture was diluted with EtOAc and washed with water, dried, concentrated and purified by FCC (0-10% MeOH in DCM) to provide the desired product (4.62 g). 1H NMR (400 MHz, DMSO-d6) δ 12.00 (s, 1H), 7.47 (dd, J=6.2, 0.9 Hz, 1H), 7.43 (d, J=10.8 Hz, 1H), 7.12 (dd, J=2.2, 0.9 Hz, 1H), 4.58-4.51 (m, 2H), 4.35 (q, J=7.1 Hz, 2H), 3.34 (s, 3H), 1.35 (t, J=7.1 Hz, 3H).

Intermediate BP: ethyl 5-fluoro-6-(methoxymethyl)-1-methyl-1H-indole-2-carboxylate

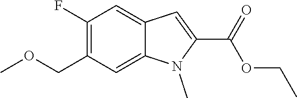 [SMILES: CCOC(=O)c1cc2cc(F)c(COC)cc2n1C]

Intermediate BP was generated by treating Intermediate BO (50. 4 mg, 0.20 mmol) in DMF (3 mL) with K₂CO₃ (69.3 mg, 0.50 mmol) and Mel (31.3 mg, 0.22 mmol). The resulting reaction mixture was stirred overnight at rt. The reaction mixture was diluted with EtOAc, washed with water, dried concentrated and purified by FCC (0-50% EtOAc in hexanes) to provide the desired product. 1H NMR (400 MHz, DMSO-d6) δ 7.64 (d, J=6.0 Hz, 1H), 7.47 (d, J=10.5 Hz, 1H), 7.24 (d, J=0.8 Hz, 1H), 4.57 (s, 2H), 4.33 (q, J=7.1 Hz, 2H), 4.04 (s, 3H), 3.36 (s, 3H), 1.34 (t, J=7.1 Hz, 3H).

### Intermediate BQ: ethyl 5-fluoro-3-formyl-6-(methoxymethyl)-1-methyl-1H-indole-2-carboxylate

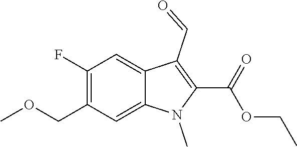 [SMILES: CCOC(=O)c1c(C=O)c2cc(F)c(COC)cc2n1C]

Intermediate BQ was generated from Intermediate BP via analogous procedure employed to generate Intermediate A. 1H NMR (400 MHz, DMSO-d6) δ 10.42 (s, 1H), 7.96 (d, J=10.5 Hz, 1H), 7.82 (d, J=6.0 Hz, 1H), 4.60 (t, J=0.9 Hz, 2H), 4.48 (qd, J=7.1, 1.7 Hz, 2H), 4.07 (s, 3H), 3.38 (s, 3H), 1.41 (td, J=7.1, 1.0 Hz, 3H).

### Intermediate BR: 8-fluoro-7-(methoxymethyl)-5-methyl-3,5-dihydro-4H-pyridazino[4,5-b]indol-4-one

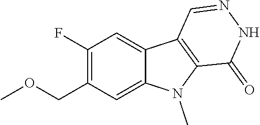 [SMILES: COCc1cc2c(cc1F)c1cn[nH]c(=O)c1n2C]

Intermediate BR was generated from Intermediate BQ via analogous procedure employed to generate Intermediate C. 1H NMR (400 MHz, DMSO-d6) δ 12.80 (s, 1H), 8.72 (s, 1H), 8.07 (d, J=10.2 Hz, 1H), 7.80 (d, J=5.9 Hz, 1H), 4.65 (d, J=1.2 Hz, 2H), 4.29 (s, 3H), 3.40 (s, 3H).

### Example 91: 8-fluoro-7-(methoxymethyl)-5-methyl-3-((6-methylpyridin-2-yl)methyl)-3,5-dihydro-4H-pyridazino[4,5-b]indol-4-one

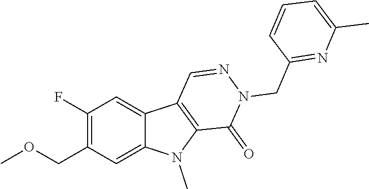

Example 91 was generated by treating Intermediate BR (25.2 mg, 0.097 mmol) in DMF (3 mL) with 2-(bromomethyl)-6-methyl-pyridine (19.7 mg, 0.106 mmol) and Cs₂CO₃ (78.6 mg, 0.241 mmol). The reaction mixture was stirred overnight at rt, diluted with EtOAc, washed with water, dried, concentrated and purified by FCC (0-100% EtOAc in hexanes) to provide the desired product (23 mg). 1H NMR (400 MHz, DMSO-d6) δ 8.82 (s, 1H), 8.10 (d, J=10.2 Hz, 1H), 7.83 (d, J=5.8 Hz, 1H), 7.60 (t, J=7.7 Hz, 1H), 7.14 (d, J=7.6 Hz, 1H), 6.85 (d, J=7.7 Hz, 1H), 5.46 (s, 2H), 4.66 (d, J=1.0 Hz, 2H), 4.30 (s, 3H), 3.39 (d, J=13.3 Hz, 3H), 2.45 (s, 3H).

Intermediate BS: 7-(bromomethyl)-8-fluoro-5-methyl-3-((6-methylpyridin-2-yl)methyl)-3,5-dihydro-4H-pyridazino[4,5-b]indol-4-one

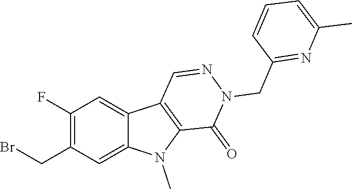 [SMILES: Cc1cccc(Cn2ncc3c4cc(F)c(CBr)cc4n(C)c3c2=O)n1]

Intermediate BS was generated by treating Example 91 (150 mg, 0.41 mmol) in DCM (5 mL) with BBr₃ (461 mg, 1.843 mmol) and the reaction mixture was stirred for 2 hours at rt. The reaction mixture was diluted with DCM, washed with saturated aqueous sodium bicarbonate, dried, concentrated and purified by FCC (0-100% EtOAc in hexanes) to provide the desire product (120 mg). 1H NMR (400 MHz, DMSO-d6) δ 8.82 (s, 1H), 8.15 (d, J=10.2 Hz, 1H), 8.04 (d, J=6.1 Hz, 1H), 7.61 (t, J=7.7 Hz, 1H), 7.15 (d, J=7.7 Hz, 1H), 6.86 (d, J=7.8 Hz, 1H), 5.46 (s, 2H), 4.92 (d, J=1.2 Hz, 2H), 4.28 (s, 3H), 2.44 (s, 3H).

### Example 92: 8-fluoro-5-methyl-3-((6-methylpyridin-2-yl)methyl)-7-(morpholinomethyl)-3,5-dihydro-4H-pyridazino[4,5-b]indol-4-one

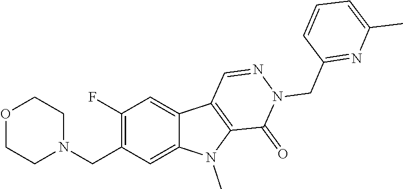 [SMILES: Cc1cccc(Cn2ncc3c4cc(F)c(CN5CCOCC5)cc4n(C)c3c2=O)n1]

Example 92 was generated by treating Intermediate BS (38.1 mg, 0.068 mmol) in DCM (3 mL) with triethylamine (27.4 mg, 0.271 mmol) and morpholine (11.8 mg, 0.135 mmol) and the reaction mixture was stirred at rt overnight. The reaction mixture was diluted with DCM, washed with saturated aqueous sodium bicarbonate, dried, concentrated and purified by FCC (0-20% MeOH in DCM) to provide the desired product (23 mg). 1H NMR (400 MHz, DMSO-d6) δ 8.81 (s, 1H), 8.08 (d, J=10.1 Hz, 1H), 7.78 (d, J=5.9 Hz, 1H), 7.60 (t, J=7.7 Hz, 1H), 7.14 (d, J=7.6 Hz, 1H), 6.84 (d, J=7.7 Hz, 1H), 4.29 (s, 3H), 3.75-3.69 (m, 2H), 3.65-3.57 (m, 6H), 2.47 (t, J=4.6 Hz, 4H), 2.44 (s, 3H).

### Example 93: 8-fluoro-7-(((2-hydroxyethyl)amino)methyl)-5-methyl-3-((6-methylpyridin-2-yl)methyl)-3,5-dihydro-4H-pyridazino[4,5-b]indol-4-one

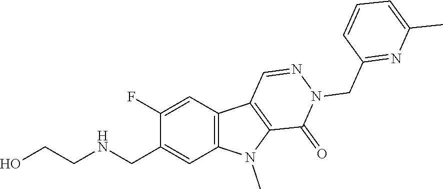 [SMILES: Cc1cccc(Cn2ncc3c4cc(F)c(CNCCO)cc4n(C)c3c2=O)n1]

Example 93 was generated from intermediate BS via an analogous procedure to that used to generate Example 92. 1H NMR (400 MHz, DMSO-d6) δ 8.80 (s, 1H), 8.05 (d, J=10.3 Hz, 1H), 7.85 (d, J=6.0 Hz, 1H), 7.60 (t, J=7.7 Hz, 1H), 7.14 (d, J=7.6 Hz, 1H), 6.84 (d, J=7.7 Hz, 1H), 5.46 (s, 2H), 4.52 (t, J=5.4 Hz, 1H), 4.29 (s, 3H), 3.95 (s, 2H), 3.52 (q, J=5.6 Hz, 2H), 2.67 (t, J=5.7 Hz, 2H), 2.53-2.51 (m, 1H), 2.45 (s, 3H).

### Example 94: 8-fluoro-5-methyl-3-((6-methylpyridin-2-yl)methyl)-7-(((tetrahydro-2H-pyran-4-yl)amino)methyl)-3,5-dihydro-4H-pyridazino[4,5-b]indol-4-one

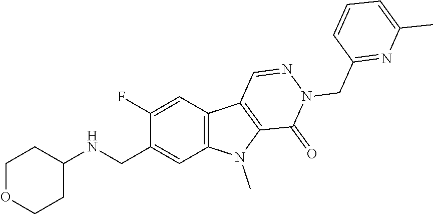 [SMILES: Cc1cccc(Cn2ncc3c4cc(F)c(CNC5CCOCC5)cc4n(C)c3c2=O)n1]

Example 94 was generated from intermediate BS via an analogous procedure to that used to generate Example 92. 1H NMR (400 MHz, Methanol-d4) δ 7.87 (s, 1H), 7.09 (d, J=10.0 Hz, 1H), 7.00 (d, J=5.9 Hz, 1H), 6.81 (t, J=7.8 Hz, 1H), 6.36 (d, J=7.6 Hz, 1H), 6.11 (d, J=7.8 Hz, 1H), 4.75 (s, 2H), 3.52 (s, 3H), 3.36-3.28 (m, 2H), 3.17 (dd, J=11.4, 4.5 Hz, 2H), 2.67-2.57 (m, 2H), 2.51 (s, 1H), 2.16-2.04 (m, 1H), 1.72 (s, 3H), 1.23-1.13 (m, 2H), 0.84-0.67 (m, 2H).

### Example 95: 8-fluoro-5-methyl-3-((6-methylpyridin-2-yl)methyl)-7-(pyrrolidin-1-ylmethyl)-3,5-dihydro-4H-pyridazino[4,5-b]indol-4-one

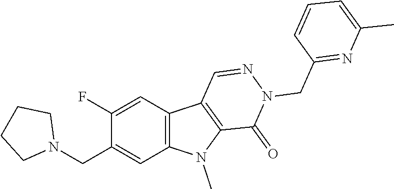 [SMILES: Cc1cccc(Cn2ncc3c4cc(F)c(CN5CCCC5)cc4n(C)c3c2=O)n1]

Example 95 was generated from intermediate BS via an analogous procedure to that used to generate Example 92. 1H NMR (400 MHz, DMSO-d6) δ 8.80 (s, 1H), 8.06 (d, J=10.2 Hz, 1H), 7.77 (d, J=5.9 Hz, 1H), 7.60 (t, J=7.7 Hz, 1H), 7.14 (d, J=7.6 Hz, 1H), 6.84 (d, J=7.7 Hz, 1H), 5.46 (s, 2H), 4.29 (s, 3H), 3.82 (s, 2H), 2.54 (s, 4H), 2.44 (s, 3H), 1.73 (s, 4H).

### Example 96: 7-((dimethylamino)methyl)-8-fluoro-5-methyl-3-((6-methylpyridin-2-yl)methyl)-3,5-dihydro-4H-pyridazino[4,5-b]indol-4-one

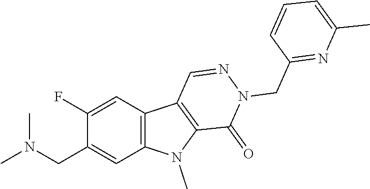

Example 96 was generated from intermediate BS via an analogous procedure to that used to generate Example 92. 1H NMR (400 MHz, DMSO-d6) δ 8.81 (s, 1H), 8.07 (d, J=10.2 Hz, 1H), 7.77 (d, J=5.9 Hz, 1H), 7.60 (t, J=7.7 Hz, 1H), 7.14 (d, J=7.7 Hz, 1H), 6.84 (d, J=7.8 Hz, 1H), 5.46 (s, 2H), 4.29 (s, 3H), 3.68-3.60 (m, 2H), 2.44 (s, 3H), 2.23 (d, J=1.5 Hz, 6H).

### Example 97: 7-((6-fluoropyridin-2-yl)oxy)-5-methyl-3-(2-morpholinoethyl)-3,5-dihydro-4H-pyridazino[4,5-b]indol-4-one

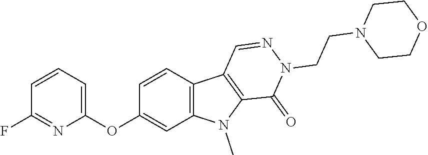 [SMILES: Cn1c2cc(Oc3cccc(F)n3)ccc2c2cnn(CCN3CCOCC3)c(=O)c21]

Example 97 was generated by treating Intermediate BJ (10.1 mg, 0.033 mmol) in DMF (3 mL) with potassium carbonate (11.2 mg, 0.081 mmol) and 4-(2-chloroethyl)morpholine (4.9 mg, 0.033 mmol) and the reaction mixture was stirred at room temperature overnight. The reaction mixture was diluted with EtOAc, washed with water, dried, concentrated and purified by FCC (0-30% MeOH, DCM) to provide the desired product (9.3 mg). 1H NMR (400 MHz, DMSO-d6) δ 8.81 (s, 1H), 8.32-8.24 (m, 1H), 8.12-8.02 (m, 1H), 7.65 (d, J=2.0 Hz, 1H), 7.22 (dd, J=8.7, 2.1 Hz, 1H), 7.00 (dd, J=7.9, 1.6 Hz, 1H), 6.92 (dd, J=7.8, 2.4 Hz, 1H), 4.36 (t, J=6.9 Hz, 2H), 4.25 (s, 3H), 3.58-3.51 (m, 4H), 2.79-2.67 (m, 2H), 2.47 (t, J=4.5 Hz, 4H).

### Example 98: 3-(2-(dimethylamino)ethyl)-7-((6-fluoropyridin-2-yl)oxy)-5-methyl-3,5-dihydro-4H-pyridazino [4,5-b]indol-4-one

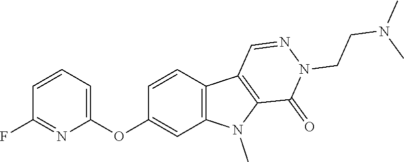 [SMILES: CN(C)CCn1ncc2c3ccc(Oc4cccc(F)n4)cc3n(C)c2c1=O]

Example 98 was generated from Intermediate BJ via analogous procedure employed to generate Example 97. 1H NMR (400 MHz, DMSO-d6) δ 8.80 (s, 1H), 8.26 (dd, J=8.6, 0.5 Hz, 1H), 8.12-7.99 (m, 1H), 7.67-7.61 (m, 1H), 7.21 (dd, J=8.6, 2.1 Hz, 1H), 6.99 (dd, J=7.9, 1.6 Hz, 1H), 6.92 (dd, J=7.8, 2.4 Hz, 1H), 4.38-4.29 (m, 2H), 4.25 (s, 3H), 2.74-2.64 (m, 2H), 2.22 (s, 6H).

### Intermediate BT: 3-(2-(benzyloxy)ethyl)-7-((6-fluoropyridin-2-yl)oxy)-5-methyl-3,5-dihydro-4H-pyridazino[4,5-b]indol-4-one

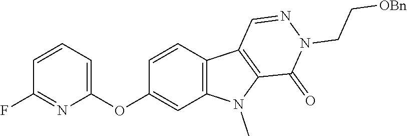 [SMILES: Cn1c2cc(Oc3cccc(F)n3)ccc2c2cnn(CCOCc3ccccc3)c(=O)c21]

Intermediate BT was generated from Intermediate via analogous procedure employed to generate Example 97.

### Example 99: 7-((6-fluoropyridin-2-yl)oxy)-3-(2-hydroxyethyl)-5-methyl-3,5-dihydro-4H-pyridazino [4,5-b]indol-4-one

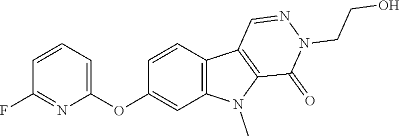 [SMILES: Cn1c2cc(Oc3cccc(F)n3)ccc2c2cnn(CCO)c(=O)c21]

Example 99 was generated by treating intermediate BS (20.2 mg, 0.045 mmol) in EtOH (5 mL) with 10% Pd/C (5.0 mg) and the reaction mixture was placed under hydrogen atmosphere and the reaction mixture was stirred overnight. The reaction mixture was filtered through celite and concentrated to provide the desired product (12 mg). 1H NMR (400 MHz,) δ 8.80 (s, 1H), 8.26 (dd, J=8.7, 0.6 Hz, 1H), 8.10-8.02 (m, 1H), 7.68-7.63 (m, 1H), 7.21 (dd, J=8.6, 2.1 Hz, 1H), 7.00 (dd, J=7.8, 1.6 Hz, 1H), 6.92 (dd, J=7.9, 2.4 Hz, 1H), 4.86-4.78 (m, 1H), 4.33-4.27 (m, 2H), 4.26 (s, 3H), 3.83-3.73 (m, 2H).

### Intermediate BU: tert-butyl 4-(2-(7-((6-fluoropyridin-2-yl)oxy)-5-methyl-4-oxo-4,5-dihydro-3H-pyridazino[4,5-b]indol-3-yl)ethyl)piperazine-1-carboxylate

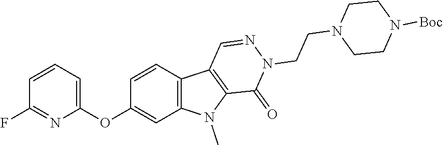 [SMILES: Cn1c2cc(Oc3cccc(F)n3)ccc2c2cnn(CCN3CCN(C(=O)OC(C)(C)C)CC3)c(=O)c21]

Intermediate BU was generated from Intermediate BJ via analogous procedure employed to generate Example 97. 1H NMR (400 MHz, DMSO-d6) δ 8.80 (s, 1H), 8.26 (dd, J=8.6, 0.5 Hz, 1H), 8.11-7.98 (m, 1H), 7.64 (dd, J=2.1, 0.5 Hz, 1H), 7.21 (dd, J=8.6, 2.1 Hz, 1H), 6.99 (dd, J=8.0, 1.6 Hz, 1H), 6.91 (dd, J=7.9, 2.5 Hz, 1H), 4.42-4.30 (m, 2H), 4.25 (s, 3H), 3.32-3.23 (m, 4H), 2.82-2.72 (m, 2H), 2.48-2.39 (m, 5H), 1.39 (s, 9H).

### Example 100: 7-((6-fluoropyridin-2-yl)oxy)-5-methyl-3-(2-(piperazin-1-yl)ethyl)-3,5-dihydro-4H-pyridazino [4,5-b]indol-4-one

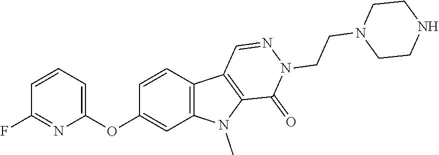 [SMILES: Cn1c2cc(Oc3cccc(F)n3)ccc2c2cnn(CCN3CCNCC3)c(=O)c21]

Example 100 was generated by treating Intermediate BU (23.1 mg, 0.044 mmol) in DCM (5 mL) with 4 M HCl in dioxane (138 mg, 1.11 mol) and the reaction mixture was stirred at room temperature for 5 hours. The reaction mixture was concentrated, dissolved in methanol (5 mL) and passed through a basic column to provide the desired compound (15.1 mg). 1H NMR (400 MHz, DMSO-d6) δ 8.80 (s, 1H), 8.26 (d, J=8.6 Hz, 1H), 8.11-8.01 (m, 1H), 7.65 (d, J=2.1 Hz, 1H), 7.22 (dd, J=8.7, 2.1 Hz, 1H), 7.00 (dd, J=7.9, 1.7 Hz, 1H), 6.92 (dd, J=7.9, 2.4 Hz, 1H), 4.40-4.29 (m, 2H), 4.25 (s, 3H), 2.76-2.63 (m, 4H), 2.56 (p, J=1.9 Hz, 2H), 2.47-2.39 (m, 4H), 2.33 (p, J=1.9 Hz, 1H).

### Example 101: 8-fluoro-3-(2-fluorobenzyl)-7-(methoxymethyl)-5-methyl-3,5-dihydro-4H-pyridazino [4,5-b]indol-4-one

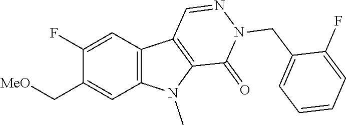 [SMILES: COCc1cc2c(cc1F)c1cnn(Cc3ccccc3F)c(=O)c1n2C]

Example 101 was generated from Intermediate BR via analogous procedure employed to generate Example 91. 1H NMR (400 MHz, DMSO-d6) δ 8.80 (s, 1H), 8.08 (d, J=10.2 Hz, 1H), 7.82 (d, J=5.9 Hz, 1H), 7.34 (tdd, J=7.4, 5.4, 2.0 Hz, 1H), 7.29-7.09 (m, 3H), 5.46 (s, 2H), 4.68-4.60 (m, 2H), 4.29 (s, 3H), 3.40 (s, 3H).

### Intermediate BU: 7-(bromomethyl)-8-fluoro-3-(2-fluorobenzyl)-5-methyl-3,5-dihydro-4H-pyridazino [4,5-b]indol-4-one

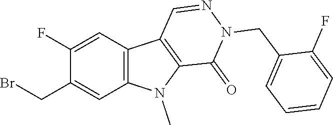 [SMILES: Cn1c2cc(CBr)c(F)cc2c2cnn(Cc3ccccc3F)c(=O)c21]

Intermediate BU was generated from Example 101 via analogous procedure employed to generate Example BS. 1H NMR (400 MHz, DMSO-d6) δ 8.80 (s, 1H), 8.13 (d, J=10.2 Hz, 1H), 8.03 (d, J=6.2 Hz, 1H), 7.41-7.30 (m, 1H), 7.30-7.07 (m, 3H), 5.46 (s, 2H), 4.91 (d, J=1.1 Hz, 2H), 4.28 (s, 3H).

### Example 102: 8-fluoro-3-(2-fluorobenzyl)-5-methyl-7-(morpholinomethyl)-3,5-dihydro-4H-pyridazino [4,5-b]indol-4-one

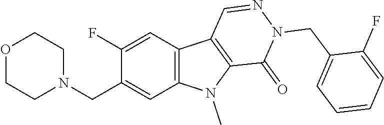 [SMILES: Cn1c2cc(CN3CCOCC3)c(F)cc2c2cnn(Cc3ccccc3F)c(=O)c21]

Example 102 was generated from intermediate BU via an analogous procedure to that used to generate Example 92. 1H NMR (400 MHz, DMSO-d6) δ 8.79 (s, 1H), 8.06 (d, J=10.1 Hz, 1H), 7.78 (d, J=5.9 Hz, 1H), 7.41-7.31 (m, 1H), 7.30-7.10 (m, 3H), 5.47 (s, 2H), 4.29 (s, 3H), 3.71 (s, 2H), 3.64-3.55 (m, 4H), 2.49-2.42 (m, 4H).

### Example 103: 8-fluoro-3-(2-fluorobenzyl)-7-(((2-hydroxyethyl)amino)methyl)-5-methyl-3,5-dihydro-4H-pyridazino[4,5-b]indol-4-one

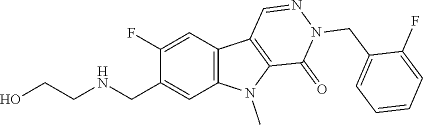 [SMILES: Cn1c2cc(CNCCO)c(F)cc2c2cnn(Cc3ccccc3F)c(=O)c21]

Example 103 was generated from intermediate BU via an analogous procedure to that used to generate Example 92. 1H NMR (400 MHz, DMSO-d6) δ 8.79 (s, 1H), 8.05 (d, J=10.2 Hz, 1H), 7.88 (d, J=6.0 Hz, 1H), 7.42-7.32 (m, 1H), 7.28-7.17 (m, 2H), 7.14 (td, J=7.4, 1.2 Hz, 1H), 5.47 (s, 2H), 4.63 (s, 1H), 4.48-2.04 (bs, 1H), 4.29 (s, 3H), 4.01 (s, 2H), 3.54 (q, J=5.5 Hz, 2H), 2.77-2.70 (m, 2H).

### Example 104: 8-fluoro-3-(2-fluorobenzyl)-5-methyl-7-(((tetrahydro-2H-pyran-4-yl)amino)methyl)-3,5-dihydro-4H-pyridazino[4,5-b]indol-4-one

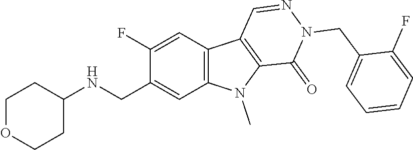 [SMILES: Cn1c2cc(CNC3CCOCC3)c(F)cc2c2cnn(Cc3ccccc3F)c(=O)c21]

Example 104 was generated from intermediate BU via an analogous procedure to that used to generate Example 92. 1H NMR (400 MHz, DMSO-d6) δ 8.78 (s, 1H), 8.02 (d, J=10.2 Hz, 1H), 7.86 (d, J=6.0 Hz, 1H), 7.40-7.29 (m, 1H), 7.27-7.10 (m, 3H), 5.47 (s, 2H), 4.28 (s, 3H), 3.96 (s, 2H), 3.91-3.79 (m, 2H), 3.30-3.23 (m, 2H), 2.74-2.63 (m, 1H), 2.18 (s, 1H), 1.83 (d, J=13.0 Hz, 2H), 1.32 (tt, J=20.4, 10.3 Hz, 2H).

### Example 105: 8-fluoro-3-(2-fluorobenzyl)-5-methyl-7-(pyrrolidin-1-ylmethyl)-3,5-dihydro-4H-pyridazino[4,5-b]indol-4-one

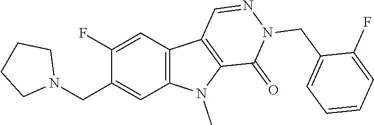 [SMILES: Cn1c2cc(CN3CCCC3)c(F)cc2c2cnn(Cc3ccccc3F)c(=O)c21]

Example 105 was generated from intermediate BU via an analogous procedure to that used to generate Example 92. 1H NMR (400 MHz, DMSO-d6) δ 8.79 (s, 1H), 8.04 (d, J=10.1 Hz, 1H), 7.77 (d, J=5.9 Hz, 1H), 7.40-7.29 (m, 1H), 7.28-7.08 (m, 3H), 5.47 (s, 2H), 4.29 (s, 3H), 3.87-3.77 (m, 2H), 2.59-2.52 (m, 4H), 1.80-1.66 (m, 4H).

### Example 106: 7-((dimethylamino)methyl)-8-fluoro-3-(2-fluorobenzyl)-5-methyl-3,5-dihydro-4H-pyridazino[4,5-b]indol-4-one

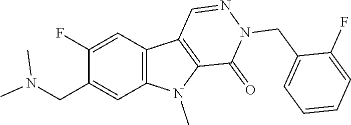 [SMILES: CN(C)Cc1cc2c(cc1F)c1cnn(Cc3ccccc3F)c(=O)c1n2C]

Example 106 was generated from intermediate BU via an analogous procedure to that used to generate Example 92. 1H NMR (400 MHz, DMSO-d6) δ 8.79 (s, 1H), 8.05 (d, J=10.2 Hz, 1H), 7.76 (d, J=5.9 Hz, 1H), 7.38-7.30 (m, 1H), 7.26-7.09 (m, 3H), 5.47 (s, 2H), 4.29 (s, 3H), 3.63 (d, J=1.2 Hz, 2H), 2.23 (s, 6H).

### Intermediate BV: tert-butyl 4-((8-fluoro-3-(2-fluorobenzyl)-5-methyl-4-oxo-4,5-dihydro-3H-pyridazino[4,5-b]indol-7-yl)methyl)-1,4-diazepane-1-carboxylate

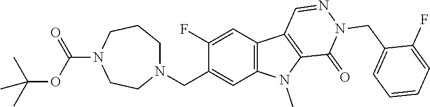 [SMILES: Cn1c2cc(CN3CCCN(C(=O)OC(C)(C)C)CC3)c(F)cc2c2cnn(Cc3ccccc3F)c(=O)c21]

Intermediate BV was generated from intermediate BU via an analogous procedure to that used to generate Example 92. 1H NMR (400 MHz, DMSO-d6) δ 8.79 (s, 1H), 8.05 (d, J=10.2 Hz, 1H), 7.79 (d, J=5.9 Hz, 1H), 7.40-7.26 (m, 1H), 7.26-7.06 (m, 3H), 5.47 (s, 2H), 4.29 (s, 3H), 3.85 (s, 2H), 3.46-3.35 (m, 4H), 2.76-2.59 (m, 4H), 1.76 (s, 2H), 1.40 (d, J=7.0 Hz, 9H).

### Example 107: 7-((1,4-diazepan-1-yl)methyl)-8-fluoro-3-(2-fluorobenzyl)-5-methyl-3,5-dihydro-4H-pyridazino[4,5-b]indol-4-one

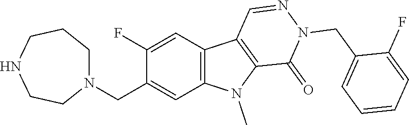 [SMILES: Cn1c2cc(CN3CCCNCC3)c(F)cc2c2cnn(Cc3ccccc3F)c(=O)c21]

Example 107 was generated by dissolving Intermediate BV (43.8 mg, 0.082 mmol) in trifluoroacetic acid (3 mL). The reaction mixture was stirred at room temperature for 30 minutes and concentrated to provide the desired product (35 mg). 1H NMR (400 MHz, DMSO-d6) δ 8.97 (s, 2H), 8.85 (s, 1H), 8.31-8.20 (m, 1H), 8.07-8.01 (m, 1H), 7.40-7.31 (m, 1H), 7.29-7.18 (m, 2H), 7.18-7.10 (m, 1H), 5.48 (s, 2H), 4.55 (s, 2H), 4.31 (s, 3H), 3.72-3.07 (m, 9H), 2.24-2.04 (m, 2H).

### Intermediate BW: tert-butyl 4-((8-fluoro-3-(2-fluorobenzyl)-5-methyl-4-oxo-4,5-dihydro-3H-pyridazino[4,5-b]indol-7-yl)methyl)piperazine-1-carboxylate

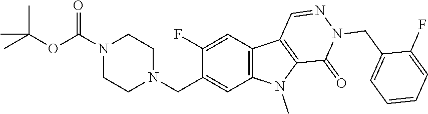 [SMILES: Cn1c2cc(CN3CCN(C(=O)OC(C)(C)C)CC3)c(F)cc2c2cnn(Cc3ccccc3F)c(=O)c21]

Intermediate BW was generated from intermediate BU via an analogous procedure to that used to generate Example 92. 1H NMR (400 MHz, DMSO-d6) δ 8.79 (s, 1H), 8.06 (d, J=10.1 Hz, 1H), 7.77 (d, J=5.8 Hz, 1H), 7.40-7.29 (m, 1H), 7.28-7.10 (m, 3H), 5.47 (s, 2H), 4.29 (s, 3H), 3.73 (s, 2H), 3.41-3.32 (m, 4H), 2.46-2.39 (m, 4H), 1.39 (s, 9H).

### Example 108: 8-fluoro-3-(2-fluorobenzyl)-5-methyl-7-(piperazin-1-ylmethyl)-3,5-dihydro-4H-pyridazino[4,5-b]indol-4-one

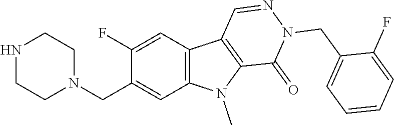 [SMILES: Cn1c2cc(CN3CCNCC3)c(F)cc2c2cnn(Cc3ccccc3F)c(=O)c21]

Example 108 was generated from intermediate BW via an analogous procedure to that used to generate Example 107. 1H NMR (400 MHz, DMSO-d6) δ 8.81 (s, 1H), 8.66 (s, 2H), 8.13 (d, J=10.1 Hz, 1H), 7.87 (d, J=5.9 Hz, 1H), 7.41-7.30 (m, 1H), 7.26-7.08 (m, 3H), 5.47 (s, 2H), 4.30 (s, 3H), 4.00 (s, 2H), 3.24-3.15 (m, 4H), 2.95-2.79 (m, 4H).

### Example 109: 5-methyl-3-((6-methylpyridin-2-yl)methyl)-7-(2-morpholinoethoxy)-3,4a,5,9b-tetrahydro-4H-pyridazino[4,5-b]indol-4-one

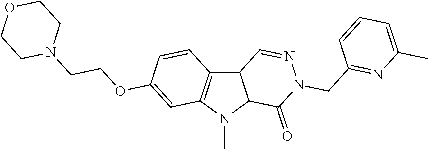 [SMILES: Cc1cccc(CN2N=CC3c4ccc(OCCN5CCOCC5)cc4N(C)C3C2=O)n1]

Example 109 was generated by treating Example 29 (30.3 mg, 0.095 mmol) in DMF (3 mL) with Cs₂CO₃ (92.5 mg, 0.284 mmol) and 4-(2-chloroethyl)-morpholine hydrochloride (17.6 mg, 0.095 mmol) and the resulting reaction mixture was allowed to stir at room temperature overnight. The reaction mixture was diluted with EtOAc, washed with water, dried, concentrated and purified by FCC (0-100% EtOAc in hexanes) to provide the desired product (32 mg). 1H NMR (400 MHz, DMSO-d6) δ 8.76 (s, 1H), 8.08 (d, J=8.7 Hz, 1H), 7.64-7.53 (m, 1H), 7.29 (d, J=2.2 Hz, 1H), 7.14 (d, J=7.6 Hz, 1H), 7.04 (dd, J=8.8, 2.2 Hz, 1H), 6.82 (d, J=7.8 Hz, 1H), 5.45 (s, 2H), 4.29-4.22 (m, 5H), 3.66-3.56 (m, 4H), 2.81-2.73 (m, 2H), 2.51 (q, J=1.9 Hz, 4H), 2.45 (s, 3H).

### Example 110: 3-(2-fluorobenzyl)-5-methyl-7-(2-morpholinoethoxy)-3,4a,5,9b-tetrahydro-4H-pyridazino[4,5-b]indol-4-one

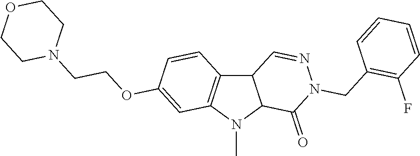 [SMILES: CN1c2cc(OCCN3CCOCC3)ccc2C2C=NN(Cc3ccccc3F)C(=O)C21]

Example 110 was generated from example 46 via an analogous procedure to that used to generate Example 109. 1H NMR (400 MHz, DMSO-d6) δ 8.75 (s, 1H), 8.07 (d, J=8.8 Hz, 1H), 7.38-7.31 (m, 1H), 7.29 (d, J=2.2 Hz, 1H), 7.27-7.09 (m, 3H), 7.03 (dd, J=8.8, 2.2 Hz, 1H), 5.46 (s, 2H), 4.29-4.19 (m, 5H), 3.65-3.57 (m, 4H), 2.81-2.74 (m, 2H), 2.56-2.50 (m, 4H).

### Example 2

### Pharmacological Examples

Pyruvate Kinase Activity Enzyme Assay. Recombinant Enzyme. A continuous, enzyme-coupled assay, which uses lactate dehydrogenase (LDH) and measures the depletion of NADH via absorbance at 340 nm was utilized to determine the pyruvate kinase activity. For AC50 measurements (concentration of activator necessary to achieve half-maximal activation) with ML-265, assays were performed in 96-well format using 200 μL/well assay volume with final concentrations of 20 nM human recombinant PKM2 (Sigma, SAE0021), differing concentrations of ML-265, 0.5 mM PEP, 1 mM ADP, 0.2 mM NADH, and 8 U of lactate dehydrogenase (LDH) in an assay buffer of 50 mM Tris-HCl (pH 7.4), 100 mM KCl, and 5 mM MgCl₂. The decrease in absorbance at 340 nm was monitored using a SPECTROstar Omega plate reader (BMG LABTECH Inc., Cary, NC, USA). Initial velocities were calculated with the MARS software. Data were normalized to DMSO (dimethyl sulfoxide)-treated PKM2 activity.

Cell culture. For 661W cell line experiments, media was replaced prior to the start of treatment with DMSO or ML-265. Cells were incubated with DMSO or different concentrations of ML-265 for 2 hours. Cells were lysed and homogenized in RIPA Lysis and Extraction Buffer (Catalog number: 89900, Life Technologies Corporation, Grand Island, NY) with protease inhibitors (Complete-Mini, Roche Diagnostics, Indianapolis, IN) and cellular debris was removed by centrifugation at 10,000 rpm for 10 minutes. Ten microliters of the supernatant was used to assess pyruvate kinase activity, and the activity was normalized to total protein content as previously described.

Results are shown in Table 1 below.

TABLE 1

| | | Max | Cell | Max |
|---|---|---|---|---|
| | EC50 | Activation | EC50 | Activation |
| Example | (nM) | (%) | (nM) | (%) |
| 1 | 117 | 212 | 15 | 431 |
| 2 | 106 | 90 | | |
| 3 | 428 | 193 | 28 | 310 |
| 4 | 527 | 267 | 22 | 309 |
| 5 | 192 | 226 | 13 | 295 |
| 6 | | | | |
| 7 | 63 | 222 | 18 | 355 |
| 8 | 117 | 271 | 25 | 324 |
| 9 | | | | |
| 10 | | | | |
| 11 | 110 | 170 | | |
| 12 | 131 | 123 | | |
| 13 | | | | |
| 14 | NA | NA | | |
| 15 | 40 | 254 | 124 | 296 |
| 16 | 40 | 231 | 58 | 279 |
| 17 | 74 | 157 | 17 | 281 |
| 18 | 41 | 264 | 23 | 292 |
| 20 | 15 | 199 | 67 | 229 |
| 21 | 25 | 299 | 75 | 273 |
| 23 | 22 | 196 | 33 | 288 |
| 27 | 26 | 307 | | |
| 52 | 97 | 225 | 66 | 305 |
| 53 | 15 | 199 | 37 | 229 |
| 54 | 53 | 299 | 75 | 273 |
| 57 | 62 | 234 | 81 | 370 |
| 60 | 67 | 243 | 54 | 334 |
| 61 | 76 | 293 | 120 | 223 |
| 62 | 82 | 221 | 134 | 376 |
| 63 | 31 | 219 | 78 | 302 |
| 64 | 69 | 165 | 73 | 272 |
| 65 | 43 | 191 | 115 | 262 |
| 66 | 36 | 154 | 121 | 323 |
| 67 | 49 | 277 | 147 | 290 |
| 70 | 82 | 191 | 162 | 210 |
| 71 | 28 | 165 | 69 | 278 |
| 72 | 55 | 206 | 72 | 222 |
| 73 | 22 | 232 | 104 | 189 |
| 74 | 62 | 234 | 81 | 370 |
| 78 | 35 | 195 | 47 | 247 |
| 80 | 45 | 286 | 235 | 325 |
| 81 | 49 | 336 | 259 | 259 |
| 85 | 63 | 330 | 76 | 315 |
| 86 | 63 | 195 | 172 | 352 |
| 87 | 50 | 270 | 95 | 317 |
| 90 | 72 | 218 | 60 | 202 |

### Example 3

Table 2 shows additional compounds. Compounds are synthesized using the methods described herein or other suitable synthesis schemes.

TABLE 2

| Cmpd | | |
|---|---|---|
| # | Structure | Name |
| 1 | 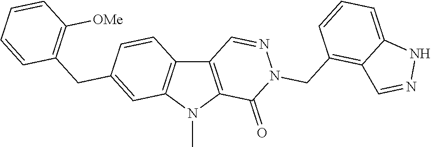 [SMILES: COc1ccccc1Cc1ccc2c3cnn(Cc4cccc5[nH]ncc45)c(=O)c3n(C)c2c1] | 3-((1H-indazol-4-yl)methyl)-7-(2- methoxybenzyl)-5-methyl-3,5- dihydro-4H-pyridazino[4,5-b]indol- 4-one |
| 2 | 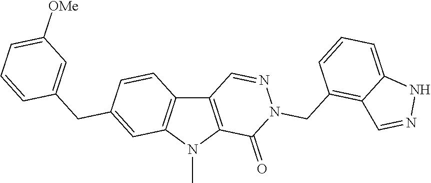 [SMILES: COc1cccc(Cc2ccc3c4cnn(Cc5cccc6[nH]ncc56)c(=O)c4n(C)c3c2)c1] | 3-((1H-indazol-4-yl)methyl)-7-(3- methoxybenzyl)-5-methyl-3,5- dihydro-4H-pyridazino[4,5-b]indol- 4-one |
| 3 | 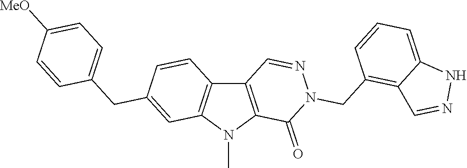 [SMILES: COc1ccc(Cc2ccc3c4cnn(Cc5cccc6[nH]ncc56)c(=O)c4n(C)c3c2)cc1] | 3-((1H-indazol-4-yl)methyl)-7-(4- methoxybenzyl)-5-methyl-3,5- dihydro-4H-pyridazino[4,5-b]indol- 4-one |
| 4 | 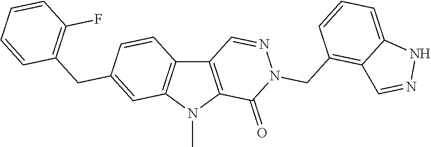 [SMILES: Cn1c2cc(Cc3ccccc3F)ccc2c2cnn(Cc3cccc4[nH]ncc34)c(=O)c21] | 3-((1H-indazol-4-yl)methyl)-7-(2- fluorobenzyl)-5-methyl-3,5- dihydro-4H-pyridazino[4,5-b]indol- 4-one |
| 5 | 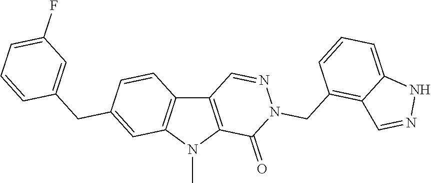 [SMILES: Cn1c2cc(Cc3cccc(F)c3)ccc2c2cnn(Cc3cccc4[nH]ncc34)c(=O)c21] | 3-((1H-indazol-4-yl)methyl)-7-(3- fluorobenzyl)-5-methyl-3,5- dihydro-4H-pyridazino[4,5-b]indol- 4-one |
| 6 | 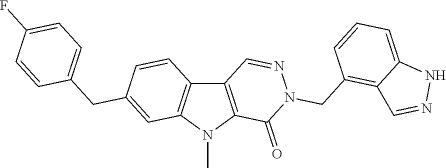 [SMILES: Cn1c2cc(Cc3ccc(F)cc3)ccc2c2cnn(Cc3cccc4[nH]ncc34)c(=O)c21] | 3-((1H-indazol-4-yl)methyl)-7-(4- fluorobenzyl)-5-methyl-3,5- dihydro-4H-pyridazino[4,5-b]indol- 4-one |
| 7 | 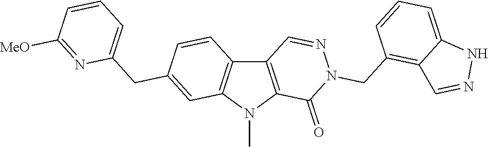 [SMILES: COc1cccc(Cc2ccc3c4cnn(Cc5cccc6[nH]ncc56)c(=O)c4n(C)c3c2)n1] | 3-((1H-indazol-4-yl)methyl)-7-((6- methoxypyridin-2-yl)methyl)-5- methyl-3,5-dihydro-4H-pyridazino [4,5-b]indol-4-one |
| 8 | 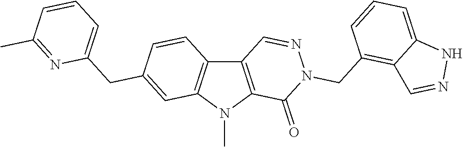 [SMILES: Cc1cccc(Cc2ccc3c4cnn(Cc5cccc6[nH]ncc56)c(=O)c4n(C)c3c2)n1] | 3-((1H-indazol-4-yl)methyl)-5- methyl-7-((6-methylpyridin-2- yl)methyl)-3,5-dihydro-4H-pyridazino [4,5-b]indol-4-one |
| 9 | 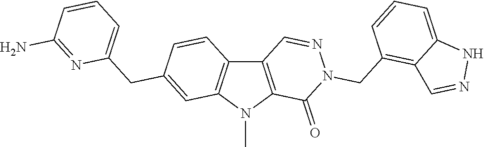 [SMILES: Cn1c2cc(Cc3cccc(N)n3)ccc2c2cnn(Cc3cccc4[nH]ncc34)c(=O)c21] | 3-((1H-indazol-4-yl)methyl)-7-((6- aminopyridin-2-yl)methyl)-5- methyl-3,5-dihydro-4H-pyridazino [4,5-b]indol-4-one |
| 10 | 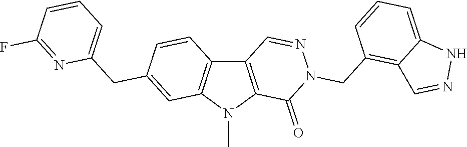 [SMILES: Cn1c2cc(Cc3cccc(F)n3)ccc2c2cnn(Cc3cccc4[nH]ncc34)c(=O)c21] | 3-((1H-indazol-4-yl)methyl)-7-((6- fluoropyridin-2-yl)methyl)-5- methyl-3,5-dihydro-4H-pyridazino [4,5-b]indol-4-one |
| 11 | 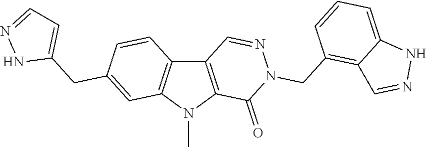 [SMILES: Cn1c2cc(Cc3ccn[nH]3)ccc2c2cnn(Cc3cccc4[nH]ncc34)c(=O)c21] | 3-((1H-indazol-4-yl)methyl)-7-((1H- pyrazol-5-yl)methyl)-5-methyl-3,5- dihydro-4H-pyridazino[4,5-b]indol- 4-one |
| 12 | 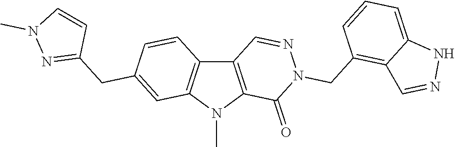 [SMILES: Cn1ccc(Cc2ccc3c4cnn(Cc5cccc6[nH]ncc56)c(=O)c4n(C)c3c2)n1] | 3-((1H-indazol-4-yl)methyl)-5- methyl-7-((1-methyl-1H-pyrazol-3- yl)methyl)-3,5-dihydro-4H-pyridazino [4,5-b]indol-4-one |
| 13 | 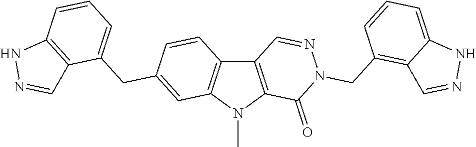 [SMILES: Cn1c2cc(Cc3cccc4[nH]ncc34)ccc2c2cnn(Cc3cccc4[nH]ncc34)c(=O)c21] | 3,7-bis((1H-indazol-4-yl)methyl)-5- methyl-3,5-dihydro-4H-pyridazino [4,5-b]indol-4-one |
| 14 | 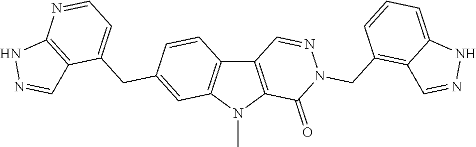 [SMILES: Cn1c2cc(Cc3ccnc4[nH]ncc34)ccc2c2cnn(Cc3cccc4[nH]ncc34)c(=O)c21] | 3-((1H-indazol-4-yl)methyl)-7-((1H- pyrazolo[3,4-b]pyridin-4- yl)methyl)-5-methyl-3,5-dihydro-4H- pyridazino[4,5-b]indol-4-one |
| 15 | 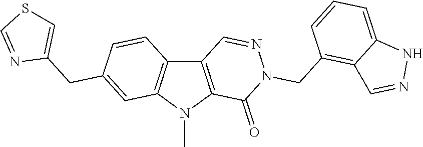 [SMILES: Cn1c2cc(Cc3cscn3)ccc2c2cnn(Cc3cccc4[nH]ncc34)c(=O)c21] | 3-((1H-indazol-4-yl)methyl)-5- methyl-7-(thiazol-4-ylmethyl)-3,5- dihydro-4H-pyridazino[4,5-b]indol- 4-one |
| 16 | 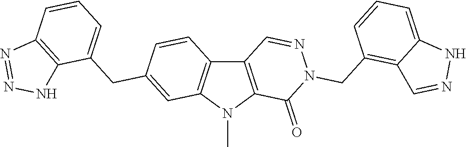 [SMILES: Cn1c2cc(Cc3cccc4nn[nH]c34)ccc2c2cnn(Cc3cccc4[nH]ncc34)c(=O)c21] | 7-((1H-benzo[d][1,2,3]triazol-7- yl)methyl)-3-((1H-indazol-4- yl)methyl)-5-methyl-3,5-dihydro- 4H-pyridazino[4,5-b]indol- 4-one |
| 17 | 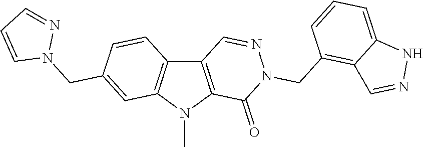 [SMILES: Cn1c2cc(Cn3cccn3)ccc2c2cnn(Cc3cccc4[nH]ncc34)c(=O)c21] | 3-((1H-indazol-4-yl)methyl)-7-((1H- pyrazol-1-yl)methyl)-5-methyl- 3,5-dihydro-4H-pyridazino[4,5-b]indol- 4-one |
| 18 | 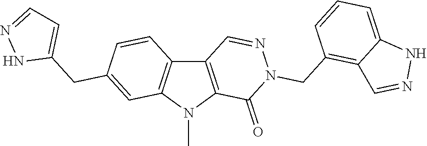 [SMILES: Cn1c2cc(Cc3ccn[nH]3)ccc2c2cnn(Cc3cccc4[nH]ncc34)c(=O)c21] | 3-((1H-indazol-4-yl)methyl)-7-((1H- pyrazol-5-yl)methyl)-5-methyl-3,5- dihydro-4H-pyridazino[4,5-b]indol- 4-one |
| 19 | 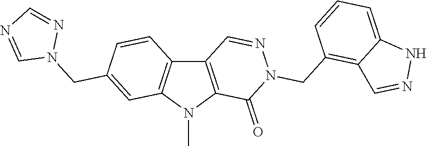 [SMILES: Cn1c2cc(Cn3cncn3)ccc2c2cnn(Cc3cccc4[nH]ncc34)c(=O)c21] | 7-((1H-12,4-triazol-1-yl)methyl)-3- ((1H-indazol-4-yl)methyl)-5- methyl-3,5-dihydro-4H-pyridazino [4,5-b]indol-4-one |
| 20 | 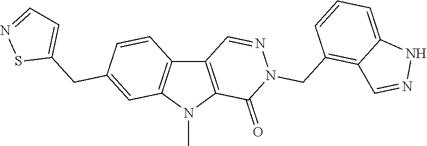 [SMILES: Cn1c2cc(Cc3ccns3)ccc2c2cnn(Cc3cccc4[nH]ncc34)c(=O)c21] | 3-((1H-indazol-4-yl)methyl)-7- (isothiazol-5-ylmethyl)-5-methyl- 3,5-dihydro-4H-pyridazino [4,5-b]indol-4-one |
| 21 | 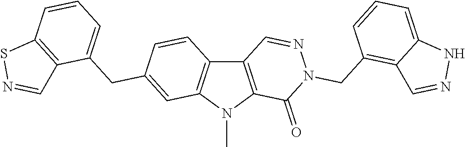 [SMILES: Cn1c2cc(Cc3cccc4sncc34)ccc2c2cnn(Cc3cccc4[nH]ncc34)c(=O)c21] | 3-((1H-indazol-4-yl)methyl)-7- (benzo[d]isothiazol-4-ylmethyl)-5- methyl-3,5-dihydro-4H-pyridazino [4,5-b]indol-4-one |
| 22 | 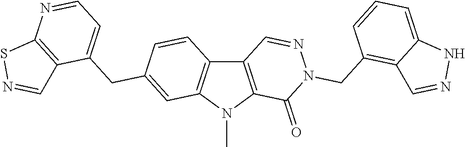 [SMILES: Cn1c2cc(Cc3ccnc4sncc34)ccc2c2cnn(Cc3cccc4[nH]ncc34)c(=O)c21] | 3-((1H-indazol-4-yl)methyl)-7- (isothiazolo[5,4-b]pyridin-4- ylmethyl)-5-methyl-3,5-dihydro- 4H-pyridazino[4,5-b]indol- 4-one |
| 23 | 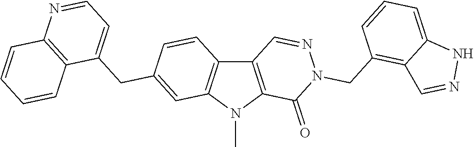 [SMILES: Cn1c2cc(Cc3ccnc4ccccc34)ccc2c2cnn(Cc3cccc4[nH]ncc34)c(=O)c21] | 3-((1H-indazol-4-yl)methyl)-5- methyl-7-(quinolin-4-ylmethyl)-3,5- dihydro-4H-pyridazino[4,5-b]indol- 4-one |
| 24 | 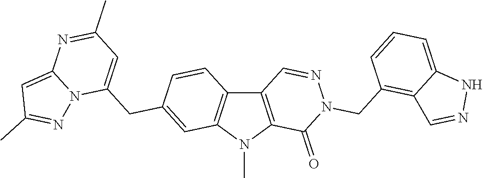 [SMILES: Cc1cc(Cc2ccc3c4cnn(Cc5cccc6[nH]ncc56)c(=O)c4n(C)c3c2)n2nc(C)cc2n1] | 3-((1H-indazol-4-yl)methyl)-7-((2,5- dimethylpyrazolo[1,5-a]pyrimidin- 7-yl)methyl)-5-methyl-3,5-dihydro- 4H-pyridazino[4,5-b]indol- 4-one |
| 25 | 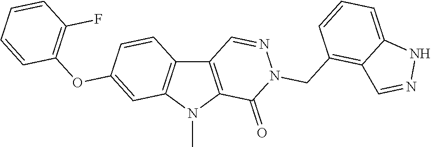 [SMILES: Cn1c2cc(Oc3ccccc3F)ccc2c2cnn(Cc3cccc4[nH]ncc34)c(=O)c21] | 3-((1H-indazol-4-yl)methyl)-7-(2- fluorophenyl)-5-methyl-3,5- dihydro-4H-pyridazino[4,5-b]indol- 4-one |
| 26 | 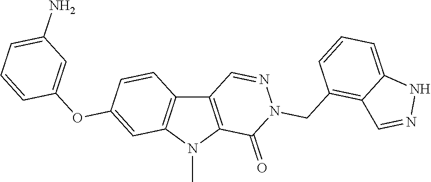 [SMILES: Cn1c2cc(Oc3cccc(N)c3)ccc2c2cnn(Cc3cccc4[nH]ncc34)c(=O)c21] | 3-((1H-indazol-4-yl)methyl)-7-(3- aminophenoxy)-5-methyl-3,5- dihydro-4H-pyridazino[4,5-b]indol- 4-one |
| 27 | 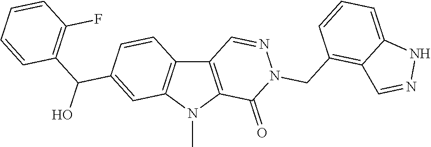 [SMILES: Cn1c2cc(C(O)c3ccccc3F)ccc2c2cnn(Cc3cccc4[nH]ncc34)c(=O)c21] | 3-((1H-indazol-4-yl)methyl)-7-((2- fluorophenyl)(hydroxy)methyl)-5- methyl-3,5- dihydro-4H-pyridazino[4,5-b]indol- 4-one |
| 28 | 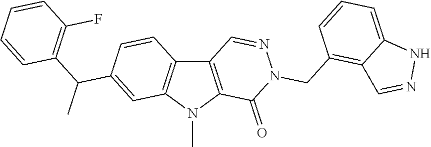 [SMILES: CC(c1ccc2c3cnn(Cc4cccc5[nH]ncc45)c(=O)c3n(C)c2c1)c1ccccc1F] | 3-((1H-indazol-4-yl)methyl)-7-(1-(2- fluorophenyl)ethyl)-5-methyl-3,5- dihydro-4H-pyridazino[4,5-b]indol- 4-one |
| 29 | 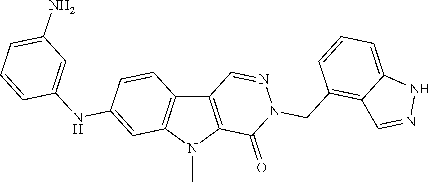 [SMILES: Cn1c2cc(Nc3cccc(N)c3)ccc2c2cnn(Cc3cccc4[nH]ncc34)c(=O)c21] | 3-((1H-indazol-4-yl)methyl)-7-((3- aminophenyl)amino)-5-methyl-3,5- dihydro-4H-pyridazino[4,5-b]indol- 4-one |
| 30 | 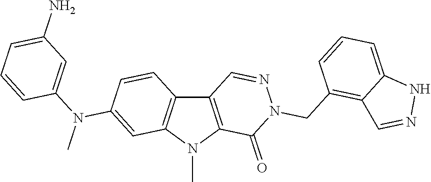 [SMILES: CN(c1cccc(N)c1)c1ccc2c3cnn(Cc4cccc5[nH]ncc45)c(=O)c3n(C)c2c1] | 3-((1H-indazol-4-yl)methyl)-7-((3- aminophenyl)(methyl)amino)-5- methyl-3,5-dihydro-4H-pyridazino [4,5-b]indol-4-one |
| 31 | 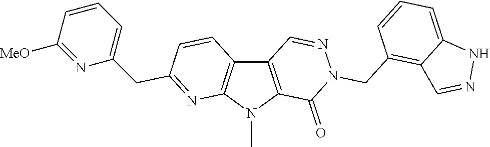 [SMILES: COc1cccc(Cc2ccc3c4cnn(Cc5cccc6[nH]ncc56)c(=O)c4n(C)c3n2)n1] | 7-((1H-indazol-4-yl)methyl)-2-((6- methoxypyridin-2-yl)methyl)-9- methyl-7,9-dihydro-8H- pyrido[3′,2′:4,5]pyrrolo[2,3- d]pyridazin-8-one |
| 32 | 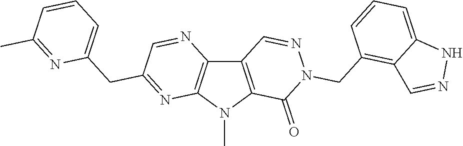 [SMILES: Cc1cccc(Cc2cnc3c4cnn(Cc5cccc6[nH]ncc56)c(=O)c4n(C)c3n2)n1] | 7-((1H-indazol-4-yl)methyl)-5- methyl-3-((6-methylpyridin-2- yl)methyl)-5,7-dihydro-6H- pyrazino[2′,3′:4,5]pyrrolo[2,3- d]pyridazin-6-one |
| 33 | 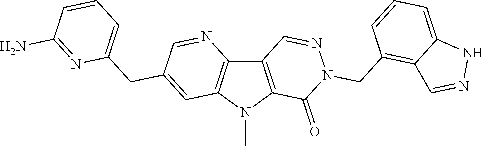 [SMILES: Cn1c2cc(Cc3cccc(N)n3)cnc2c2cnn(Cc3cccc4[nH]ncc34)c(=O)c21] | 7-((1H-indazol-4-yl)methyl)-3-((6- aminopyridin-2-yl)methyl)-5- methyl-5,7-dihydro-6H- pyrrolo[2′,3′:4,5]pyrrolo[2,3- d]pyridazin-6-one |
| 34 | 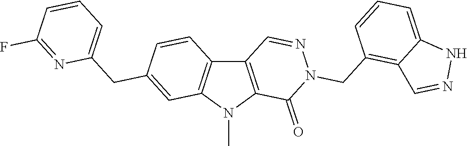 [SMILES: Cn1c2cc(Cc3cccc(F)n3)ccc2c2cnn(Cc3cccc4[nH]ncc34)c(=O)c21] | 3-((1H-indazol-4-yl)methyl)-7-((6- fluoropyridin-2-yl)methyl)-5- methyl-3,5-dihydro-4H- pyridazino[4,5-b]indol-4-one |
| 35 | 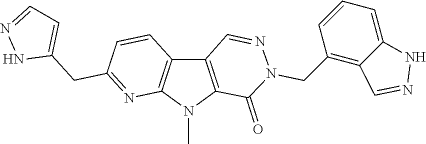 | 7-((1H-indazol-4-yl)methyl)-2-((1H- pyrazol-5-yl)methyl)-9-methyl-7,9- dihydro-8H- pyrido[3′,2′:4,5]pyrrolo[2,3- d]pyridazin-8-one |
| 36 | 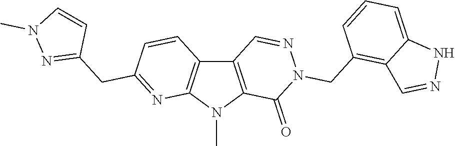 [SMILES: Cn1ccc(Cc2ccc3c4cnn(Cc5cccc6[nH]ncc56)c(=O)c4n(C)c3n2)n1] | 7-((1H-indazol-4-yl)methyl)-9- methyl-2-((1-methyl-1H-pyrazol-3- yl)methyl)-7,9-dihydro-8H- pyrido[3′,2′:4,5]pyrrolo[2,3- d]pyridazin-8-one |
| 37 | 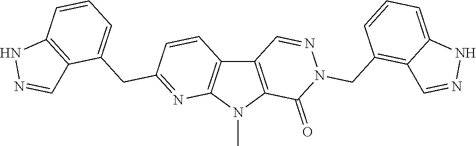 [SMILES: Cn1c2nc(Cc3cccc4[nH]ncc34)ccc2c2cnn(Cc3cccc4[nH]ncc34)c(=O)c21] | 2,7-bis((1H-indazol-4-yl)methyl)-9- methyl-7,9-dihydro-8H- pyrido[3′,2′:4,5]pyrrolo[2,3- d]pyridazin-8-one |
| 38 | 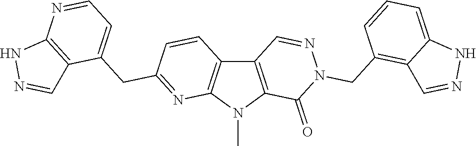 [SMILES: Cn1c2nc(Cc3ccnc4[nH]ncc34)ccc2c2cnn(Cc3cccc4[nH]ncc34)c(=O)c21] | 7-((1H-indazol-4-yl)methyl)-2-((1H- pyrazolo[3,4-b]pyridin-4- yl)methyl)-9-methyl-7,9-dihydro- 8H-pyrido[3′,2′:4,5]pyrrolo[2,3- d]pyridazin-8-one |
| 39 | 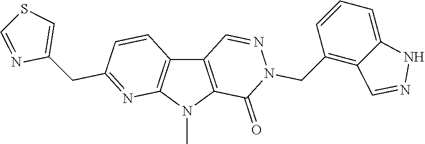 [SMILES: Cn1c2nc(Cc3cscn3)ccc2c2cnn(Cc3cccc4[nH]ncc34)c(=O)c21] | 7-((1H-indazol-4-yl)methyl)-9- methyl-2-(thiazol-4-ylmethyl)-7,9- dihydro-8H- pyrido[3′,2′:4,5]pyrrolo[2,3- d]pyridazin-8-one |
| 40 | 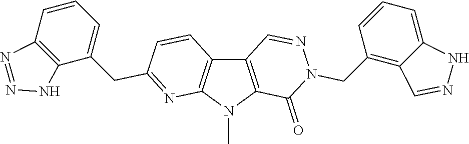 | 2-((1H-benzo[d][1,2,3]triazol-7- yl)methyl)-7-((1H-indazol-4- yl)methyl)-9-methyl-7,9-dihydro- 8H-pyrido[3′,2′:4,5]pyrrolo[2,3- d]pyridazin-8-one |
| 41 | 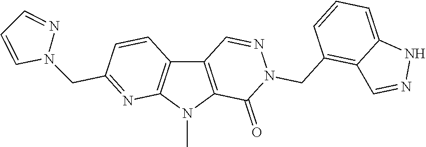 [SMILES: Cn1c2nc(Cn3cccn3)ccc2c2cnn(Cc3cccc4[nH]ncc34)c(=O)c21] | 7-((1H-indazol-4-yl)methyl)-2-((1H- pyrazol-1-yl)methyl)-9-methyl-7,9- dihydro-8H- pyrido[3′,2′:4,5]pyrrolo[2,3- d]pyridazin-8-one |
| 42 | 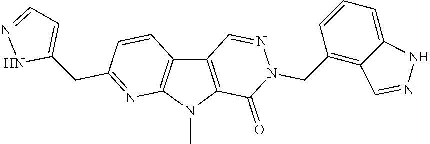 | 7-((1H-indazol-4-yl)methyl)-2-((1H- pyrazol-5-yl)methyl)-9-methyl-7,9- dihydro-8H- pyrido[3′,2′:4,5]pyrrolo[2,3- d]pyridazin-8-one |
| 43 | 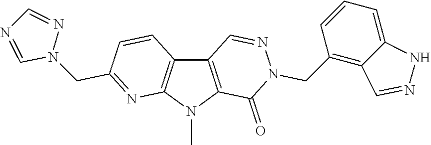 [SMILES: Cn1c2nc(Cn3cncn3)ccc2c2cnn(Cc3cccc4[nH]ncc34)c(=O)c21] | 2-((1H-1,2,4-triazol-1-yl)methyl)-7- ((1H-indazol-4-yl)methyl)-9- methyl-7,9-dihydro-8H- pyrido[3′,2′:4,5]pyrrolo[2,3- d]pyridazin-8-one |
| 44 | 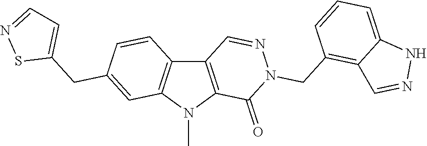 [SMILES: Cn1c2cc(Cc3ccns3)ccc2c2cnn(Cc3cccc4[nH]ncc34)c(=O)c21] | 3-((1H-indazol-4-yl)methyl)-7- (isothiazol-5-ylmethyl)-5-methyl- 3,5-dihydro-4H-pyridazino[4,5- b]indol-4-one |
| 45 | 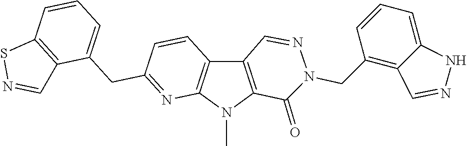 | 7-((1H-indazol-4-yl)methyl)-2- (benzo[d]isothiazol-4-ylmethyl)-9- methyl-7,9-dihydro-8H- pyrido[3′,2′:4,5]pyrrolo[2,3- d]pyridazin-8-one |
| 46 | 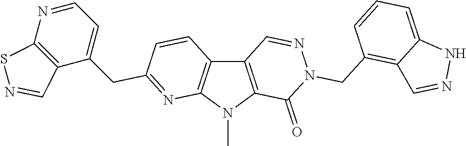 [SMILES: Cn1c2nc(Cc3ccnc4sncc34)ccc2c2cnn(Cc3cccc4[nH]ncc34)c(=O)c21] | 7-((1H-indazol-4-yl)methyl)-2- (isothiazolo[5,4-b]pyridin-4- ylmethyl)-9-methyl-7,9-dihydro- 8H-pyrido[3′,2′:4,5]pyrrolo[2,3- pyridazin-8-one |
| 47 | 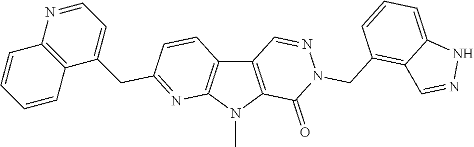 [SMILES: Cn1c2nc(Cc3ccnc4ccccc34)ccc2c2cnn(Cc3cccc4[nH]ncc34)c(=O)c21] | 7-((1H-indazol-4-yl)methyl)-9- methyl-2-(quinolin-4-ylmethyl)-7,9- dihydro-8H- pyrido[3′,2′:4,5]pyrrolo[2,3- d]pyridazin-8-one |
| 48 | 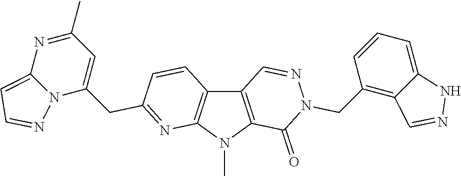 [SMILES: Cc1cc(Cc2ccc3c4cnn(Cc5cccc6[nH]ncc56)c(=O)c4n(C)c3n2)n2nccc2n1] | 7-((1H-indazol-4-yl)methyl)-9- methyl-2-((5-methylpyrazolo[1,5- a]pyrimidin-7-yl)methyl)-7,9- dihydro-8H- pyrido[3′,2′:4,5]pyrrolo[2,3- d]pyridazin-8-one |
| 49 | 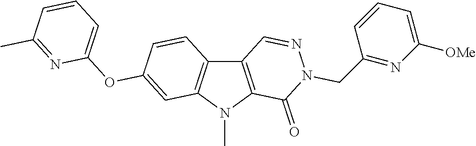 [SMILES: COc1cccc(Cn2ncc3c4ccc(Oc5cccc(C)n5)cc4n(C)c3c2=O)n1] | 3-((6-methoxypyridin-2-yl)methyl)- 5-methyl-7-((6-methylpyridin-2- yl)oxy)-3,5-dihydro-4H- pyridazino[4,5-b]indol-4-one |
| 50 | 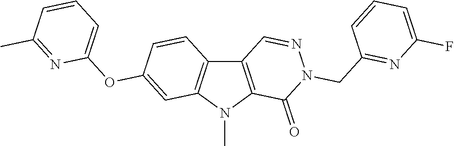 [SMILES: Cc1cccc(Oc2ccc3c4cnn(Cc5cccc(F)n5)c(=O)c4n(C)c3c2)n1] | 3-((6-fluoropyridin-2-yl)methyl)-5- methyl-7-((6-methylpyridin-2- yl)oxy)-3,5-dihydro-4H- pyridazino[4,5-b]indol-4-one |
| 51 | 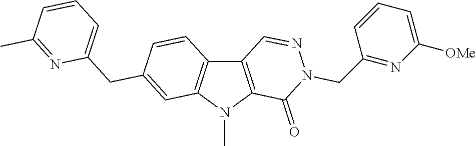 | 3-((6-methoxypyridin-2-yl)methyl)- 5-methyl-7-((6-methylpyridin-2- yl)methyl)-3,5-dihydro-4H- pyridazino[4,5-b]indol-4-one |
| 52 | 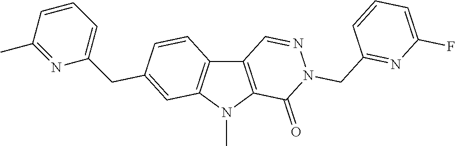 [SMILES: Cc1cccc(Cc2ccc3c4cnn(Cc5cccc(F)n5)c(=O)c4n(C)c3c2)n1] | 3-((6-fluoropyridin-2-yl)methyl)-5- methyl-7-((6-methylpyridin-2- yl)methyl)-3,5-dihydro-4H- pyridazino[4,5-b]indol-4-one |
| 53 | 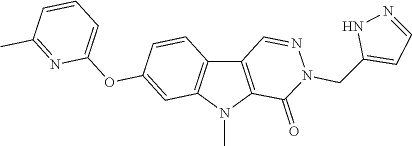 | 3-((1H-pyrazol-5-yl)methyl)-5- methyl-7-((6-methylpyridin-2- yl)oxy)-3,5-dihydro-4H- pyridazino[4,5-b]indol-4-one |
| 54 | 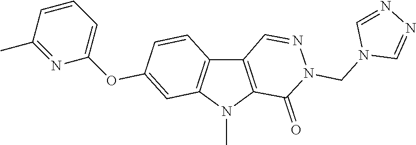 | 3-((4H-1,2,4-triazol-4-yl)methyl)-5- methyl-7-((6-methylpyridin-2- yl)oxy)-3,5-dihydro-4H- pyridazino[4,5-b]indol-4-one |
| 55 | 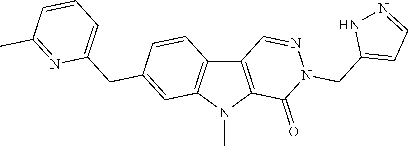 [SMILES: Cc1cccc(Cc2ccc3c4cnn(Cc5ccn[nH]5)c(=O)c4n(C)c3c2)n1] | 3-((1H-pyrazol-5-yl)methyl)-5- methyl-7-((6-methylpyridin-2- yl)methyl)-3,5-dihydro-4H- pyridazino[4,5-b]indol-4-one |
| 56 | 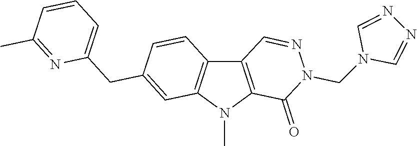 | 3-((4H-1,2,4-triazol-4-yl)methyl)-5- methyl-7-((6-methylpyridin-2- yl)methyl)-3,5-dihydro-4H- pyridazino[4,5-b]indol-4-one |
| 57 | 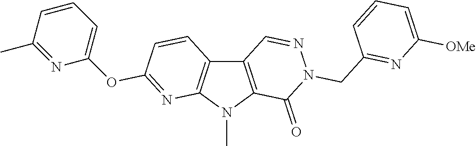 [SMILES: COc1cccc(Cn2ncc3c4ccc(Oc5cccc(C)n5)nc4n(C)c3c2=O)n1] | 7-((6-methoxypyridin-2-yl)methyl)- 9-methyl-2-((6-methylpyridin-2- yl)oxy)-7,9-dihydro-8H- pyrido[3′,2′:4,5]pyrrolo[2,3- d]pyridazin-8-one |
| 58 | 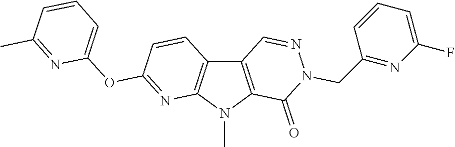 [SMILES: Cc1cccc(Oc2ccc3c4cnn(Cc5cccc(F)n5)c(=O)c4n(C)c3n2)n1] | 7-((6-fluoropyridin-2-yl)methyl)-9- methyl-2-((6-methylpyridin-2- yl)oxy)-7,9-dihydro-8H- pyrido[3′,2′:4,5]pyrrolo[2,3- d]pyridazin-8-one |
| 59 | 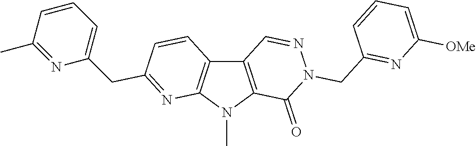 [SMILES: COc1cccc(Cn2ncc3c4ccc(Cc5cccc(C)n5)nc4n(C)c3c2=O)n1] | 7-((6-methoxypyridin-2-yl)methyl)- 9-methyl-2-((6-methylpyridin-2- yl)methyl)-7,9-dihydro-8H- pyrido[3′,2′:4,5]pyrrolo[2,3- d]pyridazin-8-one |
| 60 | 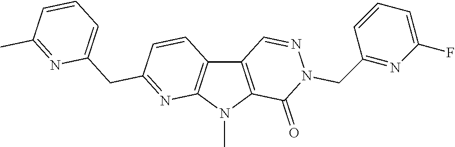 [SMILES: Cc1cccc(Cc2ccc3c4cnn(Cc5cccc(F)n5)c(=O)c4n(C)c3n2)n1] | 7-((6-fluoropyridin-2-yl)methyl)-9- methyl-2-((6-methylpyridin-2- yl)methyl)-7,9-dihydro-8H- pyrido[3′,2′:4,5]pyrrolo[2,3- d]pyridazin-8-one |
| 61 | 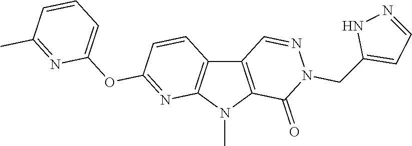 [SMILES: Cc1cccc(Oc2ccc3c4cnn(Cc5ccn[nH]5)c(=O)c4n(C)c3n2)n1] | 7-((1H-pyrazol-5-yl)methyl)-9- methyl-2-((6-methylpyridin-2- yl)oxy)-7,9-dihydro-8H- pyrido[3′,2′:4,5]pyrrolo[2,3- d]pyridazin-8-one |
| 62 | 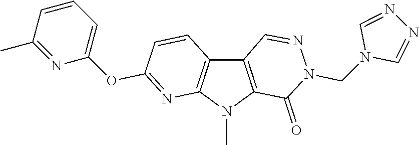 | 7-((4H-1,2,4-triazol-4-yl)methyl)-9- methyl-2-((6-methylpyridin-2- yl)oxy)-7,9-dihydro-8H- pyrido[3′,2′:4,5]pyrrolo[2,3- d]pyridazin-8-one |
| 63 | 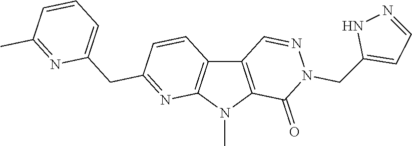 [SMILES: Cc1cccc(Cc2ccc3c4cnn(Cc5ccn[nH]5)c(=O)c4n(C)c3n2)n1] | 7-((1H-pyrazol-5-yl)methyl)-9- methyl-2-((6-methylpyridin-2- yl)methyl)-7,9-dihydro-8H- pyrido[3′,2′:4,5]pyrrolo[2,3- d]pyridazin-8-one |
| 64 | 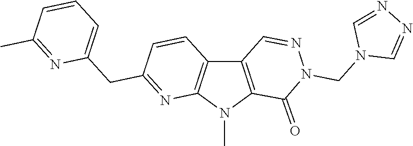 | 7-((4H-1,2,4-triazol-4-yl)methyl)-9- methyl-2-((6-methylpyridin-2- yl)methyl)-7,9-dihydro-8H- pyrido[3′,2′:4,5]pyrrolo[2,3- d]pyridazin-8-one |
| 65 | 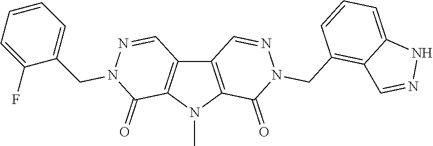 [SMILES: Cn1c2c(=O)n(Cc3ccccc3F)ncc2c2cnn(Cc3cccc4[nH]ncc34)c(=O)c21] | 3-((1H-indazol-4-yl)methyl)-7-(2- fluorobenzyl)-5-methyl-5,7- dihydro-3H-pyrrolo[2,3-d:4,5- d′]dipyridazine-4,6-dione |
| 66 | 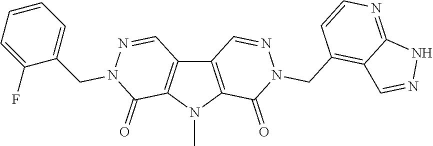 [SMILES: Cn1c2c(=O)n(Cc3ccccc3F)ncc2c2cnn(Cc3ccnc4[nH]ncc34)c(=O)c21] | 3-((1H-pyrazolo[3,4-b]pyridin-4- yl)methyl)-7-(2-fluorobenzyl)-5- methyl-5,7-dihydro-3H-pyrrolo[2,3- d:4,5-d′]dipyridazine-4,6-dione |
| 67 | 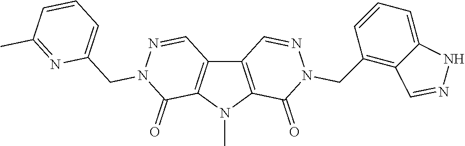 [SMILES: Cc1cccc(Cn2ncc3c4cnn(Cc5cccc6[nH]ncc56)c(=O)c4n(C)c3c2=O)n1] | 3-((1H-indazol-4-yl)methyl)-5- methyl-7-((6-methylpyridin-2- yl)methyl)-5,7-dihydro-3H- pyrrolo[2,3-d:4,5-d′]dipyridazine- 4,6-dione |
| 68 | 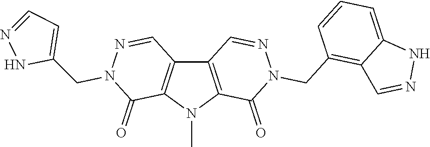 | 3-((1H-indazol-4-yl)methyl)-7-((1H- pyrazol-5-yl)methyl)-5-methyl-5,7- dihydro-3H-pyrrolo[2,3-d:4,5- d′]dipyridazine-4,6-dione |
| 69 | 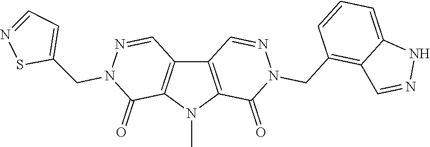 [SMILES: Cn1c2c(=O)n(Cc3ccns3)ncc2c2cnn(Cc3cccc4[nH]ncc34)c(=O)c21] | 3-((1H-indazol-4-yl)methyl)-7- (isothiazol-5-ylmethyl)-5-methyl- 5,7-dihydro-3H-pyrrolo[2,3-d:4,5- d′]dipyridazine-4,6-dione |
| 70 | 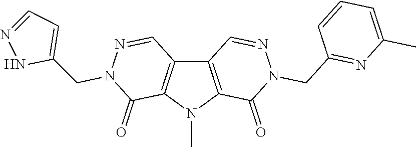 | 3-((1H-pyrazol-5-yl)methyl)-5- methyl-7-((6-methylpyridin-2- yl)methyl)-5,7-dihydro-3H- pyrrolo[2,3-d:4,5-d′]dipyridazine- 4,6-dione |
| 71 | 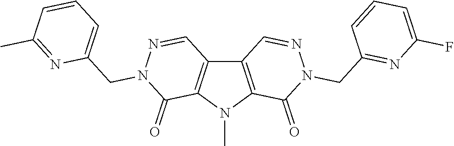 [SMILES: Cc1cccc(Cn2ncc3c4cnn(Cc5cccc(F)n5)c(=O)c4n(C)c3c2=O)n1] | 3-((6-fluoropyridin-2-yl)methyl)-5- methyl-7-((6-methylpyridin-2- yl)methyl)-5,7-dihydro-3H- pyrrolo[2,3-d:4,5-d′]dipyridazine- 4,6-dione |
| 72 | 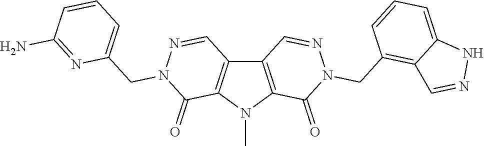 [SMILES: Cn1c2c(=O)n(Cc3cccc(N)n3)ncc2c2cnn(Cc3cccc4[nH]ncc34)c(=O)c21] | 3-((1H-indazol-4-yl)methyl)-7-((6- aminopyridin-2-yl)methyl)-5- methyl-5,7-dihydro-3H-pyrrolo[2,3- d:4,5-d′]dipyridazine-4,6-dione |
| 73 | 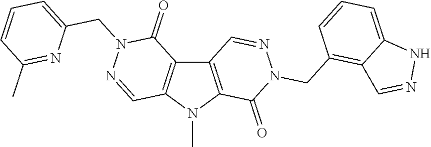 [SMILES: Cc1cccc(Cn2ncc3c(c2=O)c2cnn(Cc4cccc5[nH]ncc45)c(=O)c2n3C)n1] | 7-((1H-indazol-4-yl)methyl)-5- methyl-2-((6-methylpyridin-2- yl)methyl)-5,7-dihydro-1H- pyrrolo[2,3-d:4,5-d′]dipyridazine- 1,6(2H)-dione |
| 74 | 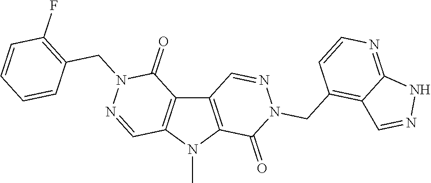 [SMILES: Cn1c2cnn(Cc3ccccc3F)c(=O)c2c2cnn(Cc3ccnc4[nH]ncc34)c(=O)c21] | 7-((1H-pyrazolo[3,4-b]pyridin-4- yl)methyl)-2-(2-fluorobenzyl)-5- methyl-5,7-dihydro-1H-pyrrolo[2,3- d:4,5-d′]dipyridazine-1,6(2H)-dione |
| 75 | 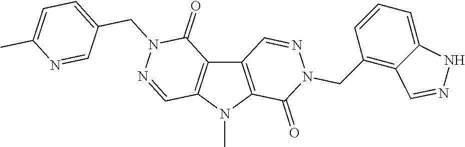 | 7-((1H-indazol-4-yl)methyl)-5- methyl-2-((6-methylpyridin-3- yl)methyl)-5,7-dihydro-1H- pyrrolo[2,3-d:4,5-d′]dipyridazine- 1,6(2H)-dione |
| 76 | 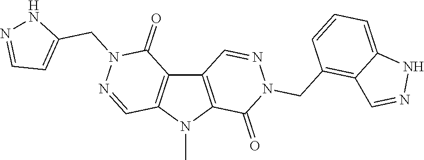 [SMILES: Cn1c2cnn(Cc3ccn[nH]3)c(=O)c2c2cnn(Cc3cccc4[nH]ncc34)c(=O)c21] | 7-((1H-indazol-4-yl)methyl)-2-((1H- pyrazol-5-yl)methyl)-5-methyl-5,7- dihydro-1H-pyrrolo[2,3-d:4,5- d′]dipyridazine-1,6(2H)-one |
| 77 | 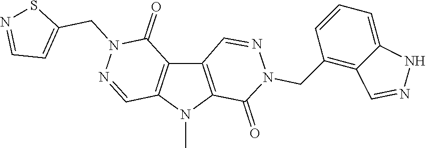 | 7-((1H-indazol-4-yl)methyl)-2- (isothiazol-5-ylmethyl)-5-methyl- 5,7-dihydro-1H-pyrrolo[2,3-d:4,5- d′]dipyridazine-1,6(2H)-one |
| 78 | 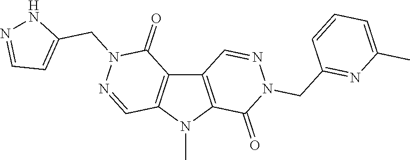 [SMILES: Cc1cccc(Cn2ncc3c4c(=O)n(Cc5ccn[nH]5)ncc4n(C)c3c2=O)n1] | 2-((1H-pyrazol-5-yl)methyl)-5- methyl-7-((6-methylpyridin-2- yl)methyl)-5,7-dihydro-1H- pyrrolo[2,3-d:4,5-d′]dipyridazine- 1,6(2H)-dione |
| 79 | 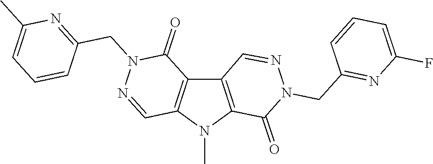 | 7-((6-fluoropyridin-2-yl)methyl)-5- methyl-2-((6-methylpyridin-2- yl)methyl)-5,7-dihydro-1H- pyrrolo[2,3-d:4,5′]dipyridazine- 1,6(2H)-dione |
| 80 | 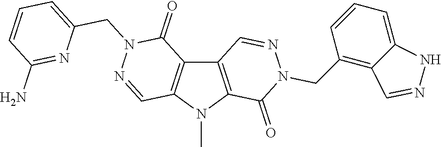 [SMILES: Cn1c2cnn(Cc3cccc(N)n3)c(=O)c2c2cnn(Cc3cccc4[nH]ncc34)c(=O)c21] | 7-((1H-indazol-4-yl)methyl)-2-((6- aminopyridin-2-yl)methyl)-5- methyl-5,7-dihydro-1H-pyrrolo[2,3- d:4,5-d′]dipyridazine-1,6(2H)-dione |
| 81 | 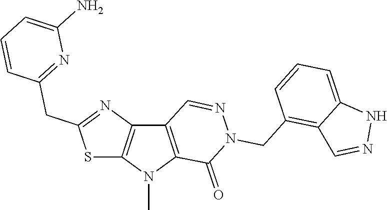 | 6-((1H-indazol-4-yl)methyl)-2-((6- aminopyridin-2-yl)methyl)-4- methyl-4,6-dihydro-5H- thiazolo[4′,5′:4,5]pyrrolo[2,3- d]pyridazin-5-one |
| 82 | 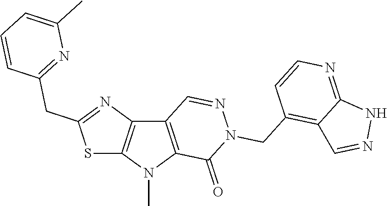 [SMILES: Cc1cccc(Cc2nc3c4cnn(Cc5ccnc6[nH]ncc56)c(=O)c4n(C)c3s2)n1] | 6-((1H-pyrazolo[3,4-b]pyridin-4- yl)methyl)-4-methyl-2-((6- methylpyridin-2-yl)methyl)-4,6- dihydro-5H- thiazolo[4′,5′:4,5]pyrrolo[2,3- d]pyridazin-5-one |
| 83 | 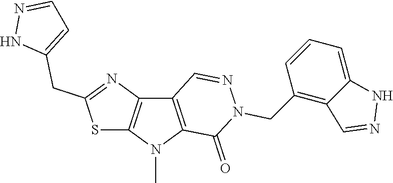 | 6-((1H-indazol-4-yl)methyl)-2-((1H- pyrazol-5-yl)methyl)-4-methyl-4,6- dihydro-5H- thiazolo[4′,5′:4,5]pyrrolo[2,3- d]pyridazin-5-one |
| 84 | 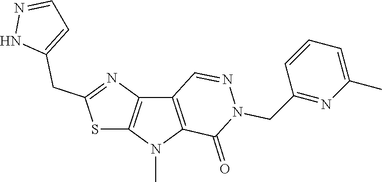 | 2-((1H-pyrazol-5-yl)methyl)-4- methyl-6-((6-methylpyridin-2- yl)methyl)-4,6-dihydro-5H- thiazolo[4′,5′:4,5]pyrrolo[2,3- d]pyridazin-5-one |
| 85 | 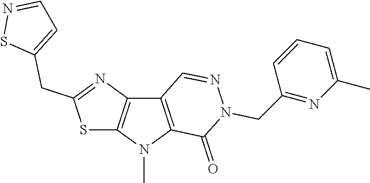 | 2-(isothiazol-5-ylmethyl)-4-methyl- 6-((6-methylpyridin-2-yl)methyl)- 4,6-dihydro-5H- thiazolo[4′,5′:4,5]pyrrolo[2,3- d]pyridazin-5-one |
| 86 | 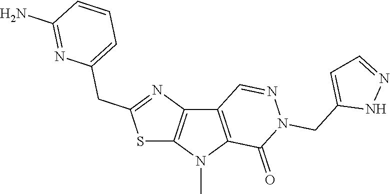 [SMILES: Cn1c2sc(Cc3cccc(N)n3)nc2c2cnn(Cc3ccn[nH]3)c(=O)c21] | 6-((1H-pyrazol-5-yl)methyl)-2-((6- aminopyridin-2-yl)methyl)-4- methyl-4,6-dihydro-5H- thiazolo[4′,5′:4,5]pyrrolo[2,3- d]pyridazin-5-one |
| 87 | 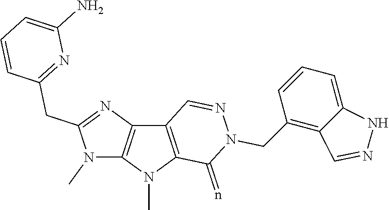 | 6-((1H-indazol-4-yl)methyl)-2-((6- aminopyridin-2-yl)methyl)-3,4- dimethyl-4,6- dihydroimidazo[4′,5′:4,5]pyrrolo[2, 3-d]pyridazin-5(3H)-one |
| 88 | 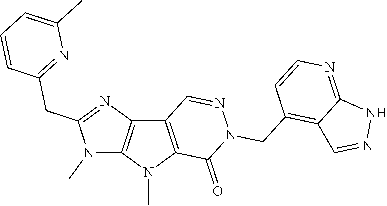 [SMILES: Cc1cccc(Cc2nc3c4cnn(Cc5ccnc6[nH]ncc56)c(=O)c4n(C)c3n2C)n1] | 6-((1H-pyrazolo[3,4-b]pyridin-4- yl)methyl)-3,4-dimethyl-2-((6- methylpyridin-2-yl)methyl)-4,6- dihydroimidazo[4′,5′,:4,5]pyrrolo[2, 3-d]pyridazin-5(3H)-one |
| 89 | 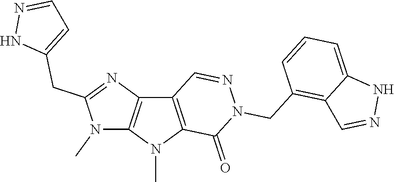 [SMILES: Cn1c(Cc2ccn[nH]2)nc2c3cnn(Cc4cccc5[nH]ncc45)c(=O)c3n(C)c21] | 6-((1H-indazol-4-yl)methyl)-2-((1H- pyrazol-5-yl)methyl)-3,4-dimethyl- 4,6- dihydroimidazo[4′,5′:4,5]pyrrolo[2, 3-d]pyridazin-5(3H)-one |
| 90 | 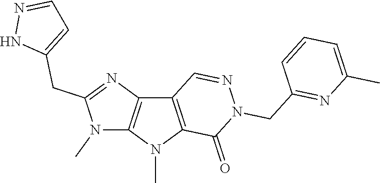 [SMILES: Cc1cccc(Cn2ncc3c4nc(Cc5ccn[nH]5)n(C)c4n(C)c3c2=O)n1] | 2-((1H-pyrazol-5-yl)methyl)-3,4- dimethyl-6-((6-methylpyridin-2- yl)methyl)-4,6- dihydroimidazo[4′,5′:4,5]pyrrolo[2, 3-d]pyridazin-5(3H)-one |
| 91 | 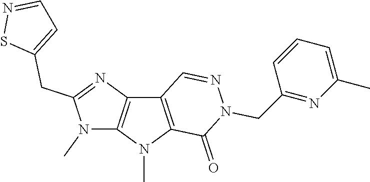 [SMILES: Cc1cccc(Cn2ncc3c4nc(Cc5ccns5)n(C)c4n(C)c3c2=O)n1] | 2-(isothiazol-5-ylmethyl)-3,4- dimethyl-6-((6-methylpyridin-2- yl)methyl)-4,6- dihydroimidazo[4′,5′:4,5]pyrrolo[2, 3-d]pyridazin-5(3H)-one |
| 92 | 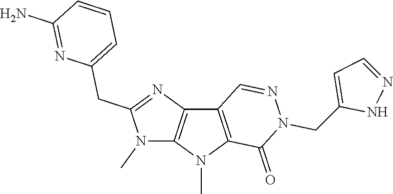 [SMILES: Cn1c(Cc2cccc(N)n2)nc2c3cnn(Cc4ccn[nH]4)c(=O)c3n(C)c21] | 6-((1H-pyrazol-5-yl)methyl)-2-((6- aminopyridin-2-yl)methyl)-3,4- dimethyl-4,6- dihydroimidazo[4′,5′:4,5]pyrrolo[2, 3-d]pyridazin-5(3H)-one |
| 93 | 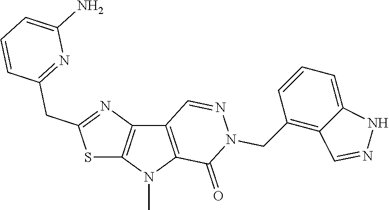 [SMILES: Cn1c2sc(Cc3cccc(N)n3)nc2c2cnn(Cc3cccc4[nH]ncc34)c(=O)c21] | 6-((1H-indazol-4-yl)methyl)-2-((6- aminopyridin-2-yl)methyl)-4- methyl-4,6-dihydro-5H- thiazolo[4′,5′:4,5]pyrrolo[2,3- d]pyridazin-5-one |
| 94 | 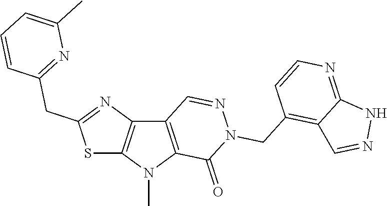 [SMILES: Cc1cccc(Cc2nc3c4cnn(Cc5ccnc6[nH]ncc56)c(=O)c4n(C)c3s2)n1] | 6-((1H-pyrazolo[3,4-b]pyridin-4- yl)methyl)-4-methyl-2-((6- methylpyridin-2-yl)methyl)-4,6- dihydro-5H- thiazolo[4′,5′:4,5]pyrrolo[2,3- d]pyridazin-5-one |
| 95 | 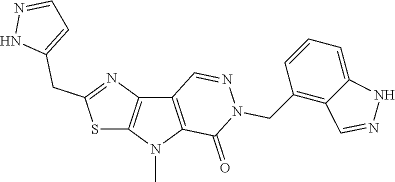 [SMILES: Cn1c2sc(Cc3ccn[nH]3)nc2c2cnn(Cc3cccc4[nH]ncc34)c(=O)c21] | 6-((1H-indazol-4-yl)methyl-2-((1H- pyrazol-5-yl)methyl)-4-methyl-4,6- dihydro-5H- thiazolo[4′,5′:4,5]pyrrolo[2,3- d]pyridazin-5-one |
| 96 | 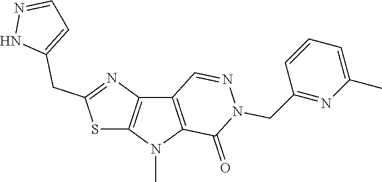 [SMILES: Cc1cccc(Cn2ncc3c4nc(Cc5ccn[nH]5)sc4n(C)c3c2=O)n1] | 2-((1H-pyrazol-5-yl)methyl)-4- methyl-6-((6-methylpyridin-2- yl)methyl)-4,6-dihydro-5H- thiazolo[4′,5′:4,5]pyrrolo[2,3- d]pyridazin-5-one |
| 97 | 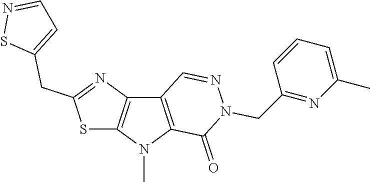 [SMILES: Cc1cccc(Cn2ncc3c4nc(Cc5ccns5)sc4n(C)c3c2=O)n1] | 2-(isothiazol-5-ylmethyl)-4-methyl- 6-((6-methylpyridin-2-yl)methyl)- 4,6-dihydro-5H- thiazolo[4′,5′:4,5]pyrrolo[2,3- d]pyridazin-5-one |
| 98 | 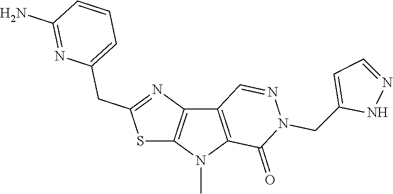 | 6-((1H-pyrazol-5-yl)methyl)-2-((6- aminopyridin-2-yl)methyl)-4- methyl-4,6-dihydro-5H- thiazolo[4′,5′:4,5]pyrrolo[2,3- d]pyridazin-5-one |
| 99 | 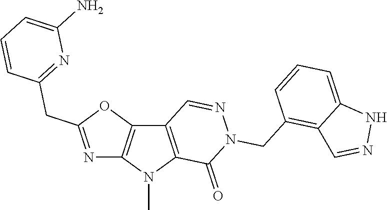 [SMILES: Cn1c2nc(Cc3cccc(N)n3)oc2c2cnn(Cc3cccc4[nH]ncc34)c(=O)c21] | 6-((1H-indazol-4-yl)methyl)-2-((6- aminopyridin-2-yl)methyl)-4- methyl-4,6-dihydro-5H- oxazolo[5′,4′:4,5]pyrrolo[2,3- d]pyridazin-5-one |
| 100 | 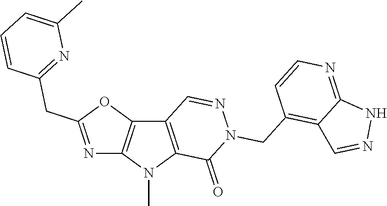 [SMILES: Cc1cccc(Cc2nc3c(o2)c2cnn(Cc4ccnc5[nH]ncc45)c(=O)c2n3C)n1] | 6-((1H-pyrazolo[3,4-b]pyridin-4- yl)methyl)-4-methyl-2-((6- methylpyridin-2-yl)methyl)-4,6- dihydro-5H- oxazolo[5′,4′:4,5]pyrrolo[2,3- d]pyridazin-5-one |
| 101 | 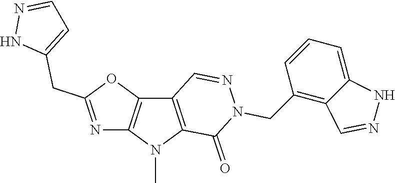 | 6-((1H-indazol-4-yl)methyl)-2-((1H- pyrazol-5-yl)methyl)-4-methyl-4,6- dihydro-5H- oxazolo[5′,4′:4,5]pyrrolo[2,3- d]pyridazin-5-one |
| 102 | 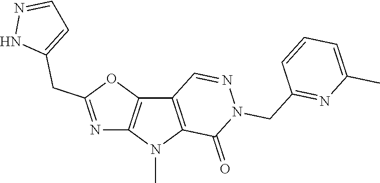 [SMILES: Cc1cccc(Cn2ncc3c4oc(Cc5ccn[nH]5)nc4n(C)c3c2=O)n1] | 2-((1H-pyrazol-5-yl)methyl)-4- methyl-6-((6-methylpyridin-2- yl)methyl)-4,6-dihydro-5H- oxazolo[5′,4′:4,5]pyrrolo[2,3- d]pyridazin-5-one |
| 103 | 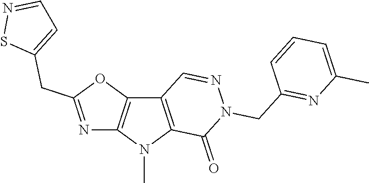 [SMILES: Cc1cccc(Cn2ncc3c4oc(Cc5ccns5)nc4n(C)c3c2=O)n1] | 2-(isothiazol-5-ylmethyl)-4-methyl- 6-((6-methylpyridin-2-yl)methyl)- 4,6-dihydro-5H- oxazolo[5′,4′:4,5]pyrrolo[2,3- d]pyridazin-5-one |
| 104 | 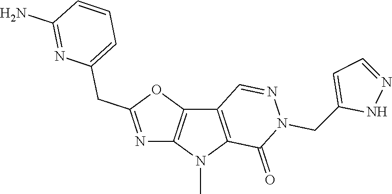 [SMILES: Cn1c2nc(Cc3cccc(N)n3)oc2c2cnn(Cc3ccn[nH]3)c(=O)c21] | 6-((1H-pyrazol-5-yl)methyl)-2-((6- aminopyridin-2-yl)methyl)-4- methyl-4,6-dihydro-5H- oxazolo[5′,4′:4,5]pyrrolo[2,3- d]pyridazin-5-one |

All publications and patents mentioned in the above specification are herein incorporated by reference. Various modifications and variations of the described method and system of the disclosure Will be apparent to those skilled in the art without departing from the scope and spirit of the disclosure. Although the disclosure has been described in connection with specific preferred embodiments, it should be understood that the disclosure as claimed should not be unduly limited to such specific embodiments. Indeed, various modifications of the described modes for carrying out the disclosure that are obvious to those skilled relevant fields are intended to be within the scope of the following claims.

1. A composition, comprising a compound: 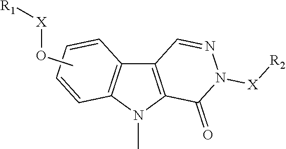
wherein
X is null or is selected from —H, —CH₂—, —CHR₃—, —CR₃R₄—, —(CH₂)ₙ—, —(CHR₃)ₙ—, —(CR₃R₄)ₙ—, wherein n-1-6;
R₁ is selected from the group consisting of —H, —CN, —NO₂, —NH₂, —NHR₃, NR₃R₄, —OH, OR₃, —SOR₃, —SO₂R₃, optionally substituted C₁-C₆ alkyl, optionally substituted monocyclic cycloalkyl, optionally substituted bicyclic cycloalkyl, optionally substituted tricyclic cycloalkyl, optionally substituted monocyclic heterocycle, optionally substituted bicyclic heterocycle, and optionally substituted tricyclic heterocycle;
R₂ is selected from the group consisting of —H, —CN, —NO₂, —NH₂, —NHR₃, NR₃R₄, —OH, OR₃, —SOR₃, —SO₂R₃, optionally substituted C₁-C₆ alkyl, optionally substituted monocyclic cycloalkyl, optionally substituted bicyclic cycloalkyl, optionally substituted monocyclic heterocycle, optionally substituted bicyclic heterocycle, and optionally substituted tricyclic heterocycle;
R₃ and R₄ are each independently selected from the group consisting of optionally substituted C₁-C₆ alkyl, optionally substituted monocyclic cycloalkyl, optionally substituted bicyclic cycloalkyl, optionally substituted tricyclic cycloalkyl, optionally substituted monocyclic heterocycle, optionally substituted bicyclic heterocycle, and optionally substituted tricyclic heterocycle.

2. The composition of claim 1, wherein R₁ is selected from the group consisting of 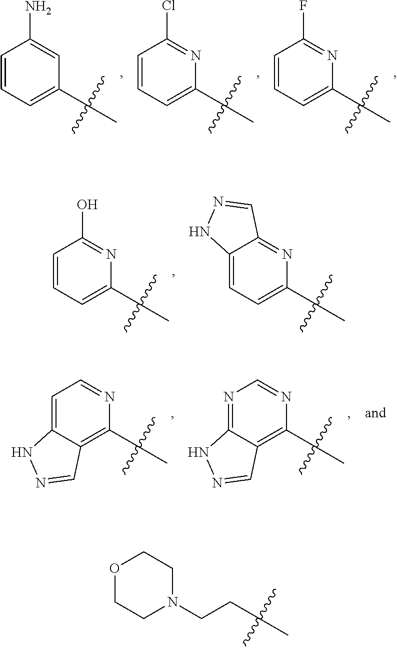
and R₂ is selected from the group consisting of

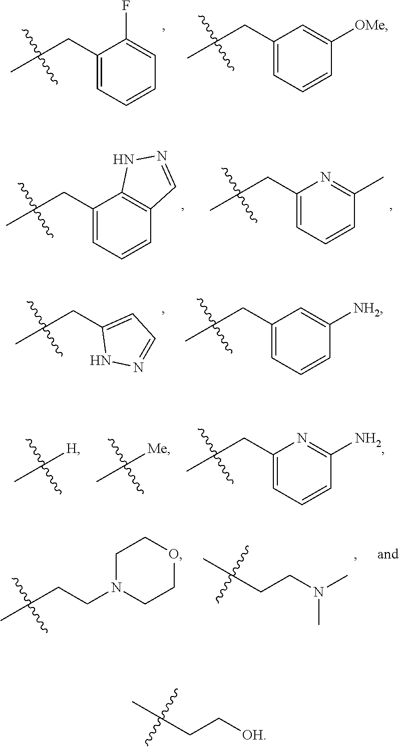

3. The composition of claim 1, wherein said compound is selected from the group consisting of

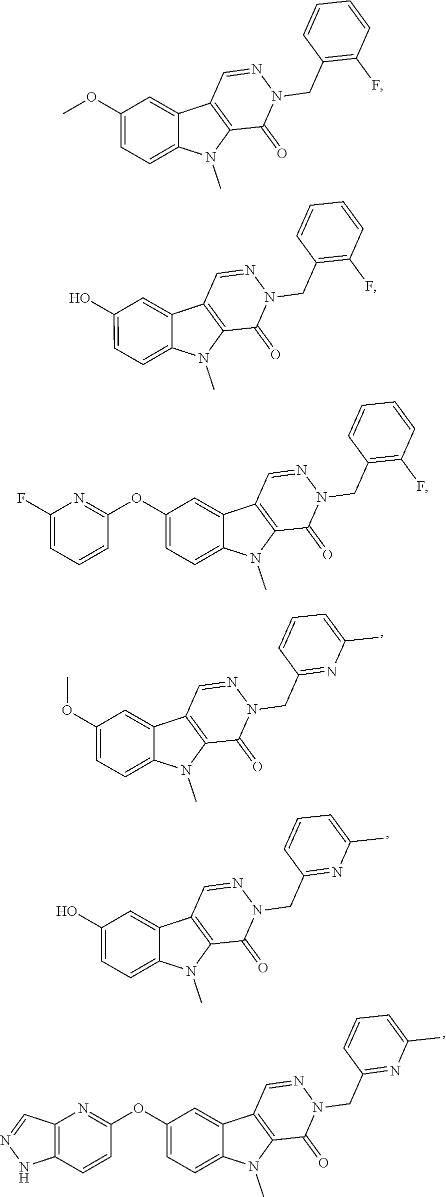

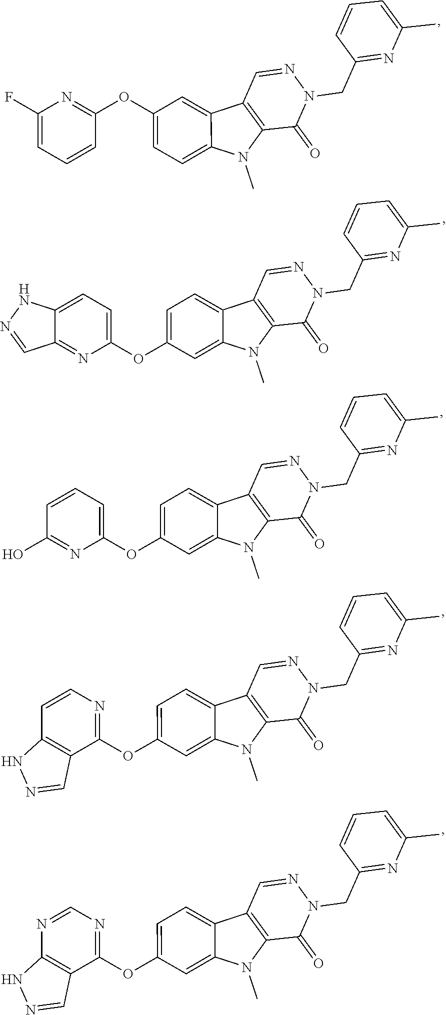 [SMILES: Cc1cccc(Cn2ncc3c4cc(Oc5cccc(F)n5)ccc4n(C)c3c2=O)n1 ; Cc1cccc(Cn2ncc3c4ccc(Oc5ccc6[nH]ncc6n5)cc4n(C)c3c2=O)n1 ; Cc1cccc(Cn2ncc3c4ccc(Oc5cccc(O)n5)cc4n(C)c3c2=O)n1 ; Cc1cccc(Cn2ncc3c4ccc(Oc5nccc6[nH]ncc56)cc4n(C)c3c2=O)n1 ; Cc1cccc(Cn2ncc3c4ccc(Oc5ncnc6[nH]ncc56)cc4n(C)c3c2=O)n1]

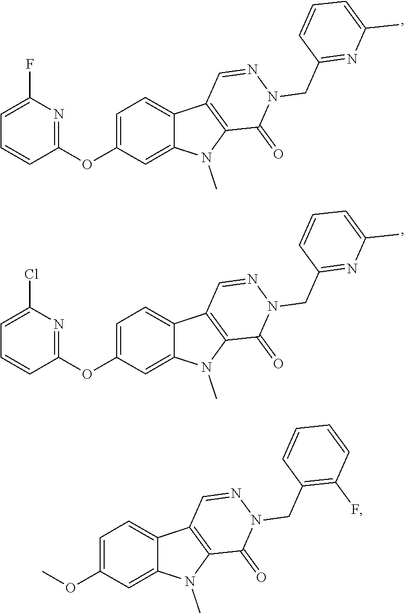

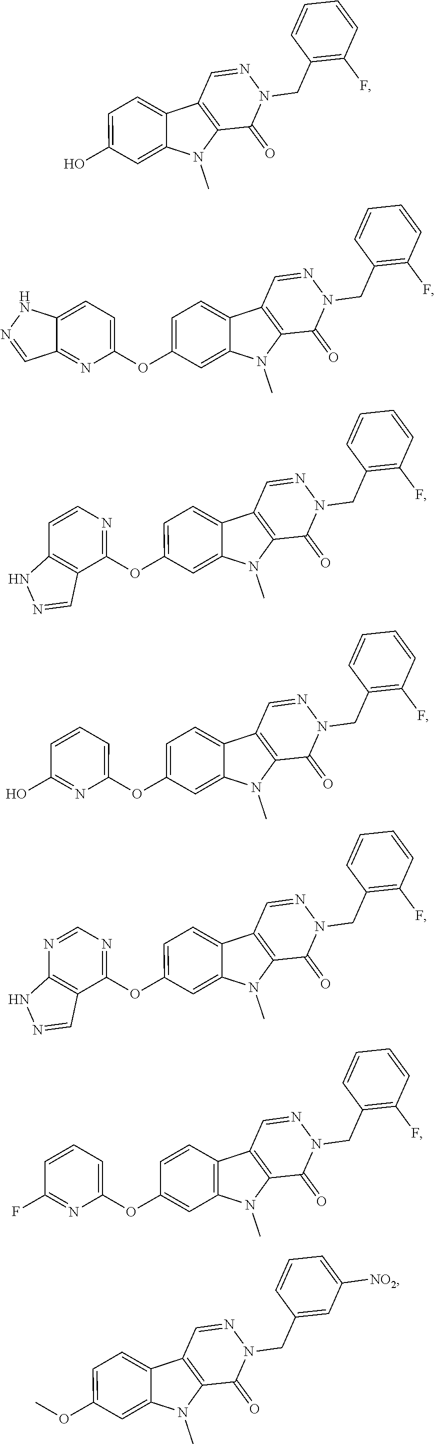

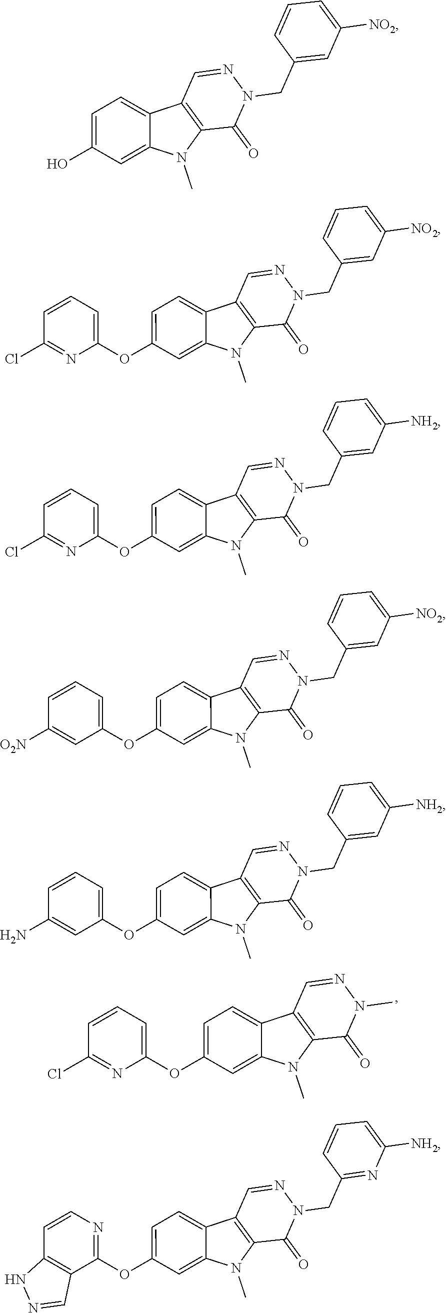

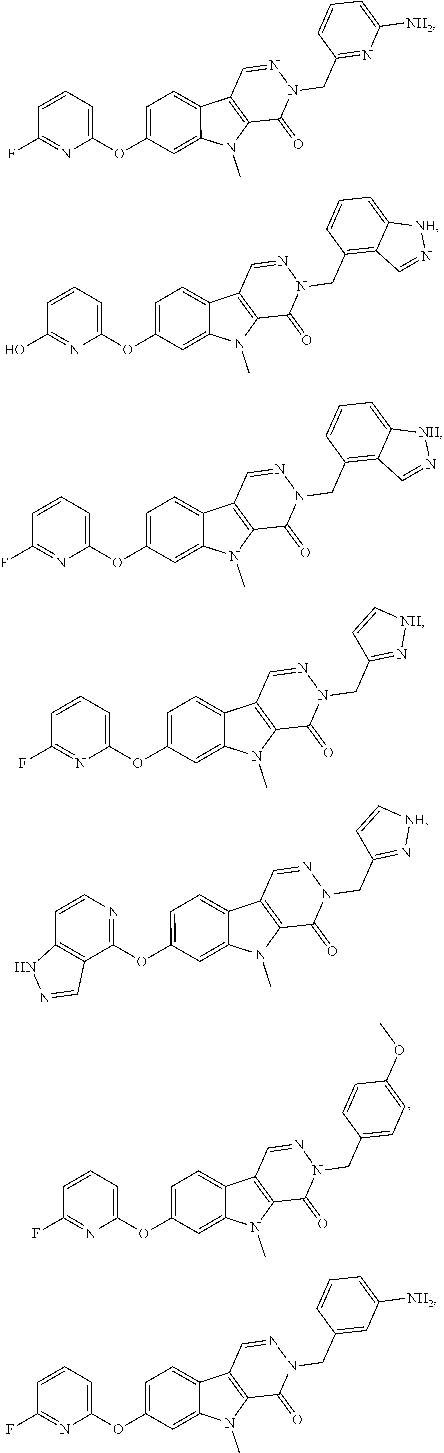

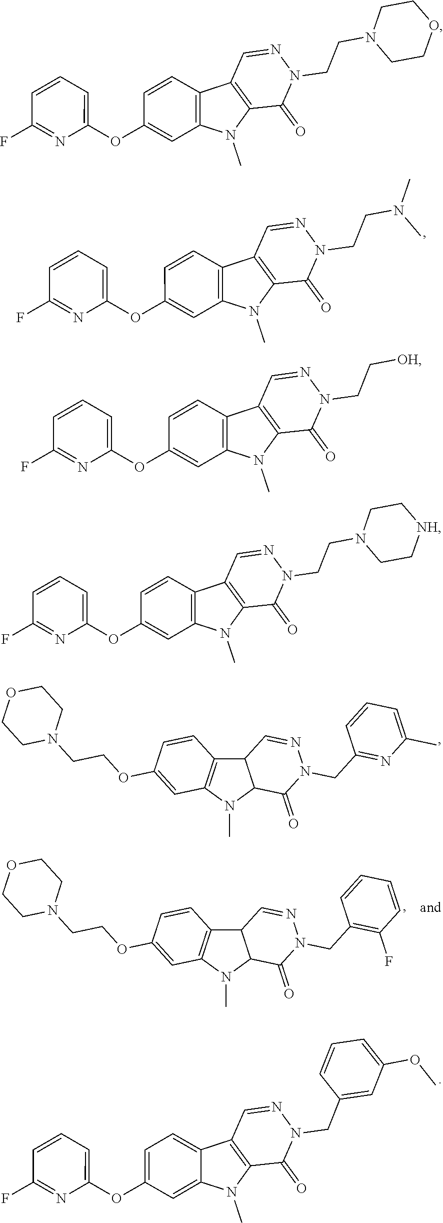

4. A composition, comprising: 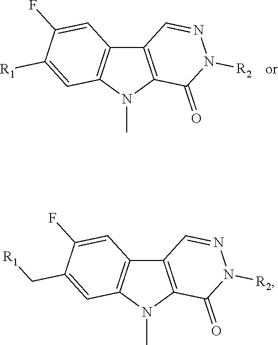
wherein R₁ is selected from the group consisting of —H, —F, —Cl, —Br, —CN, —NO₂, —NH₂, —NHR₃, —NR₃R₄, —OH, —OR₃, optionally substituted C₁-C₆ alkyl, optionally substituted monocyclic cycloalkyl, optionally substituted bicyclic cycloalkyl, optionally substituted tricyclic cycloalkyl, optionally substituted monocyclic heterocycle, optionally substituted bicyclic heterocycle, and optionally substituted tricyclic heterocycle;
R₂ is selected from the group consisting of —H, —CH₃, —(CH₂)ₙ—R₅, —(CHR₃)ₙ—R₅, —(CR₃R₄)ₙ—R₅, optionally substituted C₁-C₆ alkyl, optionally substituted monocyclic cycloalkyl, optionally substituted bicyclic cycloalkyl, optionally substituted monocyclic heterocycle, optionally substituted bicyclic heterocycle, and optionally substituted tricyclic heterocycle;
R₃ and R₄ are each independently selected from the group consisting of optionally substituted C₁-C₆ alkyl, optionally substituted monocyclic cycloalkyl, optionally substituted bicyclic cycloalkyl, optionally substituted tricyclic cycloalkyl, optionally substituted monocyclic heterocycle, optionally substituted bicyclic heterocycle, and optionally substituted tricyclic heterocycle; and
R₅ is selected from the group consisting of —H, —F, —Cl, —Br, —NO₂, —CN, —NO₂, —NH₂, —NHR₃, —NR₃R₄, —OH, —OR₃, optionally substituted C₁-C₆ alkyl, optionally substituted monocyclic cycloalkyl, optionally substituted bicyclic cycloalkyl, optionally substituted tricyclic cycloalkyl, optionally substituted monocyclic heterocycle, optionally substituted bicyclic heterocycle, and optionally substituted tricyclic heterocycle.

5. The composition of claim 4, wherein R₁ is selected from the group consisting of OCH₃, OH, NH₂, NCH₃, N(CH₃)₂, 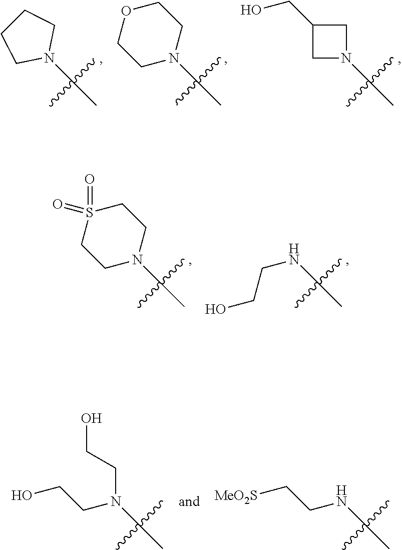
and R₂ is selected from the group consisting of

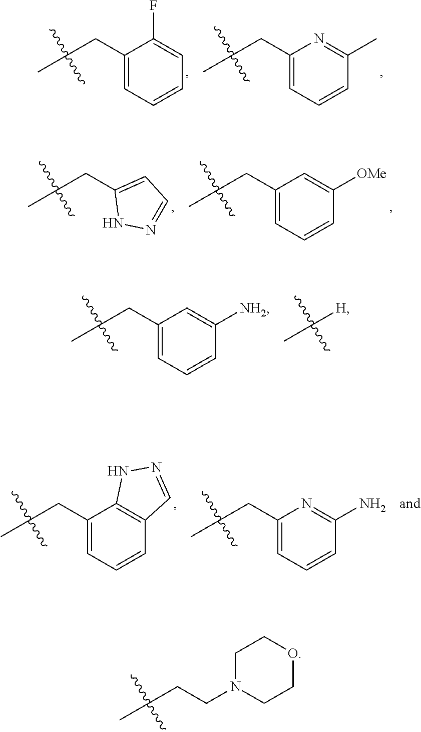

6. The composition of any claim 4, wherein said compound is selected from the group consisting of

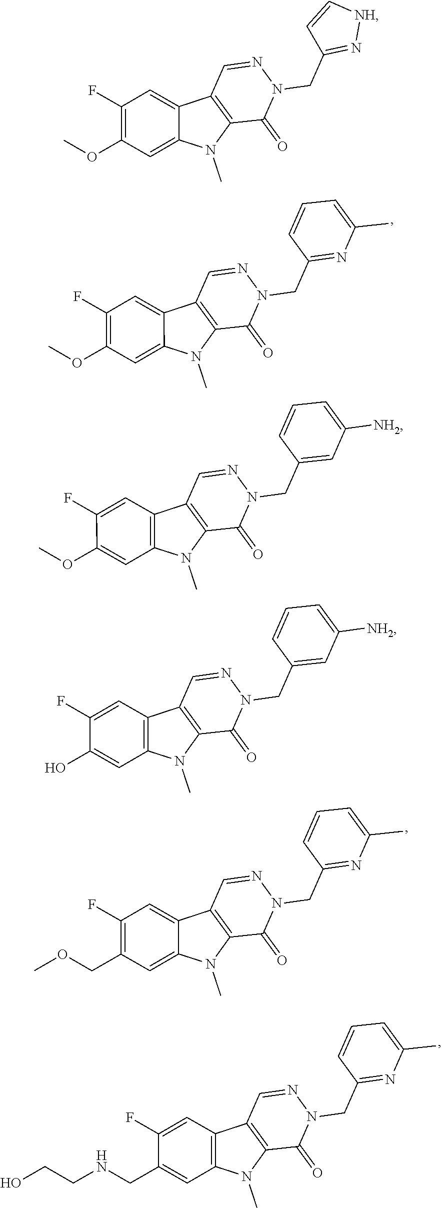

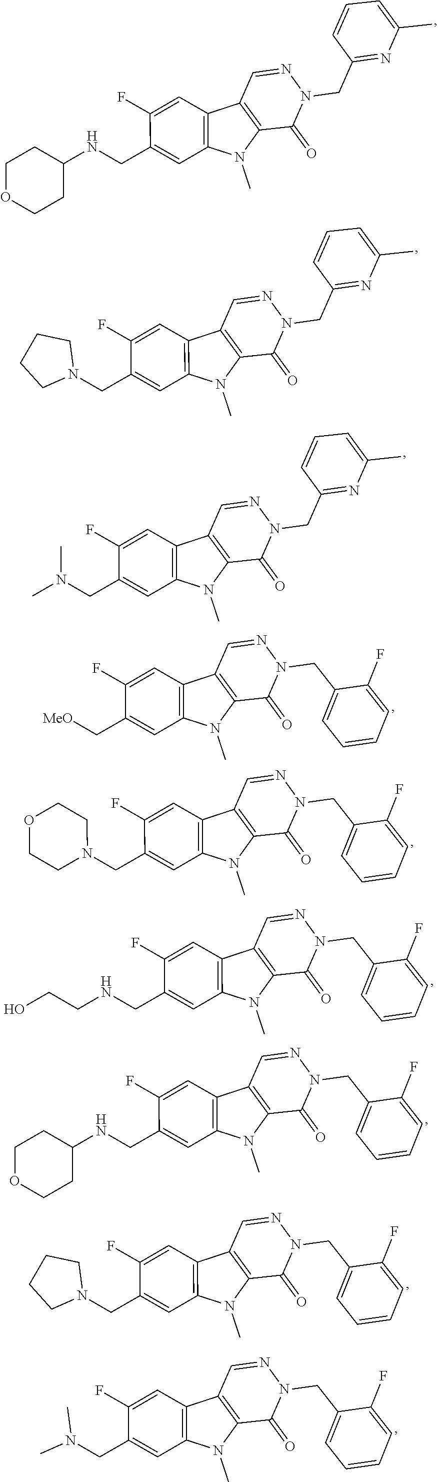

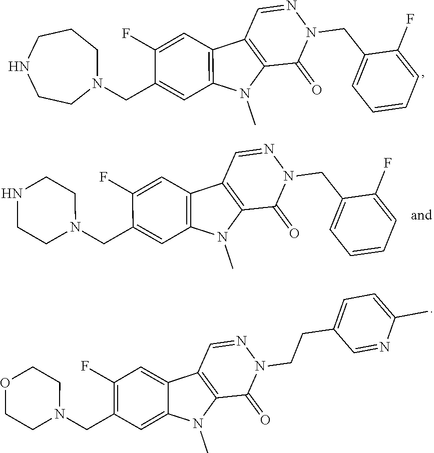

7. The composition of claim 4, wherein at least one hydrogen of said compound is replaced with deuterium.

8. The composition of claim 4, wherein said composition is a pharmaceutical composition.

9. The composition of claim 4, wherein said composition is formulated for injection, for oral delivery, or as an eye drop.

10. The composition of claim 4, wherein said composition comprises a pharmaceutically acceptable carrier.

11. The composition of claim 4, wherein said composition is a pyruvate kinase activator.

12. The composition of claim 16, wherein said pyruvate kinase is selected from the group consisting of PKM1 and PKM2.

13-19. (canceled)

20. A method of treating a disease or condition, comprising: administering a pyruvate kinase activator comprising:
a compound selected from 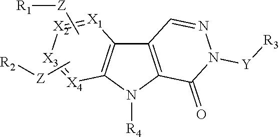
Formula I or 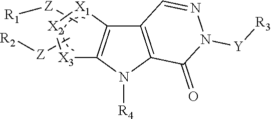
Formula II:
Wherein X₁, X₂, X₃, and X₄ are independently selected from CH, CO, N, NH, S, or O;
Z is null, a bond, optionally substituted C₁₋₆ alkyl, —O—, —S—, —CH₂—, —CHR₅—, —CR₅R₆—, —(CH₂)ₙ—, —(CHR₅)ₙ—, —(CR₅R₆)ₙ—, —S(═O)CH₂, —S(═O)₂CH₂—, —NR₅—, —NR₅C(═O)—, —C(O)NR₅—, —C(═O)—, —OC(═O)—, —C(═O)O—, —NR₅C(═O)O—, —OC(═O)NR₅—, —NR₅C(═O)NR₅—, —OC(R₅)₂—, —C(R₅)₂O—, —NR₄C(R₅)₂—, C(R₆)₂NR₅—, —S(═O)—, S(═O)₂—, —S(═O)₂O—, —OS(═O)₂—, —S(═O)₂NR₅—, —NR₅S(═O)₂—, —S(═O)NR₅—, —NR₅S(═O)—, —OS(═O)NR₅—, —NR₅S(═O)O—, or —S(═O)(═NR₅)—, where in the point of attachment to R₁ or R₂ is on the left and n=1-6;
R₅ and R₆ are each independently hydrogen, a halogen, —CN, OR₇, NR₇R₈, —N(R₇)C(═O)R₈, —C(═O)N(R₇), —C(═O)R₇, —C(═O)OR₇, —SR₇, —S(═O)R₇, —S(═O)₂R₇, or any optimally substituted —C₁-C₆ alkyl;
R₇ and R₈ are each independently hydrogen, any optimally substituted —C₁-C₆ alkyl; or alternatively R₇ and R₈ are taken together as an optionally substituted C₁-C₆ monocyclic cycloalkyl ring or an optionally substituted monocyclic heterocyclic ring;
Y is null, —CH₂—, —CHR₉—, —CR₉R₁₀—, —(CH₂)ₙ—, —(CHR₉)ₙ—, —(CR₉R₁₀)ₙ—, —C(═O)—, —S(═O)—, —S(═O)₂—, wherein n=1-6;
R₉ and R₁₀ are each independently hydrogen, a halogen, —CN, or any optimally substituted —C₁-C₆ alkyl; or alternatively R₉ and R₁₀ can be taken together as an optionally substituted C₁-C₆ monocyclic cycloalkyl ring or an optionally substituted monocyclic heterocyclic ring;
R₁ is —H, —F, —Cl, —Br, —NO₂, —CN, —NH₂, —NHR₁₁, —NR₁₁R₁₂, —OH, —OR₁₁, optionally substituted C₁-C₆ alkyl, optionally substituted monocyclic cycloalkyl, optionally substituted bicyclic cycloalkyl, optionally substituted tricyclic cycloalkyl, optionally substituted monocyclic heterocycle, optionally substituted bicyclic heterocycle, or optionally substituted tricyclic heterocycle;
R₂ is —H, —F, —Cl, —Br, —NO₂, —CN, —NO₂, —NH₂, —NHR₁₁, —NR₁₁R₁₂, —OH, —OR₁₁, optionally substituted C₁-C₆ alkyl, optionally substituted monocyclic cycloalkyl, optionally substituted bicyclic cycloalkyl, optionally substituted tricyclic cycloalkyl, optionally substituted monocyclic heterocycle, optionally substituted bicyclic heterocycle, optionally substituted tricyclic heterocycle;
R₃ is —H, —F, —Cl, —Br, —NO₂, —CN, —NO₂, —NH₂, —NHR₁₁, —NR₁₁R₁₂, —OH, —OR₁₁, optionally substituted C₁-C₆ alkyl, optionally substituted monocyclic cycloalkyl, optionally substituted bicyclic cycloalkyl, optionally substituted tricyclic cycloalkyl, optionally substituted monocyclic heterocycle, optionally substituted bicyclic heterocycle, optionally substituted tricyclic heterocycle;
R₁₁ and R₁₂ are each independently optionally substituted C₁-C₆ alkyl, optionally substituted monocyclic cycloalkyl, optionally substituted bicyclic cycloalkyl, optionally substituted tricyclic cycloalkyl, optionally substituted monocyclic heterocycle, optionally substituted bicyclic heterocycle, or optionally substituted tricyclic heterocycle;
R₄ is hydrogen, —CH₃, —CHR₁₃, —CR₁₃R₁₄, —S(═O)R₁₃, —S(═O)₂R₁₃, optionally substituted alkyl, an optimally substituted haloalkyl, an optimally substituted alkenyl, an optimally substituted alkynyl, an optimally substituted cycloalkyl, an optimally substituted heterocycle, an optimally substituted aryl, —C(═O)R₁₅, or a nitrogen protecting group; wherein;
R₁₃ and R₁₄ are each independently optionally substituted C₁-C₆ alkyl, optionally substituted monocyclic cycloalkyl, optionally substituted bicyclic cycloalkyl, optionally substituted tricyclic cycloalkyl, optionally substituted monocyclic heterocycle, optionally substituted bicyclic heterocycle, optionally substituted tricyclic heterocycle;
R₁₅ is hydrogen, —CH₃, optionally substituted alkyl, an optimally substituted haloalkyl, an optimally substituted alkenyl, an optimally substituted alkynyl, an optimally substituted cycloalkyl, an optimally substituted heterocycle, an optimally substituted aryl
or a pharmaceutically acceptable salt thereof
to a subject in need thereof, wherein said administering treats or reduces symptoms of said disease or condition in said subject.

21. The method of claim 20, wherein said disease or condition is selected from the group consisting of an eye disorder, cancer, and a blood disorder.

22. The method of claim 21, wherein said eye disorder is selected from the group consisting of vision loss, retinal dystrophy, macular degeneration, retinal degeneration, diabetic retinopathy, proliferative vitreoretinopathy, and retinal detachment.

23. The method of claim 22, wherein said blood disorder is selected from the group consisting of anemia, hemolytic anemia, sickly cell disease, thalassemia, hereditary spherocytosis, hereditary elliptocytosis, abetalipoproteinemia and Bassen-Kornzweig syndrome.

24. The method of claim 13, wherein said administering prevent and R₁—Z— s or reduces photoreceptor cell death in the eye of said subject.

25. The method of claim 13, wherein said activator is formulated for injection, for oral delivery, or as an eye drop.

26. The method of claim 25, wherein said injection is intravitreal injection.

27-43. (canceled)
